(19) United States
(12) Patent Application Publication
(10) Pub. No.: US 2006/0140889 A1
(43) Pub. Date: Jun. 29, 2006

(54) Affinity proteins for controlled application of cosmetic substances

(75) Inventors: Erwin Houtzager, Amerongen (NL); Irma Maria Caecilia Vijn, Bennekom (NL); Peter Christiaan Sijmons, Amsterdam (NL); Grant Mudge, West Redding, CT (US); Addi Fadel, Shellton, CT (US)

Correspondence Address: TRASK BRITT, P.O. BOX 2550, SALT LAKE CITY, UT, 84110, US

(73) Assignee: L-MAbs B.V., Wageningen, NL (US)

(21) Appl. No.: 11/150,871

(22) Filed: Jun. 10, 2005

Related U.S. Application Data
(63) Continuation of application No. PCT/NL03/00876, filed on Dec. 10, 2003.
(60) Provisional application No. 60/432,906, filed on Dec. 10, 2002.

(30) Foreign Application Priority Data
Dec. 10, 2002 (EP) 02080206.2

(51) Int. Cl. A61K 8/64; A61K 8/65; C11D 3/386

(52) U.S. Cl. 424/63; 424/64; 424/70.14; 510/320; 8/401

(57) ABSTRACT

Provided are means and methods for applying a cosmetic substance to a desired target. The method comprises providing a conjugate of a proteinaceous substance having a specific affinity for the target molecule linked to a cosmetic substance, whereby the resulting connection between the cosmetic substance and the target molecule can be disrupted upon the presence of a chemical and/or physical signal.

### CROSS-REFERENCE TO RELATED APPLICATIONS

This application is a continuation of PCT International Application No. PCT/NL2003/000876, filed on Dec. 10, 2003, designating the United States of America, and published, in English, as PCT International Publication No. WO 2004/069211 A2 on Aug. 19, 2004, which application claims priority to European Patent Application Serial No. 02080206.2 filed on Dec. 10, 2002, and to U.S. Provisional Application Ser. No. 60/432,906, filed Dec. 10, 2002.

### STATEMENT ACCORDING TO 37 C.F.R. § 1.52(e)(5) SEQUENCE LISTING SUBMITTED ON COMPACT DISC

Pursuant to 37 C.F.R. § 1.52(e)(1)(iii), a compact disc containing an electronic version of the Sequence Listing has been submitted concomitant with this application, the contents of which are hereby incorporated by reference. A second compact disc is submitted and is an identical copy of the first compact disc. The discs are labeled “copy 1” and “copy 2,” respectively, and each disc contains one file entitled “P62519US10.txt” which is 285 KB and was created on Sep. 13, 2005.

### TECHNICAL FIELD

The invention relates to molecular affinity bodies. It particularly relates to conjugates of such molecular affinity bodies with cosmetic substances, more in particular to molecular affinity bodies linked to fragrances and/or colored substances and/or conditioning agents. Cosmetic agents are typically delivered non-specifically to a general area of application. Such application ranges from massaging shampoo or the like in hair, applying cream, powder or ointment on the body to applying a liquid or solid composition to an amount of water in contact with textiles, etc.

### BACKGROUND

There are several problems associated with such nonspecific delivery. For instance, in many applications the majority of the cosmetic agent is never actually delivered to the target, but is rinsed out by a washing step. Also, many cosmetic agents that are delivered are released prematurely, such that color or fragrance fades or disappears rather quickly. Furthermore, in the case of coloring agents, such as dyes for hair, the nonspecific application leads to staining of clothes, etc. Moreover, the binding to hair of color, if it is to last any significant time typically requires harsh chemical treatments.

A further problem with cosmetic agents is that many of them (in particular fragrant agents) are hydrophobic, which hampers their applicability in aqueous environments. Also fragrances are often volatile. The present invention contributes to solving many of these problems and more as will become clear from the following description.

### SUMMARY OF THE INVENTION

The present invention provides in one embodiment a method for applying a cosmetic substance to a desired target molecule, comprising providing a conjugate of a proteinaceous substance having specific affinity for the target molecule linked to a cosmetic substance, whereby the resulting connection between cosmetic substance and target molecule can be disrupted upon the presence of a chemical and/or physical signal. The present invention uses conjugates of proteinaceous molecules having specific affinity for a target molecule and a cosmetic agent, linked together in such a way that the linkage can be disrupted if desired. The cosmetic agents according to the invention can be fragrances, coloring agents, conditioners and the like. Fragrances are typically delivered in an aqueous composition, a powder-like composition, an oil or a cream/ointment-like composition. Typically, the fragrance is desired to linger over longer periods of time. However, until the present invention, the typical release of a fragrance has been a burst within a very short time from application and a less than desired release for the remainder. As stated, the fragrant compositions are typically delivered nonspecifically to a desired area.

According to the invention the fragrant molecules are delivered specifically to a target associated with a desired area of delivery, such as to skin components such as keratin or microorganisms associated with skin, or to hair components, or saliva components, or to microorganisms associated with mucosal secretions or to textile fabric. Upon delivery of the conjugate of the fragrant molecule and the targeting molecule, the linkage between the two will, in one embodiment, become labile through the action of local enzymes, or the action of added enzymes and/or by a change in physical and/or chemical conditions, such as temperature, pH and the like. Thereby the fragrant molecule will be released depending on the selected half life of the bond between targeting molecule and fragrant molecule. The same targeting mechanism is applied when the cosmetic agent is a coloring agent, e.g. a dye for hair. Several different options concerning release are present. The color may be desired for only a short period of time. In this case a rinse with water or washing with a shampoo should be enough to remove the dye, either by disrupting the linkage of the dye to the targeting molecule, or by selecting a targeting molecule which has a low affinity under conditions of removal (shampoo). If the color is desired for a longer period of time, a special shampoo providing an enzyme which disrupts the link or which proteolyzes the proteinaceous targeting molecule can be provided. The release can also be provided by a chemical signal (e.g., pH) or a physical signal. However, these conjugate dyes can be used as permanent dyes also, which will only disappear because of hair growth. In the same manner as described above one can provide compositions for delivering fragrances to fabric (for softener compositions and the like), as well as conditioner compositions and any other cosmetic agents that are better when specifically delivered and/or which benefit from any form of controlled release.

According to the invention, versatile affinity proteins are used as targeting molecules. A versatile affinity protein is a molecule that comprises at least a synthetic or recombinant proteinaceous molecule comprising a binding peptide and a core, the core comprising a beta-barrel comprising at least four strands, wherein the beta-barrel comprises at least two beta-sheets, wherein each of the beta-sheets comprises two of the strands and wherein the binding peptide is a peptide connecting two strands in the beta-barrel and wherein the binding peptide is outside its natural context. Preferably, an affinity protein comprises a beta-barrel, wherein the beta-barrel comprises at least five strands, wherein at least one of the sheets comprises three of the strands. More preferably, an affinity protein comprises a beta-barrel that comprises at least six strands, wherein at least two of the sheets comprises three of the strands. Preferably, an affinity protein comprises a beta-barrel that comprises at least seven strands, wherein at least one of the sheets comprises four of the strands. Preferably, an affinity protein comprises a beta-barrel which comprises at least eight or nine strands, wherein at least one of the sheets comprises four of the strands. The various strands in the core are preferably encoded by a single open reading frame. The loops connecting the various strands may have any type of configuration. So as not to unduly limit the versatility of the core, it is preferred that loops connect strands on the same side of the core, i.e., an N-terminus of strand (a) connects to a C-terminus of strand (b) on either the closed side or the open side of the core. Loops may connect strands in the same β-sheet or cross-over to the opposing β-sheet. Preferred arrangements for connecting the various strands in the core are given in the examples and the figures, and in particular FIG. 1. Strands in the core may be in any orientation (parallel or antiparallel) with respect to each other. Preferably the strands are in the configuration as depicted in FIG. 1.

In a further preferred embodiment, the binding peptide connects two strands of the beta-barrel on the open side of the barrel. Preferably the binding peptide connects at least two beta-sheets of the barrel. In a preferred embodiment the versatile affinity protein comprises more than one, preferably three binding peptides and three peptides connecting beta-sheets and/or beta-barrels.

The versatile affinity proteins to be used in the conjugates according to the invention are typically designed to have binding properties and structural properties which are suitable for application in the delivery of cosmetic agents. These properties are obtained by a selection process as described herein below. Thus, the invention also provides a method according to the invention wherein the proteinaceous molecule has an altered binding property, the property selected for the physical and/or chemical circumstances in which the conjugate is applied, the alteration comprising introducing an alteration in the core of proteinaceous molecules according to the invention, and selecting from the proteinaceous molecules, a proteinaceous molecule with the altered binding property. The invention further provides a method according to the invention wherein the proteinaceous molecule has an altered structural property, the property selected for the physical and/or chemical circumstances in which the conjugate is applied, the alteration comprising introducing an alteration in the core of proteinaceous molecules according to the invention, and selecting from the proteinaceous molecules, a proteinaceous molecule with the altered structural property. These processes are most easily carried out by altering nucleic acid molecules which encode proteinaceous substances according to the invention. However, the alterations may also be post-translational modifications. The invention also provides the novel conjugates comprising a cosmetic agent and a versatile affinity protein liked in any way. The link may be covalent or by coordination or complexing. It may be direct or indirect. The cosmetic agent may be present in a liposome or another vehicle to which the VAP is linked. The conjugate may also be a fusion protein.

Preferably, the linkage is labile under certain conditions. Labile linkers are well known in the art of immunotoxins for the treatment of cancer and the like. Such linkers can be applied or adapted to the presently invented conjugates. Also, a linker may be a peptide or peptide-like bond, which can be broken by an enzyme. Preferably, such an enzyme is normally associated with the target of the conjugate. In an alternative embodiment, the enzyme can be added simultaneously or separately. The linker is, of course, preferably stable under storage conditions. For fragrances, the linkers need to be designed such that the disruption occurs exactly at the site that releases the original fragrant substance only. In one embodiment, the link between a proteinaceous molecule and a cosmetic substance is labile under skin and/or hair conditions. This is very advantageous in combination with a conjugate that has specific affinity for a target molecule associated with the skin, hair or other body substances exposed to the exterior of the body, in particular, keratin. In another embodiment, the conjugate has a specific affinity for a target molecule associated with textile fabric. Compositions comprising the conjugates of the invention are also part of the present invention. They include, but are not limited to, a perfume, a deodorant, a mouth wash or a cleaning composition, a hair dye composition, a lipstick, rouge or other skin-coloring composition, a detergent and/or softener composition. In a preferred embodiment, a conjugate of the invention comprises a sequence as depicted in Tables 2, 3, 10, 13, 16a, 16b, or 20 or FIGS. 22A-221.

### DESCRIPTION OF THE FIGURES

In the figures, which illustrate what is currently considered to be the best mode for carrying out the invention:

FIG. 1: Schematic 3D-topology of scaffold domains. Eight example topologies of protein structures that can be used for the presentation of antigen-binding sites are depicted. The basic core beta-elements are nominated in Example A. This basic structure contains nine beta-elements positioned in two plates. One beta-sheet contains elements 1, 2, 6 and 7 and the other contains elements 3, 4, 5, and 9. The loops that connect the beta-elements are also depicted. Bold lines are connecting loops between beta-elements that are in top position while dashed lines indicate connecting loops that are located in bottom position. A connection that starts dashed and ends solid indicates a connection between a bottom and top part of beta-elements. The numbers of the beta-elements depicted in the diagram correspond to the numbers and positions mentioned in FIGS. 1 and 2. Panel A, 9 beta-element topology, for example, all antibody light and heavy chain variable domains and T-cell receptor variable domains; Panel B, 8 beta-element topology, for example, interleukin-4 alpha receptor (1IAR); Panel C, 7a beta-element topology, for example, immunoglobulin killer receptor 2dl2 (2DLI); Panel D, 7b beta-element topology, for example, E-cadherin domain (1FF5); Panel E, 6a beta-strand topology; Panel F, 6b beta-element topology, for example, Fc epsilon receptor type alpha (1J88); Panel G, 6c beta-element topology, for example, interleukin-1 receptor type-1 (1GOY); and Panel H, 5 beta-element topology.

FIG. 2: Modular Affinity & Scaffold Transfer (MAST) Technique. Putative antigen binding proteins that contain a core structure as described here can be used for transfer operations. In addition, individual or multiple elements or regions of the scaffold or core structures can also be used for transfer actions. The transfer operation can occur between structural identical or comparable scaffolds or cores that differ in amino acid composition. Putative affinity regions can be transferred from one scaffold or core to another scaffold or core by, for example, PCR, restriction digestions, DNA synthesis or other molecular techniques. The results of such transfers is depicted here in a schematic diagram. The putative (coding) binding regions from molecule A (top part, affinity regions) and the scaffold (coding) region of molecule B (bottom part, framework regions) can be isolated by molecular means. After recombination of both elements, a new molecule appears (hybrid structure) that has binding properties of molecule A and scaffold properties of scaffold B.

FIG. 3: Domain notification of immunoglobular structures. The diagram represents the topologies of protein structures consisting of respectively 9, 7 and 6 beta-elements (indicated 1-9 from N-terminal to C-terminal). Beta-elements 1, 2, 6 and 7 and elements 3, 4, 5, 8 and 9 form two beta-sheets. Eight loops (L1-L8) are responsible for the connection of all beta-elements. Loop 2, 4, 6 and 8 are located at the top site of the diagram and this represents the physical location of these loops in example proteins. The function of loops 2,4 and 8 in light and antibody variable domains is to bind antigens, known as CDR regions. The position of L6 (also marked with a patterned region) also allows antigen binding activity, but has not been indicated as a binding region. L2, L4, L6, L8 are determined as affinity region1 (AR1), AR2, AR3 and AR4, respectively. Loops 1, 3, 5 and 7 are located at the opposite site of the proteins.

FIG. 4A: Schematic overview of vector CM126.

FIG. 4B: Schematic overview of vector CM126.

FIG. 5: Solubilization of inclusion bodies of iMab100 using heat (60° C.). Lanes: Molecular weight marker (1), isolated inclusion bodies of iMab100 (2), solubilized iMab100 upon incubation of inclusion bodies in PBS pH 8+1% Tween-20 at 60° C. for 10 minutes.

FIG. 6: Purified iMab variants containing either 6-, 7- or 9 beta-sheets. Lanes: Molecular weight marker (1), iMab1300 (2), iMab1200 (3), iMab701 (4), iMab101 (5), iMab900 (6), iMab122 (7), iMab1202 (8), iMab1602 (9), iMab1302 (10), iMab116 (11), iMab111 (12), iMab100 (13).

FIG. 7: Stability of iMab100 at 95° C. Purified iMab100 incubated for various times at 95° C. was analyzed for binding to ELK(squares) and lysozyme (circles).

FIG. 8: Stability of iMab100 at 20° C. Purified iMab100 incubated for various times at 20° C. was analyzed for binding to ELK (squares) or chicken lysozyme (circles).

FIG. 9A: far UV CD spectum (205-260 nm) of iMab100 at 20° C., 95° C. , and again at 20° C. iMab100 was dissolved in 1×PBS, pH 7.5.

FIG. 9B: iMab111, far UV spectrum determined at 20° C., (partially) denatured at 95° C., and refolded at 20° C., compared to the iMab100 spectrum at 20° C.

FIG. 9C: iMab116, far UV spectrum determined at 20° C., (partially) denatured at 95° C., and refolded at 20° C., compared to the iMab100 spectrum at 20° C.

FIG. 9D: iMab1202, far UV spectrum determined at 20° C., (partially) denatured at 95° C., and refolded at 20° C., compared to the iMab100 spectrum at 20° C.

FIG. 9E: iMab1302, far UV spectrum determined at 20° C., (partially) denatured at 95° C., and refolded at 20° C., compared to the iMab100 spectrum at 20° C.

FIG. 9F: iMab1602, far UV spectrum determined at 20° C., (partially) denatured at 95° C., and refolded at 20° C., compared to the iMab100 spectrum at 20° C.

FIG. 9G: iMab101, far UV spectrum determined at 20° C, (partially) denatured at 95° C, and refolded at 20° C.

FIG. 9H: iMab1200, far UV spectrum determined at 20° C., (partially) denatured at 95° C., and refolded at 20° C.

FIG. 91: iMab701, far UV spectrum determined at 20° C., (partially) denatured at 95° C., and refolded at 20° C.

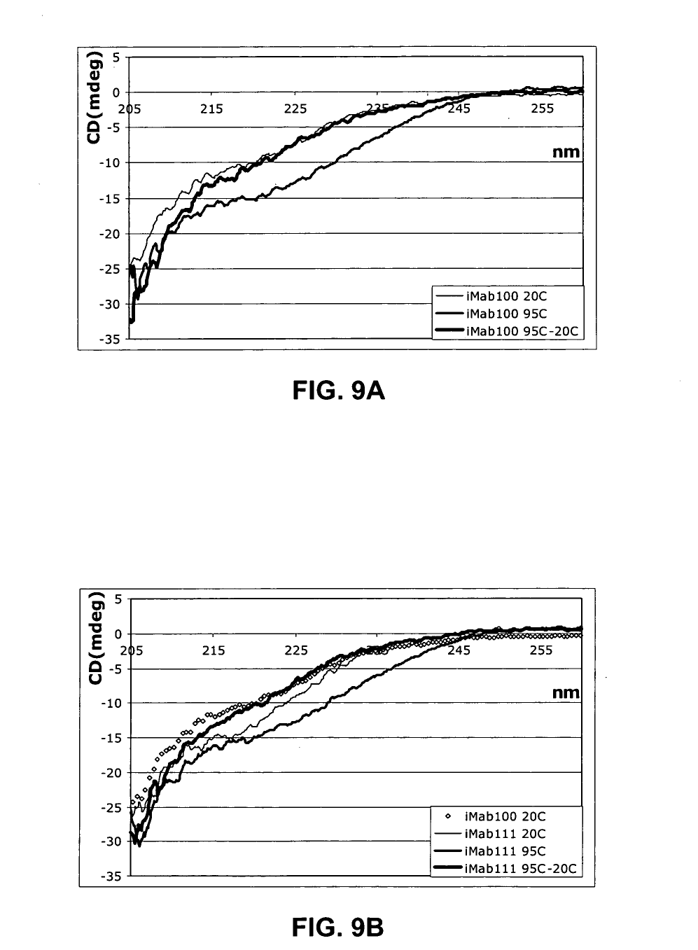

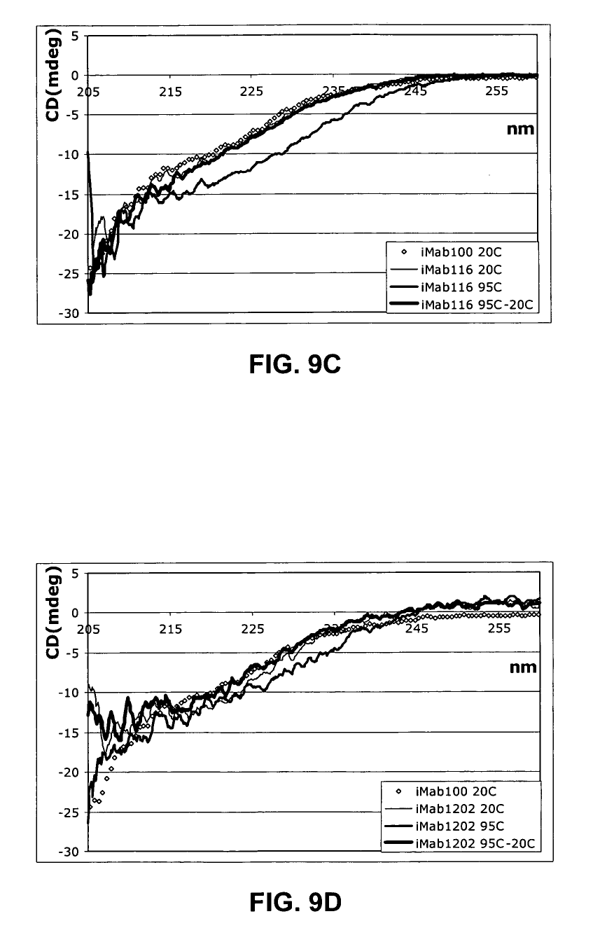

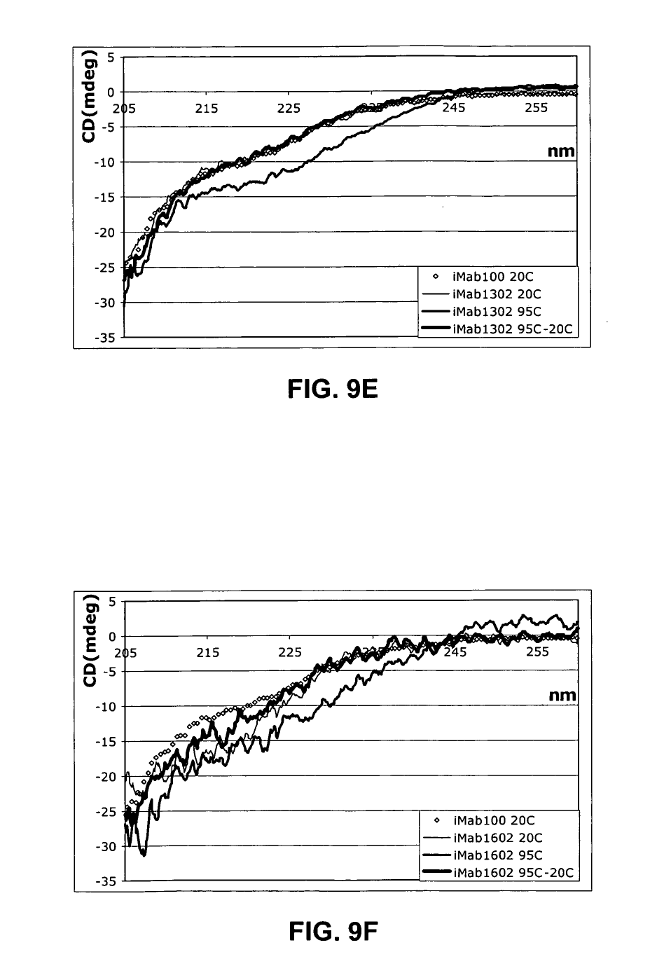

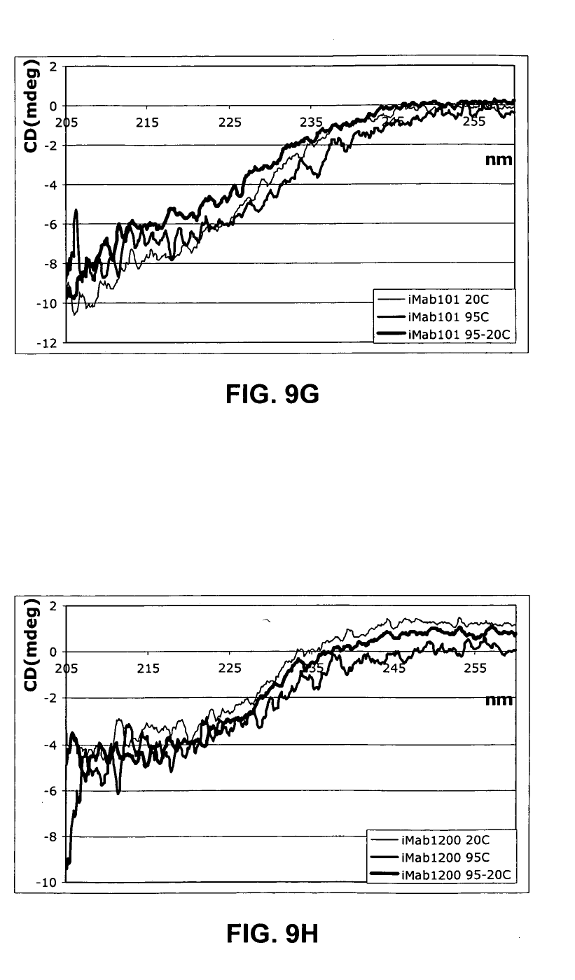

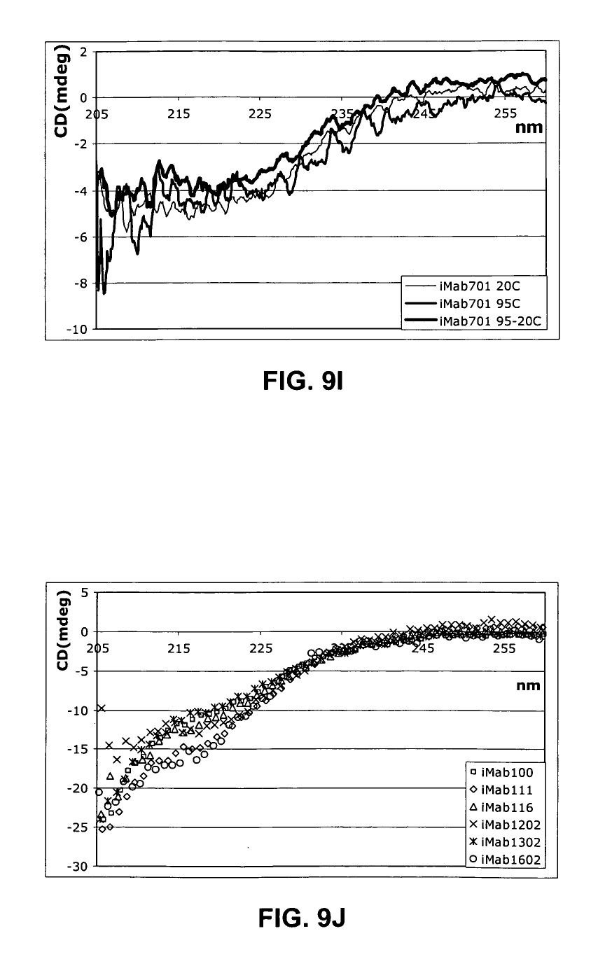

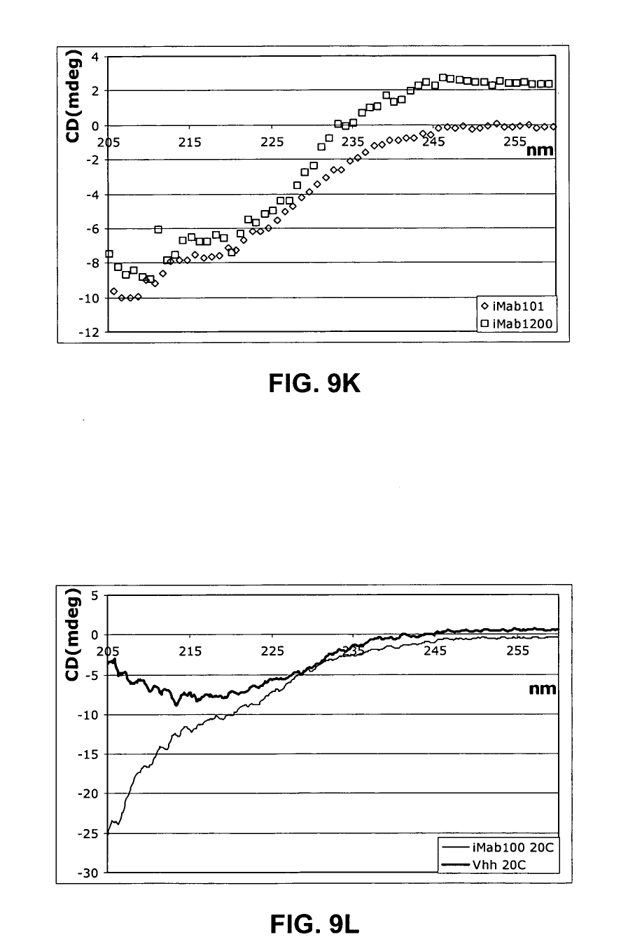

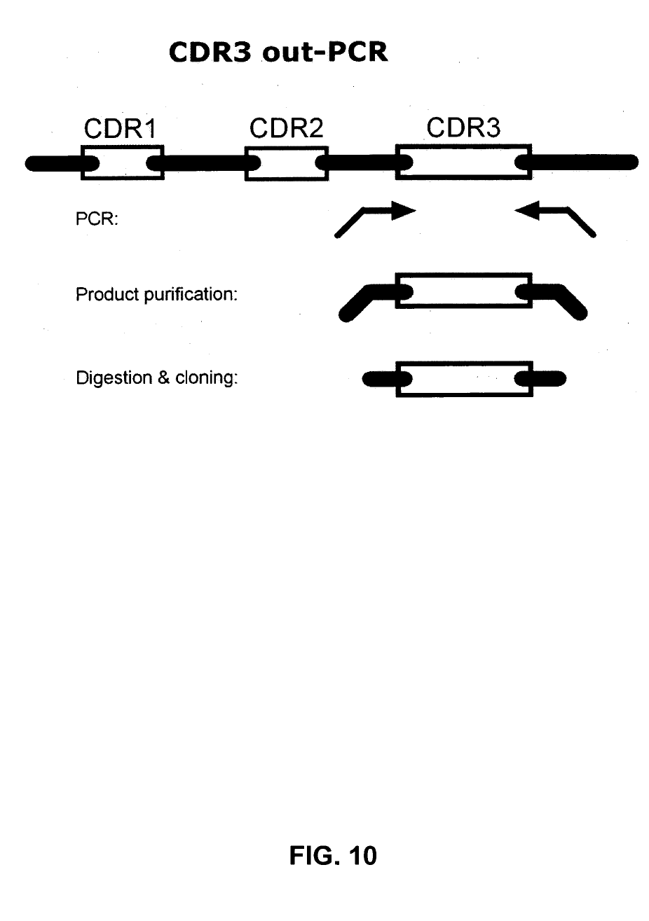

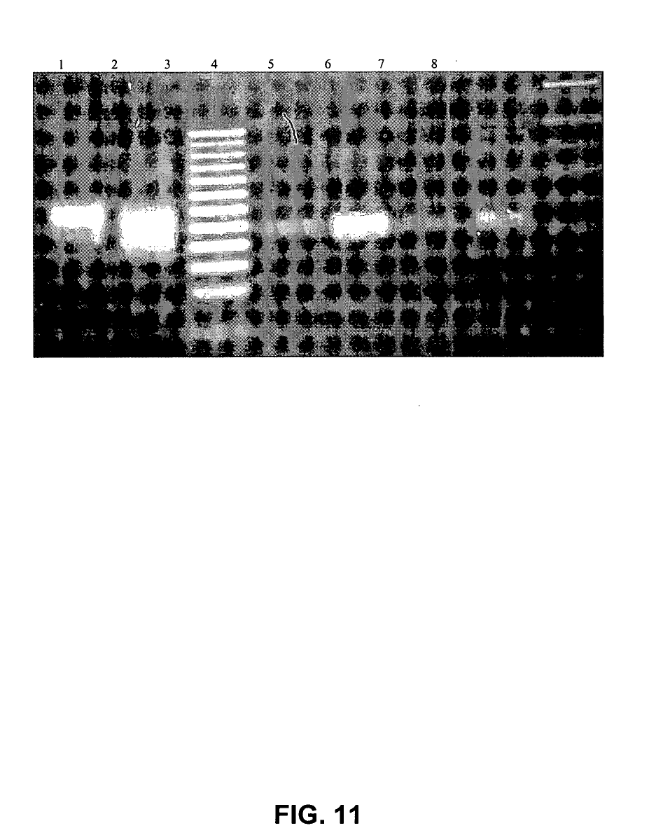

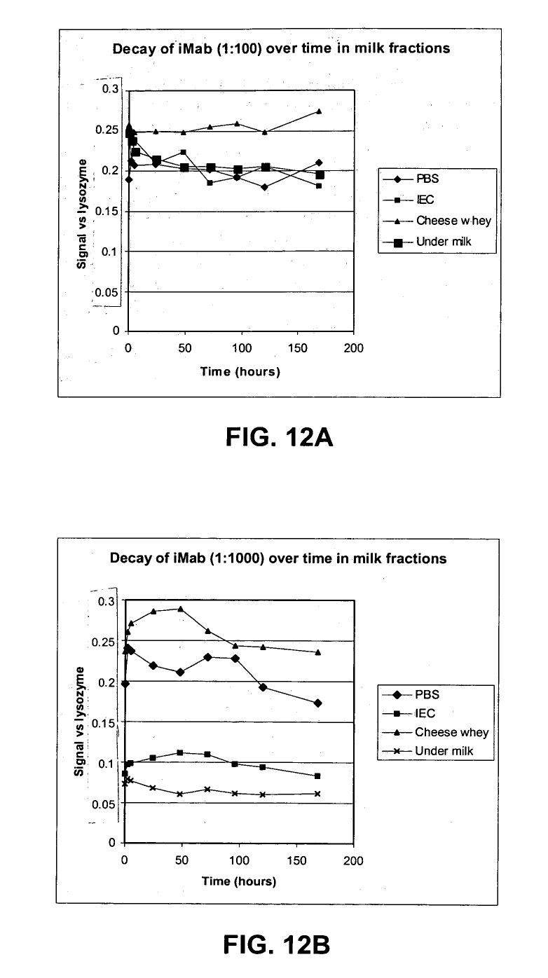

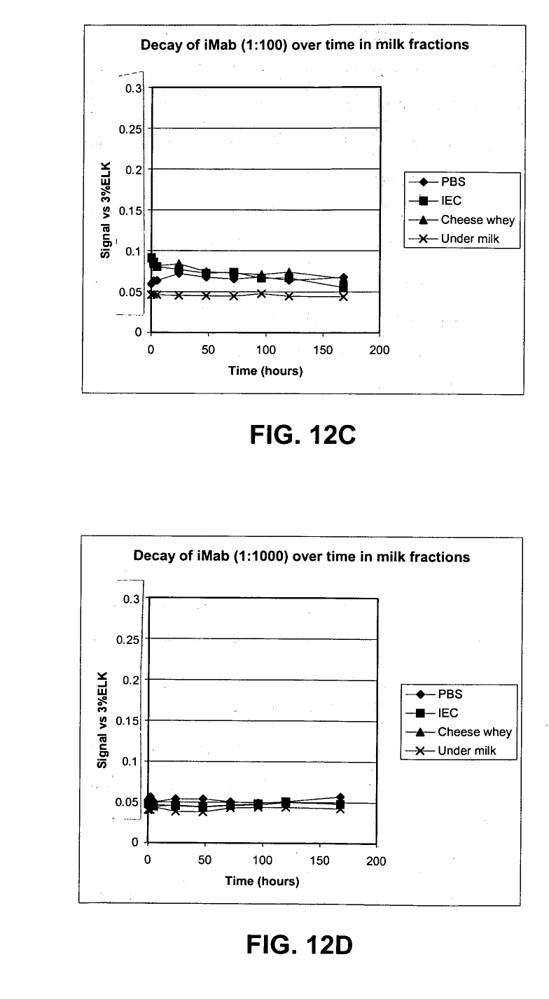

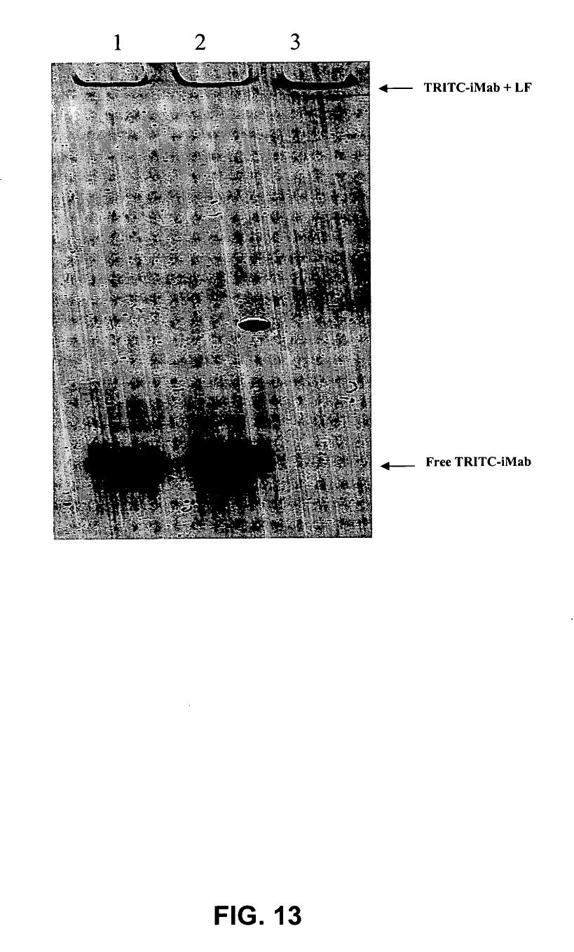

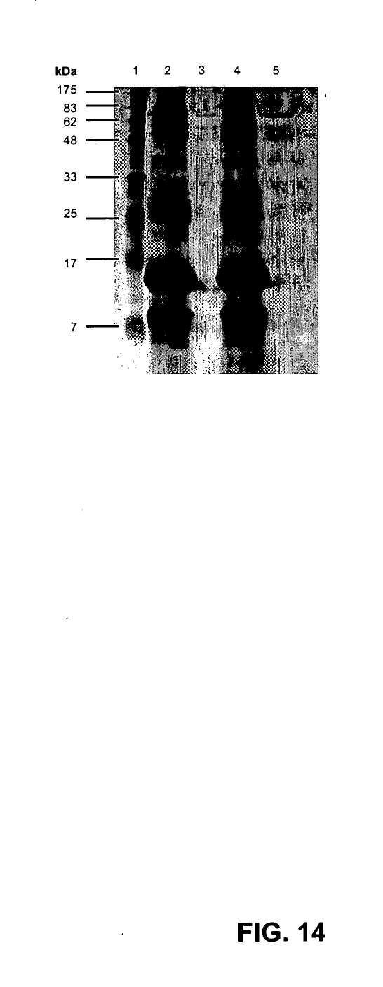

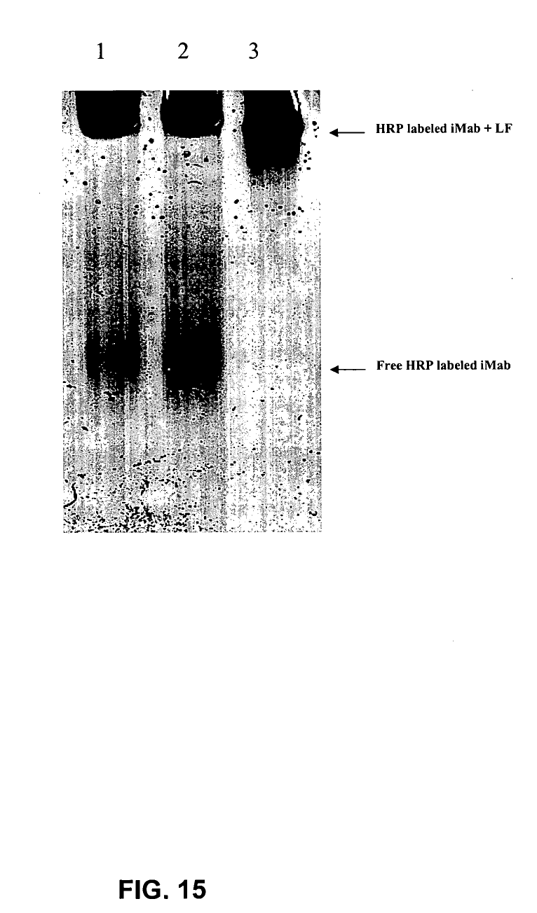

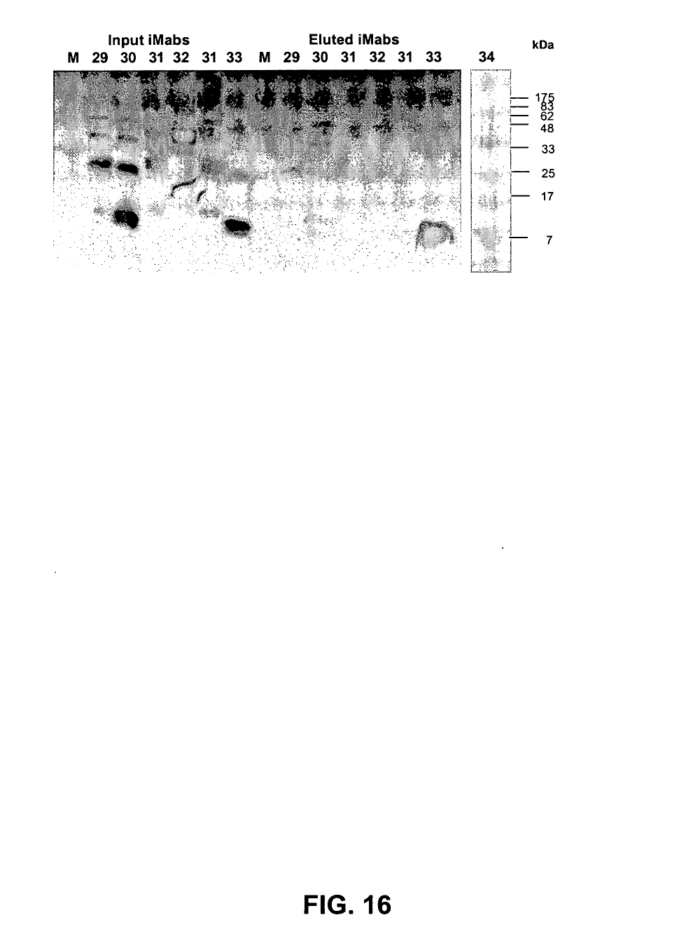

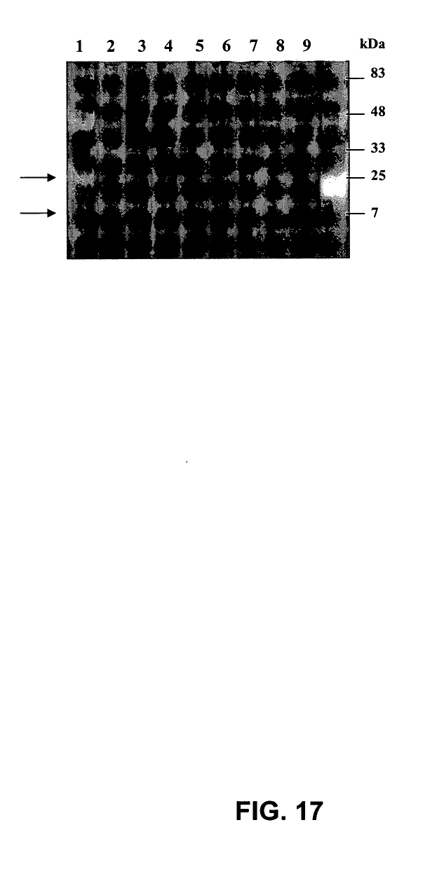

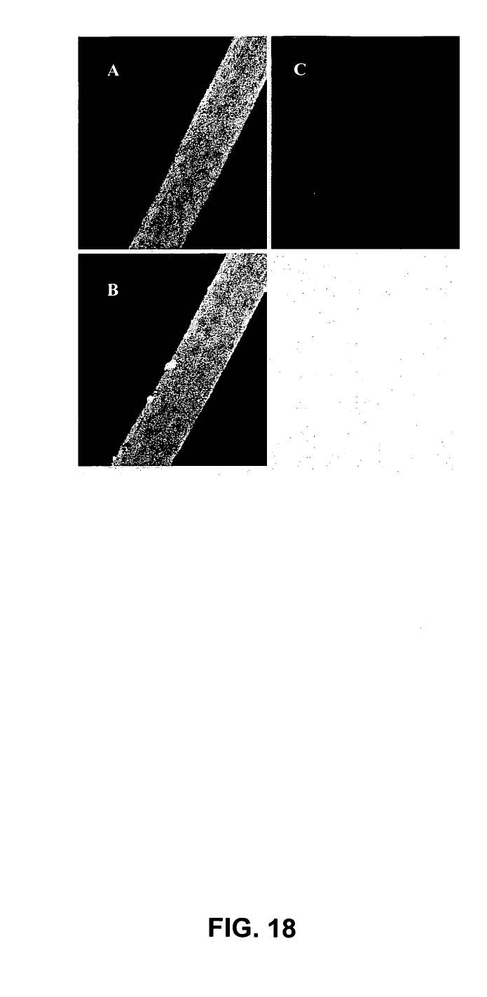

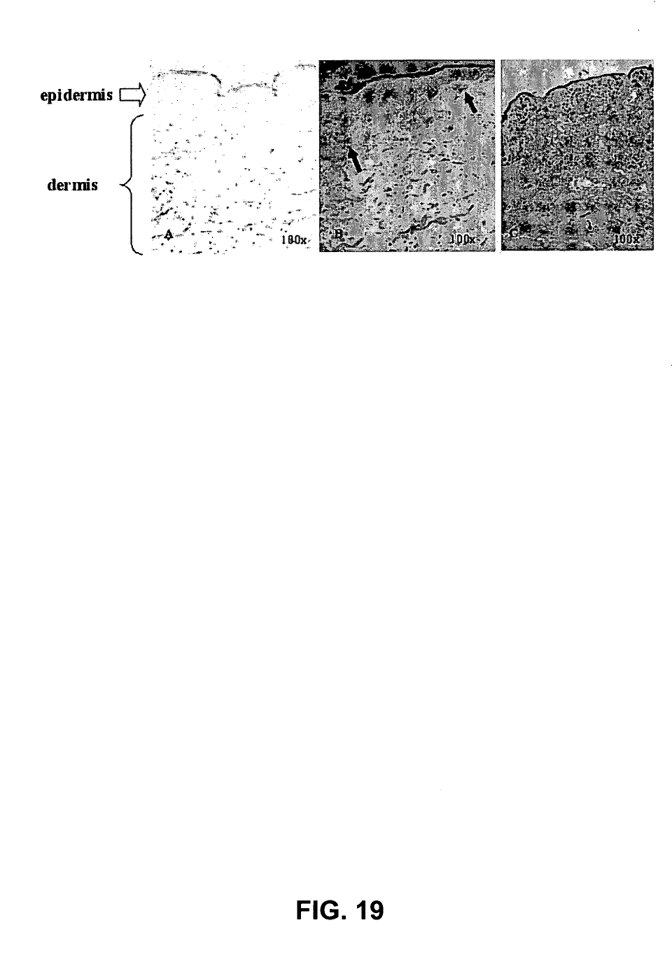

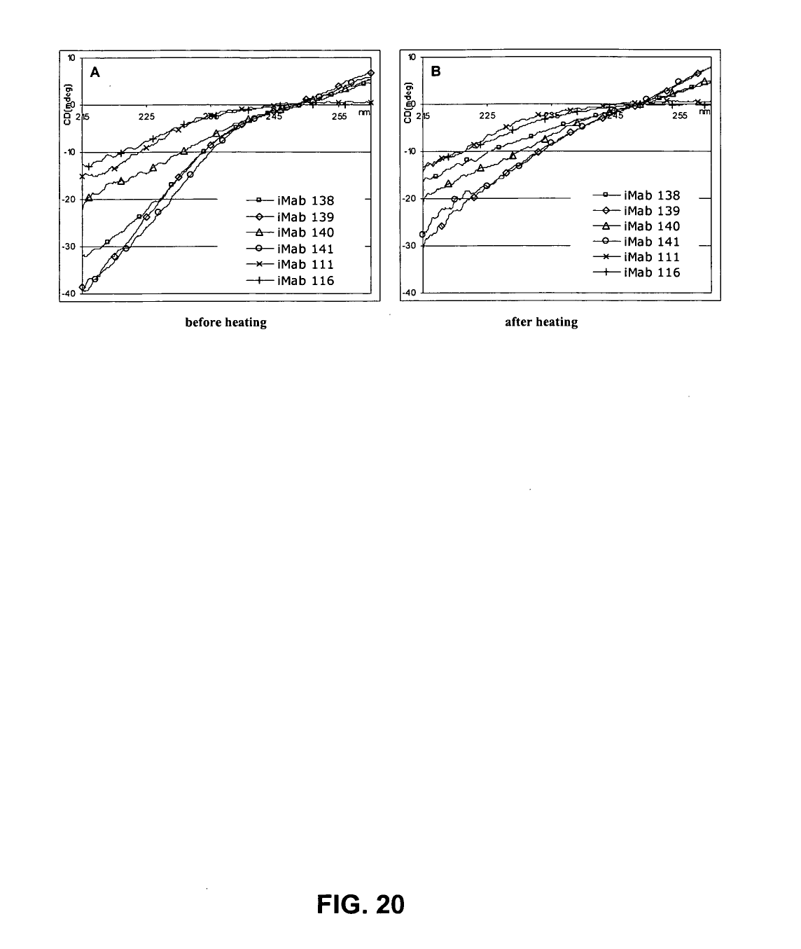

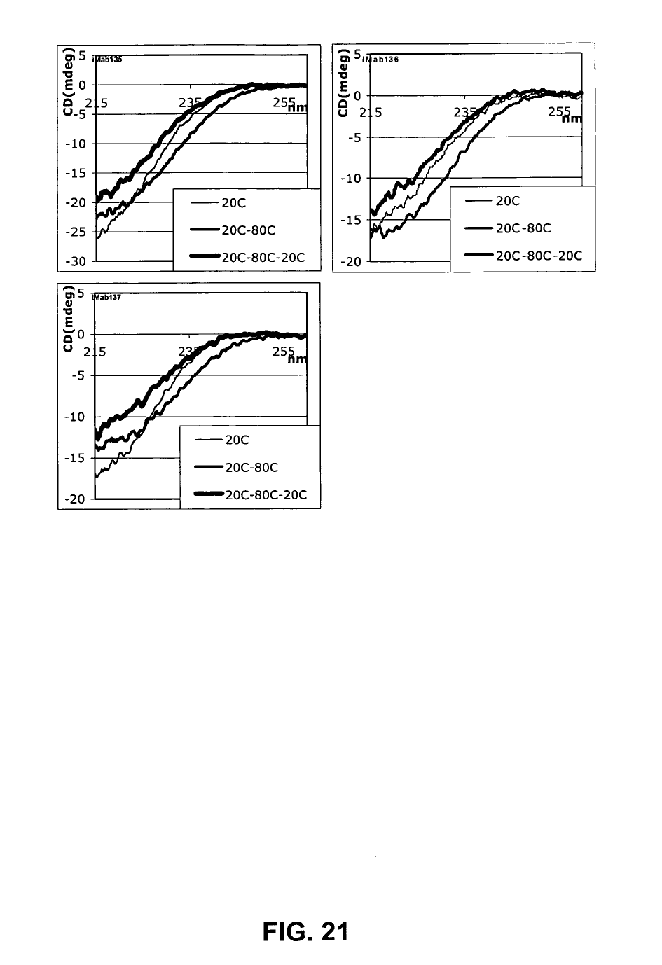

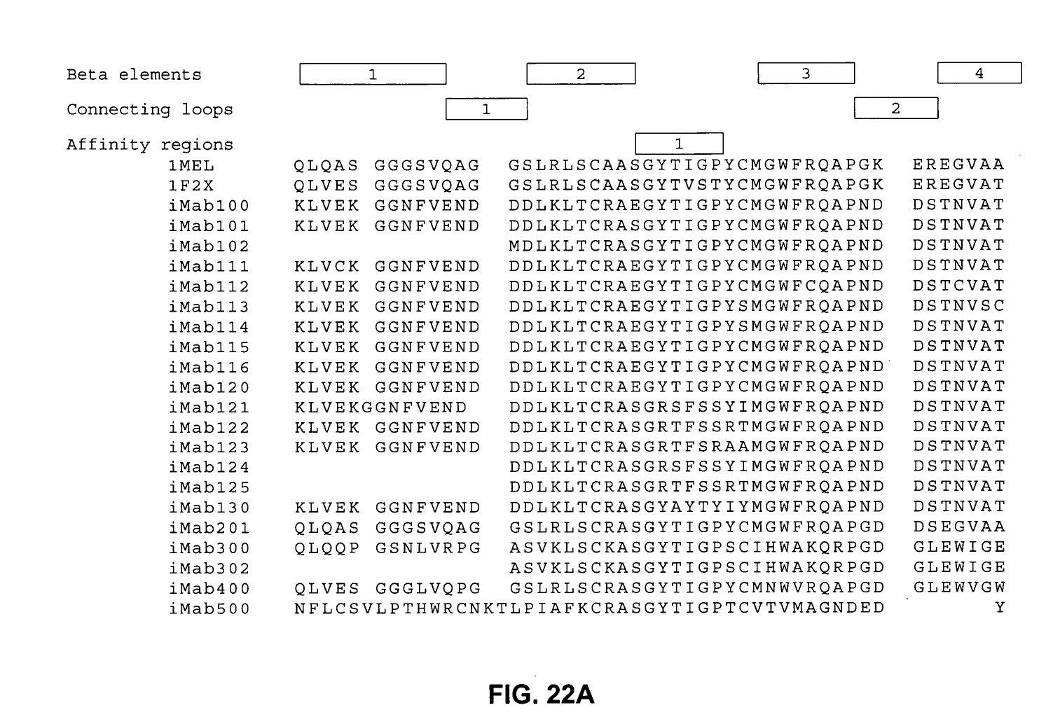

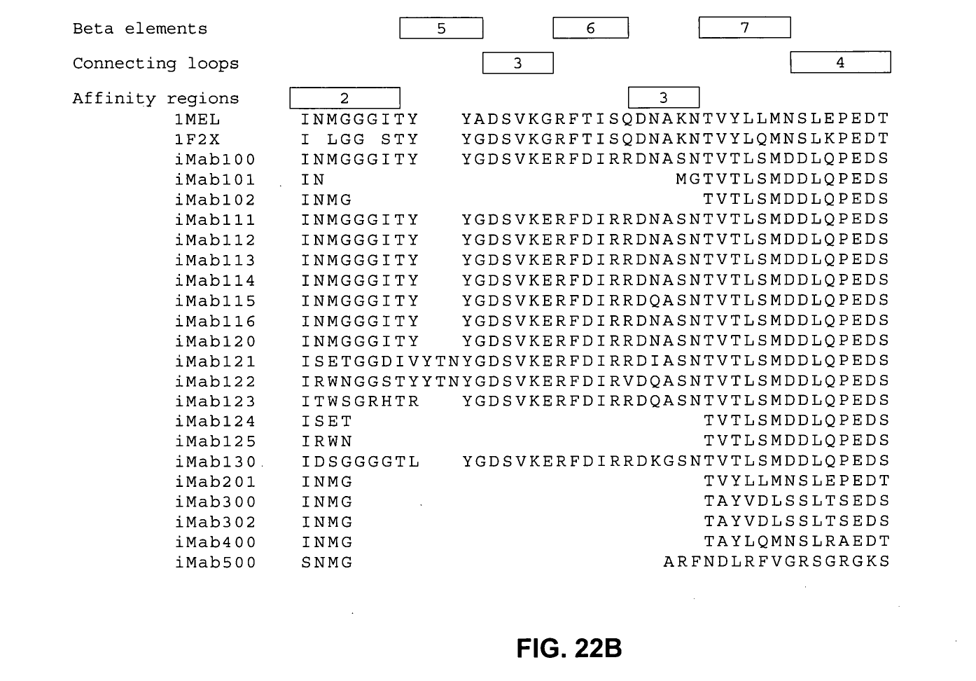

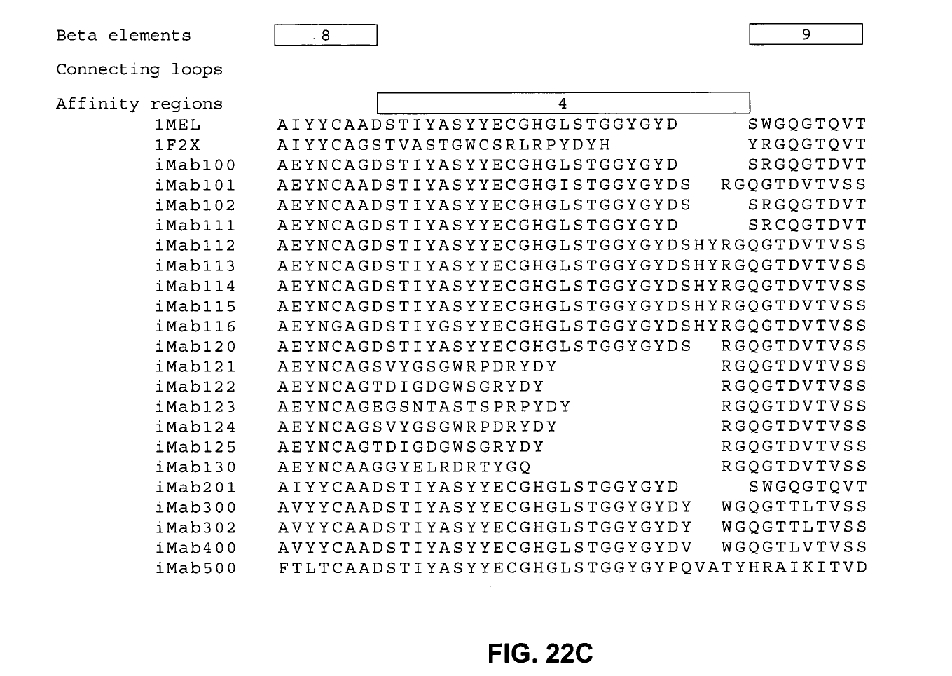

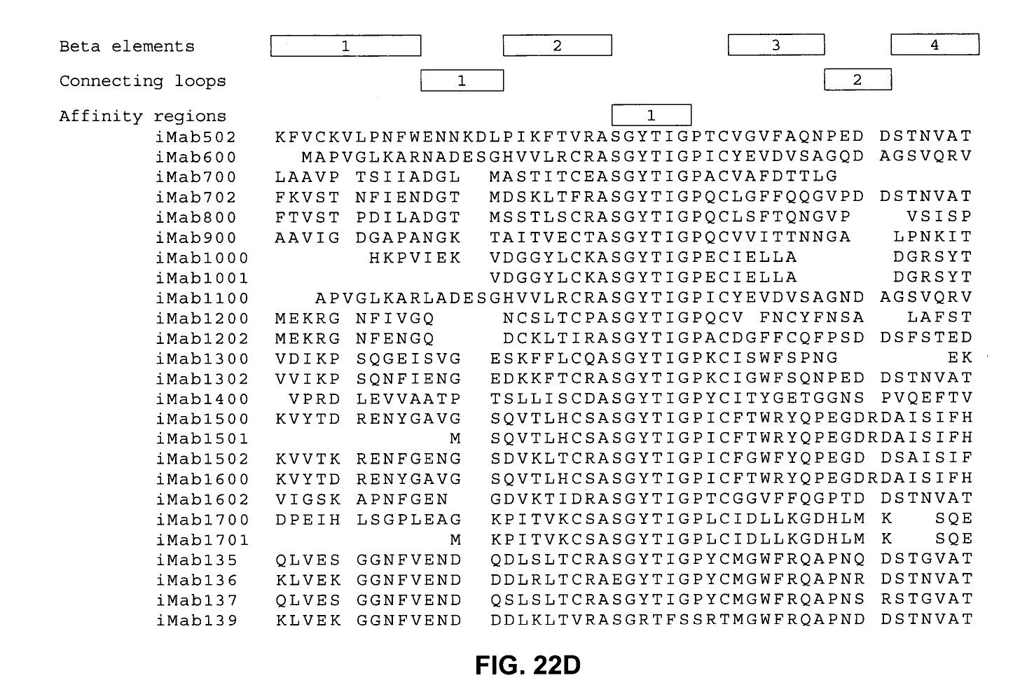

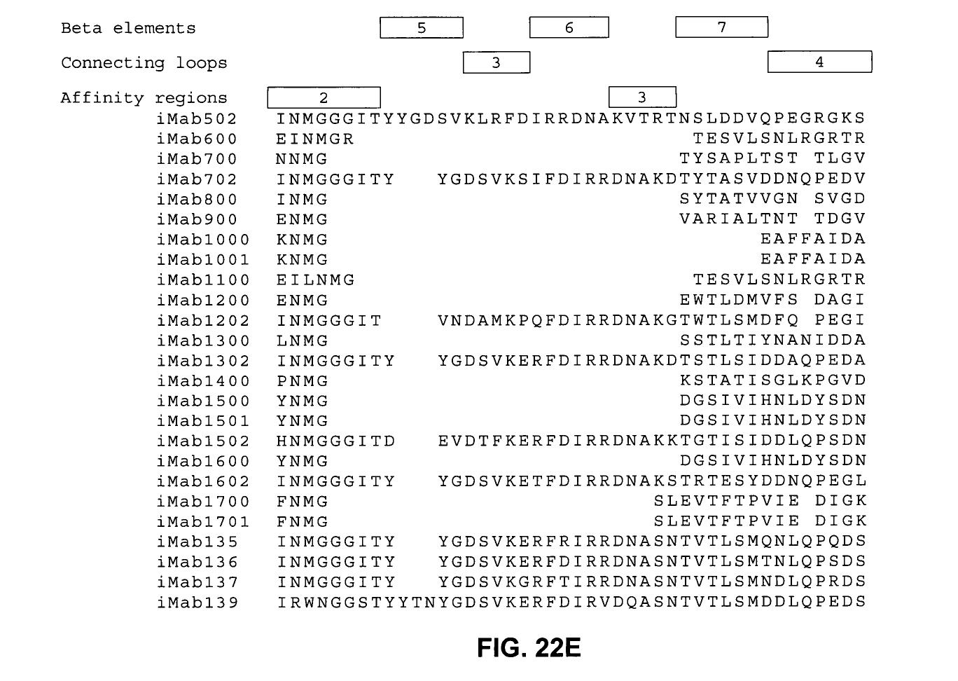

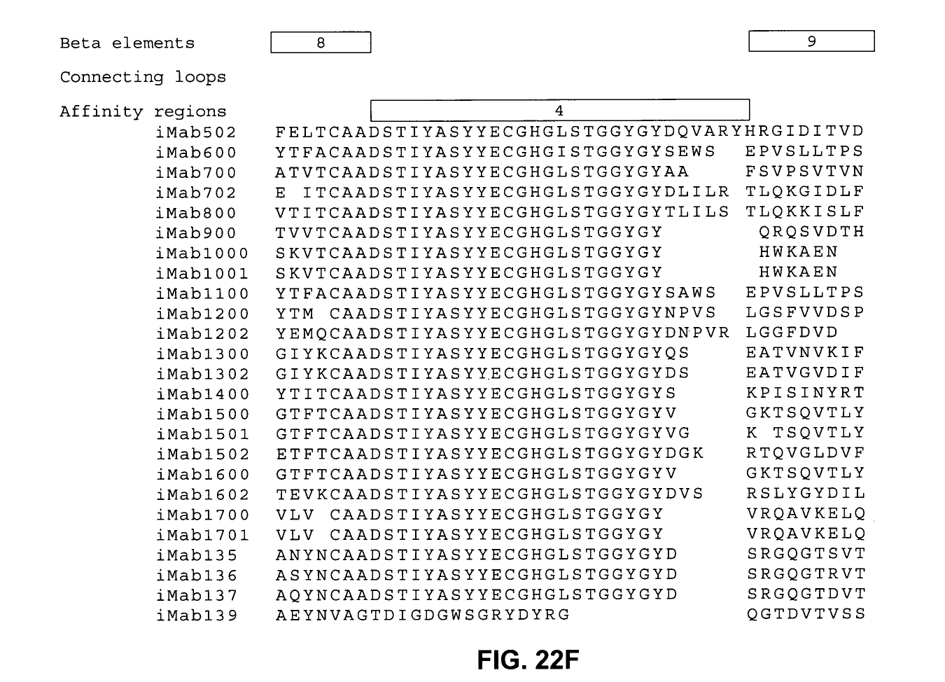

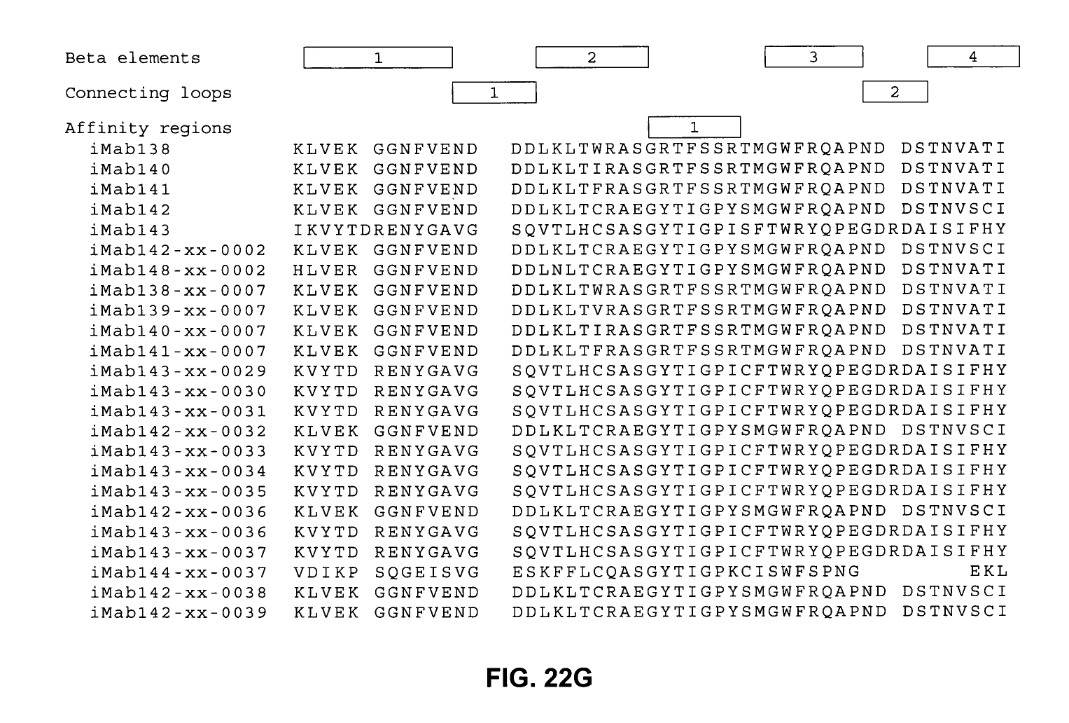

FIG. 9J: Overlay of native (undenatured) 9 strand iMab scaffolds.

FIG. 9K: Overlay of native (undenatured) 7 strand iMab scaffolds.

FIG. 9L: Far UV CD spectra of iMab100 and a V_{HH} (courtesy Kwaaitaal M, Wageningen University and Research, Wageningen, the Netherlands).

FIG. 10: Schematic overview of PCR isolation of CDR3 for MAST.

FIG. 11: Amplification Cow-derived CDR3 regions. 2% Agarose—TBE gel. Lane 1, 1 microgram Llama cDNA cyst+, PCR amplified with primers 8 and 9; Lane 2, 1 microgram Llama cDNA cyst-, PCR amplified with primers 8 and 9; Lane 3, 25 bp DNA step ladder (Promega); Lane 4, 0.75 microgram Cow cDNA PCR amplified with primers 299 and 300; Lane 5, 1.5 microgram Cow cDNA PCR amplified with primers 299 and 300; Lane 6, 0.75 microgram Cow cDNA PCR amplified with primers 299 and 301; Lane 7, 1.5 microgram Cow cDNA PCR amplified with primers 299 and 301; and Lane 8, 50 bp GeneRuler DNA ladder (MBI Fermentas).

FIG. 12: Lysozyme binding activity measured with ELISA of iMab100. Several different solutions were tested in time for proteolytic activity on iMab100 proteins. Test samples were diluted 100 times in FIGS. 12A and 12C, while samples were 1000 times diluted in FIGS. 12B and 12D. FIGS. 12A and 12B show lysozyme activity while FIGS. 12C and 12D show background activity.

FIG. 13: Specific binding of TRITC labeled iMab142-xx-0002 to lactoferrin. Lane 1, iMab-TRITC conjugate (2 mg/ml); Lane 2, iMab-TRITC (conjugate (2 mg/ml)+Bovine serum albumin (10 mg/ml); Lane 3, iMab-TRITC conjugate (2 mg/ml)+lactoferrin (10 mg/ml).

FIG. 14: Specific lactoferrin binding of iMab148-06-0002 covalently bound to Eupergit 1014F. Lane 1, protein marker; Lane 2, bovine caseine whey (input); Lane 3, eluate Eupergit column (negative control); Lane 4, bovine caseine whey (input); Lane 5, eluate Eupergit-iMab148-06-0002 column.

FIG. 15: Specific binding of iMab142-xx-0002-HRP conjugate to lactoferrin. Lane 1, iMab-HRP conjugate (0.1 mg/ml); Lane 2, iMab-HRP conjugate (0.1 mg/ml)+bovine serum albumin (10 mg/ml); Lane 3, iMab-HRP conjugate (0.1 mg/ml)+lactoferrin (10 mg/ml).

FIG. 16: Western blot analysis of VAPs bound to hair. After blotting the VAPs were blotted onto PVDF membrane and detected with anti-VSV-HRP. HRP activity was detected with a fluorescent substrate (Pierce). Fluorescence was detected with FluorChem™ 8900 (Alpha Innotech). Shown is the input of the iMabs and the eluted iMabs from the hair. M; protein weight marker; 29, iMab143-xx-0029; 30, iMab143-xx-0030; 31, iMab143-xx-0031; 32, iMab142-xx-0032; 33, iMab143-xx-0033; 34, iMab143-xx-0034; 35, iMab143-xx-0035.

FIG. 17: SDS-PAGE of Alexa-488 labeled iMabs. Fluorescence was detected with FluorChem™ 8900 (Alpha Innotech). Lane 1, iMab142-xx-0038; Lane 2, iMab 143-xx-0033; Lane 3, iMab143-xx-0034; Lane 4, iMab143-xx-0031; Lane 5, iMab143-xx-0030; Lane 6, iMab143-xx-0029; Lane 7, iMab143-xx-0030; Lane 8, iMab143-xx-0029; Lane 9, iMab142-xx-0039. Lanes 1 and 9 show iMabs with a 9 beta-strand scaffold while the other lanes show iMabs with a 7 beta-strand scaffold. The iMabs are depicted with an arrow.

FIG. 18: Confocal Laser Scanning Miroscopy (CLSM) of Alexa-488 labeled iMabs that have affinity for hair. Panel A, Alexa-488-iMab143-xx-0030 bound to hair; Panel B, Alexa-488-iMab143-xx-0034 bound to hair; and Panel C, hair in PBS.

FIG. 19: Histological staining of cross-section of human skin with iMabs. The iMabs, all containing a VSV tag, were incubated on a 6 82 m thick cross-section of human skin and allowed to bind for two hours. After washing, binding specificity and localization were visualized with anti-VSV-hrp labeled antibody and reaction-reaction with diaminobenzidine (DAB). Panel A, control, cross-section stained with only anti-VSV-hrp labeled antibody; Panel B, iMab142-xx-0032; and Panel C, iMab143-xx-0031. iMab142-xx-32 stains specifically some cells in the dermis, while iMab143-xx-0031 stains all cell nuclei and the epidermis.

FIG. 20: Panel A, far UV CD spectra (215-260 nm) of iMab138-xx-0007 (iMab138), iMab139-xx-0007 (iMab139), iMab140-xx-0007 (iMab140), iMab141-xx-0007 (iMab141), iMab111 and iMab116 at 20° C. The iMabs were dissolved in 1×PBS, pH 7.5. Panel B, far UV CD spectra (215-260 nm) of iMab138-xx-0007 (iMab138), iMab139-xx-0007 (iMab139), iMab140-xx-0007 (iMab140), iMab141-xx-0007 (iMab141), iMab111 and iMab116 after heating for ten minutes at 80° C. and refolding at 20° C.

FIG. 21: Far UV CD spectra (215-260 nm) of iMab135-xx-0001, iMab136-xx-0001 and iMab137-xx-0001 at 20° C., at 80° C. and again at 20° C. The iMab dissolved in 1×PBS, pH 7.5.

FIGS. 22A-22I: Alignment of amino acid sequences of VAPS, respectively corresponding to SEQ ID NOS:244-310, to show the beta-elements, the connecting loops and the affinity regions.

### DESCRIPTION OF THE TABLES

Table 1: Examples of nine-stranded folds (strands only) in PDB format.

Table 2: Example amino acid sequences likely to fold as nine-stranded iMab proteins, respectively corresponding to SEQ ID NOS:13-19.

Table 3: VAP (iMab) amino acid sequences. xx: number of C terminal tag not present in these sequences, respectively corresponding to SEQ ID NOS:20-72.

Table 4: iMab DNA sequences, respectively corresponding to SEQ ID NOS:73-125.

Table 5: List of primers used, respectively corresponding to SEQ ID NOS:126-170.

Table 6: Binding characteristics of purified iMab variants to lysozyme. Various purified iMabs containing either 6, 7, or 9 beta-sheets were analyzed for binding to ELK (control) and lysozyme as described in Examples 8, 15, 19 and 23. All iMabs were purified using urea and subsequent matrix-assisted refolding (Example 7), except for iMab100, which was additionally also purified by heat-induced solubilization of inclusion bodies (Example 6).

Table 7: Effect of pH shock on iMab100, measured in Elisa versus lysozyme before and after precipitation by Potassium acetate pH 4.8.

Table 8: Four examples of seven-stranded (strands only) folds in PDB 2.0 format to indicate spatial conformation.

Table 9: PROSAII results (zp-comp) and values for the objective function from MODELLER for seven-stranded iMab proteins. Lower values correspond to iMab proteins which are more likely to fold correctly.

Table 10: Example amino acid sequences less likely to fold as seven-stranded iMab proteins, respectively corresponding to SEQ ID NOS:171-200.

Table 11: Four examples of six-stranded (strands only) folds in PDB 2.0 format to indicate spatial conformation.

Table 12: PROSAII results (zp-comp) and values for the objective function from MODELLER for six-stranded iMab proteins. Lower values correspond to iMab proteins that are more likely to fold correctly.

Table 13: Example amino acid sequences likely to fold as six-stranded iMab proteins, respectively corresponding to SEQ ID NOS:201-206.

Table 14: PROSAII results (zp-comp) from iMab100 derivatives of which lysine was replaced at either position 3, 7, 19 and 65 with all other possible amino acid residues. Models were made with and without native cysteine bridges. The more favorable derivatives (which are hydrophilic) are denoted with X.

Table 15: PROSAII results (zp-comp) from iMab100 derivatives of which cysteine at position 96 was replaced with all other possible amino acid residues.

Table 16A: Amino acid sequence of iMab100 (reference), together with the possible candidates for extra cysteine bridge formation. The position where a cysteine bridge can be formed is indicated.

Table 16B: Preferred locations for cysteine bridges with their corresponding PROSAII score (zp-comp) and the corresponding iMab name.

Table 17: Effect of mutation frequency of dITP on the number of binders after panning.

Table 18: Nucleotide sequences of the phage display vector CM114-iMab100 and the expression vector CM126-iMab100, respectively corresponding to SEQ ID NOS:207-208.

Table 19: Head space analysis of release of octanal bound to iMab100.

Table 20: Amino acid sequences of hair and/or skin-binding VAPs, respectively corresponding to SEQ ID NOS:209-221.

Table 21: Nucleotide sequence of hair- and skin-binding VAPs, respectively corresponding to SEQ ID NOS:222-234. xx: number of C-terminal tags not present in these sequences.

Table 22: ELISA results of the VAPS binding to skin and hair proteins. Background signal means no iMab added.

23: Results of binding of Alexa-488-labeled iMabs to human hair. Fluorescence was measured with a Confocal Laser Scanning Microscope (LSM510, Zeiss).

Table 24: Affinity region 4 (AR4) of iMabs with affinity for hair and/or skin, respectively corresponding to SEQ ID NOS:235-243.

### DETAILED DESCRIPTION OF THE INVENTION

Through molecular modeling, structure analyses and methods based on recent advances in molecular biology, molecular, versatile affinity proteins (VAPs) were devised for specific applications in the field of cosmetics and fragrance industries. These VAPs can provide a versatile context to specifically deliver cosmetic agents to targets associated with skin, hair, nails, saliva, textile fabrics, and tissue-type materials such as diapers, hygiene pads, etc. In order to deliver the cosmetic agents to the target, the agent is coupled (linked) to a VAP, which has specific affinity for the desired target.

a) Versatile Affinity Proteins

i) VAP design and Construction

The present invention relates to the design, construction, production, screening and use of proteins that contain one or more regions that may be involved in molecular binding. The invention also relates to naturally occurring proteins provided with artificial binding domains, re-modeled natural occurring proteins provided with extra structural components and provided with one or more artificial binding sites, re-modeled natural occurring proteins disposed of some elements (structural or others) provided with one or more artificial binding sites, artificial proteins containing a standardized core structure motif provided with one or more binding sites. All such proteins are called VAPs (Versatile Affinity Proteins) herein. The invention further relates to novel VAPs identified according to the methods of the invention and the transfer of binding sites on naturally occurring proteins that contain a similar core structure. 3D modeling or mutagenesis of such natural occurring proteins can be desired before transfer in order to restore or ensure antigen binding capabilities by the affinity regions present on the selected VAP. Further, the invention relates to processes that use selected VAPs, as described in the invention, for purification, removal, masking, liberation, inhibition, stimulation, capturing, etc., of the chosen ligand capable of being bound by the selected VAP(s).

Ligand Binding Proteins

Many naturally occurring proteins that contain a (putative) molecular binding site comprise two functionally different regions: The actual displayed binding region and the region(s) that is (are) wrapped around the molecular binding site or pocket, called the scaffold herein. These two regions are different in function, structure, composition and physical properties. The scaffold structures ensure a stable three-dimensional conformation for the whole protein, and act as a steppingstone for the actual recognition region.

Two functional different classes of ligand binding proteins can be discriminated. This discrimination is based upon the presence of a genetically variable or invariable ligand binding region. In general, the invariable ligand binding proteins contain a fixed number, a fixed composition and an invariable sequence of amino acids in the binding pocket in a cell of that species. Examples of such proteins are all cell adhesion molecules, e.g., N-CAM and V-CAM, the enzyme families, e.g., kinases and proteases and the family of growth receptors, e.g., EGF-R, bFGF-R. In contrast, the genetically variable class of ligand binding proteins is under control of an active genetic shuffling, mutational or rearrangement mechanism enabling an organism or cell to change the number, composition and sequence of amino acids in, and possibly around, the binding pocket. Examples of these are all types of light and heavy chain of antibodies, B-cell receptor light and heavy chains and T-cell receptor alpha, beta, gamma and delta chains. The molecular constitution of wild type scaffolds can vary to a large extent. For example, Zinc finger containing DNA binding molecules contain a totally different scaffold (looking at the amino acid composition and structure) than antibodies although both proteins are able to bind to a specific target.

Scaffolds and Ligand Binding Domains

Antibodies Obtained via Immunizations

The class of ligand binding proteins that express variable (putative) antigen binding domains has been shown to be of great value in the search for ligand binding proteins. The classical approach to generate ligand binding proteins makes use of the animal immune system. This system is involved in the protection of an organism against foreign substances. One way of recognizing, binding and clearing the organism of such foreign highly diverse substances is the generation of antibodies against these molecules. The immune system is able to select and multiply antibody producing cells that recognize an antigen. This process can also be mimicked by means of active immunizations. After a series of immunizations antibodies may be formed that recognize and bind the antigen. The possible number of antibodies with different affinity regions that can be formed due to genetic rearrangements and mutations, exceeds the number of 10⁴⁰. However, in practice, a smaller number of antibody types will be screened and optimized by the immune system. The isolation of the correct antibody producing cells and subsequent immortalization of these cells or, alternatively, cloning of the selected antibody genes directly, antigen-antibody pairs can be conserved for future (commercial and non-commercial) use.

The use of antibodies obtained this way is restricted only to a limited number of applications. The structure of animal antibodies is different than antibodies found in human. The introduction of animal-derived antibodies in humans, e.g., for medical applications, will almost certainly cause immune responses adversing the effect of the introduced antibody (e.g., HAMA reaction). As it is not allowed to actively immunize men for commercial purposes, it is not or only rarely possible to obtain human antibodies this way. Because of these disadvantages methods have been developed to bypass the generation of animal-specific antibodies. One example is the removal of the mouse immune system and the introduction of the human immune system in such mouse. All antibodies produced after immunization are of human origin. However, the use of animals has also a couple of important disadvantages. First, animal care has a growing attention from ethologists, investigators, public opinion and government. Immunization belongs to a painful and stressful operation and must be prevented as much as possible. Second, immunizations do not always produce antibodies or do not always produce antibodies that contain required features such as binding strength, antigen specificity, etc. The reason(s) for this can be multiple: the immune system missed by co-incidence such a putative antibody; the initially formed antibody appeared to be toxic or harmful; the initially formed antibody also recognizes animal-specific molecules and consequently the cells that produce such antibodies will be destroyed; or the epitope cannot be mapped by the immune system (this can have several reasons).

Otherwise Obtained Antibodies

It is clear, as discussed above, that immunization procedures may result in the formation of ligand binding proteins but their use is limited, inflexible and uncontrollable. The invention of methods for the bacterial production of antibody fragments (Skerra and Pluckthun, 1988; Better et al., 1988) provided new powerful tools to circumvent the use of animals and immunization procedures. It is has been shown that cloned antibody fragments, (frameworks, affinity regions and combinations of these) can be expressed in artificial systems, enabling the modulation and production of antibodies and derivatives (Fab, V_{L}, V_{H}, scFv and V_{HH}) that recognize a (putative) specific target in vitro. New efficient selection technologies and improved degeneration strategies directed the development of huge artificial (among which human) antibody fragment libraries. Such libraries potentially contain antibodies fragments that can bind one or more ligands of choice. These putative ligand-specific antibodies can be retrieved by screening and selection procedures. Thus, ligand binding proteins of specific targets can be engineered and retrieved without the use of animal immunizations.

Other Immunoglobulin Superfamily-Derived Scaffolds

Although most energy and effort is put in the development and optimization of natural derived or copied human antibody-derived libraries, other scaffolds have also been described as successful scaffolds as carriers for one or more ligand binding domains. Examples of scaffolds based on natural occurring antibodies encompass minibodies (Pessi et al., 1993), Camelidae V_{HH} proteins (Davies and Riechmann, 1994; Hamers-Casterman et al., 1993) and soluble V_{H} variants (Dimasi et al., 1997; Lauwereys et al., 1998). Two other natural occurring proteins that have been used for affinity region insertions are also members of the immunoglobulin superfamily: the T-cell receptor chains (Kranz et al., WO Patent 0148145) and fibronectin domain-3 regions (Koide U.S. Pat. No. 6,462,189; Koide et al., 1998). The two T-cell receptor chains can each hold three affinity regions according to the inventors while for the fibronectin region the investigators described only two regions.

Non-Immunoglobulin-Derived Scaffolds

Besides immunoglobulin domain-derived scaffolds, non-immunoglobulin domain containing scaffolds have been investigated. All proteins investigated contain only one protein chain and one to four affinity related regions. Smith and his colleagues (1998) reported the use of knottins (a group of small disulfide bonded proteins) as a scaffold. They successfully created a library based on knottins that had seven mutational amino acids. Although the stability and length of the proteins are excellent, the low number of amino acids that can be randomized and the singularity of the affinity region make knottin proteins not very powerful. Ku and Schultz (1995) successfully introduced two randomized regions in the four-helix-bundle structure of cytochrome b₅₆₂. However, selected binders were shown to bind with micromolar K_{d} values instead of the required nanomolar or even better range. Another alternate framework that has been used belongs to the tendamistat family of proteins. McConnell and Hoess (1995) demonstrated that alpha-amylase inhibitor (74 amino acid beta-sheet protein) from Streptomyces tendae could serve as a scaffold for ligand binding libraries. Two domains were shown to accept degenerated regions and function in ligand binding. The size and properties of the binders showed that tendamistats could function very well as ligand mimickers, called mimotopes. This option has now been exploited. Lipocalin proteins have also been shown to be successful scaffolds for a maximum of four affinity regions (Beste et al., 1999; Skerra, 2000 BBA; Skerra, 2001 RMB). Lipocalins are involved in the binding of small molecules like retinoids, arachidonic acid and several different steroids. Each lipocalin has a specialized region that recognizes and binds one or more specific ligands. Skerra (2001) used the lipocalin RBP and lipocalin BBP to introduce variable regions at the site of the ligand binding domain. After the construction of a library and successive screening, the investigators were able to isolate and characterize several unique binders with nanomolar specificity for the chosen ligands. It is currently not known how effective lipocalins can be produced in bacteria or fungal cells. The size of lipocalins (about 170 amino acids) is pretty large in relation to V_{HH} chains (about 100 amino acids), which might be too large for industrial applications.

Core Structure Development

In commercial industrial applications, it is very interesting to use single chain peptides, instead of multiple chain peptides because of low costs and high efficiency of such peptides in production processes. One example that could be used in industrial applications is the V_{HH} antibodies. Such antibodies are very stable, can have high specificities and are relatively small. However, the scaffold has evolutionarily been optimized for an immune dependent function but not for industrial applications. In addition, the highly diverse pool of framework regions that are present in one pool of antibodies prevents the use of modular optimization methods. Therefore a new scaffold was designed based on the favorable stability of V_{HH} proteins.

3D-modeling and comparative modeling software was used to design a scaffold that meets the requirements of versatile affinity proteins (VAPs).

However, at this moment it is not yet possible to calculate all possible protein structures, protein stability and other features, since this would cost months of computer calculation capacity. Therefore we test the most promising computer designed scaffolds in the laboratory by using display techniques, such as phage display or the like. In this way it is possible to screen large numbers of scaffolds in a relatively short time.

Immunoglobulin-like (ig-like) folds are very common throughout nature. Many proteins, especially in the animal kingdom, have a fold region within the protein that belongs to this class. Reviewing the function of the proteins that contain an ig-like fold and reviewing the function of this ig-like fold within that specific protein, it is apparent that most of these domains, if not all, are involved in ligand binding. Some examples of ig-like fold containing proteins are: V-CAM, immunoglobulin heavy chain variable domains, immunoglobulin light chain variable domains, constant regions of immunoglobulins, T-cell receptors, fibronectin, reovirus coat protein, beta-galactosidase, integrins, EPO-receptor, CD58, ribulose carboxylase, desulphoferrodoxine, superoxide likes, biotin decarboxylase and P53 core DNA binding protein. A classification of most ig-like folds can be obtained from the SCOP database (Murzin A. G. et al., J. Mol. Biol. 247, 536-540, 1995; http://scop.mrc-lmb.cam.ac.uk/scop) and from CATH (Orengo et al., Structure 5(8), 1093-1108, 1997; http://www.biochem.ucl.ac.uk/bsm/cath_new/index.html). SCOP classifies these folds as: all beta-proteins, with an immunoglobulin-like beta-sandwich in which the sandwich contains seven strands in two sheets although some members that contain the fold have additional strands. CATH classifies these folds as: mainly beta-proteins with an architecture like a sandwich in an immunoglobulin-like fold designated with code 2.60.40. In structure databases like CE (Shindyalov et al., Protein Engineering, 11(9) 739-747, 1998; http://cl.sdsc.edu/ce.htm), VAST (Gibrat et al., Curr. Op. Struc. Biol. 6(3), 377-385, 1996; http://www.ncbi.nlm.nih.gov/Structure/VAST/vast.shtml) and FSSP (Holm et al., Nucl. Acids Res., 26, 316-319; Holm et al., Proteins, 33, 88-96, 1998; http://www.ebi.ac.uk/dali/fssp) similar classifications are used.

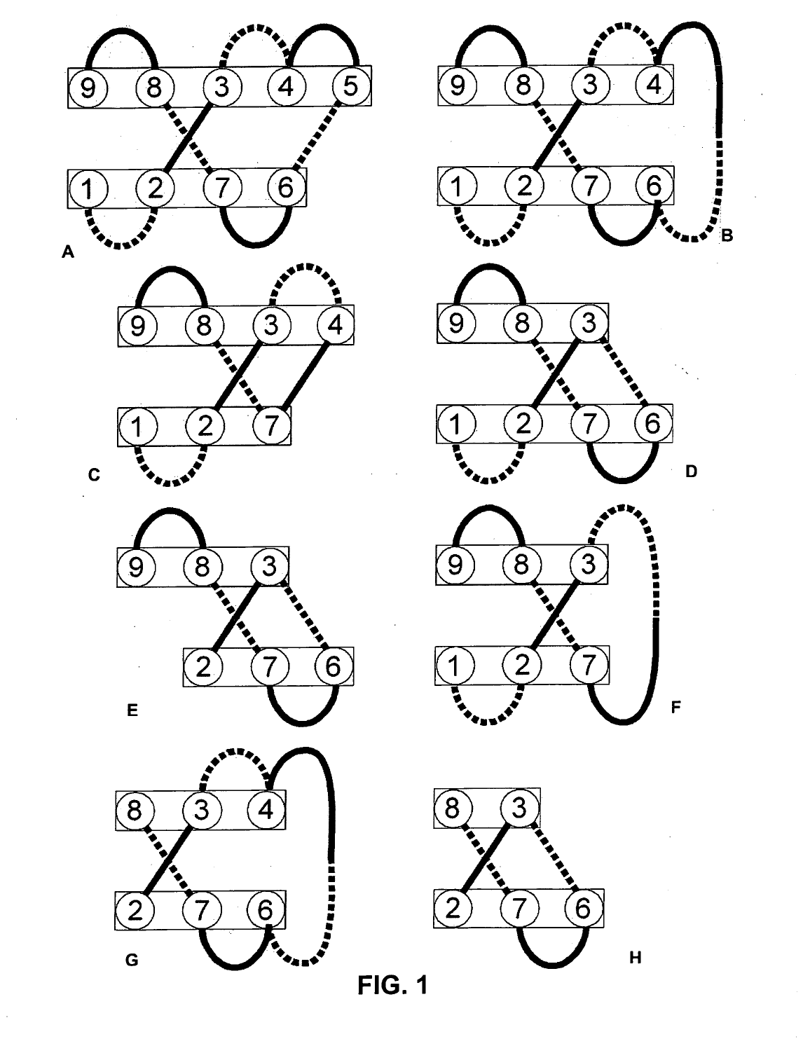

Projection of these folds from different proteins using software of Cn3D (NCBI; http://www.ncbi.nlm.nih.gov/Structure/CN3D/cn3d.shtml), InsightIII (MSI; http://www.accelrys.com/insight) and other structure viewers, showed that the ig-like folds have different sub-domains. A schematic projection of the structure is depicted in FIG. 1. The most conserved structure was observed in the center of the folds, named the core. The core structures hardly vary in length and have a relative conserved spatial constrain, but they were found to vary to a large degree in both sequence and amino acid composition. On both sides of the core, sub-domains are present. These are called connecting loops. These connecting loops are extremely variable as they can vary in amino acid content, sequence, length and configuration. The core structure is therefore designated as the far most important domain within these proteins. The number of beta-elements that form the core can vary between seven and nine, although six-stranded core structures might also be of importance. All beta-elements of the core are arranged in two beta-sheets. Each beta-sheet is built of anti-parallel oriented beta-elements. The minimum number of beta-elements in one beta-sheet that was observed was three elements. The maximum number of beta-element in one sheet that was observed was five elements, although it can not be excluded that higher number of beta-elements might be possible. Connecting loops connect the beta-elements on one side of the barrel. Some connections cross the beta-sheets while others connect beta-elements that are located within one beta-sheet. Especially the loops that are indicated as L2, L4, L6 and L8 are used in nature for ligand binding and are therefore preferred site for the introduction or modification of binding peptide/affinity region. The high variety in length, structure, sequences and amino acid compositions of the L1, L3, L5 and L7 loops clearly indicates that these loops can also be used for ligand binding, at least in an artificial format.

Amino acid side chains in the beta-elements that form the actual core that are projected towards the interior of the core, and thus fill the space in the center of the core, can interact with each other via H-bonds, covalent bonds (cysteine bridges) and other forces, and determine the stability of the fold. Because amino acid composition and sequence of the residues of the beta-element parts that line up the interior were found to be extremely variable, it was concluded that many other sequence formats can be created.

In order to obtain the basic concept of the structure as a starting point for the design of new types of proteins containing this ig-like fold, projections of domains that contain ig-like folds were used. Insight II, Cn3D and Modeller programs were used to determine the minimal elements and lengths. In addition, only C-alpha atoms of the structures were projected because these described the minimal features of the folds. Minor differences in spatial positions (coordinates) of these beta-elements were allowed.

PDB files representing the coordinates of the C-alpha atoms of the core of ig-like folds were used for the development of new 9, 8, 7, 6 and 5 beta-elements containing structures. For eight-stranded structures, beta-element 1 or 9 can be omitted but also elements 5 or 6 can be omitted. Thus an eight-stranded core preferably comprises elements 2-8, and either 1 or 9. Another preferred eight-stranded core comprises elements 1-4, 7-9, and either strand 5 or strand 6. For seven-stranded structures, two beta-elements can be removed among which combinations of element 1 and 9, 1 and 5, 6 and 9, 9 and 5 and, elements 4 and 5. The exclusion of elements 4 and 5 is preferred because of spatial constrains. Six-stranded structures lack preferably element 1, 4 and 5 or 4, 5 and 9 but also other formats were analyzed with Insight and Modeler and shown to be reliable enough for engineering purposes.

Multiple primary scaffolds were constructed and pooled. All computer designed proteins are just an estimated guess. One mutation or multiple amino acid changes in the primary scaffold may make it a successful scaffold or make it function even better than predicted. To accomplish this the constructed primary scaffolds are subjected to a mild mutational process by PCR amplification that includes error-prone PCR, such as unequimolar dNTP concentration, addition of manganese or other additives, or the addition of nucleotide analogues, such as dITP (Spee et al., Nucl. Acids Res. 21(3), 777-8, 1993) or dPTP (Zaccolo et al., J. Mol. Biol. 255(4), 589-603, 1996) in the reaction mixture which can ultimately change the amino acid compositions and amino acid sequences of the primary scaffolds. This way new (secondary) scaffolds are generated.

In order to test the functionality, stability and other characteristics required or desired features of the scaffolds, a set of known affinity regions, such as 1MEL for binding lysozyme and 1BZQ for binding RNase were inserted in the primary modularly constructed scaffolds. Functionality, heat and chemical stability of the constructed VAPs were determined by measuring unfolding conditions. Functionality after chemical or heat treatment was determined by binding assays (ELISA), while temperature-induced unfolding was measured using a circular dichroism (CD) polarimeter. Phage display techniques were used to select desired scaffolds or for optimization of scaffolds. In the present invention, variants were generated. In the course thereof, VAP molecules were generated that are not capable of forming cysteine bridges between the two beta-sheets. This is possible by replacing at least one of the couple of cysteines from at least one of the two beta-sheets. In a preferred embodiment, the invention provides a conjugate comprising a core of a sequence as depicted in Table 3 or FIGS. 22A-221, preferably a core within an amino acid sequence depicted as iMab138-xx-0007, 139-xx-0007, 140-xx-0007, or 141-xx-0007 in Table 3. Conjugates comprising such cores can differ in their temperature stability. Thus, conjugates can be generated with stability toward denaturation for ten minutes at 60° C., or preferably 80° C., and refolding at 20° C., or with instability toward such denaturation, the latter being an embodiment in which the connection between the cosmetic substance and the target molecule can be disrupted through the presence of a temperature signal, the temperature signal being an exposure to a temperature of about 60° C., preferably 80° C., preferably for a duration of ten minutes. In the present invention, further VAP molecules were generated that have different pI values. Such VAP molecules are useful in the present invention in conjugates that display a different behavior in an aqueous solution. In a preferred embodiment, a conjugate of the invention comprises at least a core of a sequence as depicted in Table 3 or FIGS. 22A-221. Preferably, the conjugate comprises a core within the amino acid sequence as depicted as iMab135-xx-0002, 136-xx-0002 or 137-xx-0002 in Table 3.

Initial Affinity Regions for Library Construction

In the present invention, new and unique affinity regions are required. Affinity regions can be obtained from natural sources, degenerated primers or stacked DNA triplets. All of these sources have certain important limitations as described above. In our new setting we designed a new and greatly improved source of affinity regions that have less restrictions, can be used in modular systems, are extremely flexible in use and optimization, are fast and easy to generate and modulate, have a low percentage of stop codons, have an extremely low percentage of frameshifts and wherein important structural features will be conserved in a large fraction of the newly formed clones and new structural elements can be introduced.

The major important affinity region (CDR3) in both light and heavy chain in normal antibodies has an average length between 11 (mouse) and 13 (human) amino acids. Because in such antibodies the CDR3 in light and heavy chain cooperatively function as antigen binders, the strength of such a binding is a result of both regions together. In contrast, the binding of antigens by V_{HH} antibodies (Camelidae) is a result of one CDR3 region due to the absence of a light chain. With an estimated average length of 16 amino acids, these CDR3 regions are significantly longer than regular CDR3 regions (Mol. Immunol., Bang Vu et al., 1997, 34, 1121-1131). It can be emphasized that long or multiple CDR3 regions have potentially more interaction sites with the ligand and can therefore be more specific and bind with more strength. Other exceptions are the CDR3 regions found in cow (Bos taurus) (Berens et al., Int. Immunol., 9(1), 189-99, 1997). Although the antibodies in cow consist of a light and a heavy chain, their CDR3 regions are much longer than found in mouse and humans and are comparable in length found for camelidae CDR3 regions. Average lengths of the major affinity region(s) should preferably be about 16 amino acids. In order to cover as much as possible potentially functional CDR lengths the major affinity region can vary between 1 and 50 or even more amino acids. As the structure and the structural classes of CDR3 regions (like for CDR1 and CDR2) have not been clarified and understood it is not possible to design long affinity regions in a way that the position and properties of crucial amino acids are correct. Therefore, most libraries were supplied with completely degenerated regions in order to find at least some correct regions.

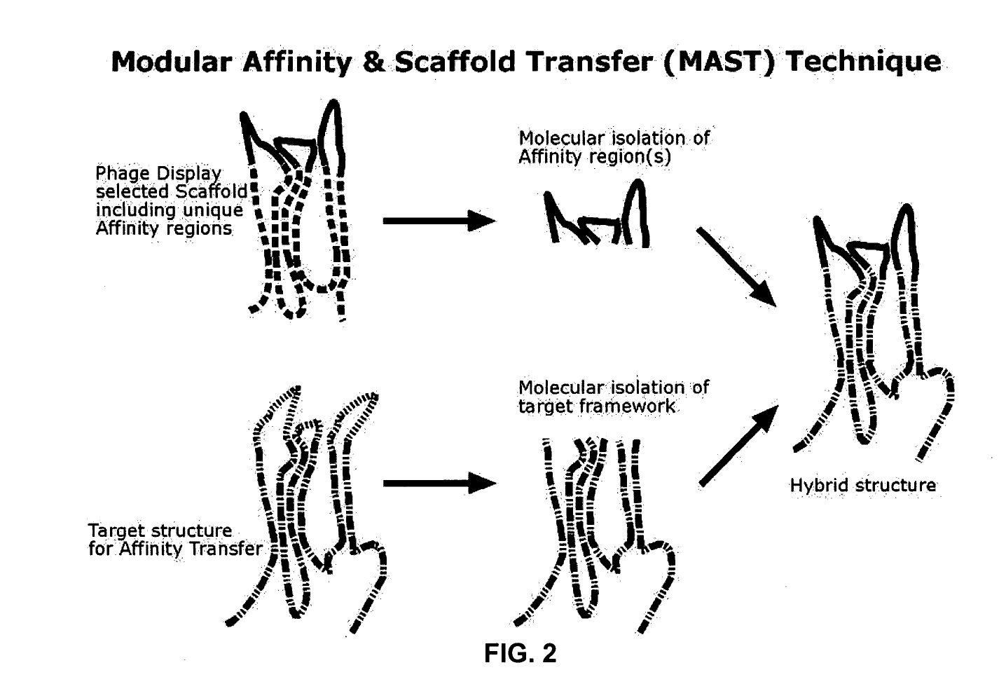

In the invention we describe the use of natural occurring camelidae V_{HH} CDR3 as well as bovine-derived VH CDR3 regions as a template for new affinity regions, but of course other CDR regions (e.g., CDR1 and CDR2) as well as other varying sequences that correspond in length might be used. CDR3 regions were amplified from mRNA coding for V_{HH} antibodies originating from various animals of the camelidae group or from various other animals containing long CDR3 regions by means of PCR techniques. Next this pool of about 10⁸ different CDR3 regions, which differ in the coding for amino acid composition, amino acid sequence, putative structural classes and length, is subjected to a mutational process by PCR as described above. The result is that most products will differ from the original templates and thus contain coding regions that potentially have different affinity regions. Other very important consequences are that the products keep their length, the pool keeps their length distribution, a significant part will keep structurally important information while others might form non-natural classes of structures, the products do not or only rarely contain frame shifts and the majority of the products will lack stop codons. These new affinity regions can be cloned into the selected scaffolds by means of the Modular Affinity and Scaffold Transfer technology (MAST). This technique is based on the fact that all designed and constructed scaffolds described above have a modular structure such that all loops connecting the beta-strands can be easily replaced by other loops without changing the overall structure of the VAP (see FIG. 2). The newly constructed library can be subjected to screening procedures similar to the screening of regular libraries known by an experienced user in the field of the art. Thus, further provided is a method for producing a library comprising artificial binding peptides, the method comprising providing at least one nucleic acid template wherein the templates encode different specific binding peptides, producing a collection of nucleic acid derivatives of the templates through mutation thereof and providing the collection or a part thereof to a peptide synthesis system to produce the library comprising artificial binding peptides. The complexity of the library increases with increasing number of different templates used to generate the library. In this way, an increasing number of different structures are used. Thus, preferably at least two nucleic acid templates, and better at least ten nucleic acid templates are provided. Mutations can be introduced using various means and methods. Preferably, the method introduces mutations by changing bases in the nucleic acid template or derivative thereof. With “derivative” is meant a nucleic acid comprising at least one introduced mutation as compared to the template. In this way, the size of the affinity region is not affected. Suitable modification strategies include amplification strategies such as PCR strategies encompassing, for example, unbalanced concentrations of dNTPs (Cadwell et al., PCR Methods Appl. (1992) 2, 28-33; Leung et al., Technique (1989) 1, 11-15; Kuipers, Methods Mol. Biol. 57 (1996) 351-356), the addition of dITP (Xu et al., Biotechniques 27 (1999) 1102-1108; Spee et al., Nucleic Acids Res. 21 (1993) 777-778; Kuipers, Methods Mol. Biol. 57 (1996) 351-356), dPTP (Zaccolo et al., J. Mol. Biol. 255 (1996) 589-603), 8-oxo-dG (Zaccolo et al., J. Mol. Biol. 255 (1996) 589-603), Mn²⁺ (Cadwell et al., PCR Methods Appl. (1992) 2, 28-33; Leung et al., Technique (1989) 1, 11-15, Xu et al., Biotechniques 27 (1999) 1102-1108), polymerases with high misincorporation levels (Mutagene ®, Stratagene). Site-specific protocols for introducing mutations can of course also be used, however, the considerable time and effort to generate a library using such methods would opt against a strategy solely based on site directed mutagenizes. Hybrid strategies can of course be used. Mutation strategies comprising dITP and/or dPTP incorporation during elongation of a nascent strand are preferred since such strategies are easily controlled with respect to the number of mutations that can be introduced in each cycle. The method does not rely on the use of degenerate primers to introduce complexity.

Therefore, in one embodiment, the amplification utilizes non-degenerate primers. However, (in part) degenerate primers can be used, thus, also provided is a method wherein at least one non-degenerate primer further comprises a degenerate region. The methods for generating libraries of binding peptides is especially suited for the generation of the above mentioned preferred larger affinity regions. In these a larger number of changes can be introduced while maintaining the same of similar structure. Thus, preferably at least one template encodes a specific binding peptide having an affinity region comprising at least 14 amino acids and preferably at least 16 amino acids.

Though non-consecutive regions can be used in this embodiment of the invention it is preferred that the region comprises at least 14 consecutive amino acids. When multiple templates are used it is preferred that the regions comprise an average length of 24 amino acids.

The method for generating a library of binding peptides may favorably be combined with core regions of the invention and method for the generation thereof. For instance, once a suitable binding region is selected a core may be designed or selected to accommodate the particular use envisaged. However, it is also possible to select a particular core region, for reasons of the intended use of the binding peptide. Subsequently libraries having the core and the mentioned library of binding peptides may be generated. Uses of such libraries are, of course, many fold. Alternatively, combinations of strategies may be used to generate a library of binding peptides having a library of cores. Complexities of the respective libraries can of course be controlled to adapt the combination library to the particular use. Thus, in a preferred embodiment, at least one of the templates encodes a proteinaceous molecule according to the invention.

The mentioned peptide, core and combination libraries may be used to select proteinaceous molecules of the invention, thus herein is further provided a method comprising providing a potential binding partner for a peptide in the library of artificial peptides and selecting a peptide capable of specifically binding to the binding partner from the library. A selected proteinaceous molecule obtained using the method is of course also provided. To allow easy recovery and production of a selected proteinaceous molecule it is preferred that at least the core and the binding peptide is displayed on a replicative package comprising nucleic acid encoding the displayed core/peptide proteinaceous molecule. Preferably, the replicative package comprises a phage, such as used in phage display strategies. Thus, also provided is a phage display library comprising at least one proteinaceous molecule of the invention. As mentioned above, the method for generating a library of binding peptides can advantageously be adapted for core regions. Thus, also provided is a method for producing a library comprising artificial cores, the method comprising providing at least one nucleic acid template wherein the templates encode different specific cores, producing a collection of nucleic acid derivatives of the templates through mutation thereof and providing the collection or a part thereof to a peptide synthesis system to produce the library of artificial cores. Preferred binding peptide libraries are derived from templates comprising CDR3 regions from cow (Bos Taurus) or camelidae (preferred lama pacos and lama glama).

Protein-ligand interactions are one of the basic principles of life. All protein-ligand-mediated interactions in nature either between proteins, proteins and nucleic acids, proteins and sugars or proteins and other types of molecules are mediated through an interface present at the surface of a protein and the molecular nature of the ligand surface. The very most of protein surfaces that are involved in protein-ligand interactions are conserved throughout the life cycle of an organism. Proteins that belong to these classes are, for example, receptor proteins, enzymes and structural proteins. The interactive surface area for a certain specific ligand is usually constant. However, some protein classes can modulate their nature of the exposed surface area through, e.g., mutations, recombinations or other types of natural genetic engineering programs. The reasons for this action is that their ligands or ligand types can vary to a great extent. Proteins that belong to such classes are, for example, antibodies, B-cell receptors and T-cell receptor proteins. Although there is in principle no difference between both classes of proteins, the speed of surface changes for both classes differ. The first class is mainly sensitive to evolutionary forces (lifespan of the species) while the second class is more sensitive to mutational forces (within the lifespan of the organism).

Binding specificity and affinity between receptors and ligands is mediated by an interaction between exposed interfaces of both molecules. Protein surfaces are dominated by the type of amino acids present at that location. The 20 different amino acids common in nature each have their own side chain with their own chemical and physical properties. It is the accumulated effect of all amino acids in a certain exposed surface area that is responsible for the possibility to interact with other molecules. Electrostatic forces, hydrophobicity, H-bridges, covalent coupling and other types of properties determine the type, specificity and strength of binding with ligands.

The most sophisticated class of proteins involved in protein-ligand interactions is those of antibodies. An ingenious system has been evolved that controls the location and level mutations, recombinations and other genetic changes within the genes that can code for such proteins. Genetic changing forces are mainly focused to these regions that form the exposed surface area of antibodies that are involved in the binding of putative ligands. The enormous numbers of different antibodies that can be formed (theoretically) indicate the power of antibodies. For example, if the number of amino acids that are directly involved in ligand binding in both the light and heavy chains of antibodies are assumed to be eight amino acids for each chain (and this is certainly not optimistic) then 20^{2*8} which approximates 10²⁰ (20 amino acids types, two chains, eight residues) different antibodies can be formed. If also indirect effects of nearby located amino acids include and/or increase the actual number of direct interaction amino acids, one ends up with an astronomically large number. Not one organism on earth is ever able to testall of these or even just a fraction of these combinations in the choice of antibody against the ligand.

Not all amino acids present at the exposed surface area are equally involved in ligand binding. Some amino acids can be changed into other amino acids without any notable, or only minor, changes in ligand binding properties. Also, most surface areas of proteins are very flexible and can under the influence of the ligand surface easily remodel resulting in a fit with the ligand surface that would not occur with an inflexible ligand-binding region. Interacting forces as mentioned above between the protein and the ligand can thus steer or catalyze this remodeling. In general, large but limited number of genetic changes together with redundancy in amino acids and the flexible nature of the surface in combination with binding forces can lead to the production of effective ligand binding proteins.

Affinity Regions (ARs)

Natural-derived antibodies and their affinity regions have been optimized to a certain degree, during immune selection procedures. These selections are based upon the action of such molecules in an immune system. Antibody applications outside immune systems can be hindered due to the nature and limitations of the immune selection criteria. Therefore, industrial, cosmetic, research and other applications demand often different properties of ligand binding proteins. The environment in which the binding molecules may be applied can be very harsh for antibody structures, e.g., extreme pH conditions, salt conditions, odd temperatures, etc. Depending on the application CDRs might or might not be transplanted from natural antibodies on to a scaffold. For at least some application unusual affinity regions will be required. Thus, artificial constructed and carefully selected scaffolds and affinity regions will be required for other applications.

Affinity regions present on artificial scaffolds can be obtained from several origins. First, natural affinity regions can be used. CDRs of cDNAs coding for antibody fragments can be isolated using PCR and inserted into the scaffold at the correct position. The source for such regions can be of immunized or non-immunized animals. Second, fully synthetic ARs can be constructed using degenerated primers. Third, semi-synthetic ARs can be constructed in which only some regions are degenerated. Fourth, triplets coding for selected amino acids (monospecific or mixtures) can be fused together in a predetermined fashion. Fifth, natural-derived affinity regions (either from immunized or naive animals) which are being mutated during amplification procedures (e.g., NASBA or PCR) by introducing mutational conditions (e.g., manganese ions) or agents (e.g., dITP) during the reaction.

Because for reasons mentioned earlier, immunization based CDRs can be successful but the majority of ligands or ligand domains will not be immunogenic. Artificial affinity regions in combinations with powerful selection and optimization strategies become more and more important if not inevitable. Primer based strategies are not very powerful due to high levels of stop codons, frameshifts, difficult sequences, too large randomizations, relative small number of mutational spots (maximum of about eight spots) and short randomization stretches (no more than eight amino acids). The power of non-natural-derived ARs depends also on the percentage of ARs that putatively folds correctly, i.e., being able to be presented on the scaffold without folding problems of the ARs or even the scaffold. Hardly any information is currently available about structures and regions that are present in ARs. Therefore, the percentage of correctly folded and presented artificial ARs constructed via randomizations, especially long ARs, will be reciprocal with the length of constructed ARs. Insight in CDR and AR structures will most likely be available in the future, but is not available yet.

Single scaffold proteins which are used in applications that require high affinity and high specificity in general require at least one long affinity region or multiple medium length ARs in order to have sufficient exposed amino acid side chains for ligand interactions. Synthetic constructed highly functional long ARs, using primer or triplet fusion strategies, will not be very efficient for reasons as discussed above. Libraries containing such synthetic ARs would either be too low in functionality or too large to handle. The only available source for long ARs is one that can be obtained from animal sources (most often CDR3s in heavy chains of antibodies). Especially cow-derived and camelidae-derived CDR3 regions of, respectively, Vh chains and Vhh chains are unusually long. The length of these regions is in average above 13 amino acids but 30 amino acids or even more are no exceptions. Libraries constructed with such ARs obtained from immunized animals can be successful for those ligands or ligand domains that are immunologically active. Non-immunogenic ligands or ligand domains and ligands that appear to be otherwise silent in immune responsiveness (toxic, self recognition, etc) will not trigger the immune system to produce ligand-specific long CDRs. Therefore, long CDRs that mediate the binding of such targets cannot or can only hardly be obtained this way and thus their exist a vacuum in technologies that provides one with specific long ARs that can be used on single scaffold proteins. A comparable conclusion has also been drawn by Muyldermans (Reviews in Molecular Biotechnology 74 (2001) 277-302) who analyzed the use of synthetic ARs on lama Vhh scaffolds.

Isolation of CDR regions, especially CDR3 regions, by means of PCR enables one to use all length variations and use all structural variations present in the available CDR regions. The introduction of minor, mild, medium level or high level random mutations via nucleic acid amplification techniques like, for example, PCR will generate new types of affinity regions. The benefits of such AR pools are that length distributions of such generated regions will be conserved. Also, stop codon introductions and frame shifts will be prevented to a large degree due to the relatively low number of mutations if compared with random primers based methods. Further, depending on the mutational percentage, a significant part or even the majority of the products will code for peptide sequences that exhibit structural information identical or at least partly identical to their original template sequence present in the animal. Due to these mutations altered amino acid sequences will be generated by a vast part of the products and consequently these will have novel binding properties. Binding properties can be altered in respect to the original template not only in strength but also in specificity and selectivity. This way libraries of long AR regions can be generated with strongly reduced technical or physical problems as mentioned above if compared with synthetic, semi-synthetic and naturally obtained ARs.

In recent years, several new and powerful in vitro mutagenesis methods and agents have been developed. One branch of mutagenizing methods produces mutations independently of the location (in contract to site directed mutagenesis methods). PCR strategies encompass, for example, unbalanced concentrations of dNTPs (Cadwell et al., PCR Methods Appl. (1992) 2, 28-33; Leung et al., Technique (1989) 1, 11-15; Kuipers, Methods Mol. Biol. 57 (1996) 351-356), the addition of dITP (Xu et al., Biotechniques 27 (1999) 1102-1108; Spee et al., Nucleic Acids Res. 21 (1993) 777-778; Kuipers, Methods Mol. Biol. 57 (1996) 351-356), dPTP (Zaccolo et al., J. Mol. Biol. 255 (1996) 589-603), 8-oxo-dG (Zaccolo et al., J. Mol. Biol. 255 (1996) 589-603), Mn2+(Cadwell et al., PCR Methods Appl. (1992) 2, 28-33; Leung et al., Technique (1989) 1, 11-15, Xu et al., Biotechniques 27 (1999) 1102-1108), polymerases with high misincorporation levels (Mutagene ®, Stratagene).

Affinity Maturation

After one or more selection rounds, an enriched population of VAPs is formed that recognizes the ligand selected for. In order to obtain better, different or otherwise changed VAPs against the ligand(s), the VAP coding regions or parts thereof can be the subject of a mutational program as described above due to its modular nature. Several strategies are possible: First, the whole VAP or VAPs can be used as a template. Second, only one or more affinity regions can be mutated. Third framework regions can be mutated. Fourth, fragments throughout the VAP can be used as a template. Of course, iterative processes can be applied to change more regions. The average number of mutations can be varied by changing PCR conditions. This way every desired region can be mutated and every desired level of mutation number can be applied independently. After the mutational procedure, the new formed pool of VAPs can be re-screened and re-selected in order to find new and improved VAPs against the ligand(s). The process of maturation can be re-started and re-applied as many times as necessary.

The effect of this mutational program is that not only affinity regions 1 and 2 with desired affinities and specificities can be found but also that minor changes in the selected affinity region 3 can be introduced. It has been shown (REF) that mutational programs in this major ligand binding region can strongly increase ligand binding properties. In conclusion, the invention described here is extremely powerful in the maturation phase.

ii) Selection for Affinity Against Target Compound

VAPs can be selected that are specific for exposed ligands on either hair, skin or nails. In case of skin, obvious targets would be proteins or lipid/protein complexes that are present in the stratum comeum, especially in the stratifying squamous keratinizing epithelium where the soft keratins of type I and type II are expressed (The keratinocyte handbook, ed. Leigh I. M., Lane B., Watt F. M., 1994, ISBN 052143416 5). Other suitable exposed ligands can be KAPs (keratin associated proteins) such as involucrin, loricrin, filagrin, elafin (trappin2), sciellin, cystatin A, annexin 1, LEP/X5, S100 A1-A13, SPRR1 and 2, and the like. Most of these proteins are specific for skin, i.e. they have not (yet) been detected in hair, but if no cross reaction with hair is desired, it is easy for someone skilled in the art to do a negative selection with naive or matured libraries expressing VAPs with different, randomized affinity regions in ways as described in this patent, thereby circumventing any cross-reactivity with hair cuticle.

For selection of hair-specific binders, preferred VAP-targets would be directed against the hair cuticle, especially ligands exposed on the fiber surface, outer beta-layer and epicuticle of hairs. For example, the hard keratins or hard keratin intermediate filaments (Langbein L. et al., J. Biol. Chem. 274 19874-84, 1999; Langbein L. et al., J. Biol. Chem. 276 35123-32, 2001), or the different classes of KAPs which are uniquely expressed in the hair cuticle (e.g. KAP 19.4 of the high glycine-tyrosine class, or KAP 13.2 and KAP 15.1 of the high sulfur class). Interestingly, some KAPs are strongly expressed in scalp hairs but are low to absent in beard hairs (M. A. Rogers et al., J. Biol. Chem. 276:19440-51, 2001; M. A. Rogers et al., J. Biol. Chem. 30 September 2002). Other potential ligands are lipids stabilized by isopeptide bonds that form part of the hydrophobic outer layer of the hairs, with methyleicosanoic acid and C16:0 fatty acid are the major lipid components. Due to its poor extractability, the protein composition of hair cuticle is only starting to be unraveled, but for the selection of hair-specific VAPs, it is not necessary to know the actual molecular target. When a truly hair-specific ligand needs to be targeted (e.g., when hair-coloring agents as described in this patent are being used), a negative selection with naive or matured libraries expressing VAPs with different, randomized affinity regions in ways as described in this patent, thereby circumventing any cross-reactivity with skin epidermis can easily be done by someone skilled in the art. VAPs can be selected that either bind hair or bind skin. However, it is also possible to select VAP molecules having binding specificity for both hair and skin. In a preferred embodiment of the invention, a conjugate of the invention comprises a sequence of affinity region 4 of iMab143-xx-0029, 143-xx-0030, 143-xx-0031, 142-xx-0032, 143-xx-0033, 143-xx-0034, 143-xx-0035, 143-xx-0036, 142-xx-0036, 143-xx-0037, 144-xx-37, 143-xx-0038, 143-xx-0039 depicted in Table 20 or FIGS. 22A-22I. Affinity regions are depicted in Table 24. In a preferred embodiment, a conjugate comprising a VAP that binds hair comprises at least an affinity region 4 of iMab143-xx-0029, 143-xx-0030, 143-xx-0031, 143-xx-0033, 143-xx-0034, 143-xx-0038, 143-xx-0039 of Table 20 or FIGS. 22A-22I. In another embodiment, a conjugate comprising a VAP that binds skin comprises at least an affinity region 4 of iMab143-xx-0029, 143-xx-0030, 143-xx-0031, 142-xx-0032, 143-xx-0033, 143-xx-0034, 143-xx-0035, of Table 20 or FIGS. 22A-22I. In another embodiment, a conjugate comprising a VAP is capable of specifically binding either hair or skin, preferably such VAP comprises at least an affinity region 4 of iMab142-xx-0032 or 143-xx-0035 (hair) or iMab142-xx-0038 or 142-xx-0039 (skin).

In a preferred embodiment, the conjugate of the invention comprises a core having a core sequence of an iMab depicted in Tables 3 or 20 or FIGS. 22A-22I, having at least an affinity region 4 of either iMab143-xx-0029 through to iMab142-xx-0039. Preferably, the core further comprises affinity regions 1, 2 and/or 3 of one of the iMabs143-xx-0029 through to iMab142-xx-0039. The mentioned ranges, of course, include the mentioned iMabs . . . 29 and . . .39. In a particular preferred embodiment, the affinity region 4 comprises a sequence

| | |
|---|---|
| AANDLLDYELDCIGMGPNEYED | (SEQ ID NO:1) |
| or | |
| AAVPGILDYELGTERQPPSCTTRRWDYDY. | (SEQ ID NO:2) |

Further provided are so-called di- or multi-valent VAP comprising an affinity for at least two target molecules wherein the epitopes recognized may be the same or different. Preferably, the di- or multi-valent VAP comprises at least two VAP that each comprise a specific affinity for hair, preferably linked through a spacer. Any spacer that does not interfere in an essential way with the binding affinity for hair of the linked VAPs is suitable for the present di- or multi-valent VAP. Preferably, the spacer comprises a sequence SGGGGSGGGGSGGGG (SEQ ID NO:3). In a preferred embodiment, the di- or multi-valent VAP comprises at least two hair-binding affinity regions 4 depicted in Table 20 or FIGS. 22A-22I, where at least two hair-binding affinity regions 4 may be the same or different. Preferably, the hair-binding affinity region comprises the affinity region 4 of iMab142-xx-0038 or iMab142-xx-0039. In yet a further embodiment, the hair-binding affinity region further comprises affinity regions 1, 2 and/or 3 of iMab142-xx-0038 or iMab142-xx-0039. In yet a further preferred embodiment, the di- or multi-valent VAP comprises at least two of the hair-binding iMab sequences depicted in Table 20 or FIGS. 22A-22I.

Besides skin or hair-specific VAPs, other affinity targets can be selected for, such as cotton fibers, flax, fibers, hemp fibers, polyester fibers and the like, being all fibers that are used in fabrics for clothes, linen, etc. Cellulose fibers are an especially preferred embodiment of the invention, as these are used in many clothes, sheets, towels, diapers, hygiene pads, etc.

iii) Bulk Production of VAPs

Direct production of VAPs in transgenic plant seeds followed by minimal seed processing (such as described in U.S. Pat. No. 571,474 or via oil bodies as described in U.S. Pat. Nos. 6,146,645 and 5,948,682) would be an economically attractive source for bulk quantities of bi-valent VAPs but good alternative sources are industrial micro-organisms where cellular protein can be used as carrier protein to introduce the conditioner agent into the consumer product.

b) Coupling Cosmetic Agents to VAPs

For the coupling of cosmetic agents to amino acids comprising the VAPs, several strategies can be chosen, using standard coupling chemistry known to those skilled in the art. Ample literature is available for such coupling chemistry, as is illustrated by the Handbook of Molecular Probes (Eugene, OR, USA), Bioconjugate Techniques (G. T. Hermanson, Associated Press 1996) and references therein where a wide range of fluorescent dyes is coupled to proteins such as antibodies. The functional groups used for chemical binding include, amino groups, carboxyl groups, aldehyde groups, thiol groups, polysaccharide side chains and the like.

Other versatile coupling methods can also be used for coupling of active agents to VAPs. Examples are Kreatech's Universal Linkage System (ULS) as described in patents or patent applications U.S. Pat. No. 5,580,990; EP 0539466; U.S. Pat. No. 5,714,327, and U.S. Pat. No. 5,985,566.

At the most distal region from the affinity region (also described as the tail end of the molecule, the C-terminus), additional amino acids can be added that do not alter the tertiary structure of the scaffold, onto which cosmetic agents can be coupled without interference with the scaffold's stability. Also, 3D-structural modeling and analysis can be used to determine which amino acids are exposed from the scaffold as they are the most hydrophilic and which side chains are available for chemical coupling. Preferably, the target amino acids should not be present in the affinity regions, a feature that can be selected for when panning the display libraries and doing sequence analyses on putative binders.

In the case of cosmetic agents that lack organic functional groups, chemical modifications are necessary to enable these molecules with binding regions. Treatments using coupling agents and silicone treatments, such as amino-modified silicone (3-aminopropyl triethoxy silane), SH-modified silicone (3-mercaptopropyl triethoxy silane) and carboxyl-modified silicone can be used for the introduction of organic functional groups on the surface of cosmetic agents. In addition, the binding of the cosmetic agents to the protein moiety has to be designed so that the release conditions will not compromise the cosmetic agent's structure. Chemical modifications have to be therefore introduced in order to ensure that the released molecule's physical, chemical and active properties remain unscathed.

Coupling may be achieved through peptide bonds, direct coupling, through chemical bonds or a combination thereof. A preferred example of a chemical bond comprises aldehyde reacting with amines. Aldehydes will react with amines to form Schiff Bases or Imines. These compounds can then be further reduced to further form more stable bonds. Release of the aldehydes upon formation of the Schiff base can be triggered by moderate acid or basic aqueous solution. A summary of these reactions is shown below: 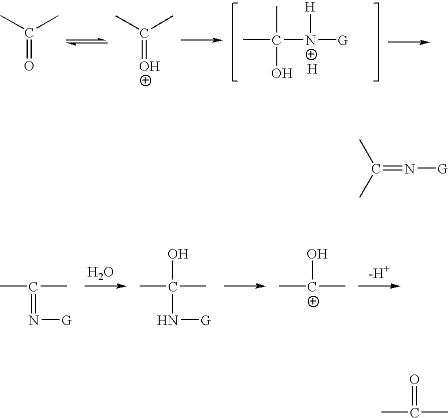
c) Classes of Cosmetic Agents

Several classes of cosmetic agents are envisioned in the invention:

i) Fragrances

One fragrant substance but preferably complexes composed of various molecules known in the classical field of fragrance are tagged to VAPs that can bind to a wide variety of target molecules with, e.g., affinity and specificity to skin, hair, textiles and tissue type materials. The fragrance molecules can then be released in a timed or conditioned fashion either by normal physiological natural processes such as enzymatic hydrolysis or by specific chemical reactions triggered by conditions brought by formulated products geared towards such conditions.

ii) Color compounds

A single colored agent or complexes composed of various molecules known in the classical field of hair dyes are tagged to VAPs that are selected to bind with high specificity and high affinity directly to, e.g., the hair surface, circumventing the need for hair dye molecules to penetrate the high and be dimerized under strong oxidative conditions.

iii) Conditioning Compounds

Single conditioning compounds and/or complexes composed of various molecules known in the classical field of cosmetic conditioners are tagged to VAPs that are selected to bind with high specificity and high affinity directly to hair, skin or nail surface. The cosmetic agents can be conditioners such as polymeric lubricants, antioxidants, dye fixative agents, conditioners, moisturizers, toners and various other compounds that improve the smell, look, feel, or overall health of the skin, hair or nails.

d) Fragrance Agents

Fragrances are usually not water-soluble and are applied in solvents such as ethanol or water/solvent mixtures to overcome the hydrophobic nature of the fragrance molecule. Release is temperature dependent and therefore a mixture of fragrances has different odor perceptions over time. A slow release mechanism provides a more controlled volatility and constant perception.

Using VAPs that are charged with various fragrance molecules as delivery agents, one can devise a slow release system based on the skin or hair physiology: sweat, heat, skin and hair natural bacterial flora's exogenous hydrolytic enzymes. In addition, the release of these fragrant molecules from the peptides can also be triggered by the addition of specially formulated products.

Various fragrance structural groups or more molecules of a single group can be attached to these affinity proteins via methods known in the field of organic chemistry. Depending on the fragrance intensity desired, the VAP can be synthetically designed to include side groups, which will optimize the binding of multiple fragrances.

The fragrant molecules that can be attached to these proteins lie in the following chemical classes (with examples, but not limited to these examples):

Acid salts, acetylenes, alcohols, aldehydes, amines, alpha-amino acids, carboxylic acids, esters, acetals, heterocycles, hydrocarbons, ketones, nitrites and cumulated double bonds, sulfides, disulfides and mercaptans and essential oils. Examples are acid salts such as non-aromatic acids salts, sodium acetate, potassium acetate, sodium citrate dihydrate or acetylenes such as 3-pentyn-1-ol, methyl 2-octynoate, methyl 2-nonynoate or alcohols such as 3-isopropylphenol, vanillyl alcohol, 1-octanol, 3-methyl-3-pentanol, pinacol, 4-hexen-1-ol, isoborneol, decahydro-2-napthol or polyols or aldehydes such as p-tolyacetaldehyde, cinnamaldehyde, 4-ethylbenzaldehyde, isobutyraldehyde, heptanal, 2-methyl-2-pentenal, dihydro-2,4,6-trimethyl-1,3,5(4h)dithiazine or alpha-amino acids such as DL-phenylalanine, DL-isoleucine, DL-methionine or carboxylic acids such as phenylacetic acid, cinnamic acid, propionic acid, isovaleric acid, fumaric acid, levulinic acid or esters, lactones such as benzyl formate, phenethyl formate, alpha-methylbenzyl butyrate, geranyl phenylacetate, ethyl p-anisate, butyl stearate, tripropionin, citronellyl acetate or ethers, acetals such as anisole, isoeugenol, vanillin propylene glycol acetal, caryophyllene oxide or heterocycles such as pyrrole, 2-pentylfuran, furfuryl mercaptan, 3-(2-furyl) acrolein, furfuryl heptanoate or hydrocarbons such as p-cymene, undecane, (r)-(+)-limonene, terpinolene, valencene, beta-caryophyllene or ketones such as 4-methyl-I -phenyl-2-pentanone, vanillylactone, propiophenone, 1-phenyl-1,2-propanedione, 2-decanone or sulfides, disulfides & mercaptans such as 2-phenethyl isothiocyanate, 2-methoxythiophenol, butyl sulfide, isopropyl disulfide, cyclopentanethiol, allyl isothiocyanate. A complete and comprehensive listing of fragrances products can be found in: Arctander's “Perfume and Flavor Chemicals and Perfume and Flavor Materials of Natural Origin” CD Rom. 1999 Edition.

i) Perfumes Deodorant, Shampoos

Perfumes, deodorants and shampoos may comprise fragrances coupled to VAP directed to skin and or hair components such as keratin and skin bacteria. Release of the fragrance can be achieved through proteolytic cleavage, oxidation or other means.

ii) Laundry Detergents Fabric Softeners

Laundry detergents and rinsing agents: fragrances coupled to VAPs that are selected for specificity against fabric materials such as cotton fibers, flax, fibers, hemp fibers, polyester fibers and the like, being all fibers that are used in fabrics for clothes, linen, towels etc. Laundry can be treated more effectively with such fragrance-VAPs as there will be very limited release after the rinse, both in wet and dry state of the laundry. Only when the fabric comes in contact with human skin, release of the fragrance molecules is triggered by either skin-or microflora-derived enzymes, or physical changes such as pH.

iii) Tissues Hygiene Pads, Diapers

Fragrances coupled to VAPs that are selected for specificity against fabric materials such as cotton fibers, flax fibers, hemp fibers, polyester fibers and the like, being all fibers that are used in fabrics for tissues, hygiene pads, diapers and the like. Only when the fabric comes in contact with human skin, release of the fragrance molecules can be triggered by either skin- or microflora-derived enzymes, or physical changes such as pH, temperature or high moisture conditions.

e) Skin and Nail Conditioning Agents

These conditioning agents can be described as materials, which improve the appearance of dry or damaged skin or nails. Many of these products are designed to remain on the skin (or nails) for a length of time in order to reduce flaking and to act as lubricants. These conditioning agents help maintain the soft, smooth, flexible nature of what is perceived as healthy, young looking skin (or nails).

i) Occlusive Agents

These agents perform in such a manner that the evaporation of water from the skin surface is substantially blocked. This occlusivity helps to increase the water content of the skin, giving it the desired supple appearance. Typically, occlusive agents are lipids, which, due to their water insolubility provide the best barrier to water vapor transport. The mechanism of skin moisturization by these lipids is based on their tendency to remain on the skin's surface over time to provide a long lasting occlusive effect. Examples such as naphtenic and isoparaffenic hydrocarbon found in petrolatum are great occlusive agents, which can be delivered and slowly released to the skin by VAPs.

ii) Humectants

These are conditioning agents that regulate water levels on the skin and hair due to their hygroscopicity (ability to attract and bind water). They have the ability of re-hydrating the skin when delivered from a cream or lotion. These humectants can potentially be tagged onto the side chains of the VAPs to near saturation. By choosing VAPs with very high skin affinity, one can then design permanent organic humectants. Classical humectants include Glycerin, propylene glycol, butylene glycol, 1,3-butylene glycol, polyethylene glycols, sorbitol, sodium pyrrolidone carboxylate, acetamide MEA and many other miscellaneous humectants based on their water-absorbing characteristics (collagens, keratins, glucose esters and ethers, etc.).

iii) Emollients

Emollients are defined as: “agents, which when applied to a dry or inflexible corneum, will affect softening of that tissue by inducing re-hydration.” Esters and oils will induce re-hydration by reducing the loss of water in a similar fashion as occlusive agents listed above. Agents such as triglycerides (animal, vegetable and marine oils), lanolin and lanolin derivatives, simple esters, straight chain esters, fatty alcohol component variations, modified chain alcohols, branched chain esters (short branched chain alcohols, branched chain fatty alcohols) and complex esters can be delivered to the skin via VAPs.

iv) Conditioning Proteins and Amino Acids

Enzymes Used as Skin Conditioners

Fusion proteins can be built using VAPs fragments fused to catalytic fragments from various enzymes with application to skin care. Catalytic fragments from enzymes such as: superoxide dismutase (a superoxide radical scavenger, which may thereby function as an anti-inflammatory agent), papain (a protease, an alternative to AHAs as an exfoliant) and various others can now be anchored to the surface of the skin using VAPs properties. Fusion proteins have been previously documented in the literature and other prior arts.

Structural Proteins

Natural protein choices for the skin are proteins, which play a part in its structural make-up. Such proteins include: collagen and its versions, hydrolyzed elastin etc.

Modified VAPs

Quaternized protein hydrolysates have higher isoionic points, enhanced substantivity, and are capable of reducing irritation of anionic surfactants in cleansing formulations. Such examples are polytrimonium gelatin, polytrimonium hydrolyzed collagen etc. By adding polytrimonium groups to the surface side chains of all VAPs, one can reduce the skin irritation to chloroxylenol-based antiseptic cleansers and sodium lauryl sulfate.

By grafting fatty acid residues on primary amino groups of VAPs, these VAPs become film formers with various cosmetic advantages or even transport facilitators across bio-membranes for various active products listed in the document or others.

Amino Acids

Collagen peptides are highly hygroscopic and substantive to the skin. The small collagen-derived peptide chains can actually be slowly released after tagging such a peptide collagen molecule to a VAP and taking advantage of the proteolytic properties of various enzymes present on the surface of the skin. The slow release of individual collagen amino acids will provide the skin with low molecular weight hydrolysates without the tacky feel of higher molecular weight hydrolysates. Hydrolyzed wheat proteins can also be used as a source of amino acids. Wheat amino acids are of smaller size, allowing penetration of the skin's outer layer, in a manner similar to that observed for the hydrolyzed collagen.

In addition to the amino acids mentioned above, individual amino acids have specific effect. Tyrosine and derivatives are used in sun care products because of their involvement in skin coloration processes, synthetic and natural. Glycine derived from gelatin enriched with lysine enhances recovery of skin elasticity and de-pigmentation of age spots. Acetyl cysteine is an alternative to alpha-hydroxy acids (AHAs) for the removal of dead skin. It can be used to improve skin suppleness and smoothness and to treat acne. All these amino acids can be delivered by VAPs by creating fusion proteins with fragments made out of repetitions of these amino acids linked to VAPs. These fragments can be engineered to be susceptible to proteolytic cleavage from endogenous skin enzymes.

Organo-Modified Siloxane Polymers

Organo-modified siloxane polymers are derived from chlorosilane monomers via hydrolysis and polymerization and/or polycondensation. They include (poly) dimethylcyclosiloxanes, linear (poly) dimethylsiloxanes, cross linked (poly) dimethylsiloxanes, and other functional siloxanes.

Specialty Silicones

These silicones have their organo-functional groups modified to limit their de-foaming properties, increase their solubility, make them less greasy, and decrease their need for emulsification for water-based systems. They include: dimethiconol, dimethycone copolyol, alkyl dimethicone copolyol, trimethylsilylamodimethicone, amodimethicone, dimethicone copolyol amine, silicone quartenium compounds, silicone esters, etc.

Cationic Surfactants and Quaternary Derivatives

By general definition, quaternary ammonium salts are “a type of organic compound in which the molecular structure includes a central nitrogen atom joined to four organic groups as well as an acid radical.” These quaternary derivatives can be based on fatty acids, proteins, sugars and silicone polymers. They include amines, amphoteric surfactants, amine oxides, amidoamines, alkylamines, alkyl imidazolines, ethoxylated amines, quaternary salts, and others.

It was reported in various previous research findings that quatemized proteins are useful in treating damaged keratinous surfaces (l'Oreal, U.S. Pat. No. 4,796,646). One could therefore genetically engineer a VAP to be quatemized by first adding as many amino acids with NH₃ side chains on the outer surface of the scaffold and/or at the tail end, and then quatemizing these side chains using techniques illustrated in the literature.

Polymers as Conditioning Agents

These polymers include cationic and non-cationic polymers. The adsorption of a cationic polymer shows a sharp initial uptake followed by a slow approach to equilibrium. The mechanism appears to involve slow penetration of the skin by the polymer, since the uptake by the polymer far exceeds that of a monolayer. Skin keratin is very reactive to these polymers. Tagging these polymers onto VAPs ensures constant replenishing of the outer skin layer, which is constantly in a state of sloughing.

These polymers include:
Cationic conditioning polymers such as polyvinylpyrrolidone (PVP), copolymers of PVP (PVP/Vinyl acetate copolymers, PVP-α-olenin copolymers, PVP/Methacrylate copolymers etc.), Dimethyl Diallyl Ammonium Chloride (DDAC) homopolymer (Polyquaternium 6), DDAC/Acrylamide (polyquaternium 7), DDC/Acrylic acid (polyquaternium 22), polymethacrylamideopropyl trimonium chloride, acrylamide/β-methacryloxyethyltrimethyl ammonium methosulfate (polyquaternium 5), adipic acid/dimethylaminohydroxypropyl/diethylenetriamine copolymer, vinyl aldohol hydroxypropyl amine (polyquaternium 19), quaternized polyvinyl octadecyl ether (polyquaternium 20), quaternized ionenes (polyquaterniums 2, 17, 18, 27), polyquaternium 8, Cellulose cationic polymers (such as hydroxypropyl trimethyl ammonium chloride ether of hydroxyethyl cellulose (polyquaternium 10), polyquaternium 24, hydroxyethylcellulose/diallyldimethyl ammonium chloride (polyquaternium 4), alkyl dimonium hydroxypropyl oxyethyl cellulose), polyquaternium 46, cationic polysaccharides such as guar hydroxypropyl trimonium chloride, quaternized lanolin (quaternium 33), quaternized CHITOSAN (polyquaternium 29);
Quaternized Proteins can also be designed or constructed to be included as additional fragments linked to the VAP. Such proteins can include quaternized collagens, quaternized keratin, quaternized vegetable proteins, quaternized wheat protein, quaternized soy proteins, hydrogenated soy proteins;
Aminosilicones can also be used for skin cleansing preparations since they interact with skin proteins to provide a re-fatting effect, imparting a smooth and supple feel to the cleansed skin.

Anti-Ageing Proteins and Vitamins

Whey protein was found to activate cytokines (immunological regulators, signaling and controlling molecules in cell-regulating pathways). Synthesis of a fusion protein composed of the activating portion of the whey protein along with the VAP will improve skin firmness, touch, smoothness and will increase skin elasticity and thickness. Anti-ageing vitamins such as Vitamin D, vitamin A (retinol) or other vitamins tagged to VAPs.

VAPs can present a stable and long-lasting alternative for the delivery of beneficial enzymes and more specifically catalytic portions of these enzymes. Fusion proteins can therefore be synthesized which will have affinity for skin and at the same time contain the catalytic portion of the beneficial enzyme. The intentional delivery of these active principles (usually in the form of a catalytic portion of an enzyme, hormone or specific peptide as well as many other materials) to living cells for cosmetic purposes, with the aim of providing noticeably “improved” skin texture and topography will be included in this patent. As an illustration, VAP linked to a catalytic fragment of serine protease would be a method to deliver lastingly an agent that will provide skin-smoothing effects.

f) Hair Conditioning Agents

i) Hair “Repair” Agents

Hair damage results from both mechanical and chemical trauma that alters any of the physical structures of the hair. Conditioning agents cannot enhance repair, since repair does not occur, but can temporarily increase the cosmetic value and functioning of the hair shaft until removal of the conditioner occurs with cleansing. VAPs can provide a slow release mechanism and a vehicle for the delivery of these conditioning agents while enhancing their resistance to washing. Potentially, these conditioners can now resist normal shampooing conditions thanks to these VAPs and hence, provide a constant stable conditioning effect to the hair cuticle onto which they are anchored.

These conditioning agents, which are tagged to the VAPs, cannot only include chemicals listed above but they also may fall in the categories listed below.
Immobilized enzyme-VAPs fusion proteins applied to damage hair to remove frayed cuticle, thereby enhancing shine, much as Cellulases are used in prior art to remove fuzz from cotton clothing during laundering and thereby brightening colors.
Modified amino acids on the VAPs targeted to keratin capable to convert cystic acid residues to moieties capable of forming disulfide bonds, thereby allowing strengthening of hair in a much gentler fashion than current methods.
Treatment for greasy hair (seborrheic conditions): the cause for such conditions is usually the disintegration of sebaceous cells (holocrine secretion). This holocrine secretion will subsequently release a sterile mixture of lipids (triglycerides (60%), wax esters of fatty acids and long chain fatty alcohols (20 - 25%); and squalene (15%)) into the pilosebaceous duct, already containing protein and lipids and inhabited by normal skin flora. This population is mainly engaged in enzymatic activity, directly toward the triglycerides releasing fatty acids. Once excreted onto the surface, the modified sebum will mix with another of lipid emanating from epidermal cells, free cholesterol, cholesterol esters, glycerides etc.
VAPs coupled with enzymatic fragments from a variety of enzymes involved in metabolism of fatty acids (notably saturated chain fatty acids) such as acyl CoA synthase etc. could present a cure to this problem.
Other methods involve using sulfur containing amino acids and thioethers linked to VAPs or VAPs engineered to contain high proportions of these amino acids (S-carboxymethyl, S-benzhydryl, S-trityl cysteine, 4-thiazoline carboxylic acid, N-acetyl homocysteine thiolactone, thioethers derived from cysteamine, glutathione and pyridoxine, tiolanediol and oxidation derivatives etc.
Substances retarding sebum recovery: this method involves the design of VAPs geared towards the slow down of sebum's uptake by hair by depositing an oleophilic film on the surface of the cuticle. By designing a very hydrophobic and lipophobic VAP, one can retard the sebum transfer from scalp to hair.
VAPs designed to be grease absorbers. These VAPs will mimic properties of gelatin or casein to absorb sebum and give it a more waxy consistency in order to make the seborrheic state less obvious.

Design of VAPs to treat dry hair caused by trauma from over-vigorous mechanical or chemical treatments. A further factor in the frailty of hair is weathering from the cumulative effect of climatic exposure, namely sunlight, air pollutants, wind, seawater and spindrift or chlorinated water. These physiochemical changes can be defined and may be measured by a loosening of cuticle scales and increase in the friction coefficient, increase in porosity, a tendency for the hair to break more easily due to disruption of salt and cysteine linkages, hydrogen bonds, sulfur content and degradation in polypeptide chains leading to the elimination of oligoproteins. VAPs will help treat dry hair by providing a vehicle for a timely release of amino acids and other microelements it has lost and in essence, restoring its biochemical balance. VAPs linked fatty elements will be a logical step in combating hair dryness. These peptides will be linked with compounds such as:
Fatty acids
Fatty alcohols
Natural triglycerides
Natural waxes
Fatty esters
Oxyethylenated or oxypropylenated derivatives of waxes, alcohol, and fatty acids
Partially saturated fatty alcohols
Lanolin and its derivatives Other agents are listed and will help restore hair to its original biochemical structure.

In general, all conditioning agents for all different forms of keratin can be tagged to this VAP carrier. The conditioning agents can be chosen from the following:
Amino acids (e.g., cysteine, lysine, alanine, N-phenylalanine, arginine, Glycine, leucine, etc.), oligopeptides, peptides, hydrolyzed or not (modified or unmodified silk or wool hydrolyzed proteins, hydrolyzed wheat proteins etc.), modified or unmodified;
Fatty acids (carboxylic acids from C₈-C₃₀, such as palmitic, oleic, linoleic, myristic, stearic, lauric, etc. and their mixture) or fatty alcohols, branched or unbranched (C₈-C₃₀ fatty alcohols such as: palmitic, myristic, stearic, lauric etc. and their mixture);
Vegetable, animal or mineral fats and their mixture;
Ceramides (e.g., Class I, II, III, and IV according to Downing's classification such as: 2-N-linoleoylamino-octadecane-1,3-diol, 2-N-oleoylamino-octadecane-1,3-diol, 2-N-palmitoylamino-octadecane-1,3 -diol, 2-N-stearoylamino-octadecane-1,3-diol, 2-N-behenylamino-octadecane-1,3-diol etc.) and their mixture and the pseudo-ceramides and their mixture;
Hydroxylated organic acids (e.g., citric acid, lactic acid, tartaric acid, malic acid and their mixture);
UV filters (e.g., UV-A and/or UV-B known for a person trained in the art. They can include derivatives of dibenzoylmethane, p-amino benzoic acid and its esters, salicylic acid salts and their derivatives, Cinnamic acid esters, benzotriazole derivatives, triazine derivatives, derivatives ofβ, β′-diphenylacrylate 2-phenylbenzimidazole-5-sulfonic acid and its salts, derivatives of benzophenone, derivatives of benzylidene-camphor, silicone filters etc. and their mixtures);
Anti-oxidants and free radicals scavengers (such as ascorbic acid, ascorbyl dipalmitate, t-butylhydroquinone, polyphenols such as phloroglucinol, sodium sulfite, erythorbic acid, flavonoids, and their mixture);
Chelating agents (EDTA and its salts, di-potassium EDTA, phosphate compounds such as sodium metaphosphate, sodium hexametaphosphate, tetra potassium pyrophosphate, phosphonic acids and their salts, etc. and their mixtures);
Regulating agents for seborrhea: succinylchitosane, poly-b-alanine, etc. and their mixture;
Anti-dandruff agents: the invention can include many agents from the following compounds: benzethonium chloride, banzalkonium chloride, chlorexidine, chloramines T, chloramines B, 1,3-dibromo-5,5dimethylhydantoine, 1,3-dichloro-5,5,-dimethylhydantoine, 3-bromo 1 chloro 5,5-dimethylhydantoine, N-chlorosuccinimide, derivatives of 1-hydroxy-2-pyridone, trihalohenocarbamides, triclosan, azole derivatives, antifungal derivatives such as amphotericine B or nystatine, sulfur derivatives of selenium, sulfur in its different forms, physiologically tolerated acid salts such as nitric, thiocyanic, phosphoric, acetic etc. and various other agents and their mixture);
Cationic tenso-cosmetic agents: primary, secondary, tertiary fatty amine (polyoxyalkylanated) salts, quaternary ammonium salts, imidazoline salts, cationic amino oxides, etc. and their mixture;
Amphoteric polymers;
Organically or non-organically modified silicones;
Mineral, vegetable or animal oils;
Esters;
Poly-isobutenes and poly-(α-olefin);
Anionic polymers;
Non-ionic polymers;

These compounds can be attached to the VAP individually or mixed together.

ii) Hair Perming Agents

One preferred example of the invention is a non-aggressive perming agent that is formed by a bi-valent VAP that has specificity for hair surface proteins, thus where a hair-specific VAP is in fact the delivery agent for a second VAP. The bi-valency would result in cross linking activity and gives the hair a permanent wave look and more substance feel or, when flexible spacers are used to make the VAP a bi-valent molecule, provide a more gelling agent feel. Many modern hair shampoos, conditioners or other forms of hair treatments already contain 0.5-3% proteinaceous material or protein hydrolysates of natural origin such as from plants for the purpose of hair protection, providing free amino acids and substance. Bi-valent VAPs with hair surface specificity would not have a tendency to be rinsed readily off such as the non-specific proteinaceous materials.

g) Hair Coloring Agents

Hair dye products come in three classes; permanent, semi-permanent and temporary dyes. The latter can be rinsed out instantly. The permanent dyes can be sub-divided into

(1) oxidation hair dye products and

(2) progressive hair dyes.

Oxidation hair dye products consist of dye intermediates and a solution of hydrogen peroxide. An example of dye intermediates is p-phenylenediamine which form hair dyes on chemical reaction. 2-nitro-p-phenylenediamine is another type of dye intermediates. They are already dyes and are added to achieve the intended shades. The dye intermediates and the hydrogen peroxide solution, often called the developer, are mixed shortly before application to the hair. The applied mixture causes the hair to swell and the dye intermediates penetrate the hair shaft to some extent before they have fully reacted with each other and the hydrogen peroxide in an oxidative condensation reaction and thus forming the hair dye. The necessarily high pH (9-10) is usually achieved through the addition of ammonia.

The active ingredient for progressive hair dye products is typically lead acetate. The most noticeable difference between oxidation and progressive hair dyes is that progressive dyes are intended to give a more gradual change in hair color.

In general, the permanent hair dyes are sensitive to UV light from which they are shielded by the keratin hair shaft. They have a quite limited spectrum of color options and gradually loose their intensity after the hair dye process. The molecules bleach and leak out of the hair during subsequent washings.

The semi-permanent dyes are more complex benzene derivatives that are weakly bound directly to the hair surface and usually administered via coal-tar carriers. The size exclusion of the hair shaft prevents deeper binding sites inside the hair. The colors that can be formed with the semi-permanent dyes cover a wider spectrum and some have more intense primary color characteristics, they are less sensitive to UV light as the permanent dyes. A major disadvantage of the semi-permanent dyes is that the binding to the hair surface is relatively weak and can be rinsed out more easily than the permanent hair dyes.

The coloring substances used in the invention included water-soluble dyes (light green SF yellow, patent blue NA, naphthol Green B, Eosine YS and the like) and water-insoluble colorants such as lakes (naphthol blue black-aluminum salt, alizurol-aluminum salt and the like), organic pigments (brilliant fast scarlet, permanent red F5R, lithol red, deep maroon, permanent red or orange, benzidine yellow G and the like) and natural coloring matter (capsanthin, chlorophyll, riboflavin, shisonin, brazillian, and the like), in addition to titanium oxides, iron oxides and magnetic particles. They can also be fluorescent, phosphorescent or luminescent dyes. Fairly complete listings of hair coloring substances can be found, e.g., in U.S. Pat. No. 5,597,386, but the present invention is not limited to the currently known coloring substances.

There are a number of reasons why, different or less frequently used color substances can now be applied in a more favorable way for hair coloring:
a) when low molecular weight water-insoluble dyes are coupled to the very hydrophilic VAPs, the water-solubility of the complex may effectively make the coloring substance now water-soluble.
b) the more UV-stable semi-permanent dyes are preferred for surface colorations, thus providing the option to use the wider color gamma of semi-permanent dyes or even the temporary dyes in a now more permanent application.

Dye mixtures can be used when coupling to VAPs when common coupling procedures can be used, or the VAPs/dye complexes can be mixed to achieve required shades. Binding affinity strength provides an additional way to control the performance of the dye treatment over time.

Other advantages of this invention for hair coloring products include:
the whole hair-dying process can now be performed under much more benign chemical conditions as there is no need for dimerization inside the hair shaft using oxidative treatments
In comparison with the permanent dye procedures, there will be no need for the expensive additives that are used to create the strong oxidative conditions
the binding will be more permanent as a result of the relatively strong affinity body to ligand binding characteristics
specific shampoos can be developed easily to remove the dye from the hairs again; analogous to methods that dissociate the affinity body to ligand binding that are well known from affinity chromatography procedures, such as a shift in pH or high salt treatments, again relatively benign chemical treatments that are non-damaging to the hair structure. This part of the invention is further described in the selection procedures for hair-specific VAPs selections.

Due to the relative small size of VAPs compared to antibodies, favorable ratios of VAP/dye substance can be obtained, providing both sufficient binding activity and color effect. The color intensity per unit VAPs will depend on the particular dye, background hair color etc. but the coupling of dyes to VAPs is flexible and allows a wide range of ratios. Also molecular modifications of the VAP can be used to increase the number of dye labeling sites; also pre-labeled peptides or other polymeric strands can be bound to VAPs. Furthermore, as the VAPs-mediated hair dyes as described in the present invention have a high and specific affinity for hair, the actual hair coloring process is much more efficient than with conventional hair dyes, where a substantial amount of dye material is lost directly with the first rinse. Also, with conventional dyes, concentration of the coloring compounds can become so high that they cause skin irritation or skin coloring or, in order to prevent these effects, dye concentrations are so low that repeated treatment is necessary before the required hair shade is reached. A much more precise treatment effect can be obtained with dye-charged VAPs. The coloring substances can be tagged on the VAP using functional groups on the macro-carrier such as amino groups, carboxyl groups, aldehyde groups, hydroxyl groups, thiol groups and the like.

In manufacturing the aforementioned Dye-VAP molecule, ratios of the VAP to the coloring substances can be changed so that their ratios can be adjusted to obtain desired proportions of their components. Depending on the color intensity desired, the VAP can be synthetically designed to include side groups, which will optimize the binding of the dyes. The weight ratio of the coloring substance to VAP will therefore be dependent on the final color intensity desired and the artificially designed chemical nature of the VAP side chains.

h) VAP-Loaded on Encapsulation Devices as Skin or Hair Conditioning Agents

Besides direct application of VAPs coupled with fragrances, colors conditioning agents and the like, the VAPs can easily be applied as an intricate part of a vesicle. The use of vesicles is widely known in the art and may include but are not limited to liposomes, oilbodies, polyethylene glycol micelles, sodium acrylates co-polymers in caprylic/capric triglyceride and water (e.g., Luvigel, BASF, Germany), nanoparticles (a phospholipid monolayer with a hydrophobic center), starch (nano)particles (WO 0069916, WO 0040617) etc. Vesicle-enhanced formulations can offer protein stabilization, prevention of oxidation, increased solubilization of normally recalcitrant compounds and targeted delivery of active ingredients. When charging such vesicles with VAPs, e.g., by addition of a hydrophobic tail region on either the carboxy- or the amino terminus of the VAP and mixing purified VAPs with the vesicles, the vesicles themselves become multivalent and can be used as improved delivery agents for hair and skin applications. Examples of such non-polar hydrophobic tails are known in the art, such as a polyleucine.

i) Release of Cosmetic Agents from VAPs

Release of the cosmetic agents from the VAPs moieties can either be dependent on a secondary formulations which will trigger conditions ideal for decoupling of the agents or on inherent skin and hair physiological conditions i.e. increase in temperature and decrease in pH through exercise, natural skin fauna secretions or even endogenous skin enzymes. Again, bonding of the peptide to the cosmetic agents should also be optimized for such conditional releases via chemical modification of both peptide VAPs and cosmetic agents.

In a healthy person, the internal tissues, e.g. blood, brain, muscle, etc., are normally free of microorganisms. On the other hand, the surface tissues, e.g. skin and mucous membranes, are constantly in contact with environmental organisms and become readily colonized by certain microbial species. The mixture of organisms regularly found at any anatomical site is referred to as the normal flora.

i) Skin Enzymes

The enzymes accumulated in the stratum granulosum and in lamellar granules involved in protein cleavage and/or activation (e.g. profilagrin, involucrin) are profilagrin endopeptidase (K. A. Resing et al., Biochemistry 32:10036-9, 1993), transglutaminase family members (M. Akiyama et al., Br. J. Dermatol. 146:968-76, 2002), cathespin B, C, D, H and L (H. Tanabe et al., Biochim. Biophys. Acta. 1094:281-7, 1991; T. Horikoshi et al., Br. J. Dermatol. 141:453-9, 1999; D. J. Tobin et al., Am. J. Pathol. 160:1807-21, 2002), proprotein convertases (PC) furin, PACE4, PC5/6 and PC7/8 (D. J. Pearton et al., Exp. Dermatol. 10:193-203, 2001), a chymotrypsin-like enzyme (T. Egelrud, Acta. Dermatol. Venerol. Supp. 208:44-45, 2000), two trypsin-like serine proteinases (M. Simon et al., J. Biol. Chem. 276:20292-99, 2001)), and stratum comeum thiol protease (A. Watkinson, Arch. Dermatol. Res. 291:260-8, 1999). The hydrolytic enzymes released from the lamellar granules into the intercellular space comprise acid phosphatase, acid lipase, sphingomyelinase, glucosidase and phospholipase A (S. Grayson et al., J. Invest. Dermatol. 85:289-94, 1985; R. K. Freinkel et al., J. Invest. Dermatol. 85:295-8, 1985). Several enzymes are present on the outer surface of the skin, probably involved in maintenance activities for the Stratum Comeum (SC); permeability barrier homeostasis, extracellular lipid processing, SC integrity and cohesion, desquamation and the like. Examples are the serine protease kallikrein, lysozyme (Ric. Clin. Lab. 1978, 8(4):211-31) or the newly discovered phospholipase (Br. J. Dermat. 2000, 142(3): 424-31, BBRC 2002, 295(2):362-9). The localization and the concentration of these enzymes vary greatly in different regions of the body. These enzymes encompass groups such as dehydrogenases, acid phosphatases, esterases, peptidases, phosphorylases and lipases amongst others (Stevens et al., Int. J. Dermatology, 1980 Vol. 19 No. 6 p 295) and, therefore, diverse release mechanisms can be applied when cosmetic substances are delivered via VAPs. These enzymes can also be used to deliver a skin benefit via the interaction of the VAP-benefit agent with the enzyme. These VAP attached to the cosmetic agents thus become enzyme-linked benefits with inherent slow release mechanisms.

Furthermore, endogenous hair fiber enzymes have not only been shown to be present, but also to be biologically active. Maturation of hair fiber results in the death of its constituent cells (Tamada et al. (1994) Br. J. Dermatol. 131) and this coupled with the increased levels of intracellular cross-linking results in a mature fiber, which is metabolically dead. Unexpectedly, the authors have found that enzyme activity is in fact preserved, rather than denatured, during the process of cellular keratinization and death that occur during fiber growth. Examples of active enzymes identified to date within the mature human fiber include transglutaminase, protease, lipase, steroid sulphatase, catalase and esterase. Ingredients suitable for use as benefiting agents for targeting hair fiber enzymes are any VAP bound to the cosmetic agents and capable of specifically interacting with the enzyme. The bond, which links cosmetic agent to VAP must ideally be recognized by the enzyme as a substrate.

ii) Microflora-Derived Enzymes

The normal flora of humans is exceedingly complex and consists of more than 400 species of bacteria and fungi. The makeup of the normal flora depends upon various factors, including genetics, age, sex, stress, nutrition and diet of the individual. The normal flora of humans consists of a few eukaryotic fungi and protists, and some methanogenic Archaea that colonize the lower intestinal tract, but the bacteria are the most numerous and obvious microbial components of the normal flora, mostly present on the skin surface. Bacteria common on skin surface include Staphylococcus epidermis, Staphylococcus aureus, Streptococcus pyogenes, Corynebacterium diphtheriae, Micrococcus luteus and Propionibacterium acnes. Examples of fungi growing on human skin are Malassezia furfur, Pityriasis versicolor, Malassezia folliculitis, Candida albicans, Trycophyton, Microsporum and Epidermophyton. The adult human is covered with approximately two square meters of skin. The density and composition of the normal flora of the skin vary with anatomical locale. The high moisture content of the axilla, groin, and areas between the toes supports the activity and growth of relatively high densities of bacterial cells, but the density of bacterial populations at most other sites is fairly low, generally in 100s or 1000s per square cm. Qualitatively, the bacteria on the skin near any body orifice may be similar to those in the orifice. The majority of skin microorganisms are found in the most superficial layers of the epidermis and the upper parts of the hair follicles. These are generally nonpathogenic and considered to be commensal, although mutualistic and parasitic roles have been assigned to them. Sometimes potentially pathogenic Staphylococcus aureus is found on the face and hands, particularly in individuals who are nasal carriers. Nails are common host tissues for fungi such as Aspergillus, Penicillium, Cladosporium, Mucor. The bacteria and other components of the natural skin flora are known to secrete a battery of hydrolytic enzymes as a mechanism of defense against pathogens and antagonistic bacteria. These hydrolytic enzymes can be used to slowly release the cosmetic agents herein mentioned.

iii) Physical Release Conditions

Non-enzymatic conditions for release of fragrances can be induced by pH-changes such as a pH-decrease on the skin resulting from sweat, both from the eccrine glands and the apocrine glands (Exog. Dermatol. 2002, 1:163-175).

### EXAMPLES

### Example 1

### Determination of Core Coordinates

Immunoglobulin-like (ig-like) folds are very common throughout nature. Many proteins, especially in the animal kingdom, have a fold region within the protein that belongs to this class. Reviewing the function of the proteins that contain an ig-like fold and reviewing the function of this ig-like fold within that specific protein, it is apparent that most of these domains, if not all, are involved in ligand binding. Some examples of ig-like fold containing proteins are: V-CAM, immunoglobulin heavy chain variable domains, immunoglobulin light chain variable domains, constant regions of immunoglobulins, T-cell receptors, fibronectin, reovirus coat protein, beta-galactosidase, integrins, EPO-receptor, CD58, ribulose carboxylase, desulphoferrodoxine, superoxide likes, biotin decarboxylase and P53 core DNA binding protein. A classification of most ig-like folds can be obtained from the SCOP database (Murzin A. G. et al., J. Mol. Biol., 247, 536-540, 1995; http://scop.mrc-lmb.cam.ac.uk/scop) and from CATH (Orengo et al., Structure, 5(8), 1093-1108, 1997; http://www.biochem.ucl.ac.uk/bsm/cath_new/index.html). SCOP classifies these folds as: all beta-proteins, with an immunoglobulin-like beta-sandwich in which the sandwich contains seven strands in two sheets although some members that contain the fold have additional strands. CATH classifies these folds as: mainly beta-proteins with an architecture like a sandwich in an immunoglobulin-like fold designated with code 2.60.40. In structure database like CE (Shindyalov et al., Protein Engineering, 11(9), 739-747, 1998; http://cl.sdsc.edu/ce.htm), VAST (Gibrat et al., Curr. Op. Struc. Biol., 6(3), 377-385, 1996; http://www.ncbi.nlm.nih.gov/Structure/VAST/vast.shtml) and FSSP (Holm et al., Nucl. Acids Res., 26, 316-319; Holm et al., Proteins, 33, 88-96, 1998; http://www.ebi.ac.uk/dali/fssp) similar classifications are used.

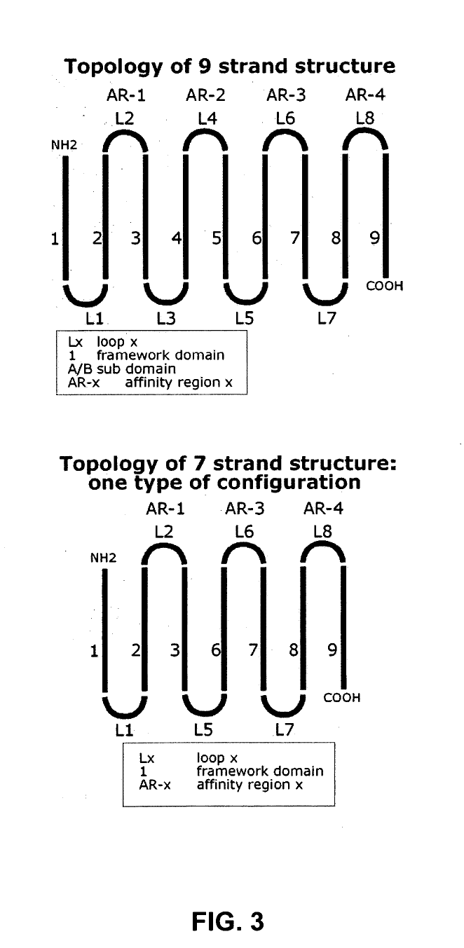

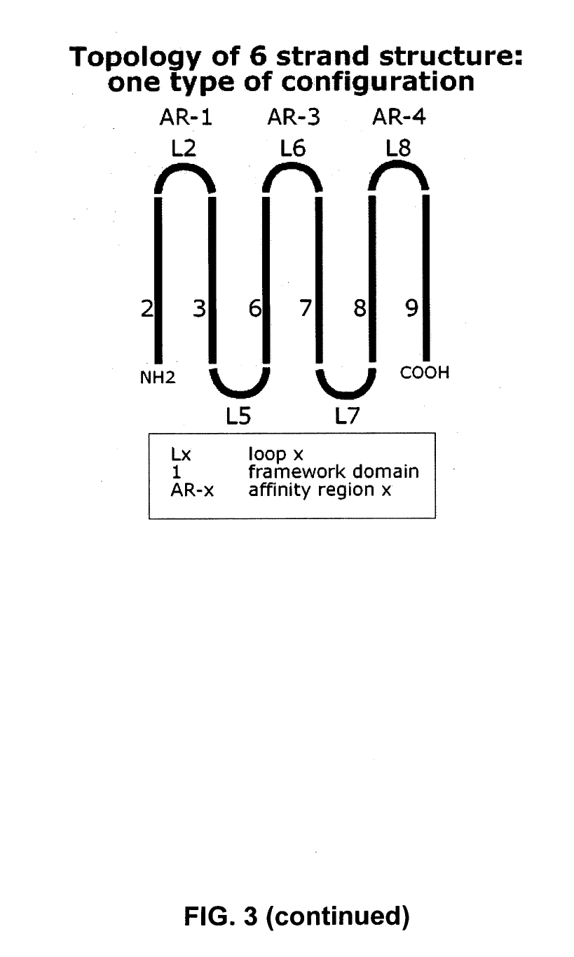

Projection of these folds from different proteins using software of Cn3D (NCBI; http://www.ncbi.nlm.nih.gov/Structure/CN3D/cn3d.shtml), InsightII (MSI; http://www.accelrys.com/insight) and other structure viewers, showed that the ig-like folds have different sub-domains. A schematic projection of the structure is depicted in FIG. 3A. The most conserved structure was observed in the center of the folds, named the core. The core structures hardly vary in length and have a relative conserved spatial constrain, but they were found to vary to a large degree in both sequence and amino acid composition. On both sides of the core, extremely variable sub-domains were present that are called connecting loops. These connecting loops can vary in amino acid content, sequence, length and configuration. The core structure is therefore designated as the far most important domain within these proteins. The number of beta-elements that form core can vary between seven and nine although six-stranded core structures might also be of importance. The beta-elements are all arranged in two beta-sheets. Each beta-sheet is built of anti-parallel beta-element orientations. The minimum number of beta-elements in one beta-sheet that was observed was three elements. The maximum number of beta-element in one sheet that was observed was five elements. Higher number of beta-elements might be possible. Connecting loops connect the beta-elements on one side of the barrel. Some connections cross the beta-sheets while others connect beta-elements that are located within one beta-sheet. Especially the loops that are indicated as L2, L4, L6 and L8 are used in nature for ligand binding. The high variety in length, structure, sequences and amino acid compositions of the L1, L3, L5 and L7 loops clearly indicates that these loops can also be used for ligand binding, at least in an artificial format.

Amino acid side chains in the beta-elements that form the actual core that are projected towards the interior of the core and thus fill the space in the center of the core, can interact with each other via H-bonds, covalent bonds (cysteine bridges) and other forces, to stabilize the fold. Because amino acid composition and sequence of the residues of the beta-element parts that line up the interior were found to be extremely variable it was concluded that many other formats can also be created.

In order to obtain the basic concept of the structure as a starting point for the design of new types of proteins containing this ig-like fold, projections of domains that contain ig-like folds were used. Insight II, Cn3D and modeller programs were used to determine the minimal elements and lengths. In addition, as amino acid identities were determined as not of any importance, only C-alpha atoms of the structures were projected because these described the minimal features of the folds. Minor differences in spatial positions (coordinates) of these beta-elements were allowed. Four examples of such structures containing nine beta-elements were determined and converted into PDB formats (coordinate descriptions; see Table 1) but many minor differences within the structure were also assumed to be of importance, as long as the fold according to the definitions of an ig-like fold (see, e.g., CATH and SCOP).

These PDB files representing the coordinates of the C-alpha atoms of the core of ig-like folds were used for the development of new 9, 8, 7 and 6 beta-elements containing structures. For eight-stranded structures, beta-element 1 or 9 can be omitted but also elements 4 or 5 can be omitted. For seven-stranded structures, beta-elements 1 and 9 were removed or, preferably, elements 4 and 5 were omitted. The exclusion of elements 4 and 5 is preferred because of spatial constrains (FIG. 3B). Six-stranded structures lack preferably element 1, 4 and 5 or 4, 5 and 9 but also other formats were analyzed with Insight and modeller and shown to be reliable enough for engineering purposes (FIG. 3C).

### Example 2

### Design of 9 Strands Folds

Protein folding depends on interaction between amino acid backbone atoms and atoms present in the side chains of amino acids. Beta-sheets depend on both types of interactions while interactions between two beta-sheets, for example, in the above-mentioned structures, are mainly mediated via amino acid side chain interactions of opposing residues. Spatial constrains, physical and chemical properties of amino acid side chains limit the possibilities for specific structures and folds and thus the types of amino acids that can be used at a certain location in a fold or structure. To obtain amino acid sequences that meet the spatial constrains and properties that fit with the 3D structure of the above described structures (Example 1), 3D analysis software (Modeller, Prosa, InsightII, What if and Procheck) was used. Current computer calculation powers and limited model accuracy and algorithm reliabilities limit the number of residues and putative structures that can be calculated and assessed.

To obtain an amino acid sequence that can form 9-beta-strand folds as described above, different levels of testing are required, starting with a C-alpha backbone trace as described in, for example, PDB file 1. First the interior of the fold needs to be designed and tested. Secondly, beta-element connecting loops need to be attached and calculated. Thirdly exterior amino acids, i.e., amino acids that expose their amino acid side chains to the environment, need to fit in without disturbing the obtained putative fold. In addition, the exterior amino acid side chains should preferably result in a soluble product. In the fourth and last phase the total model is recalculated for accidentally introduced spatial conflicts. Amino acid residues that provoked incompatibilities are exchanged by an amino acid that exhibits a more accurate and reliable fit.

In the first phase, amino acid sequences aligning the interior of correctly folded double beta-sheet structures that meet criteria as described above and also in Example 1, were obtained by submitting PDB files for structural alignments in, e.g., VAST (http://www.ncbi.nlm.nih.gov/Structure/VAST/vast.shtml). The submission of the PDB files as depicted in PDB file 1 already resulted in thousands of hits. The majority of these proteins were proteins that contained at least one domain that would be classified according to SCOP or CATH (see above) as folds meant here.

Several unique sequences aligning the interior of the submitted structure were used for the generation of product examples. Interesting sequences from this structural alignment experiment were selected on criteria of classification, rootmean square deviations (RMSD-value), VAST-score values (higher values represent more accurate fit), sequence identities, origin of species and proposed biological function of the hits. Structures as fibronectin-like protein, antibody related proteins, cell adhesion molecules, virus core proteins, and many others. The structures that are represented by the C-alpha backbones are called the core structures.

In the second phase, loops were attached to obtained products. Although several analysis methods can be applied that resolve the structure of the end products, the most challenging feature would be the presentation of affinity regions on core sequences that have full functional ligand binding properties. In order to test the functionality of the end products, affinity loops that recognize known ligands can be transplanted on the core structure. Because anti-chicken lysozyme (structure known as 1 MEL) is well documented, and the features of these affinity regions (called CDRs in antibodies) are well described, these loops were inserted at the correct position on core sequences obtained via the method described in the first phase. Correct positions were determined via structural alignments, i.e., overlap projections of the already obtained folds with the file that describes the 3D structure of 1MEL (PDB file; example). Similar projections and subsequent loop transplantations were carried out for the bovine RNase. A binding affinity region that were extracted from the structure described by 1 BZQ (PDB). The transplanted affinity loops connect one end of the beta-elements with one other. Affinity region 1 connects beta-element 2 with 3 (L2), AR2 connects beta-element 4 and 5 (L4), AR3 connec beta-elements 6 and 7 (L6) and AR4 connects beta-elements 8 and 9 (L8). The other end of each of the beta-elements was connected by loops that connect element 1 with 2 (L1), 3 with 4 (L3), 5 with 6 (L5) and 7 with 8 (L7) respectively (see schematic projection in FIG. 3A). Of course all kinds of loops can be used to connect the beta-elements. Sources of loop sequences and loop lengths encompass, for example, loops obtained via loopmodeling (software) and from available data from natural occurring loops that have been described in the indicated classes of, for example, SCOP and CATH. C-alpha backbones of loops representing loops 1 (L1), 3 (L3), 5 (L5) and 7 (L7; FIG. 3A) were selected from structures like, for example, INEU, IEPF-B, 1QHP-A, 1CWV-A, 1EJ6-A, 1E50-C, 1MEL, 1BZQ and lF2X, but many others could have been used with similar results. 3D-alignments of the core structures obtained in the first phase as described above, together with loop positions obtained from structural information that is present in the PDB files of the example structures 1EPF, 1NEU, 1CWV, 1F2X, 1QHP, 1E50 and 1EJ6 were realized using powerful computers and Cn3D, modeller and/or Insight software. Corresponding loops were inserted at the correct position in the first phase models. Loops did not have to fit exactly on to the core because a certain degree of energy and/or spatial freedom can be present. The type of amino acids that actually will form the loops and the position of these amino acids within the loop determine this energy freedom of the loops. Loops from different sources can be used to shuffle loop regions. This feature enables new features in the future protein because different loops have different properties, like physical, chemical, expressional, post translational modifications, etc. Similarly, structures that contain less loops due to reduced numbers of beta-elements can be converted into proteins with nine beta-elements and a compatible number of loops. Here it is demonstrated that the C-alpha trace backbones of the loops of seven-stranded proteins like, for example, 1EPF, 1QHP, 1E50 and 1CWV could be used as templates for nine-stranded core templates. The additional loop (L3) was in this case retrieved from the nine-stranded template 1F2X but any other loops that were reliable according to assessment analysis could also have been used. The nature of the amino acids side chains that are pointing to the interior of the protein structure was restricted and thus determined by spatial constrains. Therefore several but limited configurations were possible according to 3D-structure projections using the modeling software.

In the third phase, all possible identities of amino acid side chains that are exposed to the exterior, i.e., side chains that stick out of the structure into the environment, were calculated for each location individually. For most applications, it is preferred to use proteins that are very good soluble and therefore amino acids were chosen that are non-hydrophobic. Such amino acids are, for example, D, E, N, Q, R, S and T. Methionine was preferably omitted because the codon belonging to methionine (ATG) can result in alternative protein products due to aberrant translational starts. Also, cysteine residues were omitted because free cysteines can lead to cysteine-cysteine bonds. Thus, free cysteines can result in undesired covalent protein-protein interactions that contain free cysteines. Glycine residues can be introduced at locations that have extreme spatial constrains. These residues do not have side chains and are thus more or less neutral in activity. However, the extreme flexibility and lack of interactive side chains of glycine residues can lead to destabilization and therefore glycine residues were not commonly used.

In the fourth phase, the models were assessed using modeller. Modeller was programmed to accept cysteine-cysteine bridges when appropriate. Next all predicted protein structures were assessed with Prosall (http://www.came.sbg.ac.at/Services/prosa.html), Procheck and What if (http://www.cmbi.kun.nl/What if). ProsaIl zp-comb scores of less then −4.71 were assumed to indicate protein sequences that might fold in vivo into the desired beta-motif. The seven protein sequences depicted in Table 1 represent a collection of acceptable solutions meeting all criteria mentioned above. Procheck and What if assessments also indicated that these sequences might fit into the models and thus as being reliable (e.g., pG values larger than 0.80; Sánchez et al., Proc. Natl. Acad. Sci. U.S.A., Nov. 10; 95(23):13597-602, 1998).

### Example 3

### Assembly of Synthetic Scaffolds

Synthetic VAPs were designed on basis of their, predicted, three-dimensional structure. The amino acid sequence (Table 3) was back translated into DNA sequence (Table 4) using the preferred codon usage for enteric bacterial gene expression (Informax Vector Nti). The obtained DNA sequence was checked for undesired restriction sites that could interfere with future cloning steps. Such sites were removed by changing the DNA sequence without changing the amino acid codons. Next the DNA sequence was adapted to create an NdeI site at the 5′ end to introduce the ATG start codon and at the 3′ end SfiI site, both required for unidirectional cloning purposes. PCR assembly consists of four steps: oligo primer design (ordered at Operon's), gene assembly, gene amplification, and cloning. The scaffolds were assembled in the following manner: first both plus and minus strands of the DNA sequence were divided into oligonucleotide primers of approximately 35 bp and the oligonucleotide primer pairs that code for opposite strands were designed in such a way that they have complementary overlaps of approximately 16-17 bases. Second, all oligonucleotide primers for each synthetic scaffold were mixed in equimolar amounts, 100 pmol of this primer mix was used in a PCR assembly reaction using 1 Unit Taq polymerase (Roche), 1×PCR buffer+mgCl₂ (Roche) and 0.1 mM dNTP (Roche) in a final volume of 50 μl, 35 cycles of 30 seconds at 92° C., 30 seconds at 50° C., and 30 seconds at 72° C. Third, 5 μl of PCR assembly product was used in a standard PCR amplification reaction using, both outside primers of the synthetic scaffold, 1 Unit Taq polymerase, 1×PCR buffer+mgCl₂, and 0.1 mM dNTP in a final volume of 50 μl, 25 cycles of 30 seconds at 92° C., 30 seconds at 55° C., 1 minute at 72° C. Fourth, PCR products were an by agarose gel electrophoresis, PCR products of the correct size were digested with NdeI and SfiI and ligated into vector pCm126 linearized with NdeI and SfiI. Ligation products were transformed into TOP10-competent cells (InVitrogen) grown overnight at 37° C. on 2×TY plates containing 100 microgram/ml ampicillin and 2% glucose. Single colonies were grown in liquid medium containing 100 μg ampicillin, plasmid DNA was isolated and used for sequence analysis.

### Example 4

### Expression vector Cm126 Construction

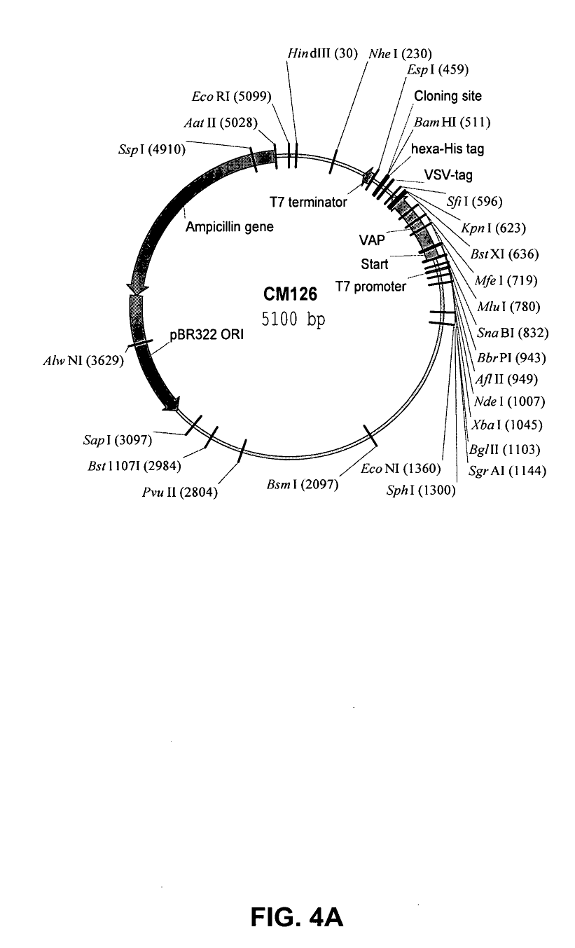

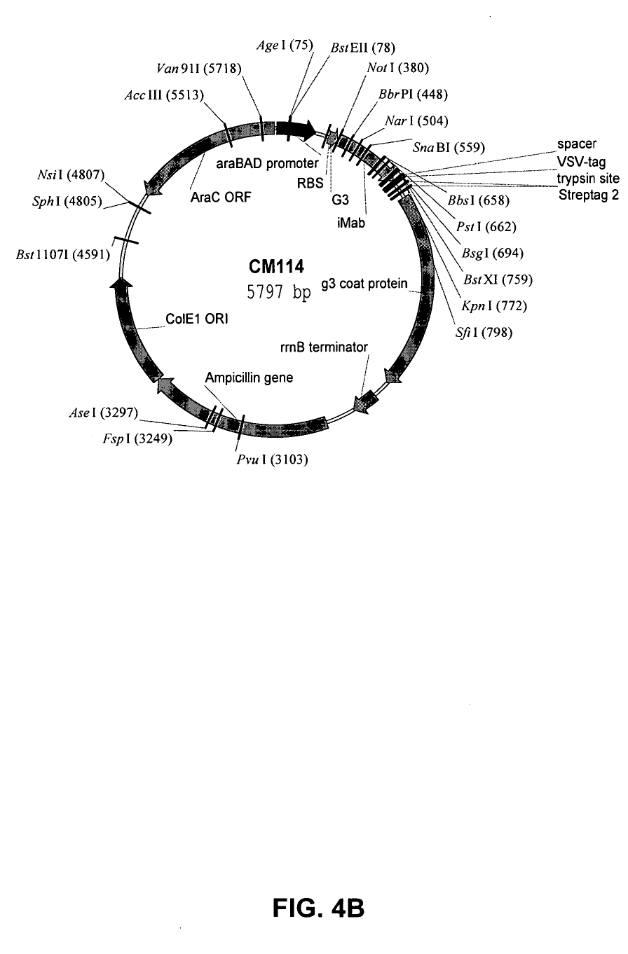

A vector for efficient protein expression (CM126; see FIG. 4A) based on pET-12a (Novagen) was constructed. A dummy VAP, iMab100, including convenient restriction sites, linker, VSV-tag, 6 times His-tag and stop codon was inserted (see Tables 4, 3). As a result the signal peptide OmpT was omitted from pET-12a. iMab100 was PCR amplified using forward primer 129 (see Table 5) that contains a 5′ NdeI overhanging sequence and a very long reverse oligonucleotide primer 306 (see Table 5) containing all linkers and tag sequences and a BamHI overhanging sequence. After amplification, the PCR product and pET-12a were digested with NdeI and BamHI. After gel purification products were purified via the Qiagen gel-elution system according to the manufacturer's procedures. The vector and PCR fragment were ligated and transformed by electroporation in E. coli TOP10 cells. Correct clones were selected and verified for their sequence by sequencing. This vector including the dummy VAP acted as the basic vector for expression analysis of other VAPs. Insertion of other VAPs was performed by amplification with primers 129 and 51 (see Table 5), digestion with NdeI and SfiI and ligation into NdeI- and SfiI-digested Cm126 (sequence see Table 18).

### Example 5

### Expression of iMab100

E. coli BL21 (DE3) (Novagen) was transformed with expression vector CM 126-iMab100. Cells were grown in 250 ml shaker flasks containing 50 ml 2*TYmedium (16 g/l tryptone, 10 g/l yeast extract, 5 g/l NaCl (Merck)) supplemented with ampicillin (200 microgram/ml) and agitated at 30° C. Isopropylthio-β-galactoside (IPTG) was added at a final concentration of 0.2 mM to initiate protein expression when OD (600 nm) reached one. The cells were harvested four hours after the addition of IPTG, centrifuged (4000g, 15 minutes at 4° C.) and pellets were stored at -20° C. until used.

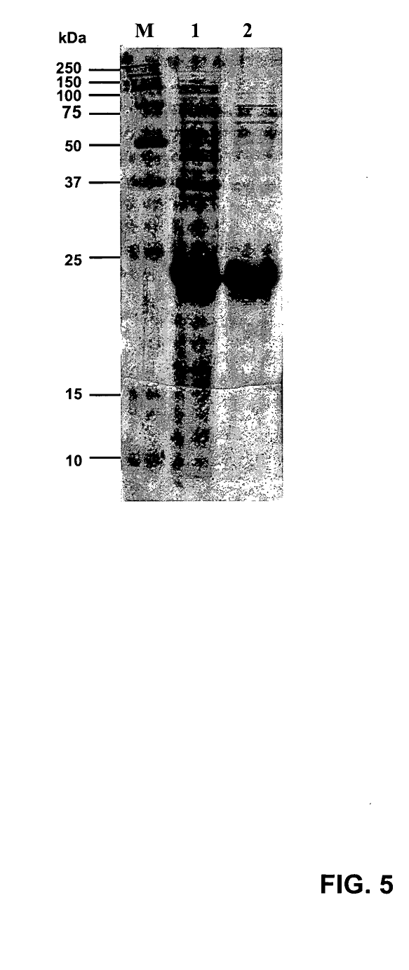

Protein expression was analyzed by Sodium Dodecyl Sulphate PolyAcrylamide Gel Electrophoresis (SDS-PAGE). This is demonstrated in FIG. 5, Lane 2 for E. coli BL21 (CM 126-iMab100) expressing iMAb100.

### Example 6

### Purification of iMab100 Proteins from Inclusion Bodies using Heat.

IMab100 was expressed in E. coli BL21 (CM 126-iMab100) as described in Example 5. Most of the expressed iMab100 was deposited in inclusion bodies. This is demonstrated in FIG. 5, Lane 2, which represents soluble proteins of E. coli BL21 (CM126) after lysis (French press) and subsequent centrifugation (12,000 g, 15 minutes). Inclusion bodies were purified as follows. Cell pellets (from a 50 ml culture) were resuspended in 5 ml PBS pH 8 up to 20 g cdw/l and lysed by two passages through a cold French pressure cell (Sim-Aminco). Inclusion bodies were collected by centrifugation (12,000 g, 15 minutes) and resuspended in PBS containing 1% Tween-20 (ICN) in order to solubilize and remove membrane-bound proteins. After centrifugation (12,000 g, 15 minutes), pellet (containing inclusion bodies) was washed two times with PBS. The isolated inclusion bodies were resuspended in PBS pH 8+1% Tween-20 and incubated at 60° C. for ten minutes. This resulted in nearly complete solubilization of iMab100 as is demonstrated in FIG. 5. Lane 2 represents isolated inclusion bodies of iMab100. Lane 3 represents solubilized iMab100 after incubation of the isolated inclusion bodies in PBS pH 8+1% Tween-20 at 60° C. for ten minutes.

The supernatant was loaded on a Nickel-Nitrilotriacetic acid (Ni-NTA) superflow column and purified according to a standard protocol as described by Qiagen (The QIAexpressionist™, fifth edition, 2001). The binding of the thus purified iMab100 to chicken lysozyme was analyzed by ELISA (according to Example 8) and is summarized in Table 6.

### Example 7

### Purification of iMab100 Proteins from Inclusion Bodies using Urea and Matrix Assisted Refolding

Alternatively, iMab100 was solubilized from inclusion bodies using 8m urea and purified into an active form by matrix assisted refolding. Inclusion bodies were prepared as described in Example 6 and solubilized in 1 ml PBS pH 8+8m urea. The solubilized proteins were clarified from insoluble material by centrifugation (12,000 g, 30 minutes) and subsequently loaded on a Ni-NTA super-flow column (Qiagen) equilibrated with PBS pH 8+8M urea. Aspecific proteins were released by washing the column with four volumes PBS pH 6.2+8M urea. The bound His-tagged iMab100 was allowed to refold on the column by a stepwise reduction of the urea concentration in PBS pH 8 at room temperature. The column was washed with two volumes of PBS+4M urea, followed by two volumes of PBS+2M urea, two volumes of PBS+1 M urea and two volumes of PBS without urea. IMab100 was eluted with PBS pH 8 containing 250 mM imidazole. The released iMab100 was dialyzed overnight against PBS pH 8 (4° C.), concentrated by freeze drying and characterized for binding and structure measurements.

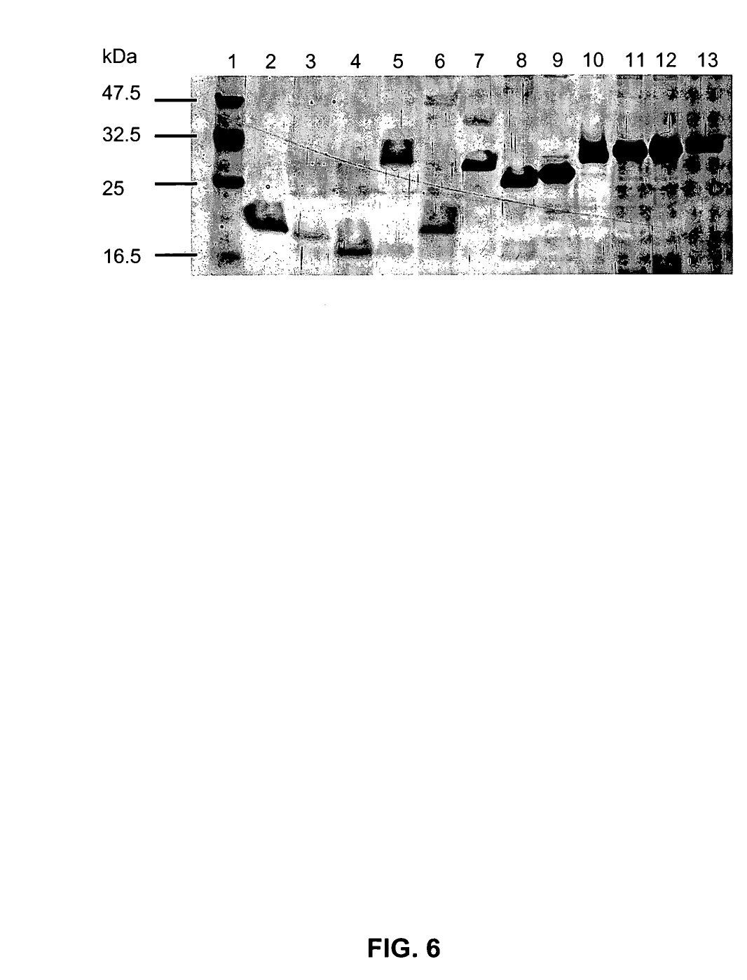

The purified fraction of iMab100 was analyzed by SDS-PAGE as is demonstrated in FIG. 6, Lane 13.

### Example 8

### Specific Binding of iMab100 Proteins to Chicken Lysozyme (ELISA)

Binding of iMab proteins to target molecules was detected using an Enzyme Linked Immuno Sorption Assay (ELISA). ELISA was performed by coating wells of microtiter plates (Nunc) with the desired antigen (such as chicken lysozyme) and blocked with an appropriate blocking agent such as 3% skim milk powder solution (ELK). Purified iMab proteins or purified phages (10⁶-10⁹) originating from a single colony were added to each well and incubated for 1 hour at room temperature. Plates were excessively washed with PBS containing 0.1 % Tween-20 using a plate washer (Bio-Tek Instruments). Bound iMab proteins or phages were detected by the standard ELISA protocol using anti-VSV-hrp conjugate (Roche) or anti-M13-hrp conjugate (Pharmacia), respectively. Colorimetric assays were performed using Turbo-TMB (3, 3′, 5, 5′-tetramethylbenzidine Pierce) as a substrate.

Binding of iMab100 to chicken lysozyme was assayed as follows. Purified iMab100 (˜50 ng) in 100 μl was added to a microtiter plate well coated with either ELK (control) or lysozyme (+ELK as a blocking agent) and incubated for one hour at room temperature on a table shaker (300 rpm). The microtiter plate was excessively washed with PBS (three times), PBS+0.1% Tween-20 (three times) and PBS (three times). Bound iMab100 was detected by incubating the wells with 100 μl ELK containing anti-VSV-HRP conjugate (Roche) for one hour at room temperature.

After excessive washing using PBS (three times), PBS+0.1 % Tween-20 (three times) and PBS (three times), wells were incubated with 100 μl Turbo-TMB for five minutes. Reaction was stopped with 100 μl 2M H₂SO₄ and absorbtion was read at 450 nm using a microtiter plate reader (Biorad).

Purified iMab100 which has been prepared as described in Example 6 and Example 7 appeared to bind strongly and specifically to chicken lysozyme which is demonstrated in Table 6.

### Example 9

### Size Exclusion Chromatography

IMab100 was purified as described in Example 7.

The purified iMab100 was analyzed for molecular weight distribution using a Shodex 803 column with 40% acetonitrile, 60% milliQ and 0.1 % TFA as mobile phase. 90% of the protein eluted at a retention time of 14.7 minutes corresponding to a molecular weight of 21.5 kD. This is in close agreement with the computer calculated molecular weight (19.5 kD) and indicates that most of the protein is present in the monomeric form.

### Example 10

### iMab100 Stability at 95° C. Over Time

iMab100 stability was determined at 95° C. by ELISA. Ten microgram/milliliter iMab100 was heated to 95° C. for ten minutes to 2.5 hours, unheated iMab was used as input control.

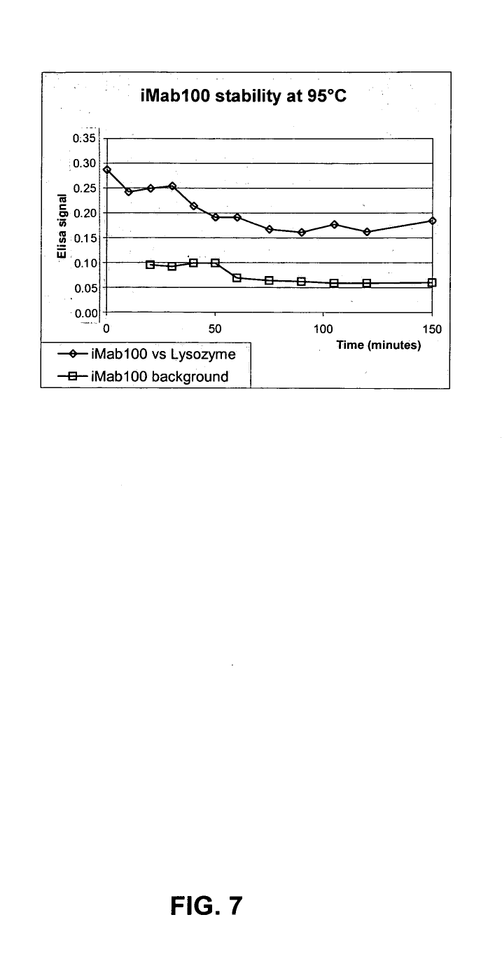

After heating, samples were placed at 20° C. and kept there until assayed. Lysozyme binding of these samples was tested by ELISA measurements using 1:2000 in PBS diluted anti-VSV-hrp (Roche). TMB-ultra (Pierce) was used as a substrate for hrp enzyme levels (FIG. 7). iMab100 was very stable at high temperatures. A very slow decrease in activity was detected.

### Example 11

### iMab100 Stability Over Time at 20° C.

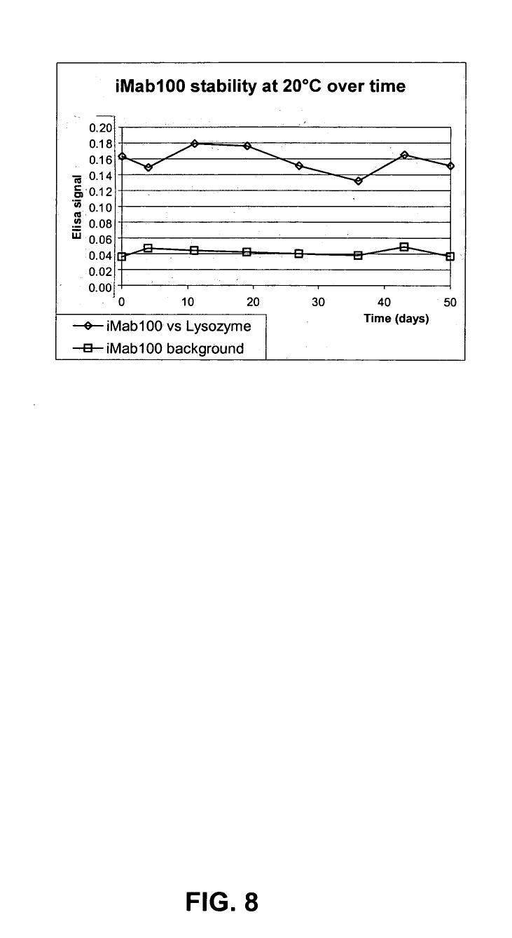

iMab100 stability was determined over a period of 50 days at 20° C. iMab100 (0.1 milligram/milliliter) was placed at 20° C. Every seven days, a sample was taken and every sample was stored at −20° C. for at least two hours to prevent breakdown and freeze the experimental condition. Samples were diluted 200 times in PBS. Lysozyme binding of these samples was tested by ELISA measurements using 1:2000 in PBS diluted anti-VSV-hrp (Roche). TMB-ultra (Pierce) was used as a substrate for hrp enzyme levels (FIG. 8). iMab100 was very stable at room temperature. Activity of iMab100 hardly decreased over time, and thus it can be concluded that the iMab scaffold and its affinity regions are extremely stable.

### Example 12

iMab100 size determination, resistance against pH 4.8 environment, testing by gel and Purified iMab100 (as described in Example 6) was brought to pH 4.8 using potassium acetate (final concentration of 50 mM) which resulted in precipitation of the protein. The precipitate was collected by centrifugation (12,000 g, 30 minutes), redissolved in PBS pH 7.5 and subsequently filtered through a 0.45 micrometer filter to remove residual precipitates.

The samples fore and after pH shock were analyzed by SDS-PAGE, western blotting and characterized for binding using ELISA Example 8).

It was demonstrated that all iMab100 was precipitated at pH 4.8 and could also be completely recovered after redissolving in PBS pH 7.5 and filtering. ELISA measurements demonstrated that precipitation and subsequent resolubilization did not result in a loss of activity (Table 7). It was confirmed that the VSV-tag is not lost during purification and precipitation and that no degradation products are formed.

### Example 13

### Structural Analysis of Scaffolds

The structure of iMab100 was analyzed and compared with another structure using a circular dichroism polarimeter (CD). As a reference, a naturally occurring 9-beta-strand containing Vhh molecule, Vhh10-2/271102 (a kind gift of M. Kwaaitaal, Wageningen University), was measured. Both proteins have tags attached to the C-terminal end. The amino acid sequence and length of these tags are identical. The only structural differences between these two proteins are present in the CDR3 (Vhh) corresponding affinity loop 4 (iMab100).

System settings were: sensitivity standard (100 mdeg); start=260 nm end=205 nm; interval=0.1 nm; delay=1 second; speed=50 nm/minute; accumulation=10.

iMab100 and Vhh10-2/271102 were prepared with a purity of 98% in PBS pH 7.5 and OD₂₈₀≈1.0. Sample was loaded in a 0.1 cm quartz cuvette and the CD spectrum measured with a computer controlled JASCO Corporation J-715 spectropolarimeter software (Spectramanager version 1.53.00, JASCO Corporation). Baseline corrections were obtained by measuring the spectrum of PBS. The obtained PBS signal was subtracted from all measurement to correct for solvent and salt effects. An initial measurement with each sample was done to determine the maximum signal. If required, the sample was diluted with 1 times PBS for optimal resolution of the photomultiplier signal. A solution in PBS of RNase A was used to verify the CD apparatus. The observed spectrum of RNase A was completely different if compared with iMab100 and the Vhh spectrum. FIG. 9L represents the CD spectrum of iMab100 and the Vhh proteins in far UV (205-260 nm). Large part of the spectral patterns were identical. Spectral differences were mainly observed at wavelengths below 220 nm. The observed differences of the spectra are probably due to differences in CDR3/AR4 structural differences. The structure of AR4 in iMab100, which was retrieved from 1MEL, can be classified as random coil-like. Also, AR4 present in iMab100 is about ten amino acids longer than the CDR3 of the Vhh protein.

The temperature stability of the iMab100 protein was determined in a similar way using the CD-meter except that the temperature at which the measurements were performed was adjusted.

In addition to measurements at room temperature, folding and refolding was assayed at 20, 50, 80 (not shown) and 95° C. Fresh iMab100 protein solution in PBS diluted was first measured at 20° C. Next, spectra at increasing temperatures were determined and lastly, the 20° C. spectrum was re-measured. Baseline corrections were applied with the spectrum of PBS (FIG. 9A). The results clearly show a gradual increase in ellipticity at increasing temperatures. The re-appearance of the 20° C. spectrum after heating strongly indicates complete refolding of the scaffold. This conclusion was also substantiated by subsequent lysozyme binding capacity detection of the samples by ELISA (data not shown).

### Example 14

E. coli BL21 (DE3) (Novagen) was transformed with expression vector CM126 containing various VAP inserts for iMab130, iMab1602, iMab1202 and iMab122 all containing nine β-strands. Growth and expression was similar as described in Example 5. All nine-stranded iMab proteins were purified by matrix assisted refolding similar as described in Example 7. The purified fractions of iMab1302, iMab1602, iMab1202 and iMab122 were analyzed by SDS-PAGE as is demonstrated in FIG. 6, Lanes 10, 9, 8 and 7 respectively.

### Example 15

### Specific Binding of Various Nine-Stranded iMab Proteins to Chicken Lysozyme (ELISA)

Purified iMab1302 (˜50 ng), iMab1602 (˜50 ng), iMab1202 (˜50 ng) and iMab122 (˜50 ng) were analyzed for binding to either ELK (control) or lysozyme (+ELK as a blocking agent) similar as is described in Example 8. ELISA confirmed specific binding of purified iMab1302, iMab1602, iMab1202 and iMab122 to chicken lysozyme as is demonstrated in Table6.

### Example 16

### CD Spectra of Various Nine-Stranded iMab

iMab100, iMab1202, Imab1302 and iMab1602 were purified as described in Example 14 and analyzed for CD spectra as described in Example 13. The spectra of iMab 1202, iMab1302 and iMab1602 were measured at 20° C., 95° C. and back at 20° C. to test scaffold stability and refolding characteristics. The corresponding spectra are demonstrated in FIGS. 9D, 9E and 9F, respectively. The spectra measured at 20° C. were compared with the spectrum of iMab100 at 20oC to determine the degree of similarity of the secondary structure (see FIG. 9J). It can be concluded that all different nine-strand scaffolds behave the same. This indicates that the basic structure of these scaffolds is identical. The data obtained after successive 20-95-20 degrees Celsius treatments clearly show that all scaffolds return to their original conformation.

### Example 17

### Design of Seven-Stranded ig-Like Folds

The procedure as described in Example 2 was used for the development of sequences that contain an ig-like fold consisting of seven beta-elements in the core and 3+3 connecting loops. The procedure involved four phases through which the development of the new sequences was guided, identical to the process as described in Example 2. In phase 1, the coordinates of C-alpha atoms as indicated in PDB Table 1 for nine-stranded core structures were adapted. C-alpha atoms representing beta-elements 4 and 5 were removed from the PDB files, resulting in a seven-stranded example of the core (PDB Table 8). Amino acid side chains that line up with the interior of the beta-sheets were obtained and inserted as described in detail in Example 2. In the second phase connecting loops were added. On one site beta-elements were connected with one other by affinity region retrieved from anti-chicken lysozyme binding region obtained from the structure I MEL or the bovine RNase A binding regions of 1 BZQ (L2, L6 and L8). On the other end of the structure, beta-elements were connected with C-alpha backbone trace loops obtained from several different origins (1E50, 1CWv, 1QHP, 1NEU, 1EPF, 1F2× or 1EJ6). The procedure for the attachment and fit of the loops is described in detail in Example 2. In the third phase, amino acid side chains that determine the solubility of the proteins located in the core and loops 1, 3, 7 were determined as described in Example 2. In the last phase, the models were built using Insight. Insight was programmed to accept cysteine-cysteine bridges when appropriate. Next all predicted protein structures built with Insight were assessed with Prosall, Procheck and WHAT IF. Prosall zp-comb scores of less then −4.71 were assumed to indicate protein sequences that might fold in vivo into the desired ig-like beta-motif fold (Table 9). A number of example sequences depicted in Table 10 represent a collection that appeared to be reliable. Procheck and What if assessments also indicated that these sequences might fit into the models and thus as being reliable (e.g., pG values larger than 0.80; Sánchez et al., 1998).

### Example 18

E. coli BL21 (DE3) (Novagen) was transformed with expression vector CM126 containing various VAP inserts for iMab1300, iMab1200, iMab101 and iMab900 all containing seven beta-strands. Growth and expression was similar as described in Example 5.

All seven-strand iMabs were purified by matrix assisted refolding similar as is described in Example 7. The purified fractions of iMab101, iMab1300, iMab1200 and iMab900 were analyzed by SDS-PAGE as is demonstrated in FIG. 6, Lanes 2, 3, 5 and 6, respectively.

### Example 19

Purified iMab1300 (˜50 ng), iMab1200 (˜5 ng), iMab101(˜20 ng) and iMab900 (˜10 ng) were analyzed for binding to either ELK (control) or lysozyme (+ELK as a blocking agent) similar as is described in Example 8. ELISA confirmed specific binding of purified iMab1300, iMab1200, iMab101 and iMab900 to chicken lysozyme as is demonstrated in Table 6.

### Example 20

### CD Spectra of Various Seven-Stranded iMab Proteins

IMab1200 and iMab101 were purified as described in Example 18 and analyzed for CD spectra as described in Example 13. The spectra of iMab1200 and iMab101 were measured at 20° C., 95° C. and back at 20° C. to test scaffold stability and refolding characteristics. The corresponding spectra are demonstrated in FIGS. 9H and 9G, respectively. The spectra of iMab1200 and iMab101 measured at 20° C. were compared with each other to determine the degree of similarity of the secondary structure (see FIG. 9K). It can be concluded that the different seven-strand scaffolds behave the same. This indicates that the basic structure of these scaffolds is identical. Even more, as the obtained signals form the nine-stranded scaffolds (Example 16) are similar to the signals observed for the seven strands as presented here, it can also be concluded that the both types of scaffolds have a similar conformations. The data obtained after successive 20-95-20 degrees Celsius treatments clearly show that all scaffolds stay in their original conformation.

### Example 21

### Design of Six-Stranded ig-Like Folds

The procedure as described in Examples 2 and 3 was used for the development of sequences that contain an ig-like fold consisting of six beta-elements in the core and 330 3 connecting loops. The procedure involved four phases through which the development of the new sequences was guided, identical to the process as described in Examples 2 and 3. In phase one, the coordinates of C-alpha atoms as indicated in PDB Table 1 for nine-stranded core structures were adapted. C-alpha atoms representing beta-elements 1, 4 and 5 were removed from the PDB files, resulting in a six-stranded example of the core (Table 11). Amino acid side chains that line up with the interior of the beta-sheets were obtained and inserted as described in detail in Examples 2 and 3. In the second phase, connecting loops were added. On one site beta-elements were connected with one other by affinity region retrieved from anti-chicken lysozyme binding region obtained from the structure 1MEL or the bovine RNase A binding regions of 1BZQ (L2, L6 and L8). On the other end of the structure, beta-elements were connected with C-alpha backbone trace loops obtained from several different origins (1E50, 1CWV, 1QHP, 1NEU, 1EPF, 1F2× or 1EJ6). The procedure for the attachment and fit of the loops is described in detail in Examples 2 and 3. In the third phase, amino acid side chains that determine the solubility of the proteins located in the core and loops L1, L3, L7 were determined as described in Examples 2 and 3. In the last phase, the models were assessed using modeller. Modeller was programmed to accept cysteine-cysteine bridges when appropriate. Next all predicted protein structures were assessed with Prosall, Procheck and WHAT IF. Prosall zp-comb scores were determined (Table 12) to indicate if the created protein sequences might fold in vivo into the desired ig-like beta-motif fold. Procheck and What if assessments were applied to check whether sequences might fit into the models Table 13).

### Example 22

### Purification of Six-Stranded iMab Proteins

E. coli BL21 (DE3) (Novagen) was transformed with expression vector Cm126 containing a VAP insert for iMab701 containing six beta-strands. Growth and expression was similar as described in Example 5.

The iMab701 proteins were purified by matrix assisted refolding similar as is described in Example 7. The purified fraction of iMab701 was analyzed by SDS-PAGE as is demonstrated in FIG. 6, Lane 4.

### Example 23

### Specific Binding of Six-Stranded iMab Proteins to Chicken Lysozyme (ELISA)

Purified iMab701 (˜10 ng) was analyzed for binding to either ELK (control) and lysozyme (+ELK as a blocking agent) similar as is described in Example 8.

ELISA confirmed specific binding of purified iMab701 to chicken lysozyme as is demonstrated in Table 6.

### Example 24

### CD Spectra of Six-Stranded iMab Proteins

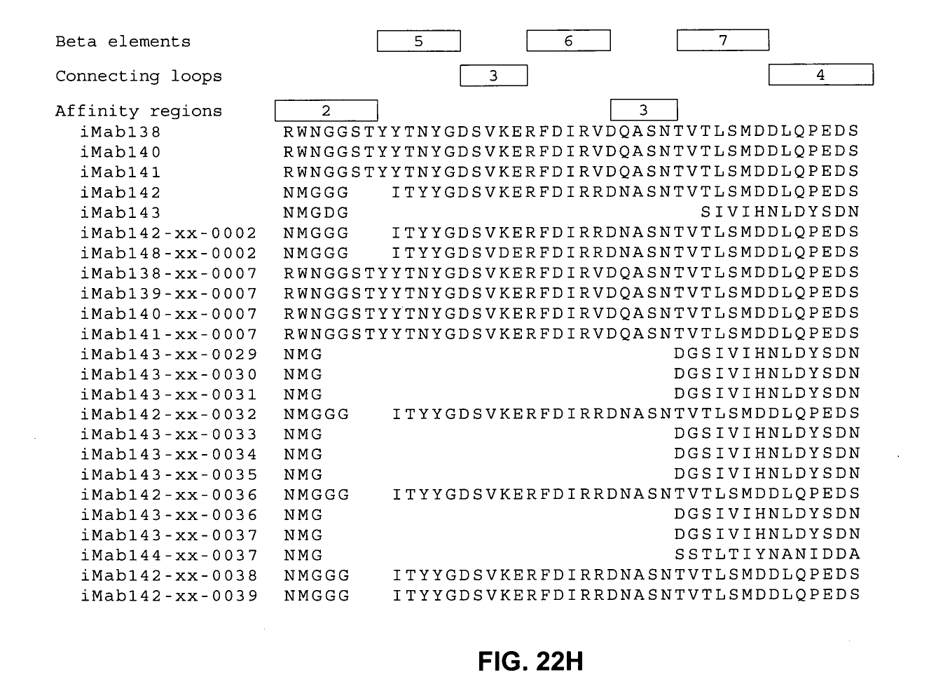

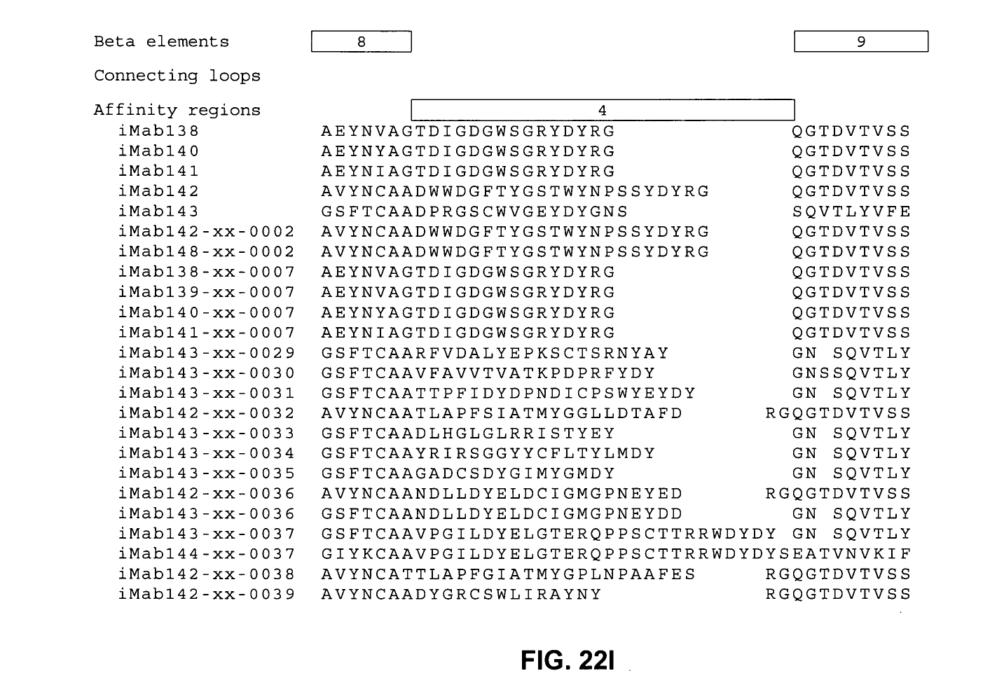

IMab701 was purified as described in Example 22 and analyzed for CD spectra as described in Example 13. The spectra of iMab701 was measured at 20° C., 95° C. and again at 20° C. to test scaffold stability and refolding characteristics. The corresponding spectra are demonstrated in FIG. 91. It can be concluded that the six-strand scaffold behaves identical to the seven-strand scaffolds as described in Example 20. This indicates that the basic structure of this scaffold is identical to the structure of the seven strand containing scaffolds. Even more, as the obtained signals form the nine-stranded scaffolds (Example 16) are similar to the signals observed for this six-strand scaffold as presented here, it can also be concluded that both types of scaffolds have similar conformations. The data obtained after successive 20-95-20 degrees Celsius treatments clearly show that all scaffolds stay in their original conformation.

### Example 25

### Design of a Minimal Primary Scaffold

A minimal scaffold is designed according to the requirements and features as described in Example 1. However now only four and five beta-elements are used in the scaffold (see FIG. 1). In the case of five beta-elements amino acids side chains of beta-elements 2, 3, 6, 7 and 8 that are forming the mantle of the new scaffold need to be adjusted for a watery environment. The immunoglobulin killer receptor 2dl2 (VAST code 2DLI) is used as a template for comparative modeling to design a new small scaffold consisting of 5 beta-elements.

### Example 26

### Procedure for Exchanging Surface Residues: Lysine Replacements

Lysine residues contain chemical active amino-groups that are convenient in, for example, covalent coupling procedures of VAPs. Covalent coupling can be used for immobilization of proteins on surfaces or irreversible coupling of other molecules to the target.

The spatial position of lysine residues within the VAP determines the positioning of the VAP on the surface after immobilization. Wrong positioning can easily happen with odd located lysine residues exposed on the surface of VAPs. Therefore, it may be required for some VAP structures to remove lysine residues from certain locations, especially from those locations that can result in diminished availability of affinity regions.

As an example of the exchange strategy for residues that are located on the outer surface, iMab100 outer surface lysine residues were changed. Three-dimensional imaging indicated that all lysine residues present in iMab100 are actually located on the outer surface. Three-dimensional modeling and analysis software (InsightII) determined the spatial consequence of such replacements.

Modeler software was programmed in such a way that either cysteine bridge formation between the beta-sheets was taken into account or the cysteine bridges were neglected in analyses. All retrieved models were built with Prosall software for more or less objective result ranking. The zp-comb parameter of ProsaII indicated the reliability of the models. Results showed that virtually all types of amino acids could replace lysine residues. However, surface-exposed amino acid side chains determine the solubility of a protein. Therefore, only amino acids that will solubilize the proteins were taken into account and marked with an X (see Table 14).

Sequence of iMab100 (SEQ ID NO:4): underlined lysine residues were exchanged

NVKLVEKGGNFVENDDDLKLTCRAEGYTIGPYCMGWFRQAPNDDSTNVAT
INMGGGITYYGDSVKERFDIRRDNASNTVTLSMDDLQPEDSAEYNCAGDS
TIYASYYECGHGLSTGGYGYDSRGQGTDVTVSS

### Example 27

### Changing Amino Acids in the Exterior: Removal of Glycosylation Site.

N-glycosylation can interfere strongly with protein functions if the glycosylation site is, for example, present in a putative ligand-binding site. iMab100 proteins were shown to be glycosylated in Pichia pastoris cells and unable to bind to the ligand. Analysis showed that there is a putative N-glycosylation site in AR3. Inspection of the iMab100 structure using template-modeling strategies with modeler software revealed that this site is potentially blocking ligand binding due to obstruction by glycosylation. This site could be removed in two different ways, by removing the residue being glycosylated or by changing the recognition motif for N-glycosylation. Here, the glycosylation site itself ( . . . RDNAS . . . ) was removed. All residues could be used to replace the amino acid, after which ProsaII, What if and Procheck could be used to check the reliability of each individual amino acid. However, some amino acids could introduce chemical or physical properties that are unfavorable. Cysteine, for example, could make the proteins susceptible to covalent dimerization with proteins that also bear a free cysteine group. Also non-hydrophilic amino acids could disturb the folding process and were omitted. Methionine, on the other hand, is coded by ATG, which can introduce aberrant start sites in DNA sequences. The introduction of ATG sequences might result in alternative protein products due to potential alternative start sites. Methionine residues were only assessed if no other amino acids would fit. All other amino acid residues were assessed with Prosall, What if and Procheck. Replacement of N with Q was considered to be feasible and reliable.

Protein sequence from iMab with glycosylation site:

NVKLVEKGGNFVENDDDLKLTCRAEGYTIGPYCMGWFRQAPNDDSTNVAT
INMGGGITYYGDSVKERFDIRRDNASNTVTLSMDDLQPEDSAEYNCAGDS
TIYASYYECGHGLSTGGYGYDSRGQGTDVTVSS

Protein sequence from iMab without glycosylation site (SEQ ID NO:5):

NVKLVEKGGNFVENDDDLKLTCRAEGYTIGPYCMGWFRQAPNDDSTNVAT
INMGGGITYYGDSVKERFDIRRDQASNTVTLSMDDLQPEDSAEYNCAGDS
TIYASYYECGHGLSTGGYGYDSRGQGTDVTVSS

Expression of iMab100 in Pichia pastoris was performed by amplification of 10 ng of CM114-iMab100 DNA in a 100 microliter PCR reaction mix comprising 2 units Taq polymerase (Roche), 200 micromol or of each dNTP (Roche), buffers (Roche Taq buffer system), 2.5 micromolar of primer 107 and 108 in a Primus96 PCR machine (MWG) with the following program 25 times (94° C. for 20 seconds, 55° C. for 25 seconds, 72° C. for 30 seconds) digestion with EcoRI and NotI and ligation in EcoRI and NotI-digested pPIC9 (InVitrogen). Constructs were checked by sequencing and showed all the correct iMab100 sequence. Transformation of Pichia pastoris was performed by electroporation according to the manufacturer's protocol. Growth and induction of protein expression by methanol was performed according to the manufacturer's protocol. Expression of iMab100 resulted in the production of a protein that on a SDS-PAGE showed a size of 50 kD, while expressed in E. coli the size of iMab100 is 21 kD. This difference is most likely due to glycosylation of the putative N-glycosylation site present in iMab100 as described above. Therefore this glycosylation site was removed by exchange of the asparagine (N) for a glutamine (Q) in a similar way as described in Example 26 except that primer 136 (Table 5) was used. This resulted in iMab115. Expression of iMab15 in E. coli resulted in the production of a 21 kD protein. ELISA experiments confirmed specificity of this iMab for lysozyme. Thus, ARs in iMab115 were positioned correctly and, more specifically, replacement of the asparagine with glutamine in AR3 did not alter AR3 properties.

### Example 28

### Changing Amino Acids in the Interior of the Core: Removal of Cysteine Residues

Obtained sequences that fold in an ig-like structure, can be used for the retrieval of similarly folded structures but aberrant amino acid sequences. Amino acids can be exchanged with other amino acids and thereby putatively changing the physical and chemical properties of the new protein if compared with the template protein. Changes on the outside of the protein structure were shown to be rather straightforward. Here we changed amino acids that are lining up with the interior of the core. Spatial constrains of neighboring amino acid side chains and the spatial constrains of the core structure itself determine and limit the types of side chains that can be present at these locations. In addition, chemical properties of neighboring side chains can also influence the outcome of the replacements. In some replacement studies, it might be necessary to replace additional amino acids that are in close proximity of the target residues in order to obtain suitable and reliable replacements.

Here were removed the potential to form cysteine bridges in the core. The removal of only one cysteine already prevents the potential to form cysteine bridges in the core. However, dual replacements can also be performed in order to prevent the free cysteine to interact with other free cysteine during folding or re-folding in vivo or in vitro. First, the individual cysteine residues were replaced by any other common amino acid (19 in total). This way, two times 19 models were retrieved. All models were assessed using ProsaII (zp-scores), What if (2^{nd} generation packing quality, backbone conformation) and Procheck (number of residues outside allowed regions). Several reliable models were obtained. Table 15 shows the zp-combined Prosa scores of the cysteine replacements at position 96. The replacement of one of the cysteines with valine was tested in vivo to validate the method. This clone was designated as iMab116 (see Table 3) and constructed (Table 4) according to the procedure as described in Example 3. The complete iMab sequence of this clone was transferred into Cm126 in the following manner. The iMab sequence, iMab116, was isolated by PCR using Cys-min iMab116 as a template together with primers pr121 and pr129 (Table 5). The resulting PCR fragment was digested with NdeI and SfiI and ligated into Cm126 linearized with NdeI and SfiI. This clone, designated CM126-iMab116, was selected and used for further testing.

### Example 29

### Purification of iMab116

E. coli BL21 (DE3) (Novagen) was transformed with expression vector Cm126 containing a VAP insert for iMab116 containing nine beta-strands and potentially lacking a cysteine bridge in the core (as described in Example 27). Growth and expression was similar as described in Example 5.

IMab 116 was purified by matrix assisted refolding similar as is described in Example 7. The purified fraction of iMab116 was analyzed by SDS-PAGE as is demonstrated in FIG. 6, Lane 11.

### Example 30

### Specific Binding of iMab116 to Chicken Lysozyme (ELISA)

Purified iMab116 (˜50 ng) was analyzed for binding to either ELK (control) and lysozyme (+ELK as a blocking agent) similar as is described in Example 8. ELISA confirmed specific binding of purified iMab116 to chicken lysozyme as is demonstrated in Table 6.

### Example 31

### CD Spectra of iMab116 Proteins

IMab116 was purified as described in Example 28 and analyzed for CD spectra as described in Example 13. The spectrum of iMab16 was measured at 20° C., 95° C. and again at 20° C. to test scaffold stability and refolding characteristics. The corresponding spectra are demonstrated in FIG. 9C. The spectra measured at 20° C. were compared with the spectrum of iMab100 and other nine-stranded iMab proteins at 20° C. to determine the degree of similarity of the secondary structure (see FIG. 9J). Because the obtained spectrum is identical to the spectrum obtained from other nine-strand scaffolds, including the iMab100 spectrum, it can be concluded that the cysteine residue removal from the internal core has no effect on the structure itself.

### Example 32

### Introduction of Extra Cysteine Bridge in the Core

Chemical bonding of two cysteine residues in a proteins structure (cysteine bridge) can dramatically stabilize a protein structure at temperatures below about 70° C. Above this temperature cysteine bridges can be broken. Some applications demand proteins that are more stable than the original protein. The spatial constrains of the core of beta-strand folds as referred to in Example 1, enable cysteine bridges. This conclusion is based on the observation that in some naturally occurring proteins with the referred fold a cysteine bridge is present in the center of the core (e.g., all heavy chain variable domains in antibodies). The distance between C-alpha backbone atoms of such cysteines is most often found to be between 6.3 and 7.4 angstrom.

The introduction of new cysteine residues that putatively form bridges in core motifs was analyzed by structural measurements. The coordinates of C-alpha atoms of a protein written in PDB files can be used to determine potential cysteine bridges. The distance between each C-alpha atom individually and all other C-alpha atoms can be calculated. The position of C-alpha atoms of the iMab100 protein obtained via comparative modeling is shown in FIG. BBB3. Insight software can be used to determine the distance between C-alpha atoms. However, standard mathematical algorithms that determine distances between two positions in space indicated by coordinates (as represented in PDB coordinates) can also be used. Excel sheets were used to determine all possible distances. Distance values that appear to be between 6.3 and 7.4 angstrom were regarded as putative cysteine locations. Analysis indicated 33 possible cysteine bridge locations within iMab100. The cys-number indicates the position of the C-alpha atom in the structure that might be used for the insertion of a cysteine (Table 16A). However, not all positions in space are very useful; some bridges might be too close to an already available cysteine bridge, two cysteines next to each other can be problematic, two cysteine bridges between identical beta-strands will not be very helpful, spatial constrains with other amino acid side chains that are located nearby. All 33 models were constructed and assayed with iMab100 as a template in modeller. Zp-scores of assessed models obtained with Prosall indicated that most cysteine residues are problematic. The best cysteine locations are indicated in Table 16B. Two models, indicated in bold, were chosen based on the spatial position of these cysteine residues and bridges in relation to the other potential cysteine bridge. Also, some models were rejected, though the zp-scores were excellent, because of their position within the fold as reviewed with Insight (MSI).

### Example 33

### Construction of an iMab100 Derivative that Contains Two Extra Cysteines in the Core

An oligonucleotide mediated site directed mutagenesis method was used to construct an iMab100 derivative, named iMab111 (Table 3), that received two extra cysteine residues. CM114-iMab100 was used as a template for the PCR reactions together with oligonucleotides pr33, pr35, pr82, pr83 (see Table 5). In the first PCR reaction, primers pr82 and pr83 were used to generate a 401 bp fragment. In this PCR fragment a glutamine and a glycine coding residue were changed into cysteine coding sequences. This PCR fragment is used as a template in two parallel PCR reactions. In one reaction, the obtained PCR fragment, CM114-iMab100 template and pr33 were used, while in the other reaction the obtained PCR fragment, CM 114-iMab100 template and primers 35 were used. The first mentioned reaction gave a 584 bp product while the second one produced a 531 bp fragment. Both PCR fragments were isolated via agarose gel separation and isolation (Qiagen gel extraction kit). The products were mixed in an equimolar relation and a fragment overlap-PCR reaction with primers pr33 and pr35 resulted in a 714 bp fragment. This PCR fragment was digested with NotI and SfiI. The resulting 411 bp fragment was isolated via an agarose gel and ligated into CM114 linearized with NotI and SfiI. Sequencing analysis confirmed the product, i.e. iMab111 (Tables 4 and 3).

### Example 34

### Expression of iMab111

iMab111 DNA was subcloned in Cm126 as described in Example 28. CM126-iMab111 transformed BL21(DE3) cells were induced with IPTG and protein was isolated as described in Example 7. Protein extracts were analyzed on 15% SDS-PAGE gels and showed a strong induction of a 21 KD protein. The expected length of iMab11 including tags is also about 21 kD indicating high production levels of this clone.

### Example 35

### Purification of iMab111

E. Coli BL21 (DE3) (Novagen) was transformed with expression vector Cm126 containing a VAP inserts for iMab111 containing 9 beta-strands potentially containing an extra cysteine bridge (as described in Examples 32 and 33).

Growth and expression was similar as described in Examples 5 and 34. iMab111 was purified by matrix assisted refolding similar as is described in Example 7. The purified fraction of iMab111 was analyzed by SDS-PAGE as is demonstrated in FIG. 6, Lane 12.

### Example 36

### Specific Binding of iMab111 to Chicken Lysozyme (ELISA)

Purified iMab111(˜50 ng) was analyzed for binding to either ELK (control) and lysozyme (+ELK as a blocking agent) similar as is described in Example 8. A 100-fold dilution of the protein extract in an ELISA assay resulted in a signal of approximately 20-fold higher than background signal. ELISA results confirmed specific binding of purified iMab111 to chicken lysozyme as is demonstrated in Table 6.

### Example 37

### CD Spectra of iMab111 Proteins

IMab111 was purified as described in Example 32 and analyzed for CD spectra as described in Example 13. The spectrum of iMab116 was measured at 20° C., 95° C. and again at 20° C. to test scaffold stability and refolding characteristics. The corresponding spectra are demonstrated in FIG. 9C. The spectra measured at 20° C. were compared with the spectrum of iMab100 and other nine-stranded iMab proteins at 20° C. to determine the degree of similarity of the secondary structure (see FIG. 9J). Because the obtained spectrum is identical to the spectrum obtained from other nine-strand scaffolds, including the iMab100 spectrum, it can be concluded that the additional cysteine residue in the center of the core has no effect on the structure itself.

### Example 38

### Improving Properties of Scaffolds for Specific Applications

For certain applications, the properties of a scaffold need to be optimized. For example, heat stability, acid tolerance or proteolytic stability can be advantageous or even required in certain environments in order to function well. A mutation and re-selection program can be applied to create a new scaffold with similar binding properties but with improved properties. In this example, a selected binding protein is improved to resist proteolytic degradation in a proteolytic environment. New scaffolds can be tested for proteolytic resistance by a treatment with a mixture of proteases or alternatively a cascade treatment with specific protease. In addition, new scaffolds can be tested for resistance by introducing the scaffolds in the environment of the future application. In order to obtain proteolytic resistant scaffolds, the gene(s) that codes for the scaffold(s) is (are) mutated using mutagenesis methods. Next, a phage display library is build from the mutated PCR products so that the new scaffolds are expressed on the outside of phages as fusion proteins with a coat protein. The phages are added to the desired proteolytic active environment for a certain time at the desired temperature. Intact phages can be used in a standard panning procedure as described. After extensive washing, bound phages are eluted, infected in E. coli cells that bear F-pili and grown overnight on an agar plate that contains appropriate antibiotics. Individual clones are re-checked for their new properties and sequenced. The process of mutation introduction and selection can be repeated several times or other selection conditions can be applied in further optimization rounds.

### Example 39

### Random Mutagenesis of Scaffold Regions

Primers annealing just 3′ and 5′ of the desired region (affinity regions, frameworks, loops or combinations of these) are used for amplification in the presence of dITP according to Spee et al. (Nucleic Acids Res. 21 (3):777-8, 1993) or dPTP according to Zaccolo et al. (J. Mol. Biol., 255(4):589-603, 1996). The mutated fragments are amplified in a second PCR reaction with primers having the identical sequence as the set of primers used in the first PCR but now containing restriction sites for recloning the fragments into the scaffold structure which can differ among each other in DNA sequence and thus also in protein sequence. Phage display selection procedures can be used for the retrieval of clones that have desired properties.

### Example 40

### Phage Display Vector CM114-iMab100 Construction

A vector for efficient phage display (CM114-iMab100; see FIG. 4B) was constructed using part of the backbone of a pBAD (InVitrogen). The required vector part from pBAD was amplified using primers 4 and 5 containing respectively AscI and BamHI overhanging restriction sites. In parallel, a synthetic constructed fragment was made containing the sequence as described in Table 4 including a new promoter, optimized g3 secretion leader, NotI site, dummy insert, SfiI site, linker, VSV-tag, trypsin-specific proteolytic site, Strep-tagII and AscI site (see FIG. 4B). After combining the digested fragment and the PCR amplified pBAD vector fragment, the coding region of them13 phage g3 core protein was amplified using AscI overhanging sites attached to primers (Table 5, primer 6 and 7) and inserted after AscI digestion. Vectors that contained correct sequences and correct orientations of the inserted fragments were used for further experiments.

### Example 41

### Phage Display Vector CM114-iMab113 Construction

Cysteine bridges between AR4 and other affinity regions (e.g., AR1 for iMab100) can be involved in certain types of structures and stabilities that are not very likely without cysteine bridge formations. Not only can AR1 be used as an attachment for cysteines present in some affinity regions 4, but also AR2 and AR3 are obvious stabilizing sites for cysteine bridge formation. Because AR2 is an attractive alternative location for cysteine bridge formation with AR4, an expression vector is constructed which is 100% identical to CM114-iMab100 with the exception of the locations of a cysteine codon in AR2 and the lack of such in AR1. 3D-modelling analysis revealed that the best suitable location for cysteine in AR2 is at the location originally determined as a threonine (.VATIN . . . into . . .VACIN . . . ). Analysis indicated that in addition to the new cysteine location ( . . . VACIN . . .), the alanine residue just before the threonine residue in AR2 was replaced with a serine residue ( . . . VSCIN . . . ). The original cysteine in AR1 was replaced by a serine that turned out to be a suitable replacement according to 3D-modelling analysis (Table 3).

The new determined sequence, named iMab113, (Table 4) was constructed according to the gene construction procedure as described above (Example 3) and inserted in CM 114 replacing iMab100.

### Example 42

### Phage Display Vector CM114-iMab114 Construction

Cysteine bridges between AR4 and other regions are not always desired because intermolecular cysteine bridge formations during folding might influence the efficiency of expression and percentage of correct folded proteins. Also, in reducing environments such ARs might become less active or even inactive. Therefore, scaffolds without cysteine bridges are required.

An expression vector lacking cysteines in AR1, 2 and 3 was constructed. This vector is 100% identical to CM114 with the exception that the cysteine in AR1 ( . . . PYCMG . . . ) has been changed to a serine ( . . . PMSMG . . . ; see Table 3). The new determined sequence, named iMab114 (Table 4) was constructed according to the gene construction procedure as described above (Example 3) and inserted in CM114 replacing iMab100.

### Example 43

### Amplification of Camelidae-Derived CDR3 Regions

Lama pacos and Lama glama blood lymphocytes were isolated according to standard procedures as described in Spinelli et al. (Biochemistry 39 (2000) 1217-1222). RNA from these cells was isolated via Qiagen RNeasy methods according to the manufacturer's protocol. cDNA was generated using muMLv or AMV (New England Biolabs) according to the manufacturer's procedure. CDR3 regions from Vhh cDNA were amplified (see FIG. 10) using 1 μl cDNA reaction in 100 microliter PCR reaction mix comprising two units Taq polymerase (Roche), 200 μM of each dNTP (Roche), buffers (Roche Taq buffer system), 2.5 μM of forward and reverse primers in a Primus96 PCR machine (MWG) with the following program 35 times (94° C. for 20 seconds, 50° C. for 25 seconds, 72° C. for 30 seconds). In order to select f for CDR3 regions containing at least one cysteine primer 56 (Table 5) was used as a forward primer and in case to select for CDR regions that do not contain cysteines primer 76 (Table 5) was used in the first PCR round. In both cases, primer 16 (Table 5) was used as reverse primer. Products were separated on a 1% Agarose gel and products of the correct length (˜250 bp) were isolated and purified using Qiagen gel extraction kit. Five μl of these products were used in a next round of PCR similar as described above in which primer 8 (Table 5) and primer 9 (Table 5) were used to amplify CDR3 regions. Products were separated on a 2% Agarose gel and products of the correct length (˜80-150 bp) were isolated and purified using Qiagen gel extraction kit. In order to adapt the environment of the camelidae CDR3 regions to scaffold iMab100 two extra rounds of PCR similar to the first PCR method was performed on 5 μl of the products with the exception that the cycle number was decreased to 15 cycles and in which primer 73 (Table 5) and 75 (Table 5) were subsequently used as forward primer and primer 49 (Table 5) was used as reverse primer.

### Example 44

### Amplification of Cow-Derived CDR3 Regions

Cow (Bos taurus) blood lymphocytes were isolated according to standard procedures as described in Spinelli et al. (Biochemistry 39 (2000) 1217-1222). RNA from these cells was isolated via Qiagen RNeasy methods according to manufacturer's protocol. cDNA was generated using muMLv or AMV (New England Biolabs) according to manufacturer's procedure. CDR3 regions from Vh cDNA was amplified using 1 μl cDNA reaction in 100 microliters PCR reaction mix comprising two units Taq polymerase (Roche), 200 μM of each dNTP (Roche), buffers (Roche Taq buffer system), 2.5 μM of primer 299 (Table 5) and 300 (Table 5) in a Primus96 PCR machine (MWG) with the following program 35 times (94° C. for 20 seconds, 50° C. for 25 seconds, 72° C. for 30 seconds). Products were separated on a 2% Agarose gel and products of the correct length were isolated and purified using Qiagen gel extraction kit. The length distribution of the PCR products observed (see FIG. 11) represents the average length of cow CDR3 regions. Correcting for framework sequences that are present in primer 299 (21 amino acids; Table 5) and 300 (27 amino acids; Table 5) it can be concluded that the average length of cow CDR3s is: 120 base average PCR product length minus 48 base frameworks determines 72 bases and thus 24 amino acids. This result corresponds very well with the results observed by Spinelli et al. (Biochemistry 39 (2000) 1217-1222). These CDR regions are therefore extremely useful for naive library constructions.

Isolated and purified products can be used to adapt the sequences around the actual CDR3/AR4 location in a way that the coding regions of the frameworks are gradually adapted via several PCR modification rounds similarly as described for llama-derived ARs (see Example 43).

### Example 45

### Libraries Containing Loop Variegations in AR4 by Insertion of Amplified CDR3 Regions

A nucleic acid phage display library having variegations in AR4 was prepared by the following method. Amplified CDR3 regions from llamas immunized with lactoperoxidase and lactoferrin was obtained as described in Example 43 and were digested with PstI and KpnI and ligated with T4 DNA ligase into the PstI- and KpnI-digested and alkaline phosphatase-treated vector CM114-iMab113 or CM114-iMab114. Cysteine-containing CDR3s were cloned into CM114-iMab114 while CDR3s without cysteines were cloned into vector CM114-iMab113. The libraries were constructed by electroporation into E. coli TG1 electrocompetent cells by using a BTX electrocell manipulator ECM 630. Cells were recovered in SOB and grown on plates that contained 4% glucose, 100 micrograms ampicillin per milliliter in 2*TY-agar. After overnight culture at 37° C., cells were harvested in 2*TY medium and stored in 50% glycerol as concentrated dispersions at -80° C. Typically, 5×10⁸ transformants were obtained with 1 μg DNA and a library contained about 109 independent clones.

### Example 46

### Libraries Containing Loop Variegations in AR4 by Insertion of Randomized CDR3 Regions

A nucleic acid phage display library having variegations in AR4 by insertion of randomized CDR3 regions was prepared by the following method. CDR3 regions from non-immunized and immunized llamas were amplified as described in Example 43 except that in the second PCR round dITP or dPTP were included as described in Example 39. Preparation of the library was performed as described in Example 45. With dITP, a mutation rate of 2% was achieved, while with dPTP included in the PCR, a mutation rate of over 20% was obtained.

### Example 47

### Enrichment of VAPs that Bind to Target Molecules

About 50 microliters of the library stocks was inoculated in 50 ml 2*TY/100 microgram ampicillin/4% glucose and grown until an OD600 of 0.5 was reached. Next, 10¹¹ VCSM13 (Stratagene) helper phages were added. The culture was left at 37° C. without shaking for 45 minutes to enable infection. Cells were pelleted by centrifugation and the supernatant was discarded. Pellets were resuspended in 400 ml 2*TY/100 micrograms ampicillin and cultured for one hour at 37° C. after which 50 μg/ml kanamycin was added. Infected cultures were grown at 30° C. for eight hours on a 200 rpm shaking platform. Next, bacteria were removed by pelleting at 5000 g at 4° C. for 30 minutes. The supernatant was filtered through a 0.45 micrometer PVDF filter membrane. Poly-ethylene-glycol and NaCl were added to the flow through with final concentrations of respectively 4% and 0.5 M. In this way, phages precipitated on ice and were pelleted by centrifugation at 6000 g. The phage pellet was solved in 50% glycerol/50% PBS and stored at -20° C.

The selection of phage-displayed VAPs was performed as follows. Approximately 1 μg of a target molecule (antigen) was immobilized in an immunotube (Nunc) or microtiter plate (Nunc) in 0.1 m sodium carbonate buffer (pH 9.4) at 4° C. o/n. After the removal of this solution, the tubes were blocked with a 3% skim milk powder solution (ELK) in PBS or a similar blocking agent for at least two hours either at room temperature or at 4° C. o/n. After removal of the blocking agent a phagemid library solution containing approximately 10¹²-10¹³ colony forming units (cfu), which was preblocked with blocking buffer for one hour at room temperature, was added in blocking buffer. Incubation was performed on a slow rotating platform for one hour at room temperature. The tubes were then washed three times with PBS, two times with PBS with 0.1% Tween and again four times with PBS. Bound phages were eluted with an appropriate elution buffer, either 300 ml 0.1 m glycine pH 2.2 or 500 μl 0.1% trypsin in PBS. Recovered phages were immediately neutralized with 700 μl 1 m Tris-HCl pH 8.5 if eluted with glycine.

Alternatively the bound phages were eluted by incubation with PBS containing the antigen (1-10 μM). Recovered phages were amplified as described above employing E. coli XLI-Blue (Stratagene) or Top10F′ (InVitrogen) cells as the host. The selection process was repeated several times to concentrate positive clones. After the final round, individual clones were picked and their binding affinities and DNA sequences were determined.

The binding affinities of VAPs were determined by ELISA as described in Example 6, either as gIII-fusion protein on the phage particles or after subcloning as an NdeI-SfiI into the expression vector Cm126 as described in Example 4. E. coli BL21(DE3) or Origami(DE3) (Novagen) were transformed by electroporation as described in Example 5 and transformants were grown in 2×TY medium supplemented with ampicillin (100 μg/ml). When the cell cultures reached an OD600˜1 protein expression was induced by adding IPTG (0.2 mM). After four hours at 37° C., cells were harvested by centrifugation. Proteins were isolated as described in Example 7.

### Example 48

### Enrichment for Lactoferrin Binding VAPs

Purified Lactoferrin (LF) was Supplied by DMV-Campina.

A phage display library with variegations in AR4 as described in Example 45 was used to select LF-binding VAPs. LF (10 micrograms in 1 ml sodium bicarbonate buffer (0.1 m, pH 9.4)) was immobilized in an immunotube (Nunc) followed by blocking with 3% chicken serum in PBS. Panning was performed as described in Example 47. 10¹³ phages were used as input. After the first round of panning, about 10,000 colonies were formed. After the second panning round, 500 to 1,000 colonies were formed. Individual clones were grown and VAPs were produced and checked by ELISA as described in Example 8. Enrichment was found for clones with the following AR4:

| | |
|---|---|
| CAAQTGGPPAPYYCTEYGSPDSW | (SEQ ID NO:6) |

### Example 49

### Enrichment for Lactoperoxidase Binding VAPs

Purified Lactoperoxidase (LP) was Supplied by DMV-Campina.

A phage display library with variegations in AR4 as described in Example 45 was used to select LP-binding VAPs. LP (10 micrograms in 1 ml sodium bicarbonate buffer (0.1 m, pH 9.4)) was immobilized in an immunotube (Nunc) followed by blocking with 3% chicken serum in PBS. Panning was performed as described in Example 47. 10¹³ phages were used as input. After the first round of panning, about 5,000 colonies were formed. After the second panning round, 500 to 1,000 colonies were formed. Individual clones were grown and VAPs were produced and checked by ELISA as described in Example 8. Positive clones were sequenced. Enrichment was found for clones with the following AR4:

| | |
|---|---|
| CAAVLGCGYCDYDDGDVGSW | (SEQ ID NO:7) |
| CAATENFRIAREGYEYDYW | (SEQ ID NO:8) |
| CAATSDFRIAREDYEYDYW | (SEQ ID NO:9) |

### Example 50

### RNase A Binder, Construction, Maturation and Panning.

A synthetic RNase A-binding iMab, iMab130, was synthesized as described in Example 3 (Tables 4 and 3, respectively) and subsequently cloned into Cm114 forming CM114-iMab130. Chimeric phages with iMab1130 as a fusion protein with the g3 coat protein were produced under conditions as described for library amplification procedure in Example 47. Panning with these chimeric phages against RNase A-coated immunotubes (see Example 47 for panning procedure) failed to show RNase A-specific binding of iMab130. Functional positioning of the RNase A-binding regions had clearly failed, probably due to minor distortions of surrounding amino acid side chains. Small modifications of the scaffold might help to displace ARs into correct positions. In order to achieve this, the iMab130-coding region was mutated using the following method: iMab130 present in vector CM114 was mutagenized using either dITP or dPTP during amplification of the scaffold with primers 120 and 121 (Table 3). Mutagenizing concentrations of 1.7 mM dITP or 300 μM, 75 μM or 10 μM dPTP were used. Resulting PCR products were isolated from an agarose gel via Qiagen's gel elution system according to the manufacturer's procedures.

Isolated products were amplified in the presence of 100 μM of dNTPs (Roche) in order to generate dITP and dPTP free products. After purification via Qiagen's PCR clean-up kit, these PCR fragments were digested with NotI and SfiI (NEB) and ligated into NotI- and SfiI-linearized Cm114. Precipitated and 70% ethanol washed ligation products were transformed into TG1 by means of electroporation and grown in 2×TY medium containing 100 μg/ml ampicillin and 2% glucose and subsequently infected with VCSM13 helper phage (Stratagene) for chimeric phage production as described in Example 32. Part of the transformation was plated on 2×TY plates containing 2% glucose and 100 micrograms/ml ampicillin to determine transformation frequency:

These phage libraries were used in RNase A panning experiments as described in Example 32 RNase A was immobilized in immunotubes and panning was performed. After panning, phages were eluted and used for infection of TOP10 F′ (InVitrogen), and grown overnight at 37° C. on 2xTY plates containing 2% glucose and 100 μg/ml ampicillin and 25 microgram/ml tetracycline. The number of retrieved colonies is indicated in Table 17.

As can be concluded from the number of colonies obtained after panning with phage libraries derived from different mutagenesis levels of iMab130, a significant increase of binders can be observed from the library with a mild mutagenesis level, being dITP (Table 17)

### Example 51

### Immobilization Procedure

One gram of epoxy activated Sepharose 6B (manufacturer Amersham Biosciences) was packed in a column and washed with ten bed volumes coupling buffer (200 mM potassium phosphate, pH 7). The protein to be coupled was dissolved in coupling buffer at a concentration of 1 mg/ml and passed over the column at a flow rate of 0.1 ml/minute. After passing 20 bed volumes of protein solution, the column was washed with coupling buffer. Passing ten bed volumes of 0.2 M ethanolamine/200 mM potassium phosphate pH 7 blocked the unreacted epoxy groups. The resin was then washed with 20 bed volumes of 50 mM potassium phosphate pH 7 after which it was ready for use.

### Example 52

### iMab100 Purification via Lysozyme Immobilized Beads

Lysozyme was immobilized on Eupergit, an activated epoxy-resin from Rohm and used in a column. A solution containing iMab100 was passed on the column and the concentration was measured in a direct bypass and the flow through from the column (A280 nm). The difference indicated the amount of iMab100 that was bound to the column. The bound iMab100 could be released with a CAPS buffer pH11. Control experiments with BSA indicated that the binding of iMab 100 to immobilized lysozyme was specific.

### Example 53

### Lysozyme Purification via iMab100 Immobilized Beads

iMab100 was immobilized on Eupergit and used in a column. A solution containing Lysozyme was passed on the column and the concentration was measured and in a direct bypass and the flow through from the column (A280 nm). The difference indicated the amount of Lysozyme that was bound to the column. The bound Lysozyme could be released with a CAPS buffer pH11. Control experiments with BSA indicated that the binding of Lysozyme to immobilized iMab100 was specific.

### Example 54

### Stability of iMab 100 in Whey Fractions

The stability of iMab100 in several milk fractions was measured by lysozyme coated plates via ELISA methods (Example 8). If the tags, scaffold regions or affinity regions were proteolytically degraded, a decreased anti-lysozyme activity would be observed. iMab100 was diluted in several different solutions: 1×PBS as a control, ion-exchange fraction from cheese-whey, gouda-cheese-whey and low pasteurized undermilk, 1.4 μm filtered to a final concentration of 40 μg/ml. All fractions were stored at 8° C., samples were taken after: 0, two and five hours and after 1, 2, 3, 4, 5 and 7 days. Samples were placed at -20° C. to prevent further degradation. ELISA detection was performed as described in Example 8 and shown in FIG. 12. The activity pattern of iMab100 remained similar throughout the experiment. Therefore it can be concluded that iMab100, including the tags, were stable in assayed milk fractions.

### Example 55

### Preparation of Ligands

Human skin samples were harvested from two female donors undergoing cosmetic surgery (buttocks and abdomen) and were processed within two to six hours after removal with transport to the laboratory on dry ice at 4° C. Before removal, the skin was disinfected with propyl-ethanol based solution and iodine-betadine. Processing was started with three times washes in PBS to remove all blood under sterile conditions. A dermatome set at 0.3 mm thickness was used to shave the epidermis with a thin layer of dermis (the splitskin). The splitskin surface integrity was not preserved during this procedure and the samples were washed three more times in sterile PBS, then frozen to −80° C. To obtain keratin enriched skin fractions, the frozen samples were grinded in liquid nitrogen, rinsed with 2% non-ionic detergent (such as Tween-20, Triton X-100 or Brij-30) or ethanol. External lipids were removed using a mixture of chloroform-methanol (2:1) for 24 hours. The delipidized hair was resuspended in an alkaline buffer (such as Tris-HCl pH 9), preferably in 6 M urea, but a range of 5-8 M urea is possible, preferably 1 M thiourea but a range of 0-3 M thiourea is possible and 5% of a reducing agent (such as P-mercaptoethanol or dithiothroetol) and stirred at 50° C. for one to three days. The mixture was filtered and centrifuged (15,000 rpm, 30 minutes). The supernatant was dialyzed against 10-50 mM of an alkaline buffer (such as Tris-HCl pH 9) to remove low molecular weight impurities. The dialyzation buffer may contain additives such as reducing agents. The obtained protein fraction was used as an antigen and may be treated with iodoacetic acid to prevent reformation of disulfide bonds. The pellet fraction (containing insoluble proteins) was washed with distilled water and grounded using a homogenizer (such as a Wiley Mill) to a small particle size (i.e., all of the particles which pass through a 40 mesh screen). The small particles were resuspended in a buffer to a stable suspension, dialyzed and used as an antigen.

Human hair samples were harvested from diverse sources, representing different ethnic backgrounds and including, blond, brown, black curly, black straight and grey hairs. Cuticle-enriched fractions were obtained in a similar extraction procedure as described for keratin-enriched skin fractions.

### Example 56

### Coupling and Release of Fragrance Molecules to VAP

A C-8 Aldehyde (Octanal) was chosen to test labeling of the VAP with a volatile compound and subsequent release by hydrolysis. Octanal (MW 128.21) occurs in several citrus oils, e.g., orange oils. It is a colorless liquid with a pungent odor, which becomes citrus-like on dilution. Octanal was first allowed to react with the amino groups of the VAP and form an Imine bond. We then used aqueous solutions of HCl and NaOH to hydrolyze the bonds and release the volatile aldehyde.

1. Formation of an Imine Bond

Labeling the VAP with Octanal: 5 mg of VAP (iMab100) were dissolved in 500 μl of phosphate buffer (1.8 gram/liter Na₂HPO₄, 0.24 gram/liter KH₂PO₄ pH 7.5). 50 ml of C-8 Aldehyde (Octanal) were then added to the mixture, which was then allowed to incubate at room temperature for 18 hours.

Purification of the VAP-Fragrance Complex: The mixture solution from above was purified using a Ni-NTA column (spin column from Qiagen, used according to standard manufacturer procedures). The mixture was purified and all unbound fragrance was eluted using phosphate buffer by centrifuging six times for two minutes at 2000 rpm at 700×g. The column was then further air dried for 30 minutes to rid the column of all background fragrance from unbound Octanal.

Release of the Fragrance: Fragrance was released from the Ni-NTA column by adding a solution of either 3.7% aqueous HCl or a SM NaOH, spinning for two minutes at 2000 rpm at 700×g in a mini-centrifuge. Using a pump, air was flushed into the column and released fragrance was evaluated by a six person-panel. All release was obtained by evaluating the difference in fragrance from the VAP-fragrance complex upon addition of releasing agents.

| | Release with HCl | Release with NaOH |
|---|---|---|
| C8-Aldehyde | ++++ | ++ |
| No Fragrance | − | − |

In addition, the above described experiment was analyzed by head space analysis:

| | Sample | | | | | |
|---|---|---|---|---|---|---|
| | 1 | 2 | 3 | 4 | 5 (control) | 6 (control) |
| iMab100 (ul) | 300 | 300 | 300 | 300 | 300 | — |
| C-8 Aldehyde (ul) | 10 | 10 | 10 | 10 | — | 10 |
| Buffer (ul) | 100 | 100 | 100 | 100 | 100 | 100 |

The reaction mixtures were left overnight at room temperature for binding to occur (Schiff base) after which the samples were loaded onto a Ni-NTA Qiagen spin column. The columns were washed three times with a phosphate buffer pH 7.4 by spinning at 2000 rpm (700×g) for two minutes in a microcentrifuge followed by two washes with 40 μl 95% ethanol to remove unbound Octanal. The lid of the columns pierced with a needle and a JSPME headspace fiber (PDMS Carboxan) was inserted and allowed to equilibrate for 45 seconds (pre-release samples). Twenty μl of different concentrations of HCl varying from 0.5 to 0.01 M were added to the column to release the bound Octanal. Samples were taken as described above (release samples). The fibers were eluted and analyzed using the following method for GC-MS:

Sample inlet: GC
Injection source: Manual
Injection location: Front inlet port

Column
Capillary column (2): Union connection at front inlet/front detector/MSD
Model Number: Phenomenex Zebron ZB-1 (non-polar 100% polydimethylsiloxane)
Nominal length: 60.0 m each/total 120.0 m
Nominal diameter: 250.00 μm
Nominal film thickness: 0.25 μm
Mode: constant pressure
Pressure: 35.8 psi
Nominal initial flow: 2.2 mL/minute
Average velocity: 39 cm/second
Inlet: front inlet
Outlet: MSD
Outlet pressure: vacuum

Front Detector
Temperature: 250° C.
Hydrogen flow: 40.0 mL/minute
Air flow: 450.0 mL/minute
Mode: constant makeup flow
Makeup gas type: helium
Data rate: 5 hz

Thermal Aux 2
Use: MSD transfer line heater
Description: MSD interface
Initial temperature: 280° C.

Oven
Initial temperature: 80° C.
Initial time: 1.00 minute
Ramps: Rate: 8.50/Final Temp: 260° C./Final Time: 2.00
Run time: 23.00 min

Front Inlet
Mode: splitless
Initial temperature: 240° C.
Pressure: 35.8 psi
Purge flow: 50.0 mL/minute
Purge time: 0.50 minute
Total flow: 54.3 mL/minute
Gas type: helium

Mass Spec MS-D
Tune file: atune.u
Aquisition mode: scan
Solvent Delay: 2.00 minutes
Electron Multiplier Voltage: 2752.9
Scan parameters: low mass: 35/high mass: 450
MS Quad: 106° C.
MS Ion Source: 230° C.

Octanal Properties:
Boiling Point: 171° C.
d²⁰₄=0.8139

Experimental Values:
Odor Detection Threshold (in water)=0.00041−0.0064 ppm
Odor Detection Threshold (in air)=0.0058−0.0136

Calculated Values:
Odor Detection Threshold (water)=0.0042 ppm
Vapor Pressure (atm)=0.00329
Diffusion Coefficient (cm second)=0.0061
cLogP=2.78

The results are shown in Table 19. A clear release of Octanal was detected after addition of HCl.

2. Formation of the Amine Bond

To further test our assumption, the imine was further reduced using sodium borohydride to form a much more stable and non-hydrolyzable amine bond.

a. Labeling of the VAP: The procedure was repeated as outlined above for the Imine bond. In addition, after 18 hours, 100 μl of sodium borohydride (0.1 M pH 9) were added and the mixture was incubated at room temperature for 1.5 hours.

b. Purification of the VAP with fragrance: The mixture solution from above was purified using a Ni-NTA column (spin column from Qiagen, used according to standard manufacturer procedures). The mixture was purified and all unbound fragrance was eluted using phosphate buffer (0.5N pH 7) by centrifuging six times for two minutes at 2000 rpm at 700×g. The column was again allowed to air-dry using an air pump for 1.5 hours.

c. Testing for Release: using the same method as previously mentioned, a very minimal release of fragrance was now discemable and the olfactory index was comparable to a control of unbound VAP.

d. Release of Octanal-VAP complex from the Ni-NTA column: The Octanal-VAP complex was eluted from the column according to the standard manufacturer procedures, and diluted with SDS-PAGE sample buffer, boiled for five minutes at 95° C.

e. Molecular Weight determination: The Octanal labeled VAP was loaded on SDS-PAGE (15%, denaturing conditions) and was run against a control unbound VAP. The heavier (est. 1 kilodalton) molecular weight shown by the slower migration confirmed binding of the Octanal to VAP through an amide bond (results not shown).

### Example 57

### Coupling of Color Compound to VAP

Fluorescent dyes such as rhodamine can be covalently coupled to VAPs whereby the active binding properties are retained. Rhodamine and its derivatives are water-soluble basic dyes used in labeling all types of bio-molecules. Tetramethyl-rhodamine-5-(and 6)-isothiocyanate (TRITC) is a derivative of tetramethyl-rhodamine, which reacts with nucleophiles such as amines, sulfihydryls, and the phenolate ion of tyrosine side chains. The only stable product however is with the primary amine groups, and so TRITC is almost entirely selective for the modification of e- and N-terminal amines in proteins. The reaction involves attack of the nucleophile on the central, electrophilic carbon of the isothiocyanate group. Binding of TRITC to a VAP without loss of binding activity is here shown for iMab142-xx-002 (for amino acid sequence see Table 3). iMab 142-xx-002 has specific binding activity for Lactoferrin.

Ten μl aliquots of a TRITC solution (1 mg/ml in DMSO) were added five times to 1 ml iMab142-xx-002 solution (10 mg/ml in 0.1 M Na₂CO₃ pH 9) with mixing in between. The mixture was incubated overnight at 4° C. in the dark under gentle stirring. The labeled iMab was purified from unreacted TRITC by dialysis against PBS pH 7.4. SDS-PAGE analysis of the purified TRITC-labeled iMab revealed a single colored protein band thus demonstrating that covalent labeling of TRITC was successful (see FIG. 13, Lane 1). Specific binding of the TRITC labeled iMab142-xx-002 to lactoferrin was demonstrated by using a gel-shift assay. iMab 142-xx-002 (2 mg/ml) was mixed with either PBS 6.5, bovine serum albumin (10 mg/ml in PBS pH 6.5) or lactoferrin (10 mg/ml in PBS pH 6.5) and analyzed for migration on a 7.5% native PAA gel after electrophoresis (100V, 90 minutes) (FIG. 13). Migration is clearly repressed if TRITC-labeled iMab142-xx-0002 is mixed with lactoferrin indicating strong and specific binding (FIG. 13, Lane 3). The retardation factor (Rf) of the samples is:

TRITC-iMab: 0.66

TRITC-iMab+BSA: 0.66

TRITC-iMab+LF: 0.006

### Example 58

### Coupling of AntiCLys-VAP to Hair

The purpose of this experiment is to directly label hair coated with Lysozyme protein using a Rhodamine-TRITCC-labeled-antiCLys-VAP.

Hair strands (approximately 0.5 grams) were rinsed with potassium phosphate buffer (0.5 M pH 7.6). The hair strands were then immersed in 1.5 ml of a 25% solution of aqueous glutaraldehyde and incubated for 18 hours at 37° C. The hair was then washed thoroughly with phosphate buffer and water.

Hair strands were then transferred to a 1 ml solution of phosphate buffer, to which was also added 100 μl of Egg White Lysozyme (0.1 g in 1 ml stock solution). The mixture was allowed to react overnight at 4° C. The hair strands were then thoroughly washed with coupling buffer and then water.

All remaining aldehydes and other double bonds were then eliminated by adding 100 ml of sodium borohydride (0.1 M). The hair strands were then washed with water and then resuspended in 1 ml phosphate buffer.

10 μl of TRITC-labelled antiCLys-VAP with the highest protein content obtained from the G-25 purification step of the previous example was then added to an Eppendorf tube containing hair samples in 100 μl of 1×PBS buffer (pH 8). The reaction conditions are summarized below, indicating specific binding of TRITC-labeled VAPs to hair, via the crosslinked lysozyme that was coupled to the hair surface:

| | Sample | fluorescence |
|---|---|---|
| 1 | Hair + blocking agent | weak |
| 2 | Hair | weak |
| 3 | Hair-Lysozyme + blocking agent | strong |
| 4 | Hair-Lysozyme | strong |

An Elisa assay with Lysozyme bound to the surface of the wells confirmed that the TRITCC-coupling did not interfere with the VAP-affinity for lysozyme. The Elisa reaction was done with anti-Vsv-horse radish peroxidase, detecting the Vsv tag that is present on the carboxy terminals of the antiCLys-VAP

| sample size | VAP - TRITC | VAP | Blocking Buffer |
|---|---|---|---|
| 1000 ng | 0.239 | 0.266 | 0.065 |
| 500 ng | 0.132 | 0.136 | 0.074 |
| 250 ng | 0.089 | 0.099 | 0.044 |
| 100 ng | 0.077 | 0.118 | 0.045 |
| 50 ng | 0.063 | 0.055 | 0.040 |

### Example 59

### Covalent Coupling of Polymeric Compounds to VAPs

Polymers such as polymethacrylate and polyethyleneglycol can be covalently coupled to VAP whereby the active binding properties are retained. This is demonstrated by coupling iMab148-xx-0002 (for amino acid sequence, see Table 3), which binds to bovine lactoferrin, covalently to Eupergit 1014F. iMab148-xx-0002 is a derivative of iMab142-xx-0002. iMab142-xx-0002 was isolated as a lactoferrin binder as described in Examples 47 and 48. In order to couple iMab148-xx-0002 covalently to polymeric compounds without loss of affinity, all lysine residues were replaced by non-reactive amino acids. For amino acid sequence, see Table 3. In addition, the 6x his tag was removed and a tag containing lysines was added. This tag has the following composition: KSSKGKSK (SEQ ID NO:10) and is numbered 06. The tag exchange was performed in vector Cm126 according to standard molecular biology procedures. The resulting iMab is iMab148-06-0002. iMab148-06-0002 was produced as described in Example 7. 25 mg iMab148-06-0002 with affinity for bovine lactoferrin was mixed with 1 g of epoxy-activated metacrylic beads (Eupergit 1014F) and incubated overnight in 10 ml PBS pH 9+0.5 M NaCl at room temperature.

The unreacted epoxy groups were blocked by incubation with PBS pH 9+0.2 M ethanolamine for four hours. The resin was subsequently washed with 10 M urea+20 mM DTT to remove non-covalently bound proteins. Subsequent washing of the resin with PBS pH 8 allows correct refolding of the immobilized iMab. With the immobilized VAP, lactoferrin was isolated from casein whey, which was prepared as follows. Fresh cow milk was heated up to 35° C. and acidified with H2SO4 (30%) to pH 4.6. The precipitated milk solution was centrifuged (12,000 rpm, 30 minutes) to remove solids. The supernatant was adjusted to pH 6.5 and further clarified by ultracentrifugation (25,000 rpm, 30 minutes) and filtration (0.45 μm filter). The clarified casein whey (100 ml, in PBS pH 6.5) was loaded on an Eupergit 1014F column (2 ml) immobilized with 7.5 mg/ml iMab148-xx-0002. After loading, the column was washed with 20-column volumes of PBS pH 6.5 to remove aspecifically bound proteins. PBS+2 M NaCl was applied to elute specific bound proteins. Eupergit 1014F (2 ml) without immobilized iMab was used as a negative control. SDS-PAGE analysis of input and eluate protein fractions (FIG. 14), showed that lactoferrin (˜80 kDa) was specifically recovered (FIG. 15, Lane 5) despite its low concentration in bovine casein whey (0.05 g/l). Only little aspecific binding of lactoferrin to resin material is observed (FIG. 14, Lane 3), indicating that the major amount of bound lactoferrin is iMab specific.

### Example 60

### Covalent Coupling of Enzymes to VAPs

Enzymes such as horseradish peroxidase (HRP) can be covalently coupled to VAPs whereby the active binding properties of VAPs are retained.

iMab142-xx-0002 (2 mg/ml in 50 mM KPi pH 7.5+1 mM EDTA) was mixed with 10 μl N-succinimidyl S-acetylthioacetate (SATA, Pierce) (15 mg/ml in DMSO) and incubated for 30 minutes at room temperature. The mixture was dialyzed overnight against 50 mM KPi+1 mM EDTA to remove unreacted SATA. The thus obtained SATA-activated iMab (1 ml) was deacetylated by addition of 100 μl hydroxylamine (0.5 M in 50 mM KPi pH 7.5+25 mM EDTA) and subsequent incubation for two hours at room temperature. The mixture was dialyzed overnight against 50 mM KPi+1 mM EDTA to remove excess hydroxylamine. The deacetylated SATA-activated iMab (2 mg/ml) was mixed with maleimide activated HRP (Pierce) in a molar ratio of (1:5) and reacted overnight at 4° C. The thus obtained iMab142-xx-0002-HRP conjugate was purified from excess HRP by nickel-nitrile acetic acid agarose (Ni-NTA) chromatography according to the manufacturer's protocol. Specific binding of iMab142-xx-0002-HRP conjugate to lactoferrin was demonstrated by using a gel-shift assay. iMab 142-xx-0002-HRP (0.1 mg/ml) was mixed with either PBS 6.5, bovine serum albumin (10 mg/ml in PBS pH 6.5) or lactoferrin (10 mg/ml in PBS pH 6.5) and analyzed for in situ peroxidase activity with migration through a 7.5% native PAA gel using electrophoresis (100V, 90 minutes) (FIG. 15). Migration is clearly repressed if iMab142-xx-0002-HRP is mixed with lactoferrin indicating strong and specific binding (FIG. 15, Lane 3). The retardation factor (Rf) of the samples is:

HRP-iMab: 0.31

HRP-iMab+BSA: 0.31

HRP-iMab+LF: 0.008

### Example 61

### Selection of Hair and Skin Binding VAPs

Phage display libraries with variegations in AR4 were constructed as described in Example 45 by using amplified CDR3 regions of lamas (Lama glama) that were immunized with hair and skin proteins obtained as described in Example 55. Amplification of the CDR3 regions was performed as described in Example 43. In addition to introduction into phage display vector CM114-iMab113 and CM114-iMab114, the CDR3 regions were also introduced into CM114-iMab1300 and CM114-iMab1500 (Table 3). These iMabs have a seven beta-strand scaffold. CM114-iMab1300 and CM114-iMab1500 were constructed by insertion of the corresponding iMabs constructed as described in Example 3 as a NotI-SfiI fragment into CM 14 replacing iMab 100. In order to introduce appropriate restriction sites and to adapt the environment of the camelidae CDR3 regions to the scaffolds of iMab1300 and 1500 three extra rounds of PCR were performed (see Example 43). For introduction into iMab1300 primer 822/823/824 were used as forward primers and 829/811 and 830 were subsequently used as reverse primers. For introduction into iMab 1500 primers 813/814 were used as forward primers and 815/816/817 were subsequently used as reverse primer. For primers see Table 5. Enrichment for VAPs binding to the target molecules was performed as described in Example 47. As target molecules either soluble hair and skin proteins were used or whole pieces of hair and skin.

After the first round of panning about 100,000 colonies were formed. After the second panning round, 50,000 colonies were formed. After the third round of panning, about 10,000 colonies were formed. After three panning rounds with each library, at least 96 clones were sequenced to determine the nucleotide sequence of the presented iMab. Clones having identical nucleotide sequences and which were present more than two times within the 96 clones that were checked were selected for subcloning into the expression vector Cm126 as described in Example 4. VAPs were produced as described in Examples 5 and 7.

To test the binding of the VAPs to hair and skin, pieces of hair and skin were incubated with the VAPs (˜20 microgram protein in 500 microliter Phosphate buffer pH 7.4). After extensive washing (˜20x the sample volume) with phosphate buffer containing 0.1% tween-20 the binding VAPs were eluted with protein sample buffer (8%SDS, 40% glycerol in 0.25 M Tris-HCl buffer pH 6.8) and analyzed with SDS-PAGE. Binding VAPs was identified by Western Blotting. After gel-electrophoresis the proteins were transferred to PVDF membrane. After blocking for one hour at RT with 2% ELK (dried skim milk) in 1×PBS pH 7.4, a 1:20,000 diluted anti-VSV-HRP conjugated antibody was added. Aspecifically bound anti-VSV-HRP conjugated antibody was removed after one hour by washing the membrane four times for 15 minutes with 1×PBS pH 7.4 at RT. HRP activity was visualized with fluorescent substrate provided by Pierce (cat# 34095) according to the manufacturer's protocol. Fluorescence was detected with FluorChem™ 8900 (Alpha Innotech). Four VAPs, iMab143-xx-0029, iMab143-xx-0030, iMab143-xx-0033 and iMab143-xx-0034 showed binding to hair (FIG. 16). The amino acid sequences of the binding iMabs are shown in Table 20. To skin none of the tested iMabs showed binding in this experiment.

The VAPs were also analyzed by ELISA as described in Example 8. Purified VAP (˜50 ng) in 100 μl blocking buffer (0.5% BSA or Seablock) was added to a microtiter plate well coated with either 0.5% BSA(control), hair or skin proteins obtained as described in Example 55 blocked with 0.5% BSA or Seablock and incubated for 1 hour at room temperature on a table shaker (300 rpm). The microtiter plate was excessively washed with PBS (three times), PBS+0.1% Tween-20 (times) and PBS (three times). Bound VAPs were detected by incubating the wells with 100 μPBS containing anti-VSV-HRP conjugate (Roche) for one hour at room temperature. After excessive washing using PBS (three times), PBS+0.1% Tween-20 (three times) and PBS (three times), wells were incubated with 100 μl Turbo-TMB for five minutes. The reaction was stopped with 100 μl 2M H₂SO, and absorption was read at 450 nm using a microtiter plate rearder (Biorad). Seven of the in total 19 tested VAPs showed binding to skin proteins and also seven showed binding to hair proteins. The results are shown in Table 22. Five of the seven skin binding iMabs showed also binding to hair. iMab142-xx-0032 and 143-xx-0035 showed only binding to skin proteins so far and iMab142-xx-38 and iMab142-xx-39 only to hair proteins. The sequences of the binding VAPs are shown in Tables 20 and 21.

### Example 62

### Binding of VAPS to Human Skin Cryosections

Several iMabs isolated as described in Example 61 were tested for their binding capacity to human skin. Abdomen skin obtained after surgical correction with informed consent was dissected into pieces of 1×0.5 cm and snap-frozen in liquid nitrogen. Frozen sections 6 μm thick were air dried and fixated in acetone for ten minutes at 4° C. Endogenous peroxidase was inactivated with a 30-minute incubation in methanol containing 0.02% H₂O₂. After rehydration in water and PBS, aspecific binding was blocked with a 20 minute preincubation in PBS containing 10% Normal Horse Serum (NHS, Vector Laboratories). Excess serum was removed and iMab PBS solution were directly applied. The iMabs were used in a dilution described in the results.

After a two-hour incubation at RT, sections were briefly washed twice in excess PBS containing 0.1% NHS and for a one-hour incubation in PBS solution containing mouse-monoclonal anti-VSV-Hrp antibody diluted 1:250 and 1% NHS. Section were washed three times for five minutes in PBS containing 0.1% NHS before color reaction was performed in DAB metal concentrate solution (Pierce) for ten minutes. Sections were washed in demi-water and mounted in glycergel (Dako). Most iMabs did not show a specific binding and will be tested further. Two iMabs, iMab142-xx-0032 and iMab143-xx-0031, did show a specific staining (see FIG. 19). iMab142-xx-0032 stained some cells in the dermis, the layer underneath the epidermis and iMab143-xx-0031 stained all nuclei and the epidermis. Whether staining is specific for skin is not yet determined. But the results show that iMabs binding to components, proteins or cells in skin were isolated.

### Example 63

### Enrichments of Hair and/or Skin Binding VAPS with Identical AR Regions in Different Scaffolds

VAPs binding to hair and/or skin were isolated and produced as described in Example 61. Identical AR regions were enriched in different scaffolds, showing that binding to the target molecule is ont dependent o the scaffold but on the AR. The AR regions that were isolated are:

| | |
|---|---|
| 1. | AANDLLDYELDCIGMGPNEYED |
| 2. | AAVPGILDYELGTERQPPSCTTRRWDYDY |

AR region 1 was isolated from the libraries made in CM114-iMab113 resulting in a nine-beta-strand containing VAP, iMab142-xx-0036 and from CM114-iMab1500 resulting in a seven-beta-strand. Containing VAP, iMab143-xx-0036 AR region 2 was isolated from the libraries made in CM114-iMab1500 and from CM114-iMab1300 resulting both in a seven-beta-strand VAP but with different amino acid sequences, iMab143-xx-0037 and iMab144-xx-37, respectively.

The sequences of the iMabs are listed in Tables 20 and 21.

### Example 64

### Coupling of Fluorescent AntiHair-VAP to Hair

iMabs that were selected for their binding to hair as described in Example 61 were labeled with fluorescent dye (Alexa Fluor 488 carboxylic acid, succinimidyl ester, Molecular Probes cat# A-20000). While stirring, 100 μl dye (10 mg/ml in dimethylsulfoxide) was added to 900 μl iMab (2 mg/ml in 0.1 M sodium bicarbonate pH 8.3). The mix was allowed to react for 14 hours at 4° C. while mixing gently. The labeling reaction was stopped by the addition of 100 μl of freshly prepared 1.5 M hydroxylamine pH 8.5 to the labeling mix, incubated at 20° C. for one hour while mixing gently. Free dye was removed by dialysis over a 7000 Dalton dialysis membrane versus 1×PBS pH 7.4 for 24 hours, refreshing the dialysis buffer three times.

To determine labeling of the iMabs small samples (˜20 microliter) of labeled iMab were mixed with one volume loading buffer (8% SDS, 40% glycerol in 0.25 M Tris-HCl buffer pH 6.8) and allowed to denature for one hour at 20° C. Ten μl samples were loaded on a 1 mm 15% SDS-PA gel and run at 100 Volt. Fluorescence was detected with FluorChem™ 8900 (Alpha Innotech). All samples were labeled with Alexa-488 (FIG. 17). Binding to hair was determined as described in Example 61 except that binding was determined with confocal laser scanning microscopy (CLSM) (LSM510, Zeiss) instead of eluting with SDS-loading buffer. Binding of Alexa-488 labeled iMabs to hair is shown in Table 23. FIG. 18 shows the results of the CLSM for iMab143-xx-0030 and iMab143-xx-34.

### Example 65

### Bivalent Hair-Conditioning Agents

A VAP with hair binding specificity was selected from phage display libraries uses methods known to those skilled in the art or as described in Example 61. Bi-valent molecules (mono-specific or bi-specific for keratin) can easily be synthesized by duplicating the corresponding DNA sequence and adding flexible or inflexible, long or short spacers. As an illustrative example, a spacer is described in the sequence SGGGGSGGGGSGGGG. Such bi-valent VAPs are non-aggressive hair-perming agents as they tend to cross-link individual hairs directly upon contact. The flexibility of the spacer will determine the strength and feel of the perming agent, ranging from permanent hair waves to slight gelling agent effects. Besides the example spacer, many other spacers are described in scientific literature to fuse proteins together. Even cross-linking agents such as glutharaldehyde can be used to couple mono-valent VAPs in ways that the affinity to hair is not entirely lost.

| | |
|---|---|
| NVKLVEKGGNFVENDDDLKLTCRAEXXXXXXMGWFR | (SEQ ID NO:11) |
| QAPNDDSTNVATIXXXXXXYGDSVKERFDIRRDXXX | |
| XXXNTVTLSMDDLQPEDSAEYNCXXXXXXDSHYRGQ | |
| GTDVTVSS (VAP1) | |
| ggggsggggsggggs (linker) | (SEQ ID NO:12) |
| NVKLVEKGGNFVENDDDLKLTCRAEXXXXXXMGWFR | (SEQ ID NO:11) |
| QAPNDDSTNVATIXXXXXXYGDSVKERFDIRRDXXX | |
| XXXNTVTLSMDDLQPEDSAEYNCXXXXXXDSHYRGQ | |
| GTDVTVSS (VAP2) | |

To apply the perm agent to the hair, the hair is contacted with an effective amount of the bi-valent VAPs as described in the invention (i.e., an amount that is sufficient to achieve a noticeable conditioning effect to the hair, depending on the affinity characteristics of the surface-binding agent that is isolated from the panning procedure). Preferably, the perm agent is formulated with a suitable diluent that does not react with the perm agent, preferably a water-based diluent. Preferably, bi-valent VAPs are applied to the hair of one human head at a rate of 0.001 g to about 1 g per usage. In another preferred example, the bi-valent VAP is applied directly in a shampoo composition as are widely known in the art.

### Example 66

### VAPS without Cysteines

iMab122 was used as a template for the design and construction of completely cysteine-less VAPS. About 400 models were generated in which each individual cysteine was replaced by any other amino acid except for cysteine. All models were assessed by Prosa II. All acceptable models suggested replacement of the cysteine with hydrophobic amino acids residues (W, V, Y, F and I). Four models that showed the best ZP-values were selected for synthesis and testing (iMab138-xx-0007, 139-xx-0007, 140-xx-0007 and 141-xx-0007, Table 3 and FIGS. 22A-22I).

An oligonucleotide-mediated site-directed mutagenesis method was used to construct the iMabs. CM114-iMab122 was used as a template for the PCR reactions, together with oligonucleotide primers pr775, pr776, pr777, pr778, pr779, pr780 and pr78 (see Table 5). In the first PCR reaction, primers pr775 and pr779 were used for the construction of iMab138-xx-0007, primers pr776 and pr779 for the construction of iMab139-xx-0007, pr777 and pr780 for the construction of iMab140-xx-0007 and pr778 and pr781 for the construction of iMab141-xx-0007. The obtained PCR fragments were used as primers in two parallel PCR reactions with CM114-iMab122 as template. In one reaction, the fragments were used in combination with pr42 as forward primer and in the other reaction, the fragments were used in combination with pr51 as reverse primer. The obtained PCR fragments were isolated via agarose gel separation and isolation (Qiagen gel extraction kit). The products were mixed in an equimolar ratio and a fragment overlap-PCR reaction with primers pr42 and pr5 1. This PCR fragment was digested with NdeI and SfiI. The resulting fragment was isolated via an agarose gel and ligated into Cm126 linearized with NdeI and SfI. Sequence analysis confirmed that in the produced iMabs the cysteine residues were replaced by other amino acids (Table 3 and 4). The iMabs were produced and purified as described in Examples 5 and 7 and analyzed for CD spectra as described in Example 13. Each iMab spectra were measured at 20° C. and after heating for ten minutes at 80° C. and cooling to 20° C. For comparison also CD spectra of iMab111 with an extra cysteine bridge (see also Example 37) and iMab 116 with only one cysteine bridge (see also Example 31) were measured. The CD spectra of these two mutant iMabs are identical to the spectrum of iMab100 (see FIG. 9). The results are shown in FIG. 20. The double cysteine mutations iMab138-xx-0007, 139-xx-0007 and 141-xx-0007 are more affected by temperature treatment (FIGS. 20A and 20B). Especially iMab138-xx-0007 shows a decrease of more than 50% in magnitude after heating. iMab140-xx-0007 displays a more flattened CD spectrum which suggests less secondary structure. The iMab 140-xx-0007 CD spectrum is identical before and after heating. This shows that removal of all cysteines from the core does have an effect on the structure of the iMab but that impact of the effect on the structure is dependent on the substituted amino acids.

### Example 67

### VAPS with a Different pI Value

iMab100 was used as a template for the design of iMabs with a different isoelectric point (pI) by exchange of exposed amino acids with more acidic or alkaline amino acids depending on the desired pI, without loss of affinity. New iMabs were designed as described in Example 2 and three were synthesized based on their pI value, pI4.99, pI6.48 and 7.99, and their ZP-values, resulting in iMab135-xx-0002, iMab136-xx-0002 and iMab137-xx-0002, respectively. For the amino acid and nucleotide sequence, see Tables 3 and 4. The iMabs were synthesized as described in Example 3 and produced as described in Examples 4, 5 and 7. Their binding affinity was tested as described in Example 8. All three iMabs still bound lysozyme (results not shown). The CD spectra of the iMabs were measured at 20° C., at 80° C. and after heating for ten minutes at 80° C. and cooling to 20° C. as described in Example 13. The spectra are shown in FIG. 21. There is no difference between the CD spectra of these iMabs and of iMab 100. Also, heating does not influence the folding of the iMabs. This shows that the exposed amino acids can be changed without influencing the affinity or structure of the iMab.

### REFERENCES

1. Anonymous. FPLC purification of 6* His-tagged proteins from E. coli using Ni-NTA Superflow under native conditions in QIAexpressionist™, Fifth edition, 83-84, (2001)
2. Berens S J, Wylie D E, Lopez O J. Int Immunol, 9(1):189-99, (1997).
3. Beste G, Schmidt F S, Stibora T, Skerra A. Proc Natl Acad Sci U S A., 96, 1898-1903, (1999).
4. Better M, Chang C P, Robinson R R, Horowitz A H. Science, 240(4855):1041-3, (1988)
5. Cadwell et al., PCR Methods Appl., 2, 28-33, (1992)
6. Crane L J, Tibtech, 8, 12-16, (1990)
7. Dimasi N, Martin F, Volpari C, Brunetti M, Biasiol G, Altamura S, Cortese R, De Francesco R,
8. Steinkuhler C, Sollazzo M. J Virol. (10):7461-9., (1997)
9. Gibrat, J F, Madej, T, Bryant, S H, Curr. Op. Struct. Biol., 6(3), 377-385, (1996)
10. Hamers-Casterman C, Atarhouch T, Muyldermans S, Robinson G, Hamers C, Songa E B,
11. Holm, L and Sander, C. Nucl. Acids Res., 26, 316-319, (1998a)
12. Holm, L and Sander, C. Proteins, 33, 88-96, (1998b)
13. Koide S., Artificial antibody polypeptides, WO 98/56915, (1998)
14. Koide, U.S. Pat. No. 6,462,189.
15. Koide A, Bailey C W, Huang X, Koide S, J. Mol. Biol. 284 (4):1141-1151, (1998).
16. Kranz et al., WO Paent 0148145
17. Ku J, Schultz P G. Proc Natl Acad Sci USA. 92(14):6552-6, (1995)
18. Kuipers, Methods Mol Biol, 57 351-356, (1996)
19. Lauwereys M, Arbabi Ghahroudi M, Desmyter A, Kinne J, Holzer W, De Genst E, Wyns L,
20. McConnell S J, Hoess R H. J Mol Biol., 250(4):460-70, (1995)
21. Leung et al., Technique 1, 11-15, (1989)
22. Muyldermans S. EMBO J.,17(13):3512-20, (1998)
23. Murzin A. G et al. J. Mol. Biol., 247, 536-540, (1995)
24. Orengo C A, Jones D T, Thornton J M. Structure, 5(8) 1093-1108, (1997)
25. Rodger A. & Nordén B. in Circular dichroism and linear dichroism, Oxford University press, Oxford., (1997)
26. Shindyalov and Bourne Protein Engineering 11(9) 739-747, (1998)
27. Skerra A. Biochim Biophys Acta. October 18;1482(1-2):337-50, 2000.
28. Skerra A. J Biotechnol. June;74(4):257-75, (2001)
29. Skerra A, Pluckthun A. Science May 20;240(4855):1038-41, (1988)
30. Smith, G P, Patel S U, Windass J D, Thornton J M, Winter G, Griffiths A D. J Mol Biol. March 27;277(2):317-32, (1998)
31. Spee, J H, de Vos W M, Kuipers O P. Nucleic Acids Res 21(3):777-8, (1993)
32. Vriend, G. J. Mol. Graph. 8, 52-56, (1990)
33. Vu, K B, Ghahroudi, M. A., Wyns, L., Muyldermans, S. Mol. Immunol. 34, 1121-1131, (1997)
34. Xu et al., Biotechniques, 27, 1102-1108, (1999)

35. Zaccolo M, Williams D M, Brown D M, Gherardi E. J Mol Biol, 255(4):589-603, (1996)

TABLE 1
1Neu

| | | | | | | | | | | |
|---|---|---|---|---|---|---|---|---|---|---|
| ATOM | 1 | CA | GLY | 2 | −9.450 | −13.069 | 10.671 | 1.00 | 25.06 | C |
| ATOM | 2 | CA | GLY | 3 | −9.868 | −10.322 | 8.019 | 1.00 | 20.77 | C |
| ATOM | 3 | CA | GLY | 4 | −6.884 | −9.280 | 5.813 | 1.00 | 19.01 | C |
| ATOM | 4 | CA | GLY | 5 | −6.047 | −5.991 | 4.016 | 1.00 | 19.75 | C |
| ATOM | 5 | CA | GLY | 6 | −2.638 | −4.349 | 3.125 | 1.00 | 22.33 | C |
| ATOM | 6 | CA | GLY | 7 | −1.382 | −1.720 | 5.647 | 1.00 | 24.80 | C |
| ATOM | 7 | CA | GLY | 8 | −0.685 | 1.080 | 3.150 | 1.00 | 28.23 | C |
| ATOM | 8 | CA | GLY | 9 | −0.917 | 1.393 | −0.623 | 1.00 | 26.37 | C |
| ATOM | 9 | CA | GLY | 10 | 0.737 | 3.923 | −2.887 | 1.00 | 29.08 | C |
| ATOM | 10 | CA | GLY | 11 | −0.741 | 5.082 | −6.156 | 1.00 | 26.48 | C |
| ATOM | 11 | CA | GLY | 12 | 0.157 | 7.450 | −8.989 | 1.00 | 27.26 | C |
| ATOM | 12 | CA | GLY | 13 | −2.216 | 10.173 | −10.146 | 1.00 | 26.73 | C |
| ATOM | 13 | CA | GLY | 15 | −3.567 | 5.434 | −12.371 | 1.00 | 23.37 | C |
| ATOM | 14 | CA | GLY | 16 | −5.492 | 2.682 | −10.482 | 1.00 | 22.86 | C |
| ATOM | 15 | CA | GLY | 17 | −4.920 | 0.709 | −7.288 | 1.00 | 20.21 | C |
| ATOM | 16 | CA | GLY | 18 | −6.462 | −2.512 | −5.933 | 1.00 | 19.23 | C |
| ATOM | 17 | CA | GLY | 19 | −7.735 | −2.659 | −2.366 | 1.00 | 17.13 | C |
| ATOM | 18 | CA | GLY | 20 | −7.524 | −6.278 | −1.141 | 1.00 | 19.06 | C |
| ATOM | 19 | CA | GLY | 21 | −9.914 | −7.812 | 1.355 | 1.00 | 15.28 | C |
| ATOM | 20 | CA | GLY | 22 | −10.325 | −11.479 | 2.238 | 1.00 | 15.88 | C |
| ATOM | 21 | CA | GLY | 23 | −11.233 | −13.572 | 5.249 | 1.00 | 18.12 | C |
| ATOM | 22 | CA | GLY | 24 | −10.228 | −16.988 | 6.550 | 1.00 | 18.67 | C |
| ATOM | 23 | CA | GLY | 25 | −11.569 | −19.457 | 9.107 | 1.00 | 20.29 | C |
| ATOM | 24 | CA | GLY | 33 | −21.431 | −13.432 | 3.640 | 1.00 | 24.64 | C |
| ATOM | 25 | CA | GLY | 34 | −21.423 | −9.665 | 3.090 | 1.00 | 21.24 | C |
| ATOM | 26 | CA | GLY | 35 | −18.613 | −7.157 | 2.347 | 1.00 | 17.90 | C |
| ATOM | 27 | CA | GLY | 36 | −18.908 | −3.427 | 3.018 | 1.00 | 16.78 | C |
| ATOM | 28 | CA | GLY | 37 | −16.219 | −0.829 | 2.103 | 1.00 | 15.77 | C |
| ATOM | 29 | CA | GLY | 38 | −16.123 | 2.573 | 3.946 | 1.00 | 16.15 | C |
| ATOM | 30 | CA | GLY | 39 | −13.870 | 5.619 | 3.251 | 1.00 | 17.11 | C |
| ATOM | 31 | CA | GLY | 40 | −12.494 | 8.314 | 5.642 | 1.00 | 19.95 | C |
| ATOM | 32 | CA | GLY | 46 | −16.461 | 10.313 | 9.058 | 1.00 | 25.44 | C |
| ATOM | 33 | CA | GLY | 47 | −16.556 | 6.820 | 7.445 | 1.00 | 21.65 | C |
| ATOM | 34 | CA | GLY | 48 | −18.735 | 6.834 | 4.274 | 1.00 | 18.17 | C |
| ATOM | 35 | CA | GLY | 49 | −19.877 | 3.539 | 2.632 | 1.00 | 16.87 | C |
| ATOM | 36 | CA | GLY | 50 | −18.781 | 3.271 | −1.024 | 1.00 | 16.20 | C |
| ATOM | 37 | CA | GLY | 51 | −19.542 | −0.410 | −1.819 | 1.00 | 18.17 | C |
| ATOM | 38 | CA | GLY | 58 | −23.016 | −6.057 | −5.656 | 1.00 | 25.27 | C |
| ATOM | 39 | CA | GLY | 59 | −24.037 | −2.432 | −4.897 | 1.00 | 25.25 | C |
| ATOM | 40 | CA | GLY | 60 | −21.801 | 0.483 | −5.960 | 1.00 | 27.11 | C |
| ATOM | 41 | CA | GLY | 61 | −22.391 | 4.033 | −4.746 | 1.00 | 33.08 | C |
| ATOM | 42 | CA | GLY | 69 | −14.428 | 4.962 | −10.168 | 1.00 | 19.37 | C |
| ATOM | 43 | CA | GLY | 70 | −15.191 | 1.646 | −8.389 | 1.00 | 17.40 | C |
| ATOM | 44 | CA | GLY | 71 | −14.920 | −1.883 | −9.862 | 1.00 | 17.48 | C |
| ATOM | 45 | CA | GLY | 72 | −15.954 | −5.092 | −8.038 | 1.00 | 17.29 | C |
| ATOM | 46 | CA | GLY | 73 | −13.296 | −7.784 | −8.446 | 1.00 | 17.82 | C |
| ATOM | 47 | CA | GLY | 74 | −13.990 | −10.198 | −5.611 | 1.00 | 20.26 | C |
| ATOM | 48 | CA | GLY | 81 | −14.142 | −8.971 | −1.381 | 1.00 | 15.95 | C |
| ATOM | 49 | CA | GLY | 82 | −11.604 | −6.836 | −3.256 | 1.00 | 14.51 | C |
| ATOM | 50 | CA | GLY | 83 | −12.322 | −3.672 | −5.337 | 1.00 | 14.59 | C |
| ATOM | 51 | CA | GLY | 84 | −10.287 | −1.441 | −7.646 | 1.00 | 15.51 | C |
| ATOM | 52 | CA | GLY | 85 | −10.204 | 2.403 | −7.291 | 1.00 | 16.32 | C |
| ATOM | 53 | CA | GLY | 86 | −9.791 | 3.931 | −10.768 | 1.00 | 17.40 | C |
| ATOM | 54 | CA | GLY | 87 | −8.338 | 7.203 | −12.126 | 1.00 | 20.53 | C |
| ATOM | 55 | CA | GLY | 89 | −6.478 | 11.899 | −7.844 | 1.00 | 33.26 | C |
| ATOM | 56 | CA | GLY | 90 | −4.328 | 13.752 | −5.293 | 1.00 | 33.41 | C |
| ATOM | 57 | CA | GLY | 91 | −7.275 | 14.318 | −3.027 | 1.00 | 28.24 | C |
| ATOM | 58 | CA | GLY | 92 | −8.033 | 10.627 | −2.715 | 1.00 | 23.61 | C |
| ATOM | 59 | CA | GLY | 93 | −5.654 | 10.161 | 0.314 | 1.00 | 22.12 | C |
| ATOM | 60 | CA | GLY | 94 | −7.413 | 8.486 | 3.205 | 1.00 | 18.21 | C |
| ATOM | 61 | CA | GLY | 95 | −8.183 | 5.294 | 5.047 | 1.00 | 18.57 | C |
| ATOM | 62 | CA | GLY | 96 | −10.462 | 2.502 | 3.647 | 1.00 | 17.72 | C |
| ATOM | 63 | CA | GLY | 97 | −12.048 | −0.109 | 5.899 | 1.00 | 18.72 | C |
| ATOM | 64 | CA | GLY | 98 | −13.364 | −3.549 | 4.893 | 1.00 | 18.53 | C |
| ATOM | 65 | CA | GLY | 99 | −16.169 | −4.934 | 7.135 | 1.00 | 18.45 | C |
| ATOM | 66 | CA | GLY | 100 | −17.005 | −8.651 | 6.806 | 1.00 | 17.71 | C |
| ATOM | 67 | CA | GLY | 108 | −18.629 | −7.767 | 11.674 | 1.00 | 32.51 | C |
| ATOM | 68 | CA | GLY | 109 | −14.846 | −7.953 | 11.718 | 1.00 | 28.62 | C |
| ATOM | 69 | CA | GLY | 110 | −12.921 | −5.079 | 10.098 | 1.00 | 23.31 | C |
| ATOM | 70 | CA | GLY | 111 | −9.483 | −4.260 | 8.692 | 1.00 | 19.82 | C |
| ATOM | 71 | CA | GLY | 112 | −8.175 | −1.005 | 7.171 | 1.00 | 19.75 | C |
| ATOM | 72 | CA | GLY | 113 | −5.635 | 0.154 | 4.554 | 1.00 | 18.42 | C |
| ATOM | 73 | CA | GLY | 114 | −4.325 | 3.731 | 4.046 | 1.00 | 18.70 | C |
| ATOM | 74 | CA | GLY | 115 | −3.915 | 5.265 | 0.576 | 1.00 | 19.95 | C |
| ATOM | 75 | CA | GLY | 116 | −1.274 | 7.843 | −0.510 | 1.00 | 25.78 | C |
| ATOM | 76 | CA | GLY | 117 | −1.158 | 9.173 | −4.076 | 1.00 | 31.06 | C |
| ATOM | 77 | CA | GLY | 118 | 1.962 | 10.836 | −5.500 | 1.00 | 38.60 | C |
| ATOM | 78 | CA | GLY | 119 | 3.251 | 11.884 | −8.972 | 1.00 | 42.50 | C |
| TER | | | | | | | | | | |

1MEL

| | | | | | | | | | | | |
|---|---|---|---|---|---|---|---|---|---|---|---|
| ATOM | 79 | CA | GLY | A | 3 | −9.610 | −12.241 | 11.306 | 1.00 | 36.96 | C |
| ATOM | 80 | CA | GLY | A | 4 | −9.672 | −9.746 | 8.420 | 1.00 | 28.80 | C |
| ATOM | 81 | CA | GLY | A | 5 | −6.352 | −8.965 | 6.761 | 1.00 | 31.64 | C |
| ATOM | 82 | CA | GLY | A | 6 | −6.225 | −6.212 | 4.154 | 1.00 | 24.68 | C |
| ATOM | 83 | CA | GLY | A | 7 | −3.391 | −5.808 | 1.630 | 1.00 | 21.60 | C |
| ATOM | 84 | CA | GLY | A | 8 | −2.679 | −3.267 | −1.055 | 1.00 | 17.27 | C |
| ATOM | 85 | CA | GLY | A | 9 | −2.837 | 0.450 | −0.715 | 1.00 | 18.58 | C |
| ATOM | 86 | CA | GLY | A | 10 | 0.112 | 2.848 | −0.895 | 1.00 | 16.78 | C |
| ATOM | 87 | CA | GLY | A | 11 | 0.663 | 5.945 | −3.023 | 1.00 | 11.36 | C |
| ATOM | 88 | CA | GLY | A | 12 | −0.001 | 6.528 | −6.640 | 1.00 | 8.84 | C |
| ATOM | 89 | CA | GLY | A | 13 | −0.394 | 9.450 | −8.944 | 1.00 | 11.16 | C |
| ATOM | 90 | CA | GLY | A | 14 | −3.805 | 10.880 | −9.667 | 1.00 | 10.62 | C |
| ATOM | 91 | CA | GLY | A | 16 | −3.498 | 5.586 | −11.682 | 1.00 | 7.90 | C |
| ATOM | 92 | CA | GLY | A | 17 | −5.029 | 2.507 | −10.132 | 1.00 | 7.54 | C |
| ATOM | 93 | CA | GLY | A | 18 | −4.536 | 0.406 | −6.998 | 1.00 | 6.47 | C |
| ATOM | 94 | CA | GLY | A | 19 | −5.823 | −2.962 | −5.868 | 1.00 | 5.43 | C |
| ATOM | 95 | CA | GLY | A | 20 | −6.773 | −3.739 | −2.263 | 1.00 | 8.09 | C |
| ATOM | 96 | CA | GLY | A | 21 | −7.534 | −7.231 | −0.907 | 1.00 | 8.76 | C |
| ATOM | 97 | CA | GLY | A | 22 | −9.056 | −8.745 | 2.173 | 1.00 | 8.33 | C |
| ATOM | 98 | CA | GLY | A | 23 | −9.100 | −12.281 | 3.393 | 1.00 | 13.77 | C |
| ATOM | 99 | CA | GLY | A | 24 | −11.485 | −13.177 | 6.222 | 1.00 | 17.53 | C |
| ATOM | 100 | CA | GLY | A | 25 | −9.970 | −15.910 | 8.360 | 1.00 | 23.23 | C |
| ATOM | 101 | CA | GLY | A | 26 | −11.324 | −17.985 | 11.176 | 1.00 | 25.25 | C |
| ATOM | 102 | CA | GLY | A | 32 | −22.537 | −12.961 | 4.478 | 1.00 | 5.00 | C |
| ATOM | 103 | CA | GLY | A | 33 | −21.061 | −9.574 | 3.450 | 1.00 | 4.64 | C |
| ATOM | 104 | CA | GLY | A | 34 | −17.557 | −8.097 | 2.923 | 1.00 | 2.48 | C |
| ATOM | 105 | CA | GLY | A | 35 | −17.173 | −4.447 | 1.958 | 1.00 | 2.00 | C |
| ATOM | 106 | CA | GLY | A | 36 | −14.942 | −1.420 | 2.147 | 1.00 | 3.40 | C |
| ATOM | 107 | CA | GLY | A | 37 | −15.248 | 1.775 | 4.153 | 1.00 | 6.64 | C |
| ATOM | 108 | CA | GLY | A | 38 | −12.980 | 4.768 | 4.009 | 1.00 | 8.74 | C |
| ATOM | 109 | CA | GLY | A | 39 | −12.174 | 7.634 | 6.381 | 1.00 | 19.24 | C |
| ATOM | 110 | CA | GLY | A | 44 | −17.378 | 11.364 | 7.293 | 1.00 | 26.93 | C |
| ATOM | 111 | CA | GLY | A | 45 | −16.728 | 7.653 | 7.090 | 1.00 | 16.63 | C |
| ATOM | 112 | CA | GLY | A | 46 | −17.836 | 6.562 | 3.651 | 1.00 | 13.26 | C |
| ATOM | 113 | CA | GLY | A | 47 | −19.278 | 3.220 | 2.664 | 1.00 | 8.30 | C |
| ATOM | 114 | CA | GLY | A | 48 | −17.484 | 2.453 | −0.627 | 1.00 | 6.32 | C |
| ATOM | 115 | CA | GLY | A | 49 | −18.387 | −0.943 | −1.936 | 1.00 | 3.71 | C |
| ATOM | 116 | CA | GLY | A | 57 | −24.217 | −9.042 | −5.792 | 1.00 | 11.04 | C |
| ATOM | 117 | CA | GLY | A | 58 | −22.300 | −5.759 | −5.582 | 1.00 | 7.10 | C |
| ATOM | 118 | CA | GLY | A | 59 | −23.147 | −2.237 | −4.440 | 1.00 | 9.00 | C |
| ATOM | 119 | CA | GLY | A | 60 | −21.067 | 0.930 | −4.751 | 1.00 | 8.36 | C |
| ATOM | 120 | CA | GLY | A | 61 | −21.147 | 4.392 | −3.266 | 1.00 | 15.85 | C |
| ATOM | 121 | CA | GLY | A | 67 | −14.348 | 3.577 | −11.091 | 1.00 | 12.68 | C |
| ATOM | 122 | CA | GLY | A | 68 | −14.176 | 0.900 | −8.416 | 1.00 | 8.15 | C |
| ATOM | 123 | CA | GLY | A | 69 | −15.003 | −2.767 | −8.799 | 1.00 | 8.39 | C |
| ATOM | 124 | CA | GLY | A | 70 | −15.301 | −5.266 | −5.949 | 1.00 | 5.47 | C |
| ATOM | 125 | CA | GLY | A | 71 | −15.018 | −8.998 | −6.510 | 1.00 | 8.48 | C |
| ATOM | 126 | CA | GLY | A | 72 | −14.299 | −12.215 | −4.617 | 1.00 | 18.00 | C |
| ATOM | 127 | CA | GLY | A | 79 | −12.288 | −10.021 | −1.938 | 1.00 | 9.55 | C |
| ATOM | 128 | CA | GLY | A | 80 | −10.619 | −7.230 | −3.968 | 1.00 | 4.94 | C |
| ATOM | 129 | CA | GLY | A | 81 | −11.319 | −3.691 | −4.814 | 1.00 | 4.92 | C |
| ATOM | 130 | CA | GLY | A | 82 | −9.808 | −2.556 | −8.096 | 1.00 | 7.44 | C |
| ATOM | 131 | CA | GLY | A | 83 | −9.608 | 1.233 | −8.010 | 1.00 | 6.55 | C |
| ATOM | 132 | CA | GLY | A | 84 | −9.109 | 2.986 | −11.359 | 1.00 | 9.49 | C |
| ATOM | 133 | CA | GLY | A | 85 | −9.157 | 6.689 | −12.211 | 1.00 | 12.54 | C |
| ATOM | 134 | CA | GLY | A | 87 | −8.265 | 11.163 | −7.900 | 1.00 | 15.46 | C |
| ATOM | 135 | CA | GLY | A | 88 | −6.724 | 12.875 | −4.855 | 1.00 | 13.94 | C |
| ATOM | 136 | CA | GLY | A | 89 | −10.223 | 13.046 | −3.286 | 1.00 | 18.68 | C |
| ATOM | 137 | CA | GLY | A | 90 | −9.896 | 9.253 | −2.924 | 1.00 | 9.55 | C |
| ATOM | 138 | CA | GLY | A | 91 | −7.043 | 9.782 | −0.407 | 1.00 | 6.54 | C |
| ATOM | 139 | CA | GLY | A | 92 | −8.201 | 8.111 | 2.809 | 1.00 | 6.38 | C |
| ATOM | 140 | CA | GLY | A | 93 | −7.841 | 5.198 | 5.205 | 1.00 | 6.33 | C |
| ATOM | 141 | CA | GLY | A | 94 | −9.509 | 2.130 | 3.746 | 1.00 | 6.08 | C |
| ATOM | 142 | CA | GLY | A | 95 | −11.083 | −0.367 | 6.028 | 1.00 | 10.12 | C |
| ATOM | 143 | CA | GLY | A | 96 | −12.264 | −3.845 | 5.241 | 1.00 | 10.76 | C |
| ATOM | 144 | CA | GLY | A | 97 | −15.411 | −5.059 | 6.995 | 1.00 | 9.08 | C |
| ATOM | 145 | CA | GLY | A | 98 | −17.534 | −8.225 | 7.154 | 1.00 | 8.28 | C |
| ATOM | 146 | CA | GLY | A | 122 | −15.862 | −7.486 | 11.967 | 1.00 | 15.49 | C |
| ATOM | 147 | CA | GLY | A | 123 | −13.351 | −4.917 | 11.000 | 1.00 | 12.37 | C |
| ATOM | 148 | CA | GLY | A | 124 | −9.811 | −4.988 | 9.801 | 1.00 | 14.34 | C |
| ATOM | 149 | CA | GLY | A | 125 | −6.730 | −2.842 | 10.015 | 1.00 | 22.49 | C |
| ATOM | 150 | CA | GLY | A | 126 | −6.910 | 0.334 | 7.957 | 1.00 | 17.72 | C |
| ATOM | 151 | CA | GLY | A | 127 | −4.732 | 0.802 | 4.921 | 1.00 | 16.51 | C |
| ATOM | 152 | CA | GLY | A | 128 | −3.822 | 4.319 | 3.804 | 1.00 | 16.84 | C |
| ATOM | 153 | CA | GLY | A | 129 | −4.119 | 5.119 | 0.160 | 1.00 | 11.90 | C |
| ATOM | 154 | CA | GLY | A | 130 | −2.710 | 8.445 | −0.901 | 1.00 | 8.75 | C |
| ATOM | 155 | CA | GLY | A | 131 | −3.277 | 9.842 | −4.344 | 1.00 | 14.37 | C |
| ATOM | 156 | CA | GLY | A | 132 | −0.480 | 12.243 | −5.478 | 1.00 | 23.32 | C |
| ATOM | 157 | CA | GLY | A | 133 | −0.447 | 15.425 | −7.580 | 1.00 | 36.14 | C |
| TER | | | | | | | | | | | |

1F97

| | | | | | | | | | | | |
|---|---|---|---|---|---|---|---|---|---|---|---|
| ATOM | 158 | CA | GLY | A | 29 | −9.830 | −13.499 | 10.551 | 1.00 | 41.25 | C |
| ATOM | 159 | CA | GLY | A | 30 | −9.746 | −10.552 | 8.150 | 1.00 | 22.43 | C |
| ATOM | 160 | CA | GLY | A | 31 | −6.475 | −9.224 | 6.722 | 1.00 | 24.73 | C |
| ATOM | 161 | CA | GLY | A | 32 | −4.787 | −7.203 | 3.981 | 1.00 | 20.95 | C |
| ATOM | 162 | CA | GLY | A | 33 | −1.574 | −7.581 | 1.983 | 1.00 | 28.77 | C |
| ATOM | 163 | CA | GLY | A | 34 | −0.760 | −3.875 | 2.262 | 1.00 | 33.48 | C |
| ATOM | 164 | CA | GLY | A | 35 | −2.198 | −1.487 | 4.855 | 1.00 | 27.47 | C |
| ATOM | 165 | CA | GLY | A | 36 | −0.223 | 1.510 | 3.544 | 1.00 | 29.20 | C |
| ATOM | 166 | CA | GLY | A | 37 | −0.984 | 1.885 | −0.160 | 1.00 | 23.99 | C |
| ATOM | 167 | CA | GLY | A | 38 | 0.681 | 4.472 | −2.392 | 1.00 | 24.19 | C |
| ATOM | 168 | CA | GLY | A | 39 | −0.199 | 4.783 | −6.071 | 1.00 | 15.35 | C |
| ATOM | 169 | CA | GLY | A | 40 | 0.260 | 7.491 | −8.737 | 1.00 | 12.64 | C |
| ATOM | 170 | CA | GLY | A | 41 | −2.766 | 9.641 | −9.587 | 1.00 | 9.24 | C |
| ATOM | 171 | CA | GLY | A | 43 | −3.890 | 4.861 | −12.131 | 1.00 | 14.44 | C |
| ATOM | 172 | CA | GLY | A | 44 | −5.807 | 1.735 | −11.160 | 1.00 | 21.52 | C |
| ATOM | 173 | CA | GLY | A | 45 | −5.444 | 0.202 | −7.726 | 1.00 | 22.48 | C |
| ATOM | 174 | CA | GLY | A | 46 | −6.964 | −2.720 | −5.861 | 1.00 | 21.22 | C |
| ATOM | 175 | CA | GLY | A | 47 | −7.458 | −2.391 | −2.118 | 1.00 | 20.06 | C |
| ATOM | 176 | CA | GLY | A | 48 | −7.106 | −5.988 | −0.927 | 1.00 | 13.56 | C |
| ATOM | 177 | CA | GLY | A | 49 | −9.118 | −7.626 | 1.851 | 1.00 | 19.14 | C |
| ATOM | 178 | CA | GLY | A | 50 | −8.738 | −11.362 | 2.401 | 1.00 | 22.49 | C |
| ATOM | 179 | CA | GLY | A | 51 | −10.667 | −13.442 | 4.932 | 1.00 | 20.58 | C |
| ATOM | 180 | CA | GLY | A | 52 | −11.032 | −17.051 | 6.076 | 1.00 | 24.55 | C |
| ATOM | 181 | CA | GLY | A | 53 | −13.512 | −18.935 | 8.234 | 1.00 | 17.82 | C |
| ATOM | 182 | CA | GLY | A | 57 | −19.932 | −13.290 | 4.102 | 1.00 | 10.39 | C |
| ATOM | 183 | CA | GLY | A | 58 | −21.330 | −9.790 | 3.738 | 1.00 | 8.00 | C |
| ATOM | 184 | CA | GLY | A | 59 | −18.524 | −7.465 | 2.656 | 1.00 | 7.62 | C |
| ATOM | 185 | CA | GLY | A | 60 | −18.773 | −3.717 | 3.244 | 1.00 | 6.84 | C |
| ATOM | 186 | CA | GLY | A | 61 | −16.384 | −0.804 | 2.777 | 1.00 | 8.48 | C |
| ATOM | 187 | CA | GLY | A | 62 | −16.301 | 2.656 | 4.292 | 1.00 | 13.92 | C |
| ATOM | 188 | CA | GLY | A | 63 | −14.245 | 5.726 | 3.449 | 1.00 | 11.89 | C |
| ATOM | 189 | CA | GLY | A | 64 | −13.094 | 8.083 | 6.189 | 1.00 | 23.18 | C |
| ATOM | 190 | CA | GLY | A | 68 | −16.689 | 12.612 | 6.413 | 1.00 | 46.37 | C |
| ATOM | 191 | CA | GLY | A | 69 | −17.861 | 8.988 | 6.532 | 1.00 | 32.33 | C |
| ATOM | 192 | CA | GLY | A | 70 | −19.096 | 7.427 | 3.276 | 1.00 | 18.88 | C |
| ATOM | 193 | CA | GLY | A | 71 | −19.976 | 3.841 | 2.373 | 1.00 | 12.11 | C |
| ATOM | 194 | CA | GLY | A | 72 | −18.208 | 2.531 | −0.728 | 1.00 | 9.54 | C |
| ATOM | 195 | CA | GLY | A | 73 | −19.659 | −0.955 | −0.492 | 1.00 | 9.17 | C |
| ATOM | 196 | CA | GLY | A | 77 | −22.346 | −4.092 | −3.568 | 1.00 | 17.96 | C |
| ATOM | 197 | CA | GLY | A | 78 | −20.630 | −0.882 | −4.664 | 1.00 | 11.41 | C |
| ATOM | 198 | CA | GLY | A | 79 | −22.720 | 2.163 | −3.704 | 1.00 | 8.01 | C |
| ATOM | 199 | CA | GLY | A | 85 | −15.239 | 3.577 | −11.142 | 1.00 | 17.93 | C |
| ATOM | 200 | CA | GLY | A | 86 | −15.128 | 0.968 | −8.358 | 1.00 | 15.44 | C |
| ATOM | 201 | CA | GLY | A | 87 | −15.569 | −2.740 | −9.020 | 1.00 | 16.79 | C |
| ATOM | 202 | CA | GLY | A | 88 | −16.272 | −5.311 | −6.312 | 1.00 | 14.07 | C |
| ATOM | 203 | CA | GLY | A | 89 | −14.727 | −8.756 | −5.888 | 1.00 | 16.75 | C |
| ATOM | 204 | CA | GLY | A | 91 | −10.820 | −10.288 | −2.524 | 1.00 | 17.13 | C |
| ATOM | 205 | CA | GLY | A | 92 | −11.033 | −6.489 | −2.552 | 1.00 | 12.84 | C |
| ATOM | 206 | CA | GLY | A | 93 | −12.232 | −3.404 | −4.409 | 1.00 | 12.33 | C |
| ATOM | 207 | CA | GLY | A | 94 | −10.610 | −2.146 | −7.602 | 1.00 | 12.43 | C |
| ATOM | 208 | CA | GLY | A | 95 | −10.518 | 1.519 | −8.590 | 1.00 | 11.12 | C |
| ATOM | 209 | CA | GLY | A | 96 | −10.279 | 1.926 | −12.355 | 1.00 | 16.95 | C |
| ATOM | 210 | CA | GLY | A | 97 | −8.644 | 5.292 | −11.532 | 1.00 | 21.11 | C |
| ATOM | 211 | CA | GLY | A | 99 | −7.443 | 11.068 | −7.844 | 1.00 | 22.37 | C |
| ATOM | 212 | CA | GLY | A | 100 | −5.937 | 13.065 | −4.977 | 1.00 | 23.75 | C |
| ATOM | 213 | CA | GLY | A | 101 | −9.515 | 13.338 | −3.639 | 1.00 | 22.84 | C |
| ATOM | 214 | CA | GLY | A | 102 | −9.383 | 9.634 | −2.783 | 1.00 | 15.51 | C |
| ATOM | 215 | CA | GLY | A | 103 | −6.718 | 10.015 | −0.070 | 1.00 | 11.57 | C |
| ATOM | 216 | CA | GLY | A | 104 | −7.876 | 8.577 | 3.237 | 1.00 | 9.52 | C |
| ATOM | 217 | CA | GLY | A | 105 | −8.530 | 5.333 | 5.050 | 1.00 | 12.64 | C |
| ATOM | 218 | CA | GLY | A | 106 | −10.727 | 2.534 | 3.753 | 1.00 | 7.32 | C |
| ATOM | 219 | CA | GLY | A | 107 | −12.073 | −0.044 | 6.175 | 1.00 | 7.52 | C |
| ATOM | 220 | CA | GLY | A | 108 | −13.078 | −3.483 | 4.952 | 1.00 | 9.97 | C |
| ATOM | 221 | CA | GLY | A | 109 | −15.819 | −4.936 | 7.140 | 1.00 | 12.91 | C |
| ATOM | 222 | CA | GLY | A | 110 | −16.559 | −8.639 | 6.792 | 1.00 | 11.27 | C |
| ATOM | 223 | CA | GLY | A | 118 | −17.046 | −10.415 | 12.491 | 1.00 | 13.29 | C |
| ATOM | 224 | CA | GLY | A | 119 | −13.800 | −8.756 | 11.482 | 1.00 | 15.55 | C |
| ATOM | 225 | CA | GLY | A | 120 | −12.342 | −5.613 | 9.917 | 1.00 | 11.36 | C |
| ATOM | 226 | CA | GLY | A | 121 | −9.139 | −4.141 | 8.532 | 1.00 | 11.36 | C |
| ATOM | 227 | CA | GLY | A | 122 | −8.151 | −0.571 | 7.641 | 1.00 | 11.14 | C |
| ATOM | 228 | CA | GLY | A | 123 | −6.080 | 0.520 | 4.643 | 1.00 | 11.58 | C |
| ATOM | 229 | CA | GLY | A | 124 | −4.595 | 3.978 | 4.206 | 1.00 | 15.52 | C |
| ATOM | 230 | CA | GLY | A | 125 | −4.504 | 5.200 | 0.621 | 1.00 | 9.61 | C |
| ATOM | 231 | CA | GLY | A | 126 | −2.079 | 7.899 | −0.493 | 1.00 | 10.84 | C |
| ATOM | 232 | CA | GLY | A | 127 | −2.415 | 9.054 | −4.098 | 1.00 | 10.58 | C |
| ATOM | 233 | CA | GLY | A | 128 | 0.985 | 10.216 | −5.373 | 1.00 | 14.41 | C |
| ATOM | 234 | CA | GLY | A | 129 | 1.308 | 13.675 | −6.915 | 1.00 | 12.32 | C |
| TER | | | | | | | | | | | |

1DQT

| | | | | | | | | | | | |
|---|---|---|---|---|---|---|---|---|---|---|---|
| ATOM | 235 | CA | GLY | C | 2 | −10.005 | −8.876 | 13.603 | 1.00 | 35.96 | C |
| ATOM | 236 | CA | GLY | C | 3 | −10.267 | −7.502 | 10.101 | 1.00 | 30.20 | C |
| ATOM | 237 | CA | GLY | C | 4 | −7.171 | −6.498 | 8.221 | 1.00 | 27.24 | C |
| ATOM | 238 | CA | GLY | C | 5 | −6.397 | −5.009 | 4.845 | 1.00 | 23.16 | C |
| ATOM | 239 | CA | GLY | C | 6 | −3.219 | −3.760 | 3.070 | 1.00 | 22.73 | C |
| ATOM | 240 | CA | GLY | C | 7 | −1.859 | −0.343 | 3.998 | 1.00 | 24.33 | C |
| ATOM | 241 | CA | GLY | C | 8 | −1.267 | 0.851 | 0.436 | 1.00 | 21.64 | C |
| ATOM | 242 | CA | GLY | C | 9 | −2.613 | 0.109 | −3.024 | 1.00 | 20.25 | C |
| ATOM | 243 | CA | GLY | C | 10 | −1.486 | 1.813 | −6.246 | 1.00 | 20.37 | C |
| ATOM | 244 | CA | GLY | C | 11 | −4.559 | 2.055 | −8.480 | 1.00 | 22.46 | C |
| ATOM | 245 | CA | GLY | C | 12 | −4.228 | 1.091 | −12.139 | 1.00 | 24.30 | C |
| ATOM | 246 | CA | GLY | C | 14 | −7.812 | 2.779 | −15.613 | 1.00 | 30.82 | C |
| ATOM | 247 | CA | GLY | C | 15 | −8.831 | 3.617 | −12.060 | 1.00 | 25.29 | C |
| ATOM | 248 | CA | GLY | C | 16 | −8.949 | 0.054 | −10.709 | 1.00 | 21.63 | C |
| ATOM | 249 | CA | GLY | C | 17 | −7.920 | −0.828 | −7.174 | 1.00 | 20.22 | C |
| ATOM | 250 | CA | GLY | C | 18 | −8.037 | −4.397 | −5.930 | 1.00 | 20.86 | C |
| ATOM | 251 | CA | GLY | C | 19 | −7.062 | −5.746 | −2.558 | 1.00 | 21.25 | C |
| ATOM | 252 | CA | GLY | C | 20 | −7.903 | −8.418 | 0.003 | 1.00 | 21.73 | C |
| ATOM | 253 | CA | GLY | C | 21 | −9.906 | −7.860 | 3.174 | 1.00 | 22.97 | C |
| ATOM | 254 | CA | GLY | C | 22 | −9.210 | −10.573 | 5.688 | 1.00 | 26.59 | C |
| ATOM | 255 | CA | GLY | C | 23 | −10.945 | −11.564 | 8.878 | 1.00 | 26.47 | C |
| ATOM | 256 | CA | GLY | C | 24 | −10.701 | −13.907 | 11.826 | 1.00 | 31.54 | C |
| ATOM | 257 | CA | GLY | C | 25 | −11.932 | −16.084 | 13.335 | 1.00 | 31.33 | C |
| ATOM | 258 | CA | GLY | C | 32 | −20.611 | −12.339 | 5.419 | 1.00 | 21.25 | C |
| ATOM | 259 | CA | GLY | C | 33 | −21.785 | −8.834 | 4.607 | 1.00 | 21.86 | C |
| ATOM | 260 | CA | GLY | C | 34 | −18.854 | −6.717 | 3.456 | 1.00 | 19.78 | C |
| ATOM | 261 | CA | GLY | C | 35 | −18.920 | −2.932 | 3.364 | 1.00 | 19.11 | C |
| ATOM | 262 | CA | GLY | C | 36 | −16.430 | −0.515 | 1.806 | 1.00 | 19.23 | C |
| ATOM | 263 | CA | GLY | C | 37 | −16.229 | 2.889 | 3.460 | 1.00 | 25.95 | C |
| ATOM | 264 | CA | GLY | C | 38 | −14.212 | 5.923 | 2.415 | 1.00 | 30.90 | C |
| ATOM | 265 | CA | GLY | C | 39 | −12.916 | 7.802 | 5.426 | 1.00 | 40.84 | C |
| ATOM | 266 | CA | GLY | C | 43 | −16.713 | 11.053 | 8.779 | 1.00 | 46.74 | C |
| ATOM | 267 | CA | GLY | C | 44 | −17.290 | 8.066 | 6.505 | 1.00 | 36.18 | C |
| ATOM | 268 | CA | GLY | C | 45 | −19.030 | 7.541 | 3.173 | 1.00 | 28.21 | C |
| ATOM | 269 | CA | GLY | C | 46 | −20.279 | 4.093 | 2.143 | 1.00 | 25.48 | C |
| ATOM | 270 | CA | GLY | C | 47 | −18.891 | 3.200 | −1.269 | 1.00 | 23.13 | C |
| ATOM | 271 | CA | GLY | C | 48 | −20.541 | −0.174 | −1.750 | 1.00 | 20.54 | C |
| ATOM | 272 | CA | GLY | C | 58 | −19.057 | −12.667 | −7.642 | 1.00 | 24.08 | C |
| ATOM | 273 | CA | GLY | C | 59 | −20.627 | −9.992 | −5.444 | 1.00 | 22.25 | C |
| ATOM | 274 | CA | GLY | C | 60 | −21.579 | −6.311 | −5.553 | 1.00 | 22.96 | C |
| ATOM | 275 | CA | GLY | C | 61 | −23.676 | −7.234 | −8.605 | 1.00 | 28.83 | C |
| ATOM | 276 | CA | GLY | C | 62 | −25.740 | −4.088 | −8.210 | 1.00 | 32.00 | C |
| ATOM | 277 | CA | GLY | C | 63 | −22.747 | −1.772 | −7.854 | 1.00 | 30.87 | C |
| ATOM | 278 | CA | GLY | C | 66 | −17.371 | −2.468 | −7.103 | 1.00 | 23.17 | C |
| ATOM | 279 | CA | GLY | C | 67 | −16.897 | −6.161 | −7.488 | 1.00 | 22.56 | C |
| ATOM | 280 | CA | GLY | C | 68 | −15.405 | −9.022 | −5.517 | 1.00 | 21.60 | C |
| ATOM | 281 | CA | GLY | C | 69 | −15.312 | −12.649 | −4.466 | 1.00 | 22.69 | C |
| ATOM | 282 | CA | GLY | C | 75 | −12.905 | −11.173 | 0.577 | 1.00 | 23.03 | C |
| ATOM | 283 | CA | GLY | C | 76 | −11.171 | −10.003 | −2.613 | 1.00 | 24.30 | C |
| ATOM | 284 | CA | GLY | C | 77 | −12.370 | −6.459 | −3.308 | 1.00 | 23.46 | C |
| ATOM | 285 | CA | GLY | C | 78 | −12.157 | −4.457 | −6.510 | 1.00 | 25.74 | C |
| ATOM | 286 | CA | GLY | C | 79 | −13.139 | −0.780 | −6.670 | 1.00 | 26.08 | C |
| ATOM | 287 | CA | GLY | C | 80 | −13.383 | 0.660 | −10.196 | 1.00 | 29.15 | C |
| ATOM | 288 | CA | GLY | C | 81 | −13.950 | 3.973 | −11.951 | 1.00 | 26.23 | C |
| ATOM | 289 | CA | GLY | C | 84 | −7.281 | 11.072 | −8.931 | 1.00 | 24.79 | C |
| ATOM | 290 | CA | GLY | C | 85 | −9.649 | 12.909 | −6.605 | 1.00 | 25.37 | C |
| ATOM | 291 | CA | GLY | C | 86 | −10.734 | 9.536 | −5.170 | 1.00 | 25.21 | C |
| ATOM | 292 | CA | GLY | C | 87 | −7.287 | 9.058 | −3.657 | 1.00 | 23.95 | C |
| ATOM | 293 | CA | GLY | C | 88 | −7.874 | 8.354 | 0.014 | 1.00 | 23.67 | C |
| ATOM | 294 | CA | GLY | C | 89 | −8.483 | 5.896 | 2.834 | 1.00 | 22.75 | C |
| ATOM | 295 | CA | GLY | C | 90 | −10.866 | 2.985 | 2.312 | 1.00 | 23.39 | C |
| ATOM | 296 | CA | GLY | C | 91 | −12.127 | 0.902 | 5.210 | 1.00 | 22.55 | C |
| ATOM | 297 | CA | GLY | C | 92 | −13.188 | −2.683 | 4.810 | 1.00 | 22.29 | C |
| ATOM | 298 | CA | GLY | C | 93 | −15.931 | −3.797 | 7.205 | 1.00 | 20.45 | C |
| ATOM | 299 | CA | GLY | C | 94 | −16.933 | −7.418 | 7.719 | 1.00 | 20.73 | C |
| ATOM | 300 | CA | GLY | C | 105 | −15.090 | −5.968 | 12.589 | 1.00 | 24.94 | C |
| ATOM | 301 | CA | GLY | C | 106 | −13.327 | −3.332 | 10.512 | 1.00 | 25.24 | C |
| ATOM | 302 | CA | GLY | C | 107 | −9.792 | −2.872 | 9.205 | 1.00 | 24.05 | C |
| ATOM | 303 | CA | GLY | C | 108 | −7.671 | 0.283 | 9.656 | 1.00 | 25.85 | C |
| ATOM | 304 | CA | GLY | C | 109 | −8.117 | 1.175 | 6.019 | 1.00 | 23.77 | C |
| ATOM | 305 | CA | GLY | C | 110 | −6.209 | 0.891 | 2.770 | 1.00 | 22.46 | C |
| ATOM | 306 | CA | GLY | C | 111 | −4.626 | 4.045 | 1.330 | 1.00 | 24.13 | C |
| ATOM | 307 | CA | GLY | C | 112 | −5.545 | 4.027 | −2.339 | 1.00 | 22.83 | C |
| ATOM | 308 | CA | GLY | C | 113 | −3.438 | 6.352 | −4.496 | 1.00 | 23.40 | C |
| ATOM | 309 | CA | GLY | C | 114 | −4.906 | 7.221 | −7.840 | 1.00 | 25.42 | C |
| END | | | | | | | | | | | |

TABLE 2

iMab100
NVKLVE--KGG-NFVEN--DDDL--KLTCRAEGYTI----GPYCMGWFRQ
APNDDSTNVATINMGGGITYYGDSVKERFDIRRDNASNTVTLSMDDLQP
ED---SAEYNCAGDSTIYASYYECGHGLSTGGYGYDSHYR--GQ-GTDVT
VSSA
iMab502
SVKFVC--KVLPNFWEN--NKDLPIKFTVRASGYTI----GPTCVGVFAQ
NPEDDSTNVATINMGGGITYYGDSVKLRFDIRRDNAKVTRTNSLDDVQP
EGRGKSFELTCAADSTIYASYYECGHGISTGGYGYDQVAR--YHRGIDIT
VDGP
iMab702
AVKSVF--KVSTNFIENDGTMDS--KLTFRASGYTI----GPQCLGFFQQ
GVPDDSTNVATINMGGGITYYGDSVKSIFDIRRDNAKDTYTASVDDNQP
E----DVEITCAADSTIYASYYECGHGISTGGYGYDLILRTLQK-GIDLF
VVPT
iMab1202 (1EJ6)
IVKLVM--EKR-GNFEN--GQDC--KLTIRASGYTI----GPACDGFFCQ
FPSDDSFSTED-NMGGGIT-VNDAMKPQFDIRRDNAKGTWTLSM-DFQP
EG---IYEMQCAADSTIYASYYECGHGISTGGYGYDNPVR--LG-GFDVD
VPDV
iMab1302
VVKVVI--KPSQNFIEN--GEDK--KFTCRASGYTI----GPKCIGWFSQ
NPEDDSTNVATINMGGGITYYGDSVKERFDIRRDNAKDTSTLSIDDAQP
ED---AGIYKCAADSTIYASYYECGHGISTGGYGYDSEA---TV-GVDIF
VKLM
iMab1502 (1NEU)
NVKVVT--KRE-NFGEN--GSDV--KLTCRASGYTI----GPICFGWFYQ
PEGDDSAISIFHNMGGGITDEVDTFKERFDIRRDNAKKTGTISIDDLQP
SD---NETFTCAADSTIYASYYECGHGISTGGYGYDGKTR--QV-GLDVF
VKVP
iMab1602
AVKPVIGSKAP-NFGEN---GDV--KTIDRASGYTI----GPTCGGVFFQ
GPTDDSTNVATINMGGGITYYGDSVKETFDIRRDNAKSTRTESYDDNQP
EG---LTEVKCAADSTIYASYYECGHGISTGGYGYDVSSR--LY-GYDIL
VGTQ

TABLE 3
VAPs amino acid sequences:

iMab100
NVKLVEKGGNFVENDDDLKLTCRAEGYTIGPYCMGWFRQAPNDDSTNVAT
INMGGGITYYGDSVKERFDIRRDNASNTVTLSMDDLQPEDSAEYNCAGDS
TIYASYYECGHGLSTGGYGYDSHYRGQGTDVTVSS
iMab101
VKLVEKGGNFVENDDDLKLTCRASGYTIGPYCMGWFRQAPNDDSTNVATI
NMGTVTLSMDDLQPEDSAEYNCAADSTIYASYYECGHGLSTGGYGYDSHY
RGQGTDVTVSS
iMab102
DLKLTCRASGYTIGPYCMGWFRQAPNDDSTNVATINMGTVTLSMDDLQPE
DSAEYNCAADSTIYASYYECGHGLSTGGYGYDSHYRGQGTDVTVSS
iMab111
NVKLVCKGGNFVENDDDLKLTCRAEGYTIGPYCMGWFRQAPNDDSTNVAT
INMGGGITYYGDSVKERFDIRRDNASNTVTLSMDDLQPEDSAEYNCAGDS
TIYASYYECGHGLSTGGYGYDSHYRCQGTDVTVSS
iMab112
NVKLVEKGGNFVENDDDLKLTCRAEGYTIGPYCMGWFCQAPNDDSTCVAT
INMGGGITYYGDSVKERFDIRRDNASNTVTLSMDDLQPEDSAEYNCAGDS
TIYASYYECGHGLSTGGYGYDSHYRGQGTDVTVSS
iMab113
NVKLVEKGGNFVENDDDLKLTCRAEGYTIGPYSMGWFRQAPNDDSTNVSC
INMGGGITYYGDSVKERFDIRRDNASNTVTLSMDDLQPEDSAEYNCAGDS
TIYASYYECGHGLSTGGYGYDSHYRGQGTDVTVSS
iMab114
NVKLVEKGGNFVENDDDLKLTCRAEGYTIGPYSMGWFRQAPNDDSTNVAT
INMGGGITYYGDSVKERFDIRRDNASNTVTLSMDDLQPEDSAEYNCAGDS
TIYASYYECGHGLSTGGYGYDSHYRGQGTDVTVSS
iMab115
NVKLVEKGGNFVENDDDLKLTCRAEGYTIGPYCMGWFRQAPNDDSTNVAT
INMGGGITYYGDSVKERFDIRRDQASNTVTLSMDDLQPEDSAEYNCAGDS
TIYASYYECGHGLSTGGYGYDSHYRGQGTDVTVSS
iMab116
NVKLVEKGGNFVENDDDLKLTCRAEGYTIGPYCMGWFRQAPNDDSTNVAT
INMGGGITYYGDSVKERFDIRRDNASNTVTLSMDDLQPEDSAEYNGAGDS
TIYGSYYECGHGLSTGGYGYDSHYRGQGTDVTVSS
iMab120
NVKLVEKGGNFVENDDDLKLTCRAEGYTIGPYCMGWFRQAPNDDSTNVAT
INMGGGITYYGDSVKERFDIRRDNASNTVTLSMDDLQPEDSAEYNCAGDS
TIYASYYECGHGLSTGGYGYDSRGQGTDVTVSS
iMab121
NVKLVEKGGNFVENDDDLKLTCRASGRSFSSYIMGWFRQAPNDDSTNVAT
ISETGGDIVYTNYGDSVKERFDIRRDIASNTVTLSMDDLQPEDSAEYNCA
GSVYGSGWRPDRYDYRGQGTDVTVSS
iMab124
DDLKLTCRASGRSFSSYIMGWFRQAPNDDSTNVATISETTVTLSMDDLQP
EDSAEYNCAGSVYGSGWRPDRYDYRGQGTDVTVSS
iMab122
NVKLVEKGGNFVENDDDLKLTCRASGRTFSSRTMGWFRQAPNDDSTNVAT
IRWNGGSTYYTNYGDSVKERFDIRVDQASNTVTLSMDDLQPEDSAEYNCA
GTDIGDGWSGRYDYRGQGTDVTVSS
iMab125
DDLKLTCRASGRTFSSRTMGWFRQAPNDDSTNVATIRWNTVTLSMDDLQP
EDSAEYNCAGTDIGDGWSGRYDYRGQGTDVTVSS
iMab123
NVKLVEKGGNFVENDDDLKLTCRASGRTFSRAAMGWFRQAPNDDSTNVAT
ITWSGRHTRYGDSVKERFDIRRDQASNTVTLSMDDLQPEDSAEYNCAGEG
SNTASTSPRPYDYRGQGTDVTVSS
iMab130
NVKLVEKGGNFVENDDDLKLTCRASGYAYTYIYMGWFRQAPNDDSTNVAT
IDSGGGGTLYGDSVKERFDIRRDKGSNTVTLSMDDLQPEDSAEYNCAAGG
YELRDRTYGQRGQGTDVTVSS
iMab201
VQLQASGGGSVQAGGSLRLSCRASGYTIGPYCMGWFRQAPGDDSEGVAAI
NMGTVYLLMNSLEPEDTAIYYCAADSTIYASYYECGHGLSTGGYGYDSWG
QGTQVTVSS
iMab300
VQLQQPGSNLVRPGASVKLSCKASGYTIGPSCIHWAKQRPGDGLEWIGEI
NMGTAYVDLSSLTSEDSAVYYCAADSTIYASYYECGHGLSTGGYGYDYWG
QGTTLTVSS
iMab302
ASVKLSCKASGYTIGPSCIHWAKQRPGDGLEWIGEINMGTAYVDLSSLTS
EDSAVYYCAADSTIYASYYECGHGLSTGGYGYDYWGQGTTLTVSS
iMab400
VQLVESGGGLVQPGGSLRLSCRASGYTIGPYCMNWVRQAPGDGLEWVGWI
NMGTAYLQMNSLRAEDTAVYYCAADSTIYASYYECGHGLSTGGYGYDVWG
QGTLVTVSS
iMab500
PNELCSVLPTHWRCNKTLPIAFKCRASGYTIGPTCVTVMAGNDEDYSNMG
ARFNDLRFVGRSGRGKSFTLTCAADSTIYASYYECGHGLSTGGYGYPQVA
TYHRAIKITVDGP
iMab502
SVKFVCKVLPNFWENNKDLPIKFTVRASGYTIGPTCVGVFAQNPEDDSTN
VATINMGGGITYYGDSVKLRFDIRRDNAKVTRTNSLDDVQPEGRGKSFEL
TCAADSTIYASYYECGHGLSTGGYGYDQVARYHRGIDITVDGP
iMab600
APVGLKARNADESGHVVLRCRASGYTIGPICYEVDVSAGQDAGSVQRVEI
NMGRTESVLSNLRGRTRYTFACAADSTIYASYYECGHGLSTGGYGYSEWS
EPVSLLTPS
iMab700
DKSTLAAVPTSIIADGLMASTITCEASGYTIGPACVAFDTTLGNNMGTYS
APLTSTTLGVATVTCAADSTIYASYYECGHGLSTGGYGYAAFSVPSVTVN
FTA
iMab702
AVKSVFKVSTNFIENDGTMDSKLTFRASGYTIGPQCLGFFQQGVPDDSTN
VATINMGGGITYYGDSVKSIFDIRRDNAKDTYTASVDDNQPEDVEITCAA
DSTIYASYYECGHGLSTGGYGYDLILRTLQKGIDLFVVPT
iMab701
MASTITCEASGYTIGPACVAFDTTLGNNMGTYSAPLTSTTLGVATVTCAA
DSTIYASYYECGHGLSTGGYGYAAFSVPSVTVNFTA
iMab800
GRSSFTVSTPDILADGTMSSTLSCRASGYTIGPQCLSFTQNGVPVSISPI
NMGSYTATVVGNSVGDVTITCAADSTIYASYYECGHGLSTGGYGYTLILS
TLQKKISLFP
iMab900
LTLTAAVIGDGAPANGKTAITVECTASGYTIGPQCVVITTNNGALPNKIT
ENMGVARIALTNTTDGVTVVTCAADSTIYASYYECGHGLSTGGYGYQRQS
VDTHFVK
iMab1000
HKPVIEKVDGGYLCKASGYTIGPECIELLADGRSYTKNMGEAFFAIDASK
VTCAADSTIYASYYECGHGLSTGGYGYHWKAEN
iMab1001
VDGGYLCKASGYTIGPECIELLADGRSYTKNMGEAFFAIDASKVTCAADS
TIYASYYECGHGLSTGGYGYHWKAEN
iMab1100
APVGLKARLADESGHVVLRCRASGYTIGPICYEVDVSAGNDAGSVQRVEI
LNMGTESVLSNLRGRTRYTFACAADSTIYASYYECGHGLSTGGYGYSAWS
EPVSLLTPS
iMab1200
HGLPMEKRGNFIVGQNCSLTCPASGYTIGPQCVFNCYFNSALAFSTENMG
EWTLDMVFSDAGIYTMCAADSTIYASYYECGHGLSTGGYGYNPVSLGSFV
VDSP
iMab1202
IVKLVMEKRGNFENGQDCKLTIRASGYTIGPACDGFFCQFPSDDSFSTED
NMGGGITVNDAMKPQFDIRRDNAKGTWTLSMDFQPEGIYEMQCAADSTIY
ASYYECGHGLSTGGYGYDNPVRLGGFDVDVPDV
iMab1300
LQVDIKPSQGEISVGESKFFLCQASGYTIGPCISWFSPNGEKLNMGSSTL
TIYNANIDDAGIYKCAADSTIYASYYECGHGLSTGGYGYQSEATVNVKIF
Q
iMab1302
VVKVVIKPSQNFIENGEDKKFTCRASGYTIGPKCIGWFSQNPEDDSTNVA
TINMGGGITYYGDSVKERFDIRRDNAKDTSTLSIDDAQPEDAGIYKCAAD
STIYASYYECGHGLSTGGYGYDSEATVGVDIFVKLM
iMab1301
ESKFFLCQASGYTIGPCISWFSPNGEKLNMGSSTLTIYNANIDDAGIYKC
AADSTIYASYYECGHGLSTGGYGYQSEATVNVKIFQ
iMab1400
VPRDLEVVAATPTSLLISCDASGYTIGPYCITYGETGGNSPVQEFTVPNMG
KSTATISGLKPGVDYTITCAADSTIYASYYECGHGLSTGGYGYSKPISINY
RT
iMab1500
IKVYTDRENYGAVGSQVTLHCSASGYTIGPICFTWRYQPEGDRDAISIFHY
NMGDGSIVIHNLDYSDNGTFTCAADSTIYASYYECGHGLSTGGYGYVGKTS
QVTLYVFE
iMab1502
NVKVVTKRENFGENGSDVKLTCRASGYTIGPICFGWFYQPEGDDSAISIFH
NMGGGITDEVDTFKERFDIRRDNAKKTGTISIDDLQPSDNETFTCAADSTI
YASYYECGHGLSTGGYGYDGKTRQVGLDVFVKVP
iMab1501
SQVTLHCSASGYTIGPICFTWRYQPEGDRDAISIFHYNMGDGSIVIHNLDY
SDNGTFTCAADSTIYASYYECGHGISTGGYGYVGKTSQVTLYVFE
iMab1600
SKPQIGSVAPNMGIPGNDVTITCRASGYTIGPTCGTVTFGGVTNMGNRIEV
YVPNMAAGLTDVKCAADSTIYASYYECGHGLSTGGYGYGVSSNLYSYNILS
iMab1602
AVKPVIGSKAPNFGENGDVKTIDRASGYTIGPTCGGVFFQGPTDDSTNVAT
INMGGGITYYGDSVKETFDIRRDNAKSTRTESYDDNQPEGLTEVKCAADST
IYASYYECGHGLSTGGYGYDVSSRLYGYDILVGTQ
iMab1700
KDPEIHLSGPLEAGKPITVKCSASGYTIGPLCIDLLKGDHLMKSQEFNMGS
LEVTFTPVIEDIGKVLVCAADSTIYASYYECGHGLSTGGYGYVRQAVKELQ
VD
iMab1701
KPITVKCSASGYTIGPLCIDLLKGDHLMKSQEFNMGSLEVTFTPVIEDIGK
VLVCAADSTIYASYYECGHGLSTGGYGYVRQAVKELQVD
iMab142-xx-0002
MNVKLVEKGGNFVENDDDLKLTCRAEGYTIGPYSMGWFRQAPNDDSTNVSC
INMGGGITYYGDSVKERFDIRRDNASNTVTLSMDDLQPEDSAVYNCAADWW
DGFTYGSTWYNPSSYDYRGQGTDVTVSS
iMab148-xx-0002
MNVHLVERGGNFVENDDDLNLTCRAEGYTIGPYSMGWFRQAPNDDSTNVAT
INMGGGITYYGDSVDERFDIRRDNASNTVTLSMDDLQPEDSAVYNCAADWW
DGFTYGSTWYNPSSYDYRGQGTDVTVSS
iMab135-xx-0001
MNVQLVESGGNFVENDQDLSLTCRASGYTIGPYCMGWFRQAPNQDSTGVAT
INMGGGITYYGDSVKERFRIRRDNASNTVTLSMQNLQPQDSANYNCAADST
IYASYYECGHGLSTGGYGYDSRGQGTSVTVSS
iMab136-xx-0001
MNVKLVEKGGNFVENDDDLRLTCRAEGYTIGPYCMGWFRQAPNRDSTNVAT
INMGGGITYYGDSVKERFDIRRDNASNTVTLSMTNLQPSDSASYNCAADST
IYASYYECGHGLSTGGYGYDSRGQGTRVTVSS
iMab137-xx-0001
MNVQLVESGGNFVENDQSLSLTCRASGYTIGPYCMGWFRQAPNSRSTGVAT
INMGGGITYYGDSVKGRFTIRRDNASNTVTLSMNDLQPRDSAQYNCAADST
IYASYYECGHGLSTGGYGYDSRGQGTDVTVSS
iMab138-xx-0007
MNVKLVEKGGNFVENDDDLKLTWRASGRTFSSRTMGWFRQAPNDDSTNVAT
IRWNGGSTYYTNYGDSVKERFDIRVDQASNTVTLSMDDLQPEDSAEYNVAG
TDIGDGWSGRYDYRGQGTDVTVSS
iMab139-xx-0007
MNVKLVEKGGNFVENDDDLKLTVRASGRTFSSRTMGWFRQAPNDDSTNVAT
IRWNGGSTYYTNYGDSVKERFDIRVDQASNTVTLSMDDLQPEDSAEYNVAG
TDIGDGWSGRYDYRGQGTDVTVSS
iMab140-xx-0007
MNVKLVEKGGNFVENDDDLKLTIRASGRTFSSRTMGWFRQAPNDDSTNVAT
IRWNGGSTYYTNYGDSVKERFDIRVDQASNTVTLSMDDLQPEDSAEYNYAG
TDIGDGWSGRYDYRGQGTDVTVSS
iMab141-xx-0007
MNVKLVEKGGNFVENDDDLKLTFRASGRTFSSRTMGWFRQAPNDDSTNVAT
IRWNGGSTYYTNYGDSVKERFDIRVDQASNTVTLSMDDLQPEDSAEYNIAG
TDIGDGWSGRYDYRGQGTDVTVSS

TABLE 4
iMab DNA sequences:

iMab D100

| | | | | | | | | |
|---|---|---|---|---|---|---|---|---|
| 1 | AATGTGAAAC | TGGTTGAAAA | AGGTGGCAAT | TTCGTCGAAA | ACGATGACGA | TCTTAAGCTC | ACGTGCCGTG | CTGAAGGTTA |
| 81 | CACCATTGGC | CCGTACTGCA | TGGGTTGGTT | CCGTCAGGCG | CCGAACGACG | ACAGTACTAA | CGTGGCCACG | ATCAACATGG |
| 161 | GTGGCGGTAT | TACGTACTAC | GGTGACTCCG | TCAAAGAGCG | CTTCGATATC | CGTCGCGACA | ACGCGTCCAA | CACCGTTACC |
| 241 | TTATCGATGG | ACGATCTGCA | ACCGGAAGAC | TCTGCAGAAT | ACAATTGTGC | AGGTGATTCT | ACCATTTACG | CGAGCTATTA |
| 321 | TGAATGTGGT | CATGGCCTGA | GTACCGGCGG | TTACGGCTAC | GATAGCCAGT | ACCGTGGTCA | GGGTACCGAC | GTTACCGTCT |
| 401 | CG | | | | | | | |

iMab D101

| | | | | | | | | |
|---|---|---|---|---|---|---|---|---|
| 1 | CATA | TGGTTAAACT | GGTTGAAAAA | GGTGGTAACT | TCCTTGAAAA | CGACGACGAC | CTGAAACTGA | CCTGCCGTGC |
| 81 | TTCCGGTTAC | ACCATCGGTC | CGTACTGCAT | GGCTTGGTTC | CGTCAGGCTC | CGAACGACGA | CTCCACCAAC | GTTGCTACCA |
| 161 | TCAACATGGG | TACCGTTACC | CTGTCCATGG | ACGACCTGCA | GCCGGAAGAC | TCCGCTGAAT | ACAACTGCGC | TGCTGACTCC |
| 241 | ACCATCTACG | CTTCCTACTA | CGAATGCGGT | CACGGTATCT | CCACCGGTGG | TTACGGTTAC | GACTCCCACT | ACCGTGGTCA |
| 321 | GGGTACCGAC | GTTACCGTTT | CCTCGGCCAG | CTCGGCC | | | | |

iMab D102

| | | | | | | | | |
|---|---|---|---|---|---|---|---|---|
| 1 | CATA | TGGACCTGAA | ACTCACCTGC | CGTGCTTCCG | GTTACACCAT | CGGTCCGTAC | TGCATGGGTT | GGTTCCGTCA |
| 81 | GGCTCCGAAC | GACGACTCCA | CCAACGTTGC | TACCATCAAC | ATGCCTACCG | TTACCCTGTC | CATGCACGAC | CTGCAGCCGG |
| 161 | AAGACTCCGC | TGAATACAAC | TGCGCTGCTG | ACTCCACCAT | CTACGCTTCC | TACTACGAAT | GCGGTCACGG | TATCTCCACC |
| 241 | GGTGGTTACG | GTTACGACTC | CCACTACCGT | GGTCAGGGTA | CCGACGTTAC | CGTTTCCTCG | GCCAGCTCGG | CC |

iMab D111

| | | | | | | | | |
|---|---|---|---|---|---|---|---|---|
| 1 | CATATG | AATGTGAAAC | TGGTTTGTAA | AGGTGGCAAT | TTCGTCGAAA | ACGATGACGA | TCTTAAGCTC | ACGTGCCGTG |
| 81 | CTGAAGGTTA | CACCATTGCC | CCGTACTGCA | TGGGTTGGTT | CCGTCAGGCG | CCCAACCACG | ACACTACTAA | CGTGGCCACG |
| 161 | ATCAACATGG | CTGGCGGTAT | TACGTACTAC | GGTGACTCCG | TCAAAGAGCG | CTTCGATATC | CGTCGCGACA | ACGCGTCCAA |
| 241 | CACCGTTACC | TTATCGATGG | ACCATCTGCA | ACCGGAAGAC | TCTGCAGAAT | ACAATTGTGC | AGGTGATTCT | ACCATTTACG |
| 321 | CGAGCTATTA | TGAATGTGGT | CATGGCCTGA | GTACCGGCGG | TTACGGCTAC | GATAGCCACT | ACCGTTGCCA | GGGTACCGAC |
| 401 | GTTACCGTCT | CGTCGGCCAG | CTCGGCC | | | | | |

iMab D112

| | | | | | | | | |
|---|---|---|---|---|---|---|---|---|
| 1 | AATGTGAAAC | TGGTTGAAAA | AGCTGGCAAT | TTCGTCGAAA | ACGATGACGA | TCTTAAGCTC | ACGTCCCGTG | CTGAAGGTTA |
| 81 | CACCATTGGC | CCGTACTGCA | TGGGTTGGTT | CTGTCAGCCG | CCGAACCACG | ACAGTACTTC | CGTGGCCACG | ATCAACATGG |
| 161 | GTGGCGGTAT | TACGTACTAC | GGTGACTCCG | TCAAAGAGCG | CTTCGATATC | CGTCGCGACA | ACGCGTCCAA | CACCGTTACC |
| 241 | TTATCCATCG | ACGATCTGCA | ACCCGAAGAC | TCTGCAGAAT | ACAATTGTGC | AGGTGATTCT | ACCATTTACG | CGAGCTATTA |
| 321 | TGAATGTGGT | CATGGCCTGA | GTACCGGCGG | TTACGGCTAC | GATAGCCACT | ACCGTGGTCA | GGGTACCGAC | GTTACCGTCT |
| 401 | CGTCG | | | | | | | |

iMab D113

| | | | | | | | | |
|---|---|---|---|---|---|---|---|---|
| 1 | AATGTGAAAC | TGGTTGAAAA | AGGTGGCAAT | TTCGTCGAAA | ACGATGACGA | TCTTAACCTC | ACGTGCCGTG | CTGAACGTTA |
| 81 | CACCATTGCC | CCGTACTCCA | TGGGTTGGTT | CCGTCAGGCG | CCGAACGACG | ACAGTACTAA | CGTGTCCTGC | ATCAACATGG |
| 161 | GTGGCGGTAT | TACGTACTAC | CGTGACTCCG | TCAAAGAGCG | CTTCGATATC | CGTCGCGACA | ACGCGTCCAA | CACCGTTACC |
| 241 | TTATCGATGG | ACGATCTGCA | ACCGGAAGAC | TCTGCAGAAT | ACAATTGTGC | AGGTGATTCT | ACCATTTACG | CGAGCTATTA |
| 321 | TCAATGTGGT | CATCGCCTGA | GTACCGGCGG | TTACGGCTAC | GATAGCCACT | ACCGTGGTCA | GGGTACCGAC | GTTACCGTCT |
| 401 | CGTCG | | | | | | | |

iMab D114

| | | | | | | | | |
|---|---|---|---|---|---|---|---|---|
| 1 | AATGTGAAAC | TGGTTGAAAA | AGGTGGCAAT | TTCGTCGAAA | ACGATGACGA | TCTTAAGCTC | ACGTGCCGTG | CTGAAGGTTA |
| 81 | CACCATTGGC | CCGTACTCCA | TGGGTTGGTT | CCGTCAGGCG | CCGAACGACG | ACAGTACTAA | CGTGGCCACG | ATCAACATGG |
| 161 | GTGGCGGTAT | TACGTACTAC | GGTGACTCCG | TCAAAGAGCG | CTTCGATATC | CGTCGCGACA | ACGCGTCCAA | CACCGTTACC |
| 241 | TTATCGATGG | ACGATCTGCA | ACCGGAAGAC | TCTGCAGAAT | ACAATTGTGC | AGGTGATTCT | ACCATTTACG | CGAGCTATTA |
| 321 | TGAATGTGGT | CATGGCCTGA | GTACCGGCGG | TTACGGCTAC | GATAGCCACT | ACCCTGGTCA | GGGTACCGAC | GTTACCGTCT |
| 401 | CGTCG | | | | | | | |

iMab D115

| | | | | | | | | |
|---|---|---|---|---|---|---|---|---|
| 1 | AATGTGAAAC | TGGTTGAAAA | AGGTGGCAAT | TTCGTCGAAA | ACGATGACGA | TCTTAAGCTC | ACGTGCCGTG | CTGAAGGTTA |
| 81 | CACCATTGGC | CCGTACTGCA | TGGGTTGGTT | CCGTCAGGCG | CCGAACGACG | ACAGTACTAA | CGTGGCCACG | ATCAACATGG |
| 161 | GTGGCGGTAT | TACGTACTAC | GGTGACTCCG | TCAAAGAGCG | CTTCGATATC | CGTCGCGACC | AGGCGTCCAA | CACCGTTACC |
| 241 | TTATCGATGG | ACGATCTGCA | ACCGGAAGAC | TCTGCAGAAT | ACAATTGTGC | AGGTGATTCT | ACCATTTACG | CGAGCTATTA |
| 321 | TGAATGTGGT | CATGGCCTGA | GTACCGGCGG | TTACGGCTAC | GATAGCCACT | ACCGTGGTCA | GGGTACCGAC | GTTACCGTCT |
| 401 | CGTCG | | | | | | | |

iMab D116

| | | | | | | | | |
|---|---|---|---|---|---|---|---|---|
| 1 | CATATG | AATGTCAAAC | TGGTTGAAAA | AGGTGGCAAT | TTCGTCGAAA | ACGATGACGA | TCTTAAGCTC | ACGTGTCGTG |
| 81 | CTGAAGGTTA | CACCATTGGC | CCGTACTGCA | TGGGTTGGTT | CCGTCAGGCG | CCGAACGACG | ACAGTACTAA | CGTGGCCACG |
| 161 | ATCAACATGG | GTGGCGGTAT | TACGTACTAC | GGTGACTCCG | TCAAAGAGCG | CTTCGATATC | CGTCGCGACA | ACGCGTCCAA |
| 241 | CACCGTTACC | TTATCGATGG | ACGATCTGCA | ACCGGAAGAC | TCTGCAGAAT | ACAATGGTGC | AGGTGATTCT | ACCATTTACG |
| 321 | GGAGCTATTA | TGAATGTGGT | CATGGCCTGA | GTACCGGCGG | TTACGGCTAC | GATAGCCACT | ACCGTGGTCA | GGGTACCGAC |
| 401 | GTTACCGTCT | CGTCGGCCAG | CTCGGCC | | | | | |

iMab D120

| | | | | | | | | |
|---|---|---|---|---|---|---|---|---|
| 1 | AATGTGAAAC | TGGTTGAAAA | AGGTGGCAAT | TTCGTCGAAA | ACGATGACGA | TCTTAAGCTC | ACGTGCCGTG | CTGAAGGTTA |
| 81 | CACCATTGGC | CCGTACTGCA | TGGGTTGGTT | CCGTCAGGCG | CCGAACGACG | ACAGTACTAA | CGTGGCCACG | ATCAACATGG |
| 161 | GTGGCGGTAT | TACGTACTAC | GGTGACTCCG | TCAAAGAGCG | CTTCGATATC | CGTCGCGACA | ACGCGTCCAA | CACCGTTACC |
| 241 | TTATCGATGG | ACGATCTGCA | ACCGGAAGAC | TCTGCAGAAT | ACAATTGTGC | AGGTGATTCT | ACCATTTACG | CGACCTATTA |
| 321 | TGAATGTGGT | CATGGCCTGA | GTACCGGCGG | TTACGGCTAC | GATAGCCGTG | GTCAGGGTAC | CGACGTTACC | GTCTCGTCG |

iMab D121

| | | | | | | | | |
|---|---|---|---|---|---|---|---|---|
| 1 | CATA | TGAACGTTAA | ACTGGTTGAA | AAAGGTGGTA | ACTTCGTTGA | AAACGACGAC | GACCTGAAAC | TGACCTGCCG |
| 81 | TGCTTCCGGT | CGTTCCTTCT | CCTCCTACAT | CATGGGTTGG | TTCCGTCAGG | CTCCGAACGA | CGACTCCACC | AACGTTGCTA |
| 161 | CCATCTCCCA | AACCGGTGGT | GACATCGTTT | ACACCAACTA | CGGTGACTCC | GTTAAAGAAC | GTTTCGACAT | CCGTCGTGAC |
| 241 | ATCGCTTCCA | ACACCGTTAC | CCTGTCCATG | GACGACCTGC | AGCCGGAAGA | CTCCGCTGAA | TACAACTGCG | CTGGTTCCGT |
| 321 | TTACGGTTCC | GGTTGGCGTC | CGGACCGTTA | CGACTACCGT | GGTCAGGGTA | CCGACGTTAC | CGTTTCCTCG | GCCAGCTCGG |
| 401 | CC | | | | | | | |

iMab D122

| | | | | | | | | |
|---|---|---|---|---|---|---|---|---|
| 1 | CATA | TGAACGTTAA | ACTGGTTGAA | AAAGGTGGTA | ACTTCGTTGA | AAACGACGAC | GACCTGAAAC | TGACCTGCCG |
| 81 | TGCTTCCGGT | CGTACCTTCT | CCTCCCGTAC | CATGGGTTGG | TTCCGTCAGG | CTCCGAACGA | CGACTCCACC | AACGTTGCTA |
| 161 | CCATCCGTTG | GAACGGTGGT | TCCACCTACT | ACACCAACTA | CGGTGACTCC | GTTAAAGAAC | GTTTCGACAT | CCGTGTTGAC |
| 241 | CAGGCTTCCA | ACACCGTTAC | CCTGTCCATG | GACGACCTGC | AGCCGGAAGA | CTCCGCTGAA | TACAACTGCG | CTGGTACCGA |
| 321 | CATCGGTGAC | GGTTGGTCCG | GTCGTTACGA | CTACCGTGGT | CAGGGTACCG | ACGTTACCGT | TTCCTCGGCC | AGCTCGGCC |

iMab D123

| | | | | | | | | |
|---|---|---|---|---|---|---|---|---|
| 1 | CATA | TGAACGTTAA | ACTGGTTGAA | AAAGGTGGTA | ACTTCGTTGA | AAACGACGAC | GACCTGAAAC | TGACCTGCCG |
| 81 | TGCTTCCGGT | CGTACCTTCT | CCCGTGCTGC | TATGGGTTGG | TTCCGTCAGG | CTCCGAACGA | CGACTCCACC | AACGTTGCTA |
| 161 | CCATCACCTG | GTCCGCTCGT | CACACCCGTT | ACGGTGACTC | CGTTAAAGAA | CGTTTCGACA | TCCGTCGTGA | CCAGGCTTCC |
| 241 | AACACCGTTA | CCCTGTCCAT | GGACGACCTG | CAGCCGGAAG | ACTCCGCTGA | ATACAACTGC | GCTGGTGAAG | GTTCCAACAC |
| 321 | CGCTTCCACC | TCCCCGCGTC | CGTACGACTA | CCGTGGTCAG | GGTACCGACG | TTACCGTTTC | CTCGGCCAGC | TCGGCC |

iMab D124

| | | | | | | | | |
|---|---|---|---|---|---|---|---|---|
| 1 | CATA | TGGACGACCT | GAAACTGACC | TGCCGTGCTT | CCGGTCGTTC | CTTCTCCTCC | TACATCATGG | GTTGGTTCCG |
| 81 | TCAGGCTCCG | AACGACGACT | CCACCAACGT | TGCTACCATC | TCCGAAACCA | CCGTTACCCT | GTCCATGGAC | GACCTGCAGC |
| 161 | CGGAAGACTC | CGCTGAATAC | AACTGCGCTG | GTTCCGTTTA | CGGTTCCGGT | TGGCGTCCGG | ACCGTTACGA | CTACCGTGGT |
| 241 | CAGGGTACCG | ACGTTACCGT | TTCCTCGGCC | AGCTCGGCC | | | | |

iMab D125

| | | | | | | | | |
|---|---|---|---|---|---|---|---|---|
| 1 | CATA | TGGACGACCT | GAAACTGACC | TGCCGTGCTT | CCGGTCGTAC | CTTCTCCTCC | CGTACCATGG | GTTGGTTCCG |
| 81 | TCAGGCTCCG | AACGACGACT | CCACCAACGT | TGCTACCATC | CGTTGGAACA | CCGTTACCCT | GTCCATGGAC | GACCTGCAGC |
| 161 | CGGAAGACTC | CGCTGAATAC | AACTGCGCTG | GTACCGACAT | CGGTGACGGT | TGGTCCGGTC | GTTACGACTA | CCGTGGTCAG |
| 241 | GGTACCGACG | TTACCGTTTC | CTCGGCCAGC | TCGGCC | | | | |

iMab D130

| | | | | | | | | |
|---|---|---|---|---|---|---|---|---|
| 1 | A | ATGTGAAACT | GGTTGAAAAA | GGTGGCAATT | TCGTCGAAAA | CGATGACGAT | CTTAAGCTCA | CGTGCCGTGC |
| 81 | TAGCGGTTAC | GCCTACACGT | ATATCTACAT | GGGTTGGTTC | CGTCAGGCGC | GGAACGACGA | CAGTACTAAC | GTGGCCACCA |
| 161 | TCGACTCGGG | TGGCGGCGGT | AGCCTGTACG | GTGACTCCGT | CAAAGAGCGC | TTCGATATCC | GTCGCGACAA | AGGCTCCAAC |
| 241 | ACCGTTACCT | TATCGATGGA | CGATCTGCAA | CCGGAAGACT | CTGCAGAATA | CAATTGTGCA | GCGGGTGGCT | ACGAACTGCG |
| 321 | CGACCGCACC | TACGGTCAGC | GTGGTCAGGG | TACCGACGTT | ACCGTCTCGT | CGGCCAGCTC | GGCC | |

iMab D201

| | | | | | | | | |
|---|---|---|---|---|---|---|---|---|
| 1 | CATA | TGGTTCAGCT | GCAGGCTTCC | GGTGGTGGTT | CCGTTCAGGC | TGGTGGTTCC | CTGCGTCTGT | CCTGCCGTGC |
| 81 | TTCCGGTTAC | ACCATCGGTC | CGTACTGCAT | GGGTTGGTTC | CGTCAGGCTC | CGGGTGACGA | CTCCGAAGGT | GTTGCTGCTA |
| 161 | TCAACATGGG | TACCGTTTAC | CTGCTGATGA | ACTCCCTGGA | ACCGGAAGAC | ACCGCTATCT | ACTACTGCGC | TGCTGACTCC |
| 241 | ACCATCTACG | CTTCCTACTA | CGAATGCGGT | CACGGTATCT | CCACCGGTGG | TTACGGTTAC | GACTCCTGGG | GTCAGGGTAC |
| 321 | CCAGGTTACC | GTTTCCTCGG | CCAGCTCGGC | C | | | | |

iMab D300

| | | | | | | | | |
|---|---|---|---|---|---|---|---|---|
| 1 | CATA | TCCTTCAGCT | GCAGCAGCCG | GGTTCCAACC | TGGTTCGTCC | GGGTGCTTCC | GTTAAACTCT | CCTGCAAAGC |
| 81 | TTCCGGTTAC | ACCATCCGTC | CGTCCTGCAT | CCACTGGGCT | AAACAGCGTC | CGGGTGACGG | TCTGGAATCG | ATCGGTGAAA |
| 161 | TCAACATGGG | TACCGCTTAC | GTTGACCTGT | CCTCCCTGAC | CTCCGAAGAC | TCCCCTGTTT | ACTACTGCGC | TGCTGACTCC |
| 241 | ACCATCTACG | CTTCCTACTA | CGAATGCGGT | CACGGTATCT | CCACCGGTGG | TTACGGTTAC | GACTACTGGG | GTCAGGGTAC |
| 321 | CACCCTGACC | GTTTCCTCGG | CCAGCTCGGC | C | | | | |

iMab D302

| | | | | | | | | |
|---|---|---|---|---|---|---|---|---|
| 1 | CATA | TGGCTTCCGT | TAAACTGTCC | TGCAAAGCTT | CCGGTTACAC | CATCGGTCCG | TCCTGCATCC | ACTGGGCTAA |
| 81 | ACAGCGTCCG | GCTGACGGTC | TGGAATGGAT | CGGTGAAATC | AACATGGGTA | CCGCTTACGT | TGACCTGTCC | TCCCTGACCT |
| 161 | CCGAAGACTC | CGCTGTTTAC | TACTGCGCTG | CTGACTCCAC | CATCTACGCT | TCCTACTACG | AATGCGGTCA | CGGTATCTCC |
| 241 | ACCGGTGGTT | ACGGTTACGA | CTACTGGGGT | CAGGGTACCA | CCCTGACCGT | TTCCTCGGCC | AGCTCGGCC | |

iMab D400

| | | | | | | | | |
|---|---|---|---|---|---|---|---|---|
| 1 | CATA | TGGTTCAGCT | GGTTGAATCC | GGTGGTGGTC | TGGTTCAGCC | GGGTGGTTCC | CTGCGTCTGT | CCTGCCGTGC |
| 81 | TTCCGGTTAC | ACCATCGGTC | CGTACTGCAT | GAACTGGGTT | CGTCAGGCTC | CGGGTGACGG | TCTGGAATGG | GTTGGTTGGA |
| 161 | TCAACATGGG | TACCGCTTAC | CTGCAGATGA | ACTCCCTGCG | TGCTGAAGAC | ACCGCTGTTT | ACTACTGCGC | TGCTGACTCC |
| 241 | ACCATCTACG | CTTCCTACTA | CGAATGCGGT | CACGGTATCT | CCACCGGTGG | TTACGGTTAC | GACGTTTGGG | GTCAGGGTAC |
| 321 | CCTGGTTACC | GTTTCCTCGG | CCAGCTCGGC | C | | | | |

iMab D500

| | | | | | | | | |
|---|---|---|---|---|---|---|---|---|
| 1 | CATA | TGCCGAACTT | CCTGTCCTCC | GTTCTGCCGA | CCCACTGGCG | TTGCAACAAA | ACCCTGCCGA | TCGCTTTCAA |
| 81 | ATGCCGTGCT | TCCGGTTACA | CCATCGGTCC | GACCTGCGTT | ACCGTTATGG | CTCGTAACGA | CGAAGACTAC | TCCAACATCG |
| 161 | GTCCTCGTTT | CAACGACCTG | CGTTTCGTTC | GTCGTTCCGG | TCGTGGTAAA | TCCTTCACCC | TCACCTGCGC | TGCTGACTCC |
| 241 | ACCATCTACG | CTTCCTACTA | CGAATGCGGT | CACGGTATCT | CCACCGGTCG | TTACGGTTAC | CCGCAGGTTG | CTACCTACCA |
| 321 | CCCTCCTATC | AAAATCACCG | TTGACGGTCC | GGCCAGCTCG | GCC | | | |

iMab D502

| | | | | | | | | |
|---|---|---|---|---|---|---|---|---|
| 1 | CATA | TGTCCGTTAA | ATTCGTTTGC | AAAGTTCTGC | CGAACTTCTG | GGAAAACAAC | AAAGACCTGC | CGATCAAATT |
| 81 | CACCGTTCGT | CCTTCCGGTT | ACACCATCGG | TCCGACCTGC | GTTGCTGTTT | TCGCTCAGAA | CCCGGAAGAC | GACTCCACCA |
| 161 | ACGTTGCTAC | CATCAACATG | CGTGGTGGTA | TCACCTACTA | CGGTGACTCC | GTTAAACTGC | GTTTCGACAT | CCGTCGTGAC |
| 241 | AACGCTAAAG | TTACCCCTAC | CAACTCCCTG | GACGACGTTC | AGCCGGAAGG | TCGTGGTAAA | TCCTTCGAAC | TGACCTGCGC |
| 321 | TGCAGACTCC | ACCATCTACG | CTTCCTACTA | CGAATGCGGT | CACGGTCTGT | CCACCGGTGG | TTACGGTTAC | GACCACGTTG |
| 401 | CTCGTTACCA | CCGTGGTATC | GACATCACCG | TCTCGTCGGC | CAGCTCGGCC | | | |

iMab D600

| | | | | | | | | |
|---|---|---|---|---|---|---|---|---|
| 1 | CATA | TGGCTCCGGT | TGGTCTGAAA | GCTCGTAACG | CTGACGAATC | CGGTCACGTT | GTTCTGCGTT | GCCGTGCTTC |
| 81 | CGGTTACACC | ATCGGTCCGA | TCTGCTACGA | AGTTGACGTT | TCCGCTGGTC | ACGACGCTGG | TTCCGTTCAG | CGTGTTGAAA |
| 161 | TCAACATGGG | TCGTACCGAA | TCCGTTCTGT | CCAACCTGCG | TGGTCGTACC | CGTTACACCT | TCGCTTGCGC | TGCTGACTCC |
| 241 | ACCATCTACG | CTTCCTACTA | CGAATGCGGT | CACGGTATCT | CCACCGGTGG | TTACGGTTAC | TCCGAATGGT | CCGAACCGGT |
| 321 | TTCCCTGCTG | ACCCCGTCGG | CCAGCTCGGC | C | | | | |

iMab D700

| | | | | | | | | |
|---|---|---|---|---|---|---|---|---|
| 1 | CATA | TGGACAAATC | CACCCTGGCT | GCTGTTCCGA | CCTCCATCAT | CGCTGACGGT | CTGATGGCTT | CCACCATCAC |
| 81 | CTGCGAAGCT | TCCGGTTACA | CCATCGGTCC | GGCTTGCGTT | GCTTTCGACA | CCACCCTGGG | TAACAACATG | GGTACCTACT |
| 161 | CCGCTCCGCT | GACCTCCACC | ACCCTGGGTG | TTGCTACCGT | TACCTGCGCT | GCTGACTCCA | CCATCTACGC | TTCCTACTAC |
| 241 | GAATGCGGTC | ACGGTATCTC | CACCGGTGGT | TACGGTTACG | CTGCTTTCTC | CGTTCCGTCC | GTTACCGTTA | ACTTCACCGC |
| 321 | GGCCAGCTCG | GCC | | | | | | |

iMab D701

| | | | | | | | | |
|---|---|---|---|---|---|---|---|---|
| 1 | CATA | TGATGGCTTC | CACCATCACC | TGCGAAGCTT | CCGGTTACAC | CATCGGTCCG | GCTTGCGTTG | CTTTCGACAC |
| 81 | CACCCTGGGT | AACAACATGG | GTACCTACTC | CGCTCCGCTG | ACCTCCACCA | CCCTGGGTGT | TGCTACCGTT | ACCTGCGCTG |
| 161 | CTGACTCCAC | CATCTACGCT | TCCTACTACG | AATGCGGTCA | CGGTATCTCC | ACCGGTGGTT | ACGGTTACGC | TGCTTTCTCC |
| 241 | GTTCCGTCCG | TTACCGTTAA | CTTCACCGCG | GCCAGCTCGG | CC | | | |

iMab D702

| | | | | | | | | |
|---|---|---|---|---|---|---|---|---|
| 1 | CATA | TGGCTGTTAA | ATCCGTTTTC | AAAGTTTCCA | CCAACTTCAT | CGAAAACGAC | GGCACCATGG | ACTCCAAACT |
| 81 | GACCTTCCGT | GCTTCCGGTT | ACACCATCGG | TCCGCAGTGC | CTGGGTTTCT | TCCAGCAGGG | TGTTCCGGAC | GACTCCACCA |
| 161 | ACGTTGCTAC | CATCAACATG | GGTGGTGGTA | TCACCTACTA | CGGTGACTCC | GTTAAATCCA | TCTTCGACAT | CCGTCGTGAC |
| 241 | AACGCTAAAG | ACACCTACAC | CGCTTCCGTT | GACGACAACC | AGCCGGAAGA | CGTTGAAATC | ACCTGCGCTG | CAGACTCCAC |
| 321 | CATCTACGCT | TCCTACTACG | AATGCGGTCA | CGGTCTGTCC | ACCGGTGGTT | ACGGTTACGA | CCTGATCCTG | CGTACCCTGC |
| 401 | AAAAAGGTAT | CGACCTGTTC | GTCTCGTCGG | CCAGCTCGGC | C | | | |

iMab D800

| | | | | | | | | |
|---|---|---|---|---|---|---|---|---|
| 1 | CATA | TGGGTCGTTC | CTCCTTCACC | GTTTCCACCC | CGGACATCCT | GGCTGACGGT | ACCATGTCCT | CCACCCTGTC |
| 81 | CTGCCGTGCT | TCCGGTTACA | CCATCGGTCC | GCAGTGCCTG | TCCTTCACCC | AGAACGGTGT | TCCGGTTTCC | ATCTCCCCGA |
| 161 | TCAACATGGG | TTCCTACACC | GCTACCGTTG | TTGGTAACTC | CGTTGGTCAC | GTTACCATCA | CCTGCGCTGC | TGACTCCACC |
| 241 | ATCTACGCTT | CCTACTACGA | ATGCGGTCAC | GGTATCTCCA | CCGGTGGTTA | CGGTTACACC | CTGATCCTGT | CCACCCTGCA |
| 321 | GAAAAAAATC | TCCCTGTTCC | CGGCCAGCTC | GGCC | | | | |

iMab D900

| | | | | | | | | |
|---|---|---|---|---|---|---|---|---|
| 1 | CATA | TGCTGACCCT | GACCGCTGCT | GTTATCGGTG | ACGGTGCTCC | GGCTAACGGT | AAAACCGCTA | TCACCGTTGA |
| 81 | ATGCACCGCT | TCCGGTTACA | CCATCGGTCC | GCAGTGCGTT | GTTATCACCA | CCAACAACGG | TGCTCTGCCG | AACAAAATCA |
| 161 | CCGAAAACAT | GGGTGTTGCT | CGTATCGCTC | TGACCAACAC | CACCGACGGT | GTTACCGTTG | TTACCTGCGC | TGCTGACTCC |
| 241 | ACCATCTACG | CTTCCTACTA | CGAATGCGGT | CACGGTATCT | CCACCGGTGG | TTACGGTTAC | CAGCGTCAGT | CCGTTGACAC |
| 321 | CCACTTCGTT | AAGGCCAGCT | CGGCC | | | | | |

iMab D1000

| | | | | | | | | |
|---|---|---|---|---|---|---|---|---|
| 1 | CATA | TGCACAAACC | GGTTATCGAA | AAAGTTGACG | GTGGTTACCT | GTGCAAAGCT | TCCGGTTACA | CCATCGGTCC |
| 81 | GGAATGCATC | GAACTGCTGG | CTGACGGTCG | TTCCTACACC | AAAAACATGG | GTGAAGCTTT | CTTCGCTATC | GACGCTTCCA |
| 161 | AAGTTACCTG | CGCTGCTGAC | TCCACCATCT | ACGCTTCCTA | CTACGAATGC | GGTCACGGTA | TCTCCACCGG | TGGTTACGGT |
| 241 | TACCACTGGA | AAGCTGAAAA | CTCGGCCAGC | TCGGCC | | | | |

iMab D1001

| | | | | | | | | |
|---|---|---|---|---|---|---|---|---|
| 1 | CATA | TGGTTGACGG | TGGTTACCTG | TGCAAAGCTT | CCGGTTACAC | CATCGGTCCG | GAATGCATCG | AACTGCTGGC |
| 81 | TGACGGTCGT | TCCTACACCA | AAAACATGGG | TGAAGCTTTC | TTCGCTATCG | ACGCTTCCAA | AGTTACCTGC | GCTGCTGACT |
| 161 | CCACCATCTA | CGCTTCCTAC | TACGAATGCG | GTCACGGTAT | CTCCACCGGT | GGTTACGGTT | ACCACTGGAA | AGCTGAAAAT |
| 241 | TCGGCCAGCT | CGGCC | | | | | | |

iMab D1100

| | | | | | | | | |
|---|---|---|---|---|---|---|---|---|
| 1 | CATA | TGGCTCCGGT | TGGTCTGAAA | GCTCGTCTGG | CTGACGAATC | CGGTCACGTT | GTTCTGCGTT | GCCGTGCTTC |
| 81 | CGGTTACACC | ATCGGTCCGA | TCTGCTACGA | AGTTGACGTT | TCCGCTGGTA | ACGACGCTGG | TTCCGTTCAG | CGTGTTGAAA |
| 161 | TCCTGAACAT | GGGTACCGAA | TCCGTTCTGT | CCAACCTGCG | TGGTCGTACC | CGTTACACCT | TCGCTTGCGC | TGCTGACTCC |
| 241 | ACCATCTACG | CTTCCTACTA | CGAATGCGGT | CACGGTATCT | CCACCGGTGG | TTACGGTTAC | TCCGCTTGGT | CCGAACCGGT |
| 321 | TTCCCTGCTG | ACCCCGTCGG | CCAGCTCGGC | C | | | | |

iMab D1200

| | | | | | | | | |
|---|---|---|---|---|---|---|---|---|
| 1 | CATA | TGCACGGTCT | GCCGATGGAA | AAACGTGGTA | ACTTCATCGT | TGGTCAGAAC | TGCTCCCTGA | CCTGCCCGGC |
| 81 | TTCCGGTTAC | ACCATCGGTC | CGCAGTGCGT | TTTCAACTGC | TACTTCAACT | CCGCTCTGGC | TTTCTCCACC | GAAAACATGG |
| 161 | GTGAATGGAC | CCTGGACATG | GTTTTCTCCG | ACGCTGGTAT | CTACACCATG | TGCGCTGCTG | ACTCCACCAT | CTACGCTTCC |
| 241 | TACTACGAAT | GCGGTCACGG | TATCTCCACC | GGTGGTTACG | GTTACAACCC | GGTTTCCCTG | GGTTCCTTCG | TTGTTGACTC |
| 321 | CCCGGCCAGC | TCGGCC | | | | | | |

iMab D1202

| | | | | | | | | |
|---|---|---|---|---|---|---|---|---|
| 1 | CATA | TGATCGTTAA | ACTGGTTATG | GAAAAACGTG | GTAACTTCGA | AAACGGTCAG | GACTGCAAAC | TGACCATCCG |
| 81 | TGCTTCCGGT | TACACCATCG | GTCCGGCTTG | CGACGGTTTC | TTCTGCCAGT | TCCCGTCCGA | CGACTCCTTC | TCCACCGAAG |
| 161 | ACAACATGGG | TGGTGGTATC | ACCGTTAACG | ACGCTATGAA | ACCGCAGTTC | GACATCCGTC | GTGACAACGC | TAAAGGCACC |
| 241 | TGGACCCTGT | CCATGGACTT | CCAGCCGGAA | GGTATCTACG | AAATGCAGTG | CGCTGCAGAC | TCCACCATCT | ACGCTTCCTA |
| 321 | CTACGAATGC | GGTCACGGTC | TGTCCACCGG | TGGTTACGGT | TACGACAACC | CGGTTCGTCT | GGGTGGTTTC | GACGTTGACG |
| 401 | TCTCGTCGGC | CAGCTCGGCC | | | | | | |

iMab D1300

| | | | | | | | | |
|---|---|---|---|---|---|---|---|---|
| 1 | CATA | TGCTGCAGGT | TGACATCAAA | CCGTCCCAGG | GTGAAATCTC | CGTTGGTGAA | TCCAAATTCT | TCCTGTGCCA |
| 81 | GGCTTCCGGT | TACACCATCG | GTCCGTGCAT | CTCCTGGTTC | TCCCCGAACG | GTGAAAAACT | GAACATGGGT | TCCTCCACCC |
| 161 | TGACCATCTA | CAACGCTAAC | ATCGACGACG | CTGGTATCTA | CAAATGCGCT | GCTGACTCCA | CCATCTACGC | TTCCTACTAC |
| 241 | GAATGCGGTC | ACGGTATCTC | CACCGGTGGT | TACGGTTACC | AGTCCGAAGC | TACCGTTAAC | GTTAAAATCT | TCCAGGCCAG |
| 321 | CTCGGCC | | | | | | | |

iMab D1301

| | | | | | | | | |
|---|---|---|---|---|---|---|---|---|
| 1 | CATA | TGGAATCCAA | ATTCTTCCTG | TGCCAGGCTT | CCGGTTACAC | CATCGGTCCG | TGCATCTCCT | GGTTCTCCCC |
| 81 | GAACGGTGAA | AAACTGAACA | TGGGTTCCTC | CACCCTGACC | ATCTACAACG | CTAACATCGA | CGACGCTGGT | ATCTACAAAT |
| 161 | GCGCTGCTGA | CTCCACCATC | TACGCTTCCT | ACTACGAATG | CGGTCACGGT | ATCTCCACCG | GTGGTTACGG | TTACCAGTCC |
| 241 | GAAGCTACCG | TTAACGTTAA | AATCTTCCAG | GCCAGCTCGG | CC | | | |

iMab D1302

| | | | | | | | | |
|---|---|---|---|---|---|---|---|---|
| 1 | CATA | TGGTTGTTAA | AGTTGTTATC | AAACCGTCCC | AGAACTTCAT | CGAAAACGGT | GAAGACAAAA | AATTCACCTG |
| 81 | CCGTGCTTCC | GGTTACACCA | TCGGTCCGAA | ATGCATCGGT | TGGTTCTCCC | AGAACCCGGA | AGACGACTCC | ACCAACGTTG |
| 161 | CTACCATCAA | CATGGGTGGT | GGTATCACCT | ACTACGGTGA | CTCCGTTAAA | GAACGTTTCG | ACATCCGTCG | TGACAACGCT |
| 241 | AAAGACACCT | CCACCCTGTC | CATCGACGAC | GCTCAGCCGG | AAGACGCTGG | TATCTACAAA | TGCGCTGCAG | ACTCCACCAT |
| 321 | CTACGCTTCC | TACTACGAAT | GCGGTCACGG | TCTGTCCACC | GGTGGTTACG | GTTACGACTC | CGAAGCTACC | GTTGGTGTTG |
| 401 | ACATCTTCGT | CTCGTCGGCC | AGCTCGGCC | | | | | |

iMab D1400

| | | | | | | | | |
|---|---|---|---|---|---|---|---|---|
| 1 | CATA | TGGTTCCGCG | TGACCTGGAA | GTTGTTGCTG | CTACCCCGAC | CTCCCTGCTG | ATCTCCTCCG | ACGCTTCCGG |
| 81 | TTACACCATC | GGTCCGTACT | GCATCACCTA | CGGTGAAACC | GGTGGTAACT | CCCCGGTTCA | GGAATTCACC | GTTCCGAACA |
| 161 | TGGGTAAATC | CACCGCTACC | ATCTCCGGTC | TGAAACCGGG | TGTTGACTAC | ACCATCACCT | GCGCTGCTGA | CTCCACCATC |
| 241 | TACGCTTCCT | ACTACGAATG | CGGTCACGGT | ATCTCCACCG | GTGGTTACGG | TTACTCCAAA | CCGATCTCCA | TCAACTACCG |
| 321 | TACGGCCAGC | TCGGCC | | | | | | |

iMab D1500

| | | | | | | | | |
|---|---|---|---|---|---|---|---|---|
| 1 | CATA | TGATCAAAGT | TTACACCGAC | CGTGAAAACT | ACGGTGCTGT | TGGTTCCCAG | GTTACCCTGC | ACTGCTCCGC |
| 81 | TTCCGGTTAC | ACCATCGGTC | CGATCTGCTT | CACCTGGCGT | TACCAGCCGG | AAGGTGACCG | TGACGCTATC | TCCATCTTCC |
| 161 | ACTACAACAT | GGGTGACGGT | TCCATCGTTA | TCCACAACCT | GGACTACTCC | GACAACGGTA | CCTTCACCTG | CGCTGCTGAC |
| 241 | TCCACCATCT | ACGCTTCCTA | CTACGAATGC | GGTCACGGTA | TCTCCACCGG | TGGTTACGGT | TACGTTGGTA | AAACCTCCCA |
| 321 | GGTTACCCTG | TACGTTTTCG | AGGCCAGCTC | GGCC | | | | |

iMab D1501

| | | | | | | | | |
|---|---|---|---|---|---|---|---|---|
| 1 | CATA | TGTCCCAGGT | TACCCTCCAC | TGCTCCGCTT | CCGGTTACAC | CATCGGTCCG | ATCTGCTTCA | CCTGGCGTTA |
| 81 | CCAGCCGGAA | GGTGACCGTG | ACGCTATCTC | CATCTTCCAC | TACAACATGG | GTGACGGTTC | CATCGTTATC | CACAACCTGG |
| 161 | ACTACTCCGA | CAACGGTACC | TTCACCTGCG | CTGCTGACTC | CACCATCTAC | GCTTCCTACT | ACGAATGCGG | TCACGGTATC |
| 241 | TCCACCGGTG | GTTACGGTTA | CGTTGGTAAA | ACCTCCCAGG | TTACCCTGTA | CGTTTTCGAG | GCCAGCTCGG | CC |

iMab D1502

| | | | | | | | | |
|---|---|---|---|---|---|---|---|---|
| 1 | CATA | TGAACGTTAA | AGTGGTTACC | AAACGTGAAA | ACTTCGGTGA | AAACGGTTCC | GACGTTAAAC | TGACCTGCCG |
| 81 | TGCTTCCGGT | TACACCATCG | GTCCGATCTG | CTTCGGTTGG | TTCTACCAGC | CGGAAGGTGA | CGACTCCGCT | ATCTCCATCT |
| 161 | TCCACAACAT | GGGTGGTGGT | ATCACCGACG | AAGTTGACAC | CTTCAAAGAA | CGTTTCGACA | TCCGTCGTGA | CAACGCTAAA |
| 241 | AAAACCGGCA | CCATCTCCAT | CGACGACCTG | CAACCGTCCG | ACAACGAAAC | CTTCACCTGC | GCTGCAGACT | CCACCATCTA |
| 321 | CGCTTCCTAC | TACGAATGCG | GTCACGGTCT | GTCCACCGGT | GGTTACGGTT | ACGACGGTAA | AACCCGTCAG | GTTGGTCTGG |
| 401 | ACGTTTTCGT | CTCGTCGGCC | AGCTCGGCC | | | | | |

iMab D1600

| | | | | | | | | |
|---|---|---|---|---|---|---|---|---|
| 1 | CATA | TGATCAAAGT | TTACACCGAC | CGTGAAAACT | ACGGTGCTGT | TGGTTCCCAG | GTTACCCTGC | ACTGCTCCGC |
| 81 | TTCCGGTTAC | ACCATCGGTC | CGATCTGCTT | CACCTGGCGT | TACCAGCCGG | AAGGTGACCG | TGACGCTATC | TCCATCTTCC |
| 161 | ACTACAACAT | GGGTGACGGT | TCCATCGTTA | TCCACAACCT | GGACTACTCC | GACAACGGTA | CCTTCACCTG | CGCTGCTGAC |
| 241 | TCCACCATCT | ACGCTTCCTA | CTACGAATGC | GGTCACGGTA | TCTCCACCGG | TGGTTACGGT | TACGTTGGTA | AAACCTCCCA |
| 321 | GGTTACCCTG | TACGTTTTCG | AGGCCAGCTC | GGCC | | | | |

iMab D1602

| | | | | | | | | |
|---|---|---|---|---|---|---|---|---|
| 1 | CATATGGCTG | TTAAACCGGT | TATCGGTTCC | AAAGCTCCGA | ACTTCGGTGA | AAACGGTGAC | GTTAAAACCA | TCGACCGTGC |
| 81 | TTCCGGTTAC | ACCATCGGTC | CGACCTGCGG | TGGTGTTTTC | TTCCAGGGTC | CGACCGACGA | CTCCACCAAC | GTTGCTACCA |
| 161 | TCAACATGGG | TGGTGGTATC | ACCTACTACG | GTGACTCCGT | TAAAGAAACC | TTCGACATCC | GTCGTGACAA | CGCTAAATCC |
| 241 | ACCCGTACCG | AATCCTACGA | CGACAACCAG | CCGGAAGGTC | TGACCGAAGT | TAAATGCGCT | GCAGACTCCA | CCATCTACGC |
| 321 | TTCCTACTAC | GAATGCGGTC | ACGGTCTGTC | CACCGGTGGT | TACGGTTACG | ACGTTTCCTC | CCGTCTGTAC | GGTTACGACA |
| 401 | TCCTGGTCTC | GTCGGCCAGC | TCGGCC | | | | | |

iMab D1700

| | | | | | | | | |
|---|---|---|---|---|---|---|---|---|
| 1 | CATA | TGAAAGACCC | GGAAATCCAC | CTGTCCGGTC | CGCTGGAAGC | TGGTAAACCG | ATCACCGTTA | AATGCTCCGC |
| 81 | TTCCGGTTAC | ACCATCGGTC | CGCTGTGCAT | CGACCTGCTG | AAAGGTGACC | ACCTGATGAA | ATCCCAGGAA | TTCAACATGG |
| 161 | GTTCCCTGGA | AGTTACCTTC | ACCCCGGTTA | TCGAAGACAT | CGGTAAAGTT | CTGGTTTGCG | CTGCTGACTC | CACCATCTAC |
| 241 | GCTTCCTACT | ACGAATGCGG | TCACGGTATC | TCCACCGGTG | GTTACGGTTA | CGTTCGTCAG | GCTGTTAAAG | AACTGCAGGT |
| 321 | TGACTCGGCC | AGCTCGGCC | | | | | | |

iMab D1701

| | | | | | | | | |
|---|---|---|---|---|---|---|---|---|
| 1 | CATA | TGAAACCGAT | CACCGTTAAA | TGCTCCGCTT | CCGGTTACAC | CATCGGTCCG | CTGTGCATCG | ACCTGCTGAA |
| 81 | AGGTGACCAC | CTGATGAAAT | CCCAGGAATT | CAACATGGGT | TCCCTGGAAG | TTACCTTCAC | CCCGGTTATC | CAAGACATCG |
| 161 | GTAAAGTTCT | GGTTTGCGCT | GCTGACTCCA | CCATCTACGC | TTCCTACTAC | GAATGCGGTC | ACGGTATCTC | CACCGGTGGT |
| 241 | TACGGTTACG | TTCGTCAGGC | TGTTAAAGAA | CTGCAGGTTG | ACTCGGCCAG | CTCGGCC | | |

iMab135-xx-0001

| | | | | | | | | |
|---|---|---|---|---|---|---|---|---|
| 1 | AACGTGC | AGCTGGTGCA | AAGCGGCGGC | AACTTTGTGG | AAAACGATCA | GGATCTGAGC | CTGACCTGCC | GCGCGAGCGG |
| 81 | CTATACCATT | GGCCCGTATT | GCATGGGCTG | GTTTCGCCAG | GCGCCGAACC | AGGATAGCAC | CGGCGTGGCG | ACCATTAACA |
| 161 | TGGGCGGCGG | CATTACCTAT | TATGGCGATA | GCGTGAAAGA | ACGCTTTCGC | ATTCGCCGCG | ATAACGCGAG | CAACACCGTG |
| 241 | ACCCTGAGCA | TGCAGAACCT | CCAGCCGCAG | GATAGCGCGA | ACTATAACTG | CGCTGCAGAT | AGCACCATTT | ATGCGAGCTA |
| 321 | TTATGAATGC | GGCCATGGCC | TGAGCACCGG | CGGCTATGGC | TATGATAGCC | GCGGCCAGGG | TACCAGCGTG | ACCGTGAGCT |
| 401 | CGGCCAGCTC | GGCC | | | | | | |

iMab136-xx-0001

| | | | | | | | | |
|---|---|---|---|---|---|---|---|---|
| 1 | AACGTGA | AACTGGTGGA | AAAAGGCGGC | AACTTTGTGG | AAAACGATGA | TGATCTGCGC | CTGACCTGCC | GCGCGGAAGG |
| 81 | CTATACCATT | GGCCCGTATT | GCATGGGCTG | GTTTCGCCAG | GCGCCGAACC | GCGATAGCAC | CAACGTGGCG | ACCATTAACA |
| 161 | TGGGCGGCGG | CATTACCTAT | TATGGCGATA | GCGTGAAAGA | ACGCTTTGAT | ATTCGCCGCG | ATAACGCGAG | CAACACCGTG |
| 241 | ACCCTGAGCA | TGACCAACCT | CCAGCCGAGC | GATAGCGCGA | GCTATAACTG | CGCTGCAGAT | AGCACCATTT | ATGCGAGCTA |
| 321 | TTATGAATGC | GGCCATGGCC | TGAGCACCGG | CGGCTATGGC | TATGATAGCC | GCGGCCAGGG | TACCCGCGTG | ACCGTGAGCT |
| 401 | CGGCCAGCTC | GGCC | | | | | | |

iMab137-xx-0001

| | | | | | | | | |
|---|---|---|---|---|---|---|---|---|
| 1 | AACGTGC | AGCTGGTGGA | AAGCGGCGGC | AACTTTGTGG | AAAACGATCA | GAGCCTGAGC | CTGACCTGCC | GCGCGAGCGG |
| 81 | CTATACCATT | GGCCCGTATT | GCATGGGCTG | GTTTCGCCAG | GCGCCGAACA | GCCGCAGCAC | CGGCGTGGCG | ACCATTAACA |
| 161 | TGGGCGGCGG | CATTACCTAT | TATGGCGATA | GCGTGAAAGG | CCGCTTTACC | ATTCGCCGCG | ATAACGCGAG | CAACACCGTG |
| 241 | ACCCTGAGCA | TGAACGATCT | CCAGCCGCGC | GATAGCGCGC | AGTATAACTG | CGCTGCAGAT | AGCACCATTT | ATGCGAGCTA |
| 321 | TTATGAATGC | GGCCATGGCC | TGAGCACCGG | CGGCTATGGC | TATGATAGCC | GCGGCCAGGG | TACCGATGTG | ACCGTGAGCT |
| 401 | CGGCCAGCTC | GGCC | | | | | | |

iMab142-xx-0002

| | | | | | | | | |
|---|---|---|---|---|---|---|---|---|
| 1 | AATGTGAA | ACTGGTTGAA | AAAGGTGGCA | ATTTCGTCGA | AAACGATGAC | GATCTTAAGC | TCACGTGCCG | TGCTGAAGGT |
| 81 | TACACCATTG | GCCCGTACTC | CATGGGTTGG | TTCCGTCAGG | CGCCGAACGA | CGACAGTACT | AACGTGTCCT | GCATCAACAT |
| 161 | GGGTGGCGGT | ATTACGTACT | ACGGTGACTC | CGTCAAAGAG | CGCTTCGATA | TCCGTCGCGA | CAACGCGTCC | AACACCGTTA |
| 241 | CCTTATCGAT | GGACGATCTG | CAACCGGAAG | ACTCTGCAGT | ATATAACTGT | GCGGCAGATT | GGTGGGATGG | ATTTACGTAC |
| 321 | GGTACAACCC | ATCTTCGTAT | GACTACCGGG | GCCAGGGTAC | CGACGTTACC | GTCTCGTCGG | CCAGCTCGGC | |

iMab148-xx-0002

| | | | | | | | | |
|---|---|---|---|---|---|---|---|---|
| 1 | AATGTGC | ACCTGGTTGA | ACGCGGTGGC | AATTTCGTCG | AAAACGATGA | CGATCTTAAC | CTCACGTGCC | GTGCTGAAGG |
| 81 | TTACACCATT | GGCCCGTACT | CTATGGGTTG | GTTCCGTCAG | GCGCCGAACG | ACGACAGTAC | TAACGTGGCC | ACGATCAACA |
| 161 | TGGGTGGCGG | TATTACGTAC | TACGGTGACT | CCGTCGACGA | GCGCTTCGAT | ATCCGTCGCG | ACAACGCGTC | CAACACCGTT |
| 241 | ACCTTATCGA | TGGACGATCT | GCAACCGGAA | GACTCTGCAG | TATATAACTG | TGCGGCAGAT | TGGTGGGATG | GATTTACGTA |
| 321 | CGGTAGTACC | TGGTACAACC | CATCTTCGTA | TGACTACCGG | GGCCAGGGTA | CCGACGTTAC | CGTCTCGTCG | GCCAGCTCGG |
| 401 | CC | | | | | | | |

iMab138-xx-0007

| | | | | | | | | |
|---|---|---|---|---|---|---|---|---|
| 1 | AACGTT | AAACTGGTTG | AAAAAGGTGG | TAACTTCGTT | GAAAACGACG | ACGACCTGAA | ACTGACCTGG | CGTGCTTCCG |
| 81 | GTCGTACCTT | CTCCTCCCGT | ACCATGGGTT | GGTTCCGTCA | GGCTCCGAAC | GACGACTCCA | CCAACGTTGC | TACCATCCGT |
| 161 | TGGAACGGTG | GTTCCACCTA | CTACACCAAC | TACGGTGACT | CCGTTAAAGA | ACGTTTCGAC | ATCCGTGTTG | ACCAGGCTTC |
| 241 | CAACACCGTT | ACCCTGTCCA | TGGACGACCT | GCAGCCGGAA | GACTCCGCTG | AATACAACGT | CGCTGGTACC | GACATCGGTG |
| 321 | ACGCTTGGTC | CGGTCGTTAC | GACTACCGTG | GTCAGGGTAC | CGACGTTACC | GTTTCCTCG | | |

iMab139-xx-0007

| | | | | | | | | |
|---|---|---|---|---|---|---|---|---|
| 1 | AACGTT | AAACTGGTTG | AAAAAGGTGG | TAACTTCGTT | GAAAACGACG | ACGACCTGAA | ACTGACCGTC | CGTGCTTCCG |
| 81 | GTCGTACCTT | CTCCTCCCGT | ACCATGGGTT | GGTTCCGTCA | GGCTCCCAAC | GACGACTCCA | CCAACGTTGC | TACCATCCGT |
| 161 | TGGAACGGTG | GTTCCACCTA | CTACACCAAC | TACGGTGACT | CCGTTAAAGA | ACGTTTCGAC | ATCCGTGTTG | ACCAGGCTTC |
| 241 | CAACACCGTT | ACCCTGTCCA | TGGACGACCT | GCAGCCGGAA | GACTCCGCTG | AATACAACGT | CGCTGGTACC | GACATCGGTG |
| 321 | ACGGTTGGTC | CCCTCGTTAC | GACTACCGTG | GTCAGGGTAC | CGACGTTACC | GTTTCCTCG | | |

iMab140-xx-0007

| | | | | | | | | |
|---|---|---|---|---|---|---|---|---|
| 1 | AACGTT | AAACTGGTTG | AAAAAGGTGG | TAACTTCGTT | GAAAACGACG | ACGACCTGAA | ACTCACCATC | CGTGCTTCCG |
| 81 | GTCGTACCTT | CTCCTCCCGT | ACCATGGGTT | GGTTCCGTCA | GGCTCCGAAC | GACGACTCCA | CCAACGTTGC | TACCATCCGT |
| 161 | TGGAACGGTG | GTTCCACCTA | CTACACCAAC | TACGGTGACT | CCGTTAAAGA | ACGTTTCGAC | ATCCGTGTTG | ACCAGGCTTC |
| 241 | CAACACCGTT | ACCCTGTCCA | TGGACGACCT | GCAGCCGGAA | GACTCCGCTG | AATACAACTA | CGCTGGTACC | GACATCGGTG |
| 321 | ACGGTTGGTC | CGGTCGTTAC | GACTACCGTG | GTCAGGGTAC | CGACGTTACC | GTTTCCTCG | | |

iMab141-xx-0007

| | | | | | | | | |
|---|---|---|---|---|---|---|---|---|
| 1 | AACGTT | AAACTGGTTG | AAAAAGGTGG | TAACTTCGTT | GAAAACGACG | ACGACCTGAA | ACTGACCTTC | CGTGCTTCCG |
| 81 | GTCGTACCTT | CTCCTCCCGT | ACCATGGGTT | GGTTCCGTCA | GGCTCCGAAC | GACGACTCCA | CCAACGTTGC | TACCATCCGT |
| 161 | TGGAACGGTG | GTTCCACCTA | CTACACCAAC | TACGGTGACT | CCGTTAAAGA | ACGTTTCGAC | ATCCGTGTTG | ACCAGGCTTC |
| 241 | CAACACCGTT | ACCCTGTCCA | TGGACGACCT | GCAGCCGGAA | GACTCCGCTG | AATACAACAT | CGCTGGTACC | GACATCGGTG |
| 321 | ACGGTTGGTC | CGGTCGTTAC | GACTACCGTG | GTCAGGGTAC | CGACGTTACC | GTTTCCTCG | | |

TABLE 5

| | |
|---|---|
| Primer | Sequence |

| | |
|---|---|
| number | 5′ → 3′ |

| | |
|---|---|
| Pr4 | CAGGAAAACAGCTATGACC |
| Pr5 | TGTAAAACGACGGCCAGT |
| Pr8 | CCTGAAACCTGAGGACACGGCC |
| Pr9 | CAGGGTCCCC/TTG/TGCCCCAG |
| Pr33 | GCTATGCCATAGCATTTTTATCC |
| Pr35 | ACAGCCAAGCTGGAGACCGT |
| Pr49 | GGTGACCTGGGTACCC/TTG/TGCCCCGG |
| Pr56 | GGAGCGC/TGAGGGGGTCTCATG |
| Pr73 | GAGGACACTGCCGTATATTAC/TTG |
| Pr75 | GAGGACACTGCAGAATATAAC/TTG |
| Pr76 | CCAGGGAAGG/CAGCGC/TGAGTT |
| Pr80 | GATGACGATCTTAAGCTCACGNNNCGTGCTGAAGGTTACACCAT |
| | TG |
| Pr81 | CGTAAATGGTAGAATCACCTGCNNNATTGTATTCTGCAGAGTCT |
| | TCC |
| Pr82 | CCGCAATGTGAAACTGGTTTGTAAAGGTGGCAATTTCGTC |
| Pr83 | CGGTAACGTCGGTACCCTGGCAACGGTAGTGGCTATCGTAG |
| Pr120 | AGGCGGGCGGCCGCAATGTGAAACTGGTTG |
| Pr121 | CACCGGCCGAGCTGGCCGACGAGACGGTAA |
| Pr129 | TATACATATGAATGTGAAACTGGTTGAAAAAG |
| Pr136 | CTTCGATATCCGTCGCGACGATGCGTCCAACACCGTTACCTTAT |
| | CG |
| Pr299 | GAGGACACGGCCACATACTACTGT |
| Pr300 | GACCAGGAGTCCTTGGCCCCAGGC |
| Pr301 | GACCAGGAGTCCTTGGCCCCA |
| Pr302 | GTTGTGGTTTTGGTGTCTTGGGTTC |
| Pr303 | CTTGGATTCTGTTGTAGGATTGGGTTG |
| Pr304 | GGGGTCTTCGCTGTGGTGC |
| Pr305 | CTTGGAGCTGGGGTCTTCGC |
| Pr306 | CCGGATCCTTAGTGGTGATGGTGATGGTGGCTTTTGCCCAGGCG |
| | GTTCATTTCTATATCGGTATAGCTCACCGCCACCGGCCGAGCTG |
| | GCCGACGAG |
| Pr775 | CCTGAAACTGACCTGGCGTGCTTCCGGTCG |
| Pr776 | CCTGAAACTGACCGTCCGTGCTTCCGGTCG |
| Pr777 | CCTGAAACTGACCATCCGTGCTTCCGGTCG |
| Pr778 | CCTGAAACTGACCTTCCGTGCTTCCGGTCG |
| Pr779 | TGTCGGTACCAGCGACGTTGTATTCAGCGG |
| Pr780 | TGTCGGTACCAGCGTAGTTGTATTCAGCGG |
| Pr781 | TGTCGGTACCAGCGATGTTGTATTCAGCGG |
| Pr811 | GACCTGGGTCCCAGKTTCCCA |
| Pr813 | GAGGACACGGCAGGYTATAAYTG |
| Pr814 | GAGGACACGGAAAGCTTTACYTG |
| Pr815 | CGGTGACCTGGGTCCCYGKGTCCCAG |
| Pr816 | CGGTGACCTGGGTCCCYGKATCCCCG |
| Pr817 | CGGTGACCTGGGTCCCYGAATTCCCG |
| Pr822 | CCTGAGGACGCGGCCATYTATTAYTG |
| Pr823 | CCTGAGGCCGCAGGCATYTATTAYTG |
| Pr824 | CCTGAGGCTGCAGGCATYTATAAYTG |
| Pr829 | CGGTGACCTGGGTCCCYGKTCCCCA |
| Pr830 | CGGTGACCTGGGTCCAAGCTTCCGA |

TABLE 6

| | | | Amount of | | |
|---|---|---|---|---|---|
| | | | iMab applied | Absorbtion (450 nm) | |
| | No. of | Purification | per well | ELK | Lysozyme |
| iMab | sheets | procedure | (in 100 μl) | (control) | (100 μg/ml) |
| 1302 | 9 | urea | ˜50 ng | 0.045 | 0.345 |
| 1602 | 9 | urea | ˜50 ng | 0.043 | 0.357 |
| 1202 | 9 | urea | ˜50 ng | 0.041 | 0.317 |
| 116 | 9 | urea | ˜50 ng | 0.042 | 0.238 |
| 101 | 7 | urea | ˜20 ng | 0.043 | 0.142 |
| 111 | 9 | urea | ˜50 ng | 0.043 | 0.420 |
| 701 | 6 | urea | ˜10 ng | 0.069 | 0.094 |
| 122 | 9 | urea | ˜50 ng | 0.051 | 0.271 |
| 1300 | 7 | urea | ˜50 ng | 0.041 | 0.325 |
| 1200 | 7 | urea | ˜5 ng | 0.040 | 0.061 |
| 900 | 7 | urea | ˜10 ng | 0.043 | 0.087 |
| 100 | 9 | urea | ˜50 ng | 0.040 | 0.494 |
| 100 | 9 | heat (60° C.) | ˜50 ng | 0.041 | 0.369 |

TABLE 7

| iMab | Signal on Elisa of | Signal on Elisa of |
|---|---|---|
| dilution | input iMab100 | pH shocked iMab100 |
| 1:10 | 0.360 | 0.390 |
| 1:100 | 0.228 | 0.263 |
| 1:1000 | 0.128 | 0.169 |
| 1:10,000 | 0.059 | 0.059 |

TABLE 8
1NEU

| | | | | | | | | | | |
|---|---|---|---|---|---|---|---|---|---|---|
| ATOM | 1 | CA | GLY | 2 | −33.839 | −10.967 | −0.688 | 1.00 | 25.06 | C |
| ATOM | 2 | CA | GLY | 3 | −31.347 | −8.590 | −2.388 | 1.00 | 20.77 | C |
| ATOM | 3 | CA | GLY | 4 | −29.325 | −6.068 | −0.288 | 1.00 | 19.01 | C |
| ATOM | 4 | CA | GLY | 5 | −27.767 | −2.669 | −1.162 | 1.00 | 19.75 | C |
| ATOM | 5 | CA | GLY | 6 | −27.109 | 0.487 | 1.010 | 1.00 | 22.33 | C |
| ATOM | 6 | CA | GLY | 7 | −29.834 | 3.204 | 0.812 | 1.00 | 24.80 | C |
| ATOM | 7 | CA | GLY | 8 | −27.542 | 6.161 | 0.057 | 1.00 | 28.23 | C |
| ATOM | 8 | CA | GLY | 9 | −23.790 | 6.593 | −0.286 | 1.00 | 26.37 | C |
| ATOM | 9 | CA | GLY | 10 | −21.750 | 9.765 | −0.074 | 1.00 | 29.08 | C |
| ATOM | 10 | CA | GLY | 11 | −18.505 | 10.289 | −1.920 | 1.00 | 26.48 | C |
| ATOM | 11 | CA | GLY | 12 | −15.859 | 12.991 | −2.286 | 1.00 | 27.26 | C |
| ATOM | 12 | CA | GLY | 13 | −14.782 | 14.286 | −5.685 | 1.00 | 26.73 | C |
| ATOM | 13 | CA | GLY | 15 | −12.221 | 9.666 | −4.538 | 1.00 | 23.37 | C |
| ATOM | 14 | CA | GLY | 16 | −13.862 | 6.196 | −4.876 | 1.00 | 22.86 | C |
| ATOM | 15 | CA | GLY | 17 | −16.948 | 4.527 | −3.422 | 1.00 | 20.21 | C |
| ATOM | 16 | CA | GLY | 18 | −18.042 | 0.875 | −3.195 | 1.00 | 19.23 | C |
| ATOM | 17 | CA | GLY | 19 | −21.543 | −0.132 | −4.244 | 1.00 | 17.13 | C |
| ATOM | 18 | CA | GLY | 20 | −22.550 | −3.266 | −2.294 | 1.00 | 19.06 | C |
| ATOM | 19 | CA | GLY | 21 | −24.854 | −5.946 | −3.635 | 1.00 | 15.28 | C |
| ATOM | 20 | CA | GLY | 22 | −25.493 | −9.401 | −2.203 | 1.00 | 15.88 | C |
| ATOM | 21 | CA | GLY | 23 | −28.333 | −11.882 | −1.980 | 1.00 | 18.12 | C |
| ATOM | 22 | CA | GLY | 24 | −29.458 | −14.458 | 0.564 | 1.00 | 18.67 | C |
| ATOM | 23 | CA | GLY | 25 | −31.806 | −17.445 | 0.594 | 1.00 | 20.29 | C |
| ATOM | 24 | CA | GLY | 33 | −26.348 | −16.618 | −10.937 | 1.00 | 24.64 | C |
| ATOM | 25 | CA | GLY | 34 | −26.032 | −13.298 | −12.772 | 1.00 | 21.24 | C |
| ATOM | 26 | CA | GLY | 35 | −25.552 | −9.691 | −11.546 | 1.00 | 17.90 | C |
| ATOM | 27 | CA | GLY | 36 | −26.440 | −6.639 | −13.630 | 1.00 | 16.78 | C |
| ATOM | 28 | CA | GLY | 37 | −25.790 | −3.001 | −12.553 | 1.00 | 15.77 | C |
| ATOM | 29 | CA | GLY | 38 | −27.841 | −0.127 | −14.139 | 1.00 | 16.15 | C |
| ATOM | 30 | CA | GLY | 39 | −27.421 | 3.671 | −13.662 | 1.00 | 17.11 | C |
| ATOM | 31 | CA | GLY | 40 | −30.023 | 6.514 | −13.788 | 1.00 | 19.95 | C |
| ATOM | 32 | CA | GLY | 69 | −13.975 | 3.798 | −13.786 | 1.00 | 19.37 | C |
| ATOM | 33 | CA | GLY | 70 | −15.517 | 0.412 | −12.834 | 1.00 | 17.40 | C |
| ATOM | 34 | CA | GLY | 71 | −13.840 | −2.419 | −10.867 | 1.00 | 17.48 | C |
| ATOM | 35 | CA | GLY | 72 | −15.422 | −5.847 | −10.205 | 1.00 | 17.29 | C |
| ATOM | 36 | CA | GLY | 73 | −14.951 | −6.863 | −6.568 | 1.00 | 17.82 | C |
| ATOM | 37 | CA | GLY | 74 | −17.604 | −9.507 | −6.001 | 1.00 | 20.26 | C |
| ATOM | 38 | CA | GLY | 81 | −21.892 | −8.819 | −6.747 | 1.00 | 15.95 | C |
| ATOM | 39 | CA | GLY | 82 | −20.250 | −5.588 | −5.572 | 1.00 | 14.51 | C |
| ATOM | 40 | CA | GLY | 83 | −18.342 | −3.035 | −7.740 | 1.00 | 14.59 | C |
| ATOM | 41 | CA | GLY | 84 | −16.254 | 0.065 | −7.050 | 1.00 | 15.51 | C |
| ATOM | 42 | CA | GLY | 85 | −16.847 | 3.424 | −8.859 | 1.00 | 16.32 | C |
| ATOM | 43 | CA | GLY | 86 | −13.490 | 5.206 | −9.235 | 1.00 | 17.40 | C |
| ATOM | 44 | CA | GLY | 87 | −12.392 | 8.860 | −9.565 | 1.00 | 20.53 | C |
| ATOM | 45 | CA | GLY | 89 | −17.022 | 13.543 | −10.254 | 1.00 | 33.26 | C |
| ATOM | 46 | CA | GLY | 90 | −19.763 | 16.019 | −9.293 | 1.00 | 33.41 | C |
| ATOM | 47 | CA | GLY | 91 | −21.946 | 14.910 | −12.147 | 1.00 | 28.24 | C |
| ATOM | 48 | CA | GLY | 92 | −22.001 | 11.307 | −11.004 | 1.00 | 23.61 | C |
| ATOM | 49 | CA | GLY | 93 | −25.084 | 11.842 | −8.710 | 1.00 | 22.12 | C |
| ATOM | 50 | CA | GLY | 94 | −27.797 | 9.318 | −9.433 | 1.00 | 18.21 | C |
| ATOM | 51 | CA | GLY | 95 | −29.408 | 6.033 | −8.549 | 1.00 | 18.57 | C |
| ATOM | 52 | CA | GLY | 96 | −27.753 | 2.594 | −9.167 | 1.00 | 17.72 | C |
| ATOM | 53 | CA | GLY | 97 | −29.778 | −0.614 | −9.279 | 1.00 | 18.72 | C |
| ATOM | 54 | CA | GLY | 98 | −28.513 | −4.176 | −8.741 | 1.00 | 18.53 | C |
| ATOM | 55 | CA | GLY | 99 | −30.558 | −6.911 | −10.517 | 1.00 | 18.45 | C |
| ATOM | 56 | CA | GLY | 100 | −29.969 | −10.529 | −9.427 | 1.00 | 17.71 | C |
| ATOM | 57 | CA | GLY | 108 | −34.816 | −10.904 | −11.290 | 1.00 | 32.51 | C |
| ATOM | 58 | CA | GLY | 109 | −34.993 | −9.224 | −7.900 | 1.00 | 28.62 | C |
| ATOM | 59 | CA | GLY | 110 | −33.628 | −5.668 | −7.622 | 1.00 | 23.31 | C |
| ATOM | 60 | CA | GLY | 111 | −32.406 | −3.176 | −5.020 | 1.00 | 19.82 | C |
| ATOM | 61 | CA | GLY | 112 | −31.140 | 0.403 | −5.467 | 1.00 | 19.75 | C |
| ATOM | 62 | CA | GLY | 113 | −28.697 | 2.839 | −3.811 | 1.00 | 18.42 | C |
| ATOM | 63 | CA | GLY | 114 | −28.461 | 6.627 | −4.417 | 1.00 | 18.70 | C |
| ATOM | 64 | CA | GLY | 115 | −25.110 | 8.413 | −4.799 | 1.00 | 19.95 | C |
| ATOM | 65 | CA | GLY | 116 | −24.286 | 12.022 | −3.753 | 1.00 | 25.78 | C |
| ATOM | 66 | CA | GLY | 117 | −20.816 | 13.493 | −4.292 | 1.00 | 31.06 | C |
| ATOM | 67 | CA | GLY | 118 | −19.616 | 16.565 | −2.380 | 1.00 | 38.60 | C |
| ATOM | 68 | CA | GLY | 119 | −16.267 | 18.356 | −1.757 | 1.00 | 42.50 | C |
| TER | | | | | | | | | | |

1MEL

| | | | | | | | | | | | |
|---|---|---|---|---|---|---|---|---|---|---|---|
| ATOM | 69 | CA | GLY | A | 3 | −34.517 | −10.371 | −1.234 | 1.00 | 36.96 | C |
| ATOM | 70 | CA | GLY | A | 4 | −31.790 | −8.022 | −2.500 | 1.00 | 28.80 | C |
| ATOM | 71 | CA | GLY | A | 5 | −30.310 | −5.603 | 0.018 | 1.00 | 31.64 | C |
| ATOM | 72 | CA | GLY | A | 6 | −27.884 | −2.957 | −1.209 | 1.00 | 24.68 | C |
| ATOM | 73 | CA | GLY | A | 7 | −25.500 | −1.042 | 1.073 | 1.00 | 21.60 | C |
| ATOM | 74 | CA | GLY | A | 8 | −23.005 | 1.710 | 0.458 | 1.00 | 17.27 | C |
| ATOM | 75 | CA | GLY | A | 9 | −23.567 | 4.843 | −1.499 | 1.00 | 18.58 | C |
| ATOM | 76 | CA | GLY | A | 10 | −23.648 | 8.381 | −0.100 | 1.00 | 16.78 | C |
| ATOM | 77 | CA | GLY | A | 11 | −21.736 | 11.497 | −1.127 | 1.00 | 11.36 | C |
| ATOM | 78 | CA | GLY | A | 12 | −18.140 | 11.942 | −1.980 | 1.00 | 8.84 | C |
| ATOM | 79 | CA | GLY | A | 13 | −16.006 | 14.459 | −3.746 | 1.00 | 11.16 | C |
| ATOM | 80 | CA | GLY | A | 14 | −15.243 | 14.091 | −7.418 | 1.00 | 10.62 | C |
| ATOM | 81 | CA | GLY | A | 16 | −12.920 | 9.782 | −4.554 | 1.00 | 7.90 | C |
| ATOM | 82 | CA | GLY | A | 17 | −14.217 | 6.244 | −4.387 | 1.00 | 7.54 | C |
| ATOM | 83 | CA | GLY | A | 18 | −17.233 | 4.430 | −2.940 | 1.00 | 6.47 | C |
| ATOM | 84 | CA | GLY | A | 19 | −18.104 | 0.790 | −2.418 | 1.00 | 5.43 | C |
| ATOM | 85 | CA | GLY | A | 20 | −21.615 | −0.610 | −2.878 | 1.00 | 8.09 | C |
| ATOM | 86 | CA | GLY | A | 21 | −22.724 | −4.116 | −1.837 | 1.00 | 8.76 | C |
| ATOM | 87 | CA | GLY | A | 22 | −25.644 | −6.399 | −2.433 | 1.00 | 8.33 | C |
| ATOM | 88 | CA | GLY | A | 23 | −26.642 | −9.585 | −0.745 | 1.00 | 13.77 | C |
| ATOM | 89 | CA | GLY | A | 24 | −29.318 | −11.732 | −2.396 | 1.00 | 17.53 | C |
| ATOM | 90 | CA | GLY | A | 25 | −31.340 | −13.525 | 0.256 | 1.00 | 23.23 | C |
| ATOM | 91 | CA | GLY | A | 26 | −33.969 | −16.194 | 0.081 | 1.00 | 25.25 | C |
| ATOM | 92 | CA | GLY | A | 32 | −27.171 | −16.809 | −12.135 | 1.00 | 5.00 | C |
| ATOM | 93 | CA | GLY | A | 33 | −26.411 | −13.068 | −12.502 | 1.00 | 4.64 | C |
| ATOM | 94 | CA | GLY | A | 34 | −26.109 | −10.036 | −10.166 | 1.00 | 2.48 | C |
| ATOM | 95 | CA | GLY | A | 35 | −25.386 | −6.603 | −11.615 | 1.00 | 2.00 | C |
| ATOM | 96 | CA | GLY | A | 36 | −25.845 | −2.895 | −11.151 | 1.00 | 3.40 | C |
| ATOM | 97 | CA | GLY | A | 37 | −28.032 | −0.410 | −12.986 | 1.00 | 6.64 | C |
| ATOM | 98 | CA | GLY | A | 38 | −28.158 | 3.311 | −12.472 | 1.00 | 8.74 | C |
| ATOM | 99 | CA | GLY | A | 39 | −30.731 | 6.025 | −13.179 | 1.00 | 19.24 | C |
| ATOM | 100 | CA | GLY | A | 67 | −12.972 | 2.698 | −13.036 | 1.00 | 12.68 | C |
| ATOM | 101 | CA | GLY | A | 68 | −15.482 | 0.261 | −11.584 | 1.00 | 8.15 | C |
| ATOM | 102 | CA | GLY | A | 69 | −14.843 | −3.306 | −10.511 | 1.00 | 8.39 | C |
| ATOM | 103 | CA | GLY | A | 70 | −17.520 | −5.831 | −9.556 | 1.00 | 5.47 | C |
| ATOM | 104 | CA | GLY | A | 71 | −16.742 | −8.900 | −7.482 | 1.00 | 8.48 | C |
| ATOM | 105 | CA | GLY | A | 72 | −18.459 | −11.484 | −5.287 | 1.00 | 18.00 | C |
| ATOM | 106 | CA | GLY | A | 79 | −21.342 | −8.788 | −4.615 | 1.00 | 9.55 | C |
| ATOM | 107 | CA | GLY | A | 80 | −19.553 | −5.399 | −4.519 | 1.00 | 4.94 | C |
| ATOM | 108 | CA | GLY | A | 81 | −18.901 | −2.601 | −6.858 | 1.00 | 4.92 | C |
| ATOM | 109 | CA | GLY | A | 82 | −15.755 | −0.638 | −6.085 | 1.00 | 7.44 | C |
| ATOM | 110 | CA | GLY | A | 83 | −16.081 | 2.748 | −7.765 | 1.00 | 6.55 | C |
| ATOM | 111 | CA | GLY | A | 84 | −12.869 | 4.759 | −8.177 | 1.00 | 9.49 | C |
| ATOM | 112 | CA | GLY | A | 85 | −12.245 | 8.019 | −10.028 | 1.00 | 12.54 | C |
| ATOM | 113 | CA | GLY | A | 87 | −16.853 | 12.035 | −11.452 | 1.00 | 15.46 | C |
| ATOM | 114 | CA | GLY | A | 88 | −20.054 | 14.055 | −10.955 | 1.00 | 13.94 | C |
| ATOM | 115 | CA | GLY | A | 89 | −21.497 | 12.384 | −14.095 | 1.00 | 18.68 | C |
| ATOM | 116 | CA | GLY | A | 90 | −21.637 | 9.217 | −11.955 | 1.00 | 9.55 | C |
| ATOM | 117 | CA | GLY | A | 91 | −24.288 | 10.888 | −9.734 | 1.00 | 6.54 | C |
| ATOM | 118 | CA | GLY | A | 92 | −27.349 | 8.637 | −9.936 | 1.00 | 6.38 | C |
| ATOM | 119 | CA | GLY | A | 93 | −29.573 | 6.105 | −8.204 | 1.00 | 6.33 | C |
| ATOM | 120 | CA | GLY | A | 94 | −27.866 | 2.727 | −8.154 | 1.00 | 6.08 | C |
| ATOM | 121 | CA | GLY | A | 95 | −29.928 | −0.377 | −8.312 | 1.00 | 10.12 | C |
| ATOM | 122 | CA | GLY | A | 96 | −28.884 | −3.923 | −7.638 | 1.00 | 10.76 | C |
| ATOM | 123 | CA | GLY | A | 97 | −30.439 | −6.641 | −9.794 | 1.00 | 9.08 | C |
| ATOM | 124 | CA | GLY | A | 98 | −30.321 | −10.442 | −10.097 | 1.00 | 8.28 | C |
| ATOM | 125 | CA | GLY | A | 122 | −35.231 | −9.331 | −9.016 | 1.00 | 15.49 | C |
| ATOM | 126 | CA | GLY | A | 123 | −34.520 | −5.802 | −8.079 | 1.00 | 12.37 | C |
| ATOM | 127 | CA | GLY | A | 124 | −33.455 | −4.050 | −4.953 | 1.00 | 14.34 | C |
| ATOM | 128 | CA | GLY | A | 125 | −33.918 | −0.696 | −3.317 | 1.00 | 22.49 | C |
| ATOM | 129 | CA | GLY | A | 126 | −32.054 | 2.128 | −5.022 | 1.00 | 17.72 | C |
| ATOM | 130 | CA | GLY | A | 127 | −29.137 | 3.817 | −3.342 | 1.00 | 16.51 | C |
| ATOM | 131 | CA | GLY | A | 128 | −28.275 | 7.401 | −4.265 | 1.00 | 16.84 | C |
| ATOM | 132 | CA | GLY | A | 129 | −24.678 | 8.216 | −4.904 | 1.00 | 11.90 | C |
| ATOM | 133 | CA | GLY | A | 130 | −23.878 | 11.873 | −5.299 | 1.00 | 8.75 | C |
| ATOM | 134 | CA | GLY | A | 131 | −20.508 | 13.061 | −6.466 | 1.00 | 14.37 | C |
| ATOM | 135 | CA | GLY | A | 132 | −19.632 | 16.597 | −5.198 | 1.00 | 23.32 | C |
| ATOM | 136 | CA | GLY | A | 133 | −17.732 | 19.533 | −6.720 | 1.00 | 36.14 | C |
| TER | | | | | | | | | | | |

1F97

| | | | | | | | | | | | |
|---|---|---|---|---|---|---|---|---|---|---|---|
| ATOM | 137 | CA | GLY | A | 29 | −33.679 | −11.517 | −0.808 | 1.00 | 41.25 | C |
| ATOM | 138 | CA | GLY | A | 30 | −31.468 | −8.740 | −2.170 | 1.00 | 22.43 | C |
| ATOM | 139 | CA | GLY | A | 31 | −30.250 | −5.886 | 0.038 | 1.00 | 24.73 | C |
| ATOM | 140 | CA | GLY | A | 32 | −27.706 | −3.105 | 0.530 | 1.00 | 20.95 | C |
| ATOM | 141 | CA | GLY | A | 33 | −25.811 | −1.723 | 3.523 | 1.00 | 28.77 | C |
| ATOM | 142 | CA | GLY | A | 34 | −26.349 | 1.878 | 2.419 | 1.00 | 33.48 | C |
| ATOM | 143 | CA | GLY | A | 35 | −29.027 | 3.067 | −0.012 | 1.00 | 27.47 | C |
| ATOM | 144 | CA | GLY | A | 36 | −27.980 | 6.732 | 0.249 | 1.00 | 29.20 | C |
| ATOM | 145 | CA | GLY | A | 37 | −24.279 | 6.955 | −0.586 | 1.00 | 23.99 | C |
| ATOM | 146 | CA | GLY | A | 38 | −22.275 | 10.179 | −0.393 | 1.00 | 24.19 | C |
| ATOM | 147 | CA | GLY | A | 39 | −18.592 | 10.286 | −1.302 | 1.00 | 15.35 | C |
| ATOM | 148 | CA | GLY | A | 40 | −16.117 | 13.059 | −2.218 | 1.00 | 12.64 | C |
| ATOM | 149 | CA | GLY | A | 41 | −15.286 | 13.515 | −5.906 | 1.00 | 9.24 | C |
| ATOM | 150 | CA | GLY | A | 43 | −12.412 | 8.992 | −4.540 | 1.00 | 14.44 | C |
| ATOM | 151 | CA | GLY | A | 44 | −13.115 | 5.267 | −4.685 | 1.00 | 21.52 | C |
| ATOM | 152 | CA | GLY | A | 45 | −16.460 | 3.862 | −3.630 | 1.00 | 22.48 | C |
| ATOM | 153 | CA | GLY | A | 46 | −18.082 | 0.444 | −3.532 | 1.00 | 21.22 | C |
| ATOM | 154 | CA | GLY | A | 47 | −21.817 | 0.219 | −4.135 | 1.00 | 20.06 | C |
| ATOM | 155 | CA | GLY | A | 48 | −22.797 | −2.825 | −2.072 | 1.00 | 13.56 | C |
| ATOM | 156 | CA | GLY | A | 49 | −25.390 | −5.432 | −3.034 | 1.00 | 19.14 | C |
| ATOM | 157 | CA | GLY | A | 50 | −25.724 | −8.537 | −0.876 | 1.00 | 22.49 | C |
| ATOM | 158 | CA | GLY | A | 51 | −28.046 | −11.470 | −1.549 | 1.00 | 20.58 | C |
| ATOM | 159 | CA | GLY | A | 52 | −28.951 | −14.871 | −0.105 | 1.00 | 24.55 | C |
| ATOM | 160 | CA | GLY | A | 53 | −30.893 | −17.875 | −1.353 | 1.00 | 17.82 | C |
| ATOM | 161 | CA | GLY | A | 57 | −26.875 | −15.797 | −9.701 | 1.00 | 10.39 | C |
| ATOM | 162 | CA | GLY | A | 58 | −26.674 | −13.408 | −12.632 | 1.00 | 8.00 | C |
| ATOM | 163 | CA | GLY | A | 59 | −25.845 | −9.939 | −11.318 | 1.00 | 7.62 | C |
| ATOM | 164 | CA | GLY | A | 60 | −26.653 | −6.841 | −13.371 | 1.00 | 6.84 | C |
| ATOM | 165 | CA | GLY | A | 61 | −26.457 | −3.109 | −12.711 | 1.00 | 8.48 | C |
| ATOM | 166 | CA | GLY | A | 62 | −28.184 | −0.168 | −14.336 | 1.00 | 13.92 | C |
| ATOM | 167 | CA | GLY | A | 63 | −27.611 | 3.567 | −14.043 | 1.00 | 11.89 | C |
| ATOM | 168 | CA | GLY | A | 64 | −30.532 | 5.981 | −14.200 | 1.00 | 23.18 | C |
| ATOM | 169 | CA | GLY | A | 85 | −12.888 | 2.268 | −13.813 | 1.00 | 17.93 | C |
| ATOM | 170 | CA | GLY | A | 86 | −15.508 | −0.149 | −12.447 | 1.00 | 15.44 | C |
| ATOM | 171 | CA | GLY | A | 87 | −14.603 | −3.542 | −11.016 | 1.00 | 16.79 | C |
| ATOM | 172 | CA | GLY | A | 88 | −17.118 | −6.318 | −10.381 | 1.00 | 14.07 | C |
| ATOM | 173 | CA | GLY | A | 89 | −17.389 | −8.592 | −7.349 | 1.00 | 16.75 | C |
| ATOM | 174 | CA | GLY | A | 91 | −20.798 | −8.262 | −3.202 | 1.00 | 17.13 | C |
| ATOM | 175 | CA | GLY | A | 92 | −20.995 | −5.059 | −5.247 | 1.00 | 12.84 | C |
| ATOM | 176 | CA | GLY | A | 93 | −19.287 | −2.826 | −7.795 | 1.00 | 12.33 | C |
| ATOM | 177 | CA | GLY | A | 94 | −16.243 | −0.709 | −6.986 | 1.00 | 12.43 | C |
| ATOM | 178 | CA | GLY | A | 95 | −15.485 | 2.596 | −8.696 | 1.00 | 11.12 | C |
| ATOM | 179 | CA | GLY | A | 96 | −11.765 | 3.339 | −8.675 | 1.00 | 16.95 | C |
| ATOM | 180 | CA | GLY | A | 97 | −12.855 | 7.004 | −8.899 | 1.00 | 21.11 | C |
| ATOM | 181 | CA | GLY | A | 99 | −16.935 | 12.349 | −10.689 | 1.00 | 22.37 | C |
| ATOM | 182 | CA | GLY | A | 100 | −19.974 | 14.613 | −10.361 | 1.00 | 23.75 | C |
| ATOM | 183 | CA | GLY | A | 101 | −21.190 | 13.009 | −13.619 | 1.00 | 22.84 | C |
| ATOM | 184 | CA | GLY | A | 102 | −21.820 | 9.789 | −11.695 | 1.00 | 15.51 | C |
| ATOM | 185 | CA | GLY | A | 103 | −24.651 | 11.224 | −9.565 | 1.00 | 11.57 | C |
| ATOM | 186 | CA | GLY | A | 104 | −27.817 | 9.170 | −9.882 | 1.00 | 9.52 | C |
| ATOM | 187 | CA | GLY | A | 105 | −29.401 | 5.897 | −8.871 | 1.00 | 12.64 | C |
| ATOM | 188 | CA | GLY | A | 106 | −27.851 | 2.484 | −9.413 | 1.00 | 7.32 | C |
| ATOM | 189 | CA | GLY | A | 107 | −30.057 | −0.590 | −9.334 | 1.00 | 7.52 | C |
| ATOM | 190 | CA | GLY | A | 108 | −28.588 | −3.984 | −8.524 | 1.00 | 9.97 | C |
| ATOM | 191 | CA | GLY | A | 109 | −30.576 | −6.743 | −10.211 | 1.00 | 12.91 | C |
| ATOM | 192 | CA | GLY | A | 110 | −29.973 | −10.300 | −9.043 | 1.00 | 11.27 | C |
| ATOM | 193 | CA | GLY | A | 118 | −35.528 | −12.495 | −8.616 | 1.00 | 13.29 | C |
| ATOM | 194 | CA | GLY | A | 119 | −34.748 | −9.395 | −6.594 | 1.00 | 15.55 | C |
| ATOM | 195 | CA | GLY | A | 120 | −33.436 | −5.837 | −6.855 | 1.00 | 11.36 | C |
| ATOM | 196 | CA | GLY | A | 121 | −32.267 | −2.894 | −4.778 | 1.00 | 11.36 | C |
| ATOM | 197 | CA | GLY | A | 122 | −31.635 | 0.758 | −5.660 | 1.00 | 11.14 | C |
| ATOM | 198 | CA | GLY | A | 123 | −28.791 | 2.935 | −4.379 | 1.00 | 11.58 | C |
| ATOM | 199 | CA | GLY | A | 124 | −28.626 | 6.699 | −4.773 | 1.00 | 15.52 | C |
| ATOM | 200 | CA | GLY | A | 125 | −25.128 | 8.065 | −5.281 | 1.00 | 9.61 | C |
| ATOM | 201 | CA | GLY | A | 126 | −24.275 | 11.677 | −4.483 | 1.00 | 10.84 | C |
| ATOM | 202 | CA | GLY | A | 127 | −20.739 | 12.778 | −5.330 | 1.00 | 10.58 | C |
| ATOM | 203 | CA | GLY | A | 128 | −19.667 | 15.540 | −2.929 | 1.00 | 14.41 | C |
| ATOM | 204 | CA | GLY | A | 129 | −18.355 | 18.818 | −4.335 | 1.00 | 12.32 | C |
| TER | | | | | | | | | | | |

1DQT

| | | | | | | | | | | | |
|---|---|---|---|---|---|---|---|---|---|---|---|
| ATOM | 205 | CA | GLY | C | 2 | −37.000 | −7.803 | −3.232 | 1.00 | 35.96 | C |
| ATOM | 206 | CA | GLY | C | 3 | −33.582 | −6.481 | −4.122 | 1.00 | 30.20 | C |
| ATOM | 207 | CA | GLY | C | 4 | −31.887 | −3.962 | −1.908 | 1.00 | 27.24 | C |
| ATOM | 208 | CA | GLY | C | 5 | −28.640 | −2.044 | −1.950 | 1.00 | 23.16 | C |
| ATOM | 209 | CA | GLY | C | 6 | −27.069 | 0.720 | 0.216 | 1.00 | 22.73 | C |
| ATOM | 210 | CA | GLY | C | 7 | −28.256 | 4.289 | −0.273 | 1.00 | 24.33 | C |
| ATOM | 211 | CA | GLY | C | 8 | −24.800 | 5.874 | −0.329 | 1.00 | 21.64 | C |
| ATOM | 212 | CA | GLY | C | 9 | −21.252 | 4.822 | −1.130 | 1.00 | 20.25 | C |
| ATOM | 213 | CA | GLY | C | 10 | −18.186 | 7.087 | −0.970 | 1.00 | 20.37 | C |
| ATOM | 214 | CA | GLY | C | 11 | −15.855 | 5.961 | −3.761 | 1.00 | 22.46 | C |
| ATOM | 215 | CA | GLY | C | 12 | −12.159 | 5.548 | −2.990 | 1.00 | 24.30 | C |
| ATOM | 216 | CA | GLY | C | 14 | −8.661 | 5.520 | −6.931 | 1.00 | 30.82 | C |
| ATOM | 217 | CA | GLY | C | 15 | −12.218 | 5.495 | −8.241 | 1.00 | 25.29 | C |
| ATOM | 218 | CA | GLY | C | 16 | −13.342 | 2.240 | −6.604 | 1.00 | 21.63 | C |
| ATOM | 219 | CA | GLY | C | 17 | −16.853 | 1.719 | −5.287 | 1.00 | 20.22 | C |
| ATOM | 220 | CA | GLY | C | 18 | −17.869 | −1.533 | −3.647 | 1.00 | 20.86 | C |
| ATOM | 221 | CA | GLY | C | 19 | −21.187 | −2.475 | −2.147 | 1.00 | 21.25 | C |
| ATOM | 222 | CA | GLY | C | 20 | −23.544 | −5.395 | −1.581 | 1.00 | 21.73 | C |
| ATOM | 223 | CA | GLY | C | 21 | −26.665 | −6.116 | −3.611 | 1.00 | 22.97 | C |
| ATOM | 224 | CA | GLY | C | 22 | −29.032 | −8.318 | −1.684 | 1.00 | 26.59 | C |
| ATOM | 225 | CA | GLY | C | 23 | −32.087 | −10.258 | −2.722 | 1.00 | 26.47 | C |
| ATOM | 226 | CA | GLY | C | 24 | −34.892 | −12.390 | −1.373 | 1.00 | 31.54 | C |
| ATOM | 227 | CA | GLY | C | 25 | −36.216 | −14.994 | −1.386 | 1.00 | 31.33 | C |
| ATOM | 228 | CA | GLY | C | 32 | −28.221 | −15.396 | −10.763 | 1.00 | 21.25 | C |
| ATOM | 229 | CA | GLY | C | 33 | −27.582 | −12.861 | −13.499 | 1.00 | 21.86 | C |
| ATOM | 230 | CA | GLY | C | 34 | −26.676 | −9.507 | −11.974 | 1.00 | 19.78 | C |
| ATOM | 231 | CA | GLY | C | 35 | −26.814 | −6.238 | −13.884 | 1.00 | 19.11 | C |
| ATOM | 232 | CA | GLY | C | 36 | −25.505 | −2.810 | −12.890 | 1.00 | 19.23 | C |
| ATOM | 233 | CA | GLY | C | 37 | −27.371 | 0.131 | −14.384 | 1.00 | 25.95 | C |
| ATOM | 234 | CA | GLY | C | 38 | −26.592 | 3.830 | −14.106 | 1.00 | 30.90 | C |
| ATOM | 235 | CA | GLY | C | 39 | −29.761 | 5.879 | −13.906 | 1.00 | 40.84 | C |
| ATOM | 236 | CA | GLY | C | 66 | −16.463 | −4.323 | −12.728 | 1.00 | 23.17 | C |
| ATOM | 237 | CA | GLY | C | 67 | −15.869 | −7.277 | −10.506 | 1.00 | 22.56 | C |
| ATOM | 238 | CA | GLY | C | 68 | −17.716 | −9.180 | −7.811 | 1.00 | 21.60 | C |
| ATOM | 239 | CA | GLY | C | 69 | −18.545 | −12.367 | −5.959 | 1.00 | 22.69 | C |
| ATOM | 240 | CA | GLY | C | 75 | −23.757 | −10.273 | −4.597 | 1.00 | 23.03 | C |
| ATOM | 241 | CA | GLY | C | 76 | −20.713 | −8.179 | −3.648 | 1.00 | 24.30 | C |
| ATOM | 242 | CA | GLY | C | 77 | −20.192 | −5.631 | −6.425 | 1.00 | 23.46 | C |
| ATOM | 243 | CA | GLY | C | 78 | −17.130 | −3.554 | −7.209 | 1.00 | 25.74 | C |
| ATOM | 244 | CA | GLY | C | 79 | −17.159 | −0.822 | −9.864 | 1.00 | 26.08 | C |
| ATOM | 245 | CA | GLY | C | 80 | −13.722 | 0.567 | −10.770 | 1.00 | 29.15 | C |
| ATOM | 246 | CA | GLY | C | 81 | −12.154 | 3.299 | −12.879 | 1.00 | 26.23 | C |
| ATOM | 247 | CA | GLY | C | 84 | −15.857 | 12.510 | −10.546 | 1.00 | 24.79 | C |
| ATOM | 248 | CA | GLY | C | 85 | −18.200 | 12.785 | −13.517 | 1.00 | 25.37 | C |
| ATOM | 249 | CA | GLY | C | 86 | −19.382 | 9.218 | −12.817 | 1.00 | 25.21 | C |
| ATOM | 250 | CA | GLY | C | 87 | −20.993 | 10.374 | −9.582 | 1.00 | 23.95 | C |
| ATOM | 251 | CA | GLY | C | 88 | −24.588 | 9.210 | −9.761 | 1.00 | 23.67 | C |
| ATOM | 252 | CA | GLY | C | 89 | −27.227 | 6.570 | −9.098 | 1.00 | 22.75 | C |
| ATOM | 253 | CA | GLY | C | 90 | −26.436 | 2.913 | −9.751 | 1.00 | 23.39 | C |
| ATOM | 254 | CA | GLY | C | 91 | −29.150 | 0.276 | −9.841 | 1.00 | 22.55 | C |
| ATOM | 255 | CA | GLY | C | 92 | −28.491 | −3.332 | −9.011 | 1.00 | 22.29 | C |
| ATOM | 256 | CA | GLY | C | 93 | −30.706 | −5.811 | −10.865 | 1.00 | 20.45 | C |
| ATOM | 257 | CA | GLY | C | 94 | −30.958 | −9.487 | −9.970 | 1.00 | 20.73 | C |
| ATOM | 258 | CA | GLY | C | 105 | −35.975 | −7.679 | −9.086 | 1.00 | 24.94 | C |
| ATOM | 259 | CA | GLY | C | 106 | −34.133 | −4.376 | −8.832 | 1.00 | 25.24 | C |
| ATOM | 260 | CA | GLY | C | 107 | −32.992 | −2.157 | −5.970 | 1.00 | 24.05 | C |
| ATOM | 261 | CA | GLY | C | 108 | −33.717 | 1.590 | −5.665 | 1.00 | 25.85 | C |
| ATOM | 262 | CA | GLY | C | 109 | −30.127 | 2.411 | −6.479 | 1.00 | 23.77 | C |
| ATOM | 263 | CA | GLY | C | 110 | −26.942 | 3.329 | −4.666 | 1.00 | 22.46 | C |
| ATOM | 264 | CA | GLY | C | 111 | −25.759 | 6.950 | −4.824 | 1.00 | 24.13 | C |
| ATOM | 265 | CA | GLY | C | 112 | −22.065 | 6.752 | −5.605 | 1.00 | 22.83 | C |
| ATOM | 266 | CA | GLY | C | 113 | −20.136 | 9.957 | −4.898 | 1.00 | 23.40 | C |
| ATOM | 267 | CA | GLY | C | 114 | −16.799 | 10.239 | −6.594 | 1.00 | 25.42 | C |
| END | | | | | | | | | | | |

TABLE 9

| | zp-comb | objective function |
|---|---|---|
| molecule | (lower is better) | (lower is better) |
| iMab 101 | −5.63 | 853 |
| iMab 201 | −5.34 | 683 |
| iMab IS003 | −4.29 | 860 |
| iMab IS004 | −1.49 | 2744 |
| iMab 300 | −6.28 | 854 |
| iMab IS006 | −3.29 | 912 |
| iMab IS007 | −2.71 | 1558 |
| iMab IS008 | −1.10 | 808 |
| iMab IS009 | −3.70 | 1623 |
| iMab IS0010 | −2.85 | 2704 |
| iMab 400 | −5.58 | 734 |
| iMab IS0012 | −5.35 | 889 |
| iMab IS0013 | −2.85 | 1162 |
| iMab IS0014 | −2.92 | 924 |
| iMab IS0015 | −3.48 | 925 |
| iMab IS0016 | −3.23 | 837 |
| iMab 500 | −3.94 | 1356 |
| iMab IS0018 | −2.97 | 867 |
| iMab IS0019 | −3.11 | 1366 |
| iMab 600 | −4.15 | 880 |
| iMab 700 | −3.94 | 1111 |
| iMab 800 | −3.68 | 653 |
| iMab 900 | −4.65 | 833 |
| iMab 1000 | −3.57 | 631 |
| iMab IS0025 | −2.79 | 1080 |
| iMab 1100 | −4.07 | 823 |
| iMab IS0027 | −3.59 | 809 |
| iMab IS0028 | −3.51 | 1431 |
| iMab 1200 | −2.66 | 783 |
| iMab 1300 | −3.18 | 1463 |
| iMab IS0031 | −2.98 | 1263 |
| iMab IS0032 | −3.84 | 896 |
| iMab 1400 | −5.17 | 939 |
| iMab IS0034 | −4.38 | 966 |
| iMab IS0035 | −3.86 | 966 |
| iMab IS0036 | −3.29 | 862 |
| iMab IS0037 | −3.45 | 874 |
| iMab IS0038 | −2.80 | 792 |
| iMab IS0039 | −4.44 | 1858 |
| iMab IS0040 | −5.01 | 751 |
| iMab IS0041 | −2.70 | 907 |
| iMab IS0042 | −3.14 | 837 |
| iMab IS0043 | −2.80 | 1425 |
| iMab IS0044 | −3.27 | 1492 |
| iMab IS0045 | −3.56 | 1794 |
| iMab IS0046 | −3.79 | 832 |

TABLE 10

iMabIS003
IVLTQS-P--ASLAV-S-----LGQRATISCRASGYTIGPS-FMNWFQQK
P------G--Q--PP-K--LLIYANMGDFSLNI-H--P--M-EE---EDT
A---MYFCAADSTIYASYYECGHGISTGGYGYLTFGAGTKVELKR
iMabIS004
PTVSIF-P--P-SSE-QL----TSGGASVVCFASGYTIGPI-NVKWKIDG
S------E----------------NMGSSTLTL-T--K--D-E---YERH
N---SYTCAADSTIYASYYECGHGISTGGYGYPIVKSFNRNE---
iMabIS006
TPPSVY-P--L-APG-SAAQTNSMVTLGCLVKASGYTIGPE-PVTVTWNS
G------S--L--SS-G--VHTFPNMGTLSSSV-T--V--P-SSTWPSET
V---TCNCAADSTIYASYYECGHGISTGGYGY-STKVDKKIVPK-
iMabIS007
IASPAKTH--E-KTP-I-----EGRPFQLDCVASGYTIGP--LITWKKRL
SGADPN------------------NMG-GNLYF-T--I--V-TK---EDV
SDIYKYVCAADSTIYASYYECGHGISTGGYGYEVVLVEYEIKGVT
iMabIS008
PVLKDQPA--E-VLF-R-----ENNPTVLECIASGYTIGPV-KYSWKKDG
KSYNW-----Q--EH-N--AALRKNMGEGSLVF-L--R--P-QA---SDE
G---HYQCAADSTIYASYYECGHGISTGGYGYVASSRVISFRKTY
iMabIS009
KYEQKPEK--V-IVV-K-----QGQDVTIPCKASGYTIGPP-NVVWSHNA
KP----------------------NMGDSGLVI-K--G--V-KN---GDK
G---YYGCAADSTIYASYYECGHGISTGGYGY-DKYFETLVQVN-
iMabIS010
VPQYVS-K--D-MMA-K-----AGDVTMIYCMASGYTIGPG-YPNYFKNG
KDVN--------------------NMGGKRLLF-K--T--T-LP---EDE
G---VYTCAADSTIYASYYECGHGISTGGYGY-PQKHSLKLTVVS
iMabIS012
IQMTQS-P-SS-LSA-S-----VGDRVTITCSASGYTIGPN-YLNWYQQK
P------G--K--AP-K--VLIYFNMGDFTLTI-S--S--L-QP---EDF
A---TYYCAADSTIYASYYECGHGISTGGYGYWTFGQGTKVEIKR
iMabIS013
PSVFIF-P--P-SDE-Q----LKSGTASVVCLASGYTIGPA-KVQWKVD-
--------------N-A--LQS--NMGSSTLTL-S--K--A-DY---EKH
K---VYACAADSTIYASYYECGHGISTGGYGYPVTKSFNRGEC--
iMabIS014
KGPSVF-P--L-APS-SKSTSGGTAALGCLVKASGYTIGPE-PVTVSWNS
G------A--L--TS-G--VHTFPNMGSLSSVV-T--V--P-SSSLGTQT
Y---ICNCAADSTIYASYYECGHGISTGGYGY-NTKVDKKVEPKS
iMabIS015
NPPHNL-S--V-INSEE-----LSSILKLTWTASGYTIGPL-KYNIQYRT
KD-----A--S--TW-S--QIPP-NMGRSSFTV-Q--D--L-KP---FTE
Y---VFRCAADSTIYASYYECGHGISTGGYGYSDWSEEASGITYE
iMabIS016
EKPKNL-S--C-IV--N-----EGKKMRCEWDASGYTIGPT-NFTLKSEW
A------T--H--K--F--ADCKANMGPTSCTVDY--S--T-VY---FVN
I---EVWCAADSTIYASYYECGHGISTGGYGYKVTSDHINFDPVY
iMabIS018
NAPKLT-G-IT-CQA-D--------KAEIHWEASGYTIGPL-HYTIQFNT
S------F--TPASW-D--AAYEKNMGDSSFVV-Q--M--S--P---WAN
Y---TFRCAADSTIYASYYECGHGISTGGYGYSPPSAHSDSCT--
iMabIS019
GPEELL-C--F-TE--------RLEDLVCFWEASGYTIGPG-QYSFSYQL
E------D--E--PW-K--LCR--NMGRFWCSL-P--TADT-SS---FVP
L---ELRCAADSTIYASYYECGHGISTGGYGYGAPRYHRVIHINE
iMabIS020
APVGLV-A--R-LA--D-----ESGHVVLRWLASGYTIGPI-RYEVDVSA
G------Q--GAG-S-V--QRVEINMGRTECVL-S--N--L-RG---RTR
Y---TFACAADSTIYASYYECGHGISTGGYGYSEWSEPVSLLTPS
iMabIS025
GPEELL-C--F-TE--------RLEDLVCFWEASGYTIGPPGNYSFSYQL
E------D--E--PW-K--LCR--NMGRFWCSL-P--TADT-SS---FVP
L---ELRCAADSTIYASYYECGHGISTGGYGYGAPRYHRVIHINE
iMabIS027
APVGLV-A--R-LAD-E------SGHVVLRWLASGYTIGPI-RYEVDVSA
G------QGAG--SV-Q--RVEILNMG-TECVL-S--N--L-RG---RTR
Y---TFACAADSTIYASYYECGHGISTGGYGYSEWSEPVSLLTPS
IMABIS028
GPEELL-C--F-TE--------RLEDLVCFWEASGYTIGPG-QYSFSYQL
E------D--E--PW-K--LCR--NMGRFWCSL-PTAD--T-SS---FVP
L---ELRCAADSTIYASYYECGHGISTGGYGYGAPRYHRVIHINE
iMabIS031
LMFKNAPT-PQ-EFK-------EGEDAVIVCDASGYTIGPP-TIIWKHKG
RDV---------------------NMGNNYLQI-R--G--I-KK---TDE
G---TYRCAADSTIYASYYECGHGISTGGYGYINFK-DIQVIV--
iMabIS032
DSPTGI-D--F-SD--I-----TANSFTVHWIASGYTIGPT-GYRIRHHP
E------H--F--SGRP--REDRVNMGRNSITL-T--N--L-TP---GTE
Y---VVSCAADSTIYASYYECGHGISTGGYGYSPL-LIGQQSTVS
iMabIS034
SPPTNL-H--L-EAN-P-----DTGVLTVSWEASGYTIGPT-GYRITTTP
T------N--G--QQGN-SLEEVVNMGQSSCTF-D--N--L-SP---GLE
Y---NVSCAADSTIYASYYECGHGISTGGYGYSVP-ISDTIIPAV
iMabIS035
PPTDLR-F--T-NIG-P-----D--TMRVTWAASGYTIGPT-NFLVRYSP
V------K--N--EEDV--AELSINMGDNAVVL-T--N--L-LP---GTE
Y---VVSCAADSTIYASYYECGHGISTGGYGYSTPL-RGRQKTGL
iMabIS036
NPPHNL-S--V-INSEE-----LSSILKLTWTASGYTIGPL-KYNIQYRT
KD-----A--S--TW-S--QIPPENMGRSSFTV-Q--D--L-KP---FTE
Y---VFRCAADSTIYASYYECGHGISTGGYGYSDWSEEASGITYE
iMabIS037
PCGYIS-P--ESPVV-Q-----LHSNFTAVCVASGYTIGPN-YIVWKTN-
--------------H-F--TIPK-NMGASSVTF-T--D--I-AS---L-N
I---QLTCAADSTIYASYYECGHGISTGGYGYEQNVYGITIISGL
iMabIS038
EKPKNL-S--CIVN--------EGKKMRCEWDASGYTIGPT-NFTLKSEW
A------T--H--KF----ADCKANMGPTSCTV-D--Y--STVY---FVN
I---EVWCAADSTIYASYYECGHGISTGGYGYKVTSDHINFDPVY
iMabIS039
RFIVKP-Y--G-TEV-G-----EGQSANFYCRASGYTIGPP-VVTWHKD-
----------D--RE-L--K----NMGDYGLTI-N--R--V-KG---DDK
G---EYTCAADSTIYASYYECGHGISTGGYGYGTKEEIVFLNVTR
iMabIS041
SEPGRL-A--FNV---V-----SSTVTQLSWAASGYTIGPT-AYEVCYGL
VNDDNRPI--G--PM-K--KVLVDNMGNRMLLI-E--N--L-RE---SQP
Y---RYTCAADSTIYASYYECGHGISTGGYGYWGPEREAIINLAT
iMabIS042
APQNPN-A--K-AA--------GSRKIHFNWLASGYTIGPM-GYRVKYWIQ
------G--D--SE-SEAHLLDSNMGVPSVEL-T--N--L-YP---YCDY-
--EMKCAADSTIYASYYECGHGISTGGYGYGPYSSLVSCRTHQ
iMabIS044
IEVEKP--LYG-VEV-F-----VGETAHFEIEASGYTIGPV-HGQWKLKGQ
P----------------------NMGKHILIL-H--N--C-QL---GMTG-
--EVSCAADSTIYASYYECGHGISTGGYGY-NAKSAANLKVKE
iMabIS045
FKIETT-PESR-YLA-Q-----IGDSVSLTCSASGYTIGPP-FFSWRTQID
S----------------------NMGTSTLTM-N--P--V-SF---GNEH-
--SYLCAADSTIYASYYECGHGISTGGYGYRKLEKGIQVEIYS

TABLE 11
1NEU

| | | | | | | | | | | |
|---|---|---|---|---|---|---|---|---|---|---|
| ATOM | 1 | CA | GLY | 15 | −13.154 | 9.208 | −3.380 | 1.00 | 23.37 | C |
| ATOM | 2 | CA | GLY | 16 | −14.293 | 5.561 | −3.888 | 1.00 | 22.86 | C |
| ATOM | 3 | CA | GLY | 17 | −16.782 | 3.259 | −2.179 | 1.00 | 20.21 | C |
| ATOM | 4 | CA | GLY | 18 | −17.260 | −0.530 | −2.245 | 1.00 | 19.23 | C |
| ATOM | 5 | CA | GLY | 19 | −20.702 | −2.004 | −2.834 | 1.00 | 17.13 | C |
| ATOM | 6 | CA | GLY | 20 | −20.862 | −5.442 | −1.161 | 1.00 | 19.06 | C |
| ATOM | 7 | CA | GLY | 21 | −22.944 | −8.326 | −2.443 | 1.00 | 15.28 | C |
| ATOM | 8 | CA | GLY | 22 | −22.792 | −11.964 | −1.378 | 1.00 | 15.88 | C |
| ATOM | 9 | CA | GLY | 23 | −25.143 | −14.899 | −1.017 | 1.00 | 18.12 | C |
| ATOM | 10 | CA | GLY | 24 | −25.395 | −17.869 | 1.327 | 1.00 | 18.67 | C |
| ATOM | 11 | CA | GLY | 25 | −27.226 | −21.197 | 1.356 | 1.00 | 20.29 | C |
| ATOM | 12 | CA | GLY | 33 | −24.118 | −18.315 | −10.706 | 1.00 | 24.64 | C |
| ATOM | 13 | CA | GLY | 34 | −24.637 | −14.823 | −12.130 | 1.00 | 21.24 | C |
| ATOM | 14 | CA | GLY | 35 | −24.488 | −11.328 | −10.549 | 1.00 | 17.90 | C |
| ATOM | 15 | CA | GLY | 36 | −26.181 | −8.277 | −12.056 | 1.00 | 16.78 | C |
| ATOM | 16 | CA | GLY | 37 | −25.898 | −4.707 | −10.644 | 1.00 | 15.77 | C |
| ATOM | 17 | CA | GLY | 38 | −28.607 | −2.078 | −11.499 | 1.00 | 16.15 | C |
| ATOM | 18 | CA | GLY | 39 | −28.680 | 1.671 | −10.617 | 1.00 | 17.11 | C |
| ATOM | 19 | CA | GLY | 40 | −31.657 | 4.031 | −9.957 | 1.00 | 19.95 | C |
| ATOM | 20 | CA | GLY | 69 | −15.648 | 4.079 | −12.895 | 1.00 | 19.37 | C |
| ATOM | 21 | CA | GLY | 70 | −16.470 | 0.404 | −12.145 | 1.00 | 17.40 | C |
| ATOM | 22 | CA | GLY | 71 | −14.063 | −2.286 | −10.851 | 1.00 | 17.48 | C |
| ATOM | 23 | CA | GLY | 72 | −14.971 | −5.980 | −10.384 | 1.00 | 17.29 | C |
| ATOM | 24 | CA | GLY | 73 | −13.707 | −7.259 | −7.030 | 1.00 | 17.82 | C |
| ATOM | 25 | CA | GLY | 74 | −15.790 | −10.357 | −6.383 | 1.00 | 20.26 | C |
| ATOM | 26 | CA | GLY | 81 | −20.196 | −10.332 | −6.333 | 1.00 | 15.95 | C |
| ATOM | 27 | CA | GLY | 82 | −18.870 | −7.004 | −5.037 | 1.00 | 14.51 | C |
| ATOM | 28 | CA | GLY | 83 | −17.786 | −3.962 | −7.142 | 1.00 | 14.59 | C |
| ATOM | 29 | CA | GLY | 84 | −16.094 | −0.638 | −6.409 | 1.00 | 15.51 | C |
| ATOM | 30 | CA | GLY | 85 | −17.498 | 2.735 | −7.656 | 1.00 | 16.32 | C |
| ATOM | 31 | CA | GLY | 86 | −14.567 | 5.088 | −8.340 | 1.00 | 17.40 | C |
| ATOM | 32 | CA | GLY | 87 | −14.105 | 8.889 | −8.375 | 1.00 | 20.53 | C |
| ATOM | 33 | CA | GLY | 89 | −19.429 | 12.768 | −7.705 | 1.00 | 33.26 | C |
| ATOM | 34 | CA | GLY | 90 | −22.291 | 14.637 | −6.007 | 1.00 | 33.41 | C |
| ATOM | 35 | CA | GLY | 91 | −24.761 | 13.465 | −8.588 | 1.00 | 28.24 | C |
| ATOM | 36 | CA | GLY | 92 | −24.071 | 9.808 | −7.919 | 1.00 | 23.61 | C |
| ATOM | 37 | CA | GLY | 93 | −26.736 | 9.584 | −5.107 | 1.00 | 22.12 | C |
| ATOM | 38 | CA | GLY | 94 | −29.127 | 6.723 | −5.698 | 1.00 | 18.21 | C |
| ATOM | 39 | CA | GLY | 95 | −30.045 | 3.141 | −4.991 | 1.00 | 18.57 | C |
| ATOM | 40 | CA | GLY | 96 | −28.033 | 0.110 | −6.301 | 1.00 | 17.72 | C |
| ATOM | 41 | CA | GLY | 97 | −29.544 | −3.367 | −6.491 | 1.00 | 18.72 | C |
| ATOM | 42 | CA | GLY | 98 | −27.685 | −6.700 | −6.623 | 1.00 | 18.53 | C |
| ATOM | 43 | CA | GLY | 99 | −29.584 | −9.550 | −8.379 | 1.00 | 18.45 | C |
| ATOM | 44 | CA | GLY | 100 | −28.276 | −13.106 | −7.867 | 1.00 | 17.71 | C |
| ATOM | 45 | CA | GLY | 108 | −33.268 | −14.108 | −8.956 | 1.00 | 32.51 | C |
| ATOM | 46 | CA | GLY | 109 | −33.081 | −12.828 | −5.395 | 1.00 | 28.62 | C |
| ATOM | 47 | CA | GLY | 110 | −32.234 | −9.138 | −4.891 | 1.00 | 23.31 | C |
| ATOM | 48 | CA | GLY | 111 | −30.950 | −6.748 | −2.225 | 1.00 | 19.82 | C |
| ATOM | 49 | CA | GLY | 112 | −30.333 | −2.979 | −2.412 | 1.00 | 19.75 | C |
| ATOM | 50 | CA | GLY | 113 | −28.024 | −0.343 | −0.875 | 1.00 | 18.42 | C |
| ATOM | 51 | CA | GLY | 114 | −28.469 | 3.472 | −1.024 | 1.00 | 18.70 | C |
| ATOM | 52 | CA | GLY | 115 | −25.546 | 5.827 | −1.713 | 1.00 | 19.95 | C |
| ATOM | 53 | CA | GLY | 116 | −25.095 | 9.402 | −0.363 | 1.00 | 25.78 | C |
| ATOM | 54 | CA | GLY | 117 | −22.037 | 11.484 | −1.264 | 1.00 | 31.06 | C |
| ATOM | 55 | CA | GLY | 118 | −20.985 | 14.508 | 0.805 | 1.00 | 38.60 | C |
| ATOM | 56 | CA | GLY | 119 | −17.884 | 16.768 | 1.101 | 1.00 | 42.50 | C |
| TER | | | | | | | | | | |

1MEL

| | | | | | | | | | | | |
|---|---|---|---|---|---|---|---|---|---|---|---|
| ATOM | 57 | CA | GLY | A | 16 | −13.853 | 9.205 | −3.269 | 1.00 | 7.90 | C |
| ATOM | 58 | CA | GLY | A | 17 | −14.557 | 5.500 | −3.346 | 1.00 | 7.54 | C |
| ATOM | 59 | CA | GLY | A | 18 | −16.958 | 3.068 | −1.674 | 1.00 | 6.47 | C |
| ATOM | 60 | CA | GLY | A | 19 | −17.168 | −0.701 | −1.485 | 1.00 | 5.43 | C |
| ATOM | 61 | CA | GLY | A | 20 | −20.455 | −2.621 | −1.546 | 1.00 | 8.09 | C |
| ATOM | 62 | CA | GLY | A | 21 | −20.823 | −6.351 | −0.794 | 1.00 | 8.76 | C |
| ATOM | 63 | CA | GLY | A | 22 | −23.429 | −9.023 | −1.196 | 1.00 | 8.33 | C |
| ATOM | 64 | CA | GLY | A | 23 | −23.620 | −12.484 | 0.211 | 1.00 | 13.77 | C |
| ATOM | 65 | CA | GLY | A | 24 | −26.198 | −14.877 | −1.245 | 1.00 | 17.53 | C |
| ATOM | 66 | CA | GLY | A | 25 | −27.419 | −17.241 | 1.448 | 1.00 | 23.23 | C |
| ATOM | 67 | CA | GLY | A | 26 | −29.608 | −20.285 | 1.362 | 1.00 | 25.25 | C |
| ATOM | 68 | CA | GLY | A | 32 | −25.105 | −18.522 | −11.770 | 1.00 | 5.00 | C |
| ATOM | 69 | CA | GLY | A | 33 | −24.991 | −14.689 | −11.775 | 1.00 | 4.64 | C |
| ATOM | 70 | CA | GLY | A | 34 | −24.729 | −11.897 | −9.152 | 1.00 | 2.48 | C |
| ATOM | 71 | CA | GLY | A | 35 | −24.799 | −8.264 | −10.249 | 1.00 | 2.00 | C |
| ATOM | 72 | CA | GLY | A | 36 | −25.716 | −4.753 | −9.249 | 1.00 | 3.40 | C |
| ATOM | 73 | CA | GLY | A | 37 | −28.543 | −2.502 | −10.376 | 1.00 | 6.64 | C |
| ATOM | 74 | CA | GLY | A | 38 | −29.128 | 1.074 | −9.379 | 1.00 | 8.74 | C |
| ATOM | 75 | CA | GLY | A | 39 | −32.163 | 3.371 | −9.309 | 1.00 | 19.24 | C |
| ATOM | 76 | CA | GLY | A | 67 | −14.374 | 3.095 | −12.462 | 1.00 | 12.68 | C |
| ATOM | 77 | CA | GLY | A | 68 | −16.189 | 0.136 | −10.946 | 1.00 | 8.15 | C |
| ATOM | 78 | CA | GLY | A | 69 | −14.842 | −3.360 | −10.453 | 1.00 | 8.39 | C |
| ATOM | 79 | CA | GLY | A | 70 | −16.896 | −6.384 | −9.408 | 1.00 | 5.47 | C |
| ATOM | 80 | CA | GLY | A | 71 | −15.309 | −9.468 | −7.894 | 1.00 | 8.48 | C |
| ATOM | 81 | CA | GLY | A | 72 | −16.197 | −12.511 | −5.798 | 1.00 | 18.00 | C |
| ATOM | 82 | CA | GLY | A | 79 | −19.282 | −10.422 | −4.331 | 1.00 | 9.55 | C |
| ATOM | 83 | CA | GLY | A | 80 | −18.031 | −6.806 | −4.095 | 1.00 | 4.94 | C |
| ATOM | 84 | CA | GLY | A | 81 | −18.236 | −3.718 | −6.133 | 1.00 | 4.92 | C |
| ATOM | 85 | CA | GLY | A | 82 | −15.331 | −1.340 | −5.635 | 1.00 | 7.44 | C |
| ATOM | 86 | CA | GLY | A | 83 | −16.455 | 2.094 | −6.795 | 1.00 | 6.55 | C |
| ATOM | 87 | CA | GLY | A | 84 | −13.706 | 4.649 | −7.462 | 1.00 | 9.49 | C |
| ATOM | 88 | CA | GLY | A | 85 | −13.919 | 8.136 | −8.959 | 1.00 | 12.54 | C |
| ATOM | 89 | CA | GLY | A | 87 | −19.256 | 11.437 | −9.096 | 1.00 | 15.46 | C |
| ATOM | 90 | CA | GLY | A | 88 | −22.580 | 12.828 | −7.836 | 1.00 | 13.94 | C |
| ATOM | 91 | CA | GLY | A | 89 | −24.298 | 11.258 | −10.888 | 1.00 | 18.68 | C |
| ATOM | 92 | CA | GLY | A | 90 | −23.577 | 7.916 | −9.175 | 1.00 | 9.55 | C |
| ATOM | 93 | CA | GLY | A | 91 | −26.004 | 8.885 | −6.360 | 1.00 | 6.54 | C |
| ATOM | 94 | CA | GLY | A | 92 | −28.681 | 6.180 | −6.349 | 1.00 | 6.38 | C |
| ATOM | 95 | CA | GLY | A | 93 | −30.154 | 3.149 | −4.618 | 1.00 | 6.33 | C |
| ATOM | 96 | CA | GLY | A | 94 | −27.980 | 0.121 | −5.274 | 1.00 | 6.08 | C |
| ATOM | 97 | CA | GLY | A | 95 | −29.551 | −3.256 | −5.491 | 1.00 | 10.12 | C |
| ATOM | 98 | CA | GLY | A | 96 | −27.885 | −6.624 | −5.451 | 1.00 | 10.76 | C |
| ATOM | 99 | CA | GLY | A | 97 | −29.379 | −9.337 | −7.656 | 1.00 | 9.08 | C |
| ATOM | 100 | CA | GLY | A | 98 | −28.752 | −13.014 | −8.455 | 1.00 | 8.28 | C |
| ATOM | 101 | CA | GLY | A | 122 | −33.497 | −12.862 | −6.462 | 1.00 | 15.49 | C |
| ATOM | 102 | CA | GLY | A | 123 | −33.164 | −9.374 | −5.211 | 1.00 | 12.37 | C |
| ATOM | 103 | CA | GLY | A | 124 | −31.827 | −7.789 | −2.102 | 1.00 | 14.34 | C |
| ATOM | 104 | CA | GLY | A | 125 | −32.483 | −4.741 | 0.002 | 1.00 | 22.49 | C |
| ATOM | 105 | CA | GLY | A | 126 | −31.400 | −1.487 | −1.608 | 1.00 | 17.72 | C |
| ATOM | 106 | CA | GLY | A | 127 | −28.513 | 0.495 | −0.221 | 1.00 | 16.51 | C |
| ATOM | 107 | CA | GLY | A | 128 | −28.376 | 4.247 | −0.807 | 1.00 | 16.84 | C |
| ATOM | 108 | CA | GLY | A | 129 | −25.116 | 5.718 | −1.910 | 1.00 | 11.90 | C |
| ATOM | 109 | CA | GLY | A | 130 | −24.954 | 9.478 | −1.961 | 1.00 | 8.75 | C |
| ATOM | 110 | CA | GLY | A | 131 | −22.066 | 11.329 | −3.496 | 1.00 | 14.37 | C |
| ATOM | 111 | CA | GLY | A | 132 | −21.513 | 14.817 | −1.947 | 1.00 | 23.32 | C |
| ATOM | 112 | CA | GLY | A | 133 | −20.378 | 18.169 | −3.369 | 1.00 | 36.14 | C |
| TER | | | | | | | | | | | |

1F97

| | | | | | | | | | | | |
|---|---|---|---|---|---|---|---|---|---|---|---|
| ATOM | 113 | CA | GLY | A | 43 | −13.239 | 8.515 | −3.437 | 1.00 | 14.44 | C |
| ATOM | 114 | CA | GLY | A | 44 | −13.393 | 4.758 | −3.940 | 1.00 | 21.52 | C |
| ATOM | 115 | CA | GLY | A | 45 | −16.246 | 2.710 | −2.546 | 1.00 | 22.48 | C |
| ATOM | 116 | CA | GLY | A | 46 | −17.296 | −0.926 | −2.623 | 1.00 | 21.22 | C |
| ATOM | 117 | CA | GLY | A | 47 | −21.001 | −1.717 | −2.638 | 1.00 | 20.06 | C |
| ATOM | 118 | CA | GLY | A | 48 | −21.128 | −5.074 | −0.848 | 1.00 | 13.56 | C |
| ATOM | 119 | CA | GLY | A | 49 | −23.434 | −7.972 | −1.703 | 1.00 | 19.14 | C |
| ATOM | 120 | CA | GLY | A | 50 | −22.907 | −11.288 | 0.067 | 1.00 | 22.49 | C |
| ATOM | 121 | CA | GLY | A | 51 | −24.848 | −14.490 | −0.589 | 1.00 | 20.58 | C |
| ATOM | 122 | CA | GLY | A | 52 | −24.961 | −18.121 | 0.538 | 1.00 | 24.55 | C |
| ATOM | 123 | CA | GLY | A | 53 | −26.625 | −21.271 | −0.752 | 1.00 | 17.82 | C |
| ATOM | 124 | CA | GLY | A | 57 | −24.531 | −17.722 | −9.307 | 1.00 | 10.39 | C |
| ATOM | 125 | CA | GLY | A | 58 | −25.219 | −15.054 | −11.903 | 1.00 | 8.00 | C |
| ATOM | 126 | CA | GLY | A | 59 | −24.694 | −11.642 | −10.310 | 1.00 | 7.62 | C |
| ATOM | 127 | CA | GLY | A | 60 | −26.312 | −8.537 | −11.794 | 1.00 | 6.84 | C |
| ATOM | 128 | CA | GLY | A | 61 | −26.560 | −4.910 | −10.704 | 1.00 | 8.48 | C |
| ATOM | 129 | CA | GLY | A | 62 | −28.971 | −2.156 | −11.641 | 1.00 | 13.92 | C |
| ATOM | 130 | CA | GLY | A | 63 | −28.917 | 1.574 | −10.971 | 1.00 | 11.89 | C |
| ATOM | 131 | CA | GLY | A | 64 | −32.146 | 3.463 | −10.346 | 1.00 | 23.18 | C |
| ATOM | 132 | CA | GLY | A | 85 | −14.367 | 2.765 | −13.291 | 1.00 | 17.93 | C |
| ATOM | 133 | CA | GLY | A | 86 | −16.308 | −0.184 | −11.839 | 1.00 | 15.44 | C |
| ATOM | 134 | CA | GLY | A | 87 | −14.665 | −3.500 | −11.017 | 1.00 | 16.79 | C |
| ATOM | 135 | CA | GLY | A | 88 | −16.581 | −6.711 | −10.341 | 1.00 | 14.07 | C |
| ATOM | 136 | CA | GLY | A | 89 | −15.959 | −9.289 | −7.620 | 1.00 | 16.75 | C |
| ATOM | 137 | CA | GLY | A | 91 | −18.578 | −9.954 | −2.969 | 1.00 | 17.13 | C |
| ATOM | 138 | CA | GLY | A | 92 | −19.615 | −6.643 | −4.531 | 1.00 | 12.84 | C |
| ATOM | 139 | CA | GLY | A | 93 | −18.746 | −3.911 | −7.017 | 1.00 | 12.33 | C |
| ATOM | 140 | CA | GLY | A | 94 | −15.956 | −1.401 | −6.447 | 1.00 | 12.43 | C |
| ATOM | 141 | CA | GLY | A | 95 | −16.021 | 2.137 | −7.822 | 1.00 | 11.12 | C |
| ATOM | 142 | CA | GLY | A | 96 | −12.511 | 3.493 | −8.309 | 1.00 | 16.95 | C |
| ATOM | 143 | CA | GLY | A | 97 | −14.158 | 6.925 | −7.885 | 1.00 | 21.11 | C |
| ATOM | 144 | CA | GLY | A | 99 | −19.244 | 11.655 | −8.297 | 1.00 | 22.37 | C |
| ATOM | 145 | CA | GLY | A | 100 | −22.479 | 13.329 | −7.197 | 1.00 | 23.75 | C |
| ATOM | 146 | CA | GLY | A | 101 | −24.007 | 11.875 | −10.393 | 1.00 | 22.84 | C |
| ATOM | 147 | CA | GLY | A | 102 | −23.793 | 8.420 | −8.818 | 1.00 | 15.51 | C |
| ATOM | 148 | CA | GLY | A | 103 | −26.377 | 9.137 | −6.093 | 1.00 | 11.57 | C |
| ATOM | 149 | CA | GLY | A | 104 | −29.205 | 6.618 | −6.153 | 1.00 | 9.52 | C |
| ATOM | 150 | CA | GLY | A | 105 | −30.075 | 3.041 | −5.324 | 1.00 | 12.64 | C |
| ATOM | 151 | CA | GLY | A | 106 | −28.157 | 0.010 | −6.540 | 1.00 | 7.32 | C |
| ATOM | 152 | CA | GLY | A | 107 | −29.828 | −3.384 | −6.497 | 1.00 | 7.52 | C |
| ATOM | 153 | CA | GLY | A | 108 | −27.747 | −6.545 | −6.374 | 1.00 | 9.97 | C |
| ATOM | 154 | CA | GLY | A | 109 | −29.572 | −9.419 | −8.055 | 1.00 | 12.91 | C |
| ATOM | 155 | CA | GLY | A | 110 | −28.245 | −12.921 | −7.462 | 1.00 | 11.27 | C |
| ATOM | 156 | CA | GLY | A | 118 | −33.242 | −16.054 | −6.426 | 1.00 | 13.29 | C |
| ATOM | 157 | CA | GLY | A | 119 | −32.582 | −13.085 | −4.179 | 1.00 | 15.55 | C |
| ATOM | 158 | CA | GLY | A | 120 | −31.884 | −9.348 | −4.193 | 1.00 | 11.36 | C |
| ATOM | 159 | CA | GLY | A | 121 | −30.813 | −6.471 | −1.975 | 1.00 | 11.36 | C |
| ATOM | 160 | CA | GLY | A | 122 | −30.903 | −2.696 | −2.475 | 1.00 | 11.14 | C |
| ATOM | 161 | CA | GLY | A | 123 | −28.232 | −0.209 | −1.404 | 1.00 | 11.58 | C |
| ATOM | 162 | CA | GLY | A | 124 | −28.703 | 3.550 | −1.338 | 1.00 | 15.52 | C |
| ATOM | 163 | CA | GLY | A | 125 | −25.598 | 5.531 | −2.225 | 1.00 | 9.61 | C |
| ATOM | 164 | CA | GLY | A | 126 | −25.164 | 9.137 | −1.123 | 1.00 | 10.84 | C |
| ATOM | 165 | CA | GLY | A | 127 | −22.043 | 10.899 | −2.383 | 1.00 | 10.58 | C |
| ATOM | 166 | CA | GLY | A | 128 | −20.981 | 13.549 | 0.145 | 1.00 | 14.41 | C |
| ATOM | 167 | CA | GLY | A | 129 | −20.447 | 17.126 | −1.025 | 1.00 | 12.32 | C |
| TER | | | | | | | | | | | |

1DQT

| | | | | | | | | | | | |
|---|---|---|---|---|---|---|---|---|---|---|---|
| ATOM | 168 | CA | GLY | C | 14 | −9.505 | 5.982 | −6.825 | 1.00 | 30.82 | C |
| ATOM | 169 | CA | GLY | C | 15 | −13.195 | 5.487 | −7.536 | 1.00 | 25.29 | C |
| ATOM | 170 | CA | GLY | C | 16 | −13.507 | 1.942 | −6.167 | 1.00 | 21.63 | C |
| ATOM | 171 | CA | GLY | C | 17 | −16.606 | 0.707 | −4.377 | 1.00 | 20.22 | C |
| ATOM | 172 | CA | GLY | C | 18 | −16.814 | −2.817 | −3.021 | 1.00 | 20.86 | C |
| ATOM | 173 | CA | GLY | C | 19 | −19.629 | −4.451 | −1.137 | 1.00 | 21.25 | C |
| ATOM | 174 | CA | GLY | C | 20 | −21.383 | −7.769 | −0.574 | 1.00 | 21.73 | C |
| ATOM | 175 | CA | GLY | C | 21 | −24.675 | −8.800 | −2.148 | 1.00 | 22.97 | C |
| ATOM | 176 | CA | GLY | C | 22 | −26.301 | −11.552 | −0.160 | 1.00 | 26.59 | C |
| ATOM | 177 | CA | GLY | C | 23 | −29.169 | −13.867 | −0.930 | 1.00 | 26.47 | C |
| ATOM | 178 | CA | GLY | C | 24 | −31.335 | −16.565 | 0.573 | 1.00 | 31.54 | C |
| ATOM | 179 | CA | GLY | C | 25 | −32.236 | −19.342 | 0.443 | 1.00 | 31.33 | C |
| ATOM | 180 | CA | GLY | C | 32 | −26.091 | −17.451 | −10.077 | 1.00 | 21.25 | C |
| ATOM | 181 | CA | GLY | C | 33 | −26.340 | −14.584 | −12.535 | 1.00 | 21.86 | C |
| ATOM | 182 | CA | GLY | C | 34 | −25.686 | −11.294 | −10.762 | 1.00 | 19.78 | C |
| ATOM | 183 | CA | GLY | C | 35 | −26.651 | −7.922 | −12.193 | 1.00 | 19.11 | C |
| ATOM | 184 | CA | GLY | C | 36 | −25.711 | −4.438 | −10.995 | 1.00 | 19.23 | C |
| ATOM | 185 | CA | GLY | C | 37 | −28.233 | −1.721 | −11.782 | 1.00 | 25.95 | C |
| ATOM | 186 | CA | GLY | C | 38 | −27.978 | 2.010 | −11.165 | 1.00 | 30.90 | C |
| ATOM | 187 | CA | GLY | C | 39 | −31.328 | 3.464 | −10.196 | 1.00 | 40.84 | C |
| ATOM | 188 | CA | GLY | C | 66 | −16.664 | −4.410 | −12.490 | 1.00 | 23.17 | C |
| ATOM | 189 | CA | GLY | C | 67 | −15.247 | −7.428 | −10.788 | 1.00 | 22.56 | C |
| ATOM | 190 | CA | GLY | C | 68 | −16.273 | −9.875 | −8.095 | 1.00 | 21.60 | C |
| ATOM | 191 | CA | GLY | C | 69 | −16.270 | −13.324 | −6.554 | 1.00 | 22.69 | C |
| ATOM | 192 | CA | GLY | C | 75 | −21.405 | −12.287 | −4.112 | 1.00 | 23.03 | C |
| ATOM | 193 | CA | GLY | C | 76 | −18.587 | −9.814 | −3.409 | 1.00 | 24.30 | C |
| ATOM | 194 | CA | GLY | C | 77 | −18.961 | −6.951 | −5.886 | 1.00 | 23.46 | C |
| ATOM | 195 | CA | GLY | C | 78 | −16.435 | −4.318 | −6.884 | 1.00 | 25.74 | C |
| ATOM | 196 | CA | GLY | C | 79 | −17.349 | −1.380 | −9.129 | 1.00 | 26.08 | C |
| ATOM | 197 | CA | GLY | C | 80 | −14.377 | 0.652 | −10.395 | 1.00 | 29.15 | C |
| ATOM | 198 | CA | GLY | C | 81 | −13.639 | 3.807 | −12.365 | 1.00 | 26.23 | C |
| ATOM | 199 | CA | GLY | C | 84 | −18.194 | 11.980 | −8.310 | 1.00 | 24.79 | C |
| ATOM | 200 | CA | GLY | C | 85 | −21.048 | 12.151 | −10.804 | 1.00 | 25.37 | C |
| ATOM | 201 | CA | GLY | C | 86 | −21.540 | 8.383 | −10.383 | 1.00 | 25.21 | C |
| ATOM | 202 | CA | GLY | C | 87 | −22.696 | 8.922 | −6.809 | 1.00 | 23.95 | C |
| ATOM | 203 | CA | GLY | C | 88 | −26.050 | 7.191 | −6.551 | 1.00 | 23.67 | C |
| ATOM | 204 | CA | GLY | C | 89 | −28.103 | 4.091 | −5.812 | 1.00 | 22.75 | C |
| ATOM | 205 | CA | GLY | C | 90 | −26.905 | 0.703 | −7.044 | 1.00 | 23.39 | C |
| ATOM | 206 | CA | GLY | C | 91 | −29.167 | −2.332 | −7.030 | 1.00 | 22.55 | C |
| ATOM | 207 | CA | GLY | C | 92 | −27.838 | −5.841 | −6.783 | 1.00 | 22.29 | C |
| ATOM | 208 | CA | GLY | C | 93 | −29.956 | −8.462 | −8.555 | 1.00 | 20.45 | C |
| ATOM | 209 | CA | GLY | C | 94 | −29.490 | −12.198 | −8.107 | 1.00 | 20.73 | C |
| ATOM | 210 | CA | GLY | C | 105 | −34.479 | −11.361 | −6.201 | 1.00 | 24.94 | C |
| ATOM | 211 | CA | GLY | C | 106 | −33.136 | −7.836 | −5.829 | 1.00 | 25.24 | C |
| ATOM | 212 | CA | GLY | C | 107 | −31.842 | −5.752 | −2.931 | 1.00 | 24.05 | C |
| ATOM | 213 | CA | GLY | C | 108 | −33.051 | −2.231 | −2.038 | 1.00 | 25.85 | C |
| ATOM | 214 | CA | GLY | C | 109 | −29.830 | −0.739 | −3.310 | 1.00 | 23.77 | C |
| ATOM | 215 | CA | GLY | C | 110 | −26.544 | 0.520 | −1.934 | 1.00 | 22.46 | C |
| ATOM | 216 | CA | GLY | C | 111 | −25.963 | 4.285 | −1.818 | 1.00 | 24.13 | C |
| ATOM | 217 | CA | GLY | C | 112 | −22.482 | 4.793 | −3.205 | 1.00 | 22.83 | C |
| ATOM | 218 | CA | GLY | C | 113 | −20.958 | 8.192 | −2.417 | 1.00 | 23.40 | C |
| ATOM | 219 | CA | GLY | C | 114 | −18.061 | 9.201 | −4.580 | 1.00 | 25.42 | C |
| END | | | | | | | | | | | |

TABLE 12

| iMab nummer | objective function | zp-comb |
|---|---|---|
| iMabis050 | 617 | −1.83 |
| iMabis051 | 636 | −0.5 |
| iMab102 | 598 | −0.38 |
| iMabis052 | 586 | −0.88 |
| iMabis053 | 592 | −0.73 |
| iMabis054 | 540 | −0.42 |

TABLE 13

iMab102
DDLKLTCRASGYTIGPYCMGWFRQAPNDDSTNVATINMGTVTLSMDDLQP
EDSAEYNCAADSTIYASYYECGHGLSTGGYGYDSHYRGT
iMabis050
DDLKLTSRASGYTIGPYCMGWFRQAPNDDSTNVATINMGTVTLSMDDLQP
EDSAEYNSACDSTIYASYYECGHGLSTGGYGYDCRGQGT
iMabis051
DDLKLTSRASGYTIGPYCMGWFRQAPNDDSTNVATINMGTVTLSMDDLQP
EDSAEYNSCADSTIYASYYECGHGLSTGGYGYDSCGQGT
iMabis052
GSLRLSSAASGYTIGPYCMGWFRQAPGDDREGVAAINMGTVYLLMNSLEP
EDTAICYSAADSTIYASYYECGHGLSTGGYGYDSWGQGC
iMabis053
GSLRLSSAASGYTIGPYCMGWFRQAPGDDREGVAAINMGTVYLLMNSLEP
EDTAIYYSCADSTIYASYYECGHGLSTGGYGYDSCGQGT
iMabis054
GSLRLSSAASGYTIGPYCMGWFRQAPGDDREGVAAINMGTVYLLMNSLEP
EDTAIYYCAADSTIYASYYECGHGLSTGGYGYDSWGCGG

TABLE 14

| | | without | | |
|---|---|---|---|---|
| | | cysteine | with cysteine | |
| results | | bridges | bridges | |
| Residue replacement | solubility | zp-comp | zp-comb | |
| iMabA_K_3_A | | −6.61 | −6.85 | |
| iMabA_K_3_C | | −6.72 | −6.62 | |
| iMabA_K_3_D X | X | −6.65 | −6.54 | |
| iMabA_K_3_E X | X | −6.63 | −6.48 | |
| iMabA_K_3_F | | −6.61 | −6.44 | |
| iMabA_K_3_G | | −6.70 | −6.63 | |
| iMabA_K_3_H | | −6.70 | −6.79 | |
| iMabA_K_3_I | | −6.65 | −6.47 | |
| iMabA_K_3_L | | −6.42 | −6.55 | |
| iMabA_K_3_M | | −6.34 | −6.57 | |
| iMabA_K_3_N X | X | −6.57 | −6.41 | |
| iMabA_K_3_P | | −6.74 | −6.46 | |
| iMabA_K_3_Q X | X | −6.64 | −6.56 | |
| iMabA_K_3_R X | X | −6.91 | −6.56 | best fit |
| iMabA_K_3_S X | X | −6.52 | −6.61 | |
| iMabA_K_3_T X | X | −6.69 | −6.61 | |
| iMabA_K_3_V | | −6.60 | −6.63 | |
| iMabA_K_3_W | | −6.61 | −6.57 | |
| iMabA_K_3_Y | | −6.54 | −6.54 | |
| iMabA_K_7_A | | −6.58 | −6.51 | |
| iMabA_K_7_C | | −6.73 | −6.6 | |
| iMabA_K_7_D X | X | −6.47 | −6.67 | |
| iMabA_K_7_E X | X | −6.83 | −6.61 | |
| iMabA_K_7_F | | −6.66 | −6.58 | |
| iMabA_K_7_G | | −6.65 | −6.76 | |
| iMabA_K_7_H | | −6.80 | −6.57 | |
| iMabA_K_7_I | | −6.62 | −6.28 | |
| iMabA_K_7_L | | −6.76 | −6.66 | |
| iMabA_K_7_M | | −6.69 | −6.62 | |
| iMabA_K_7_N X | X | −6.34 | −6.61 | |
| iMabA_K_7_P | | −6.48 | −6.94 | |
| iMabA_K_7_Q X | X | −6.72 | −6.61 | |
| iMabA_K_7_R X | X | −6.63 | −6.94 | best fit |
| iMabA_K_7_S X | X | −6.83 | −6.61 | |
| iMabA_K_7_T X | X | −6.80 | −6.54 | |
| iMabA_K_7_V | | −6.78 | −6.58 | |
| iMabA_K_7_W | | −6.87 | −6.61 | |
| iMabA_K_7_W | | −6.60 | −6.63 | |
| iMabA_K_19_A | | −6.67 | −6.47 | |
| iMabA_K_19_C | | −6.46 | −6.41 | |
| iMabA_K_19_D X | X | −6.5 | −6.41 | |
| iMabA_K_19_E X | X | −6.69 | −6.77 | |
| iMabA_K_19_F | | −6.61 | −6.55 | |
| iMabA_K_19_G | | −6.94 | −6.54 | |
| iMabA_K_19_H | | −6.48 | −6.62 | |
| iMabA_K_19_I | | −6.74 | −6.51 | |
| iMabA_K_19_L | | −6.52 | −6.72 | |
| iMabA_K_19_M | | −6.60 | −6.16 | |
| iMabA_K_19_N X | X | −6.49 | −6.84 | |
| iMabA_K_19_P | | −6.56 | −6.41 | |
| iMabA_K_19_Q X | X | −6.91 | −6.65 | |
| iMabA_K_19_R X | X | −6.72 | −6.73 | |
| iMabA_K_19_S X | X | −6.57 | −6.61 | |
| iMabA_K_19_T X | X | −6.85 | −6.61 | best fit |
| iMabA_K_19_V | | −6.75 | −6.81 | |
| iMabA_K_19_W | | −6.4 | −6.43 | |
| iMabA_K_19_Y | | −6.53 | −6.48 | |
| iMabA_K_65_A | | −6.52 | 6.22 | |
| iMabA_K_65_C | | −6.43 | 6.23 | |
| iMabA_K_65_D X | X | −6.79 | 6.72 | |
| iMabA_K_65_E X | X | −6.82 | 6.70 | best fit |
| iMabA_K_65_F | | −6.52 | 6.26 | |
| iMabA_K_65_G | | −6.66 | 6.58 | |
| iMabA_K_65_H | | −6.54 | 6.33 | |
| iMabA_K_65_I | | −6.35 | 6.18 | |
| iMabA_K_65_L | | −6.23 | 6.34 | |
| iMabA_K_65_M | | −6.44 | 6.72 | |
| iMabA_K_65_N X | X | −6.74 | 6.62 | |
| iMabA_K_65_P | | −6.50 | 6.42 | |
| iMabA_K_65_Q X | X | −6.62 | 6.59 | |
| iMabA_K_65_R X | X | −6.63 | 6.53 | |
| iMabA_K_65_S X | X | −6.68 | 6.41 | |
| iMabA_K_65_T X | X | −6.47 | 6.41 | |
| iMabA_K_65_V | | −6.30 | 6.25 | |
| iMabA_K_65_W | | −6.50 | 6.39 | |
| iMabA_K_65_Y | | −6.48 | 6.72 | |

TABLE 15

| molecule | zp-comb |
|---|---|
| iMab_C_96_A | −6.52 |
| iMab_C_96_C | −7.11 |
| iMab_C_96_D | −6.26 |
| iMab_C_96_E | −5.75 |
| iMab_C_96_F | −6.70 |
| iMab_C_96_G | −6.38 |
| iMab_C_96_H | −6.26 |
| iMab_C_96_I | −6.66 |
| iMab_C_96_K | −5.56 |
| iMab_C_96_L | −6.37 |
| iMab_C_96_M | −6.51 |
| iMab_C_96_N | −6.53 |
| iMab_C_96_P | −6.48 |
| iMab_C_96_Q | −6.19 |
| iMab_C_96_R | −6.08 |
| iMab_C_96_S | −6.39 |
| iMab_C_96_T | −6.38 |
| iMab_C_96_V | −6.75 |
| iMab_C_96_W | −6.22 |
| iMab_C_96_Y | −6.60 |

TABLE 16A

iMab100 sequence

| | |
|---|---|
| 1 | NVKLVEKGGN FVENDDDLKL TCRAEGYTIG PYCMGWFRQA |
| | PNDDSTNVAT INMGGGITYY |
| 161 | GDSVKERFDI RRDNASNTVT LSMDDLQPED SAEYNCAGDS |
| | TIYASYYECG HGLSTGGYGY |
| 221 | DSHYRGQGTD VTVSS |

Possible candidates:
CYS2_CYS24
CYS4_CYS22
CYS4_CYS111
CYS5_CYS24
CYS6_CYS22
CYS6_CYS112
CYS6_CYS115
CYS7_CYS22
CYS7_CYS115
CYS16_CYS84
CYS18_CYS82
CYS18_CYS84
CYS20_CYS82
CYS21_CYS81
CYS22_CYS80
CYS23_CYS79
CYS34_CYS79
CYS35_CYS98
CYS36_CYS94
CYS39_CYS97
CYS37_CYS45
CYS37_CYS96
CYS38_CYS47
CYS38_CYS48
CYS39_CYS94
CYS92_CYS118
CYS94_CYS116
CYS95_CYS111
CYS95_CYS113
CYS95_CYS115
CYS98_CYS109
CYS98_CYS111
CYS99_CYS110

TABLE 16B
Preferred cysteine residues:

| Cysteine locations | zp-score | iMab name |
|---|---|---|
| CYS6 CYS11 | −7.81 | iMab111 |
| CYS35 CYS98 | −7.54 | |
| CYS99 CYS110 | −7.50 | |
| CYS5 CYS24 | −7.32 | |
| CYS23 CYS79 | −7.23 | |
| CYS38 CYS47 | −7.11 | iMab112 |

TABLE 17

| Mutation | number of | number of |
|---|---|---|
| frequency | transformants | binders |
| 0 | 9.3 * 10⁶ | 50 |
| 2 | 8.1 * 10⁶ | 1000 |
| 3.5 | 5.4 * 10⁶ | 75 |
| 8 | 7.4 * 10⁶ | 100 |
| 13 | 22 * 10⁶ | 100 |

TABLE 18

CM114-iMab100
AAGAAACCAATTCTCCATATTGCATCAGACATTGGCGTCACTGCGTCTTT
TACTGGCTCTTCTCGCTAACCAAACCGGTAACCCCGCTTATTAAAAGCAT
TCTGTAACAAAGCGGGACCAAAGCCATGACAAAAACGCGTAACAAAAGTG
TCTATAATCACGGCAGAAAAGTCCACATTGATTATTTGCACGGCGTCACA
CTTTGCTATGCCATAGCATTTTTATCCATAAGATTAGCGGATCCTACCTG
ACGCTTTTTATCGCAACTCTCTACTGTTTCTCCATACCCGTTTTTTGGGC
TAACAGGAGAAGATATACCATGAAAAAACTGTTATTTGCGATTCCGCTGG
TGGTGCCGTTTTATAGCCATAGCGCGGGCGGCGGCAATGTGAAACTGGTT
GAAAAAGGTGGCAATTTCGTCGAAAACGATGAGGATCTTAAGCTCACGTG
CCGTGCTGAAGGTTACACCATTGGCCCGTACTGCATGGGTTGGTTCCGTC
AGGCGCCGAACGACGACAGTACTAACGTGGCCACGATCAACATGGGTGGC
GGTATTACGTACTAGGGTGACTCCGTCAAAGAGCGGTTCGATATCCGTCG
CGACAACGCGTCCAACACCGTTACCTTATCGATGGACGATCTGCAACCGG
AAGACTCTGCAGAATACAATTGTGCAGGTGATTCTACCATTTACGCGAGC
TATTATGAATGTGGTCATGGCCTGAGTACCGGCGGTTACGGCTACGATAG
CCACTACCGTGGTCAGGGTACCGACGTTACCGTCTCGTCGGCCAGCTCGG
CCGGTGGCGGTGGCAGCTATACCGATATTGAAATGAACCGCCTGGGCAAA
ACCGGCAGCAGTGGTGATTCGGGCAGCGCGTGGAGTCATCCGCAGTTTGA
GAAAGCGGCGCGCCTGGAAACTGTTGAAAGTTGTTTAGCAAAACCCCATA
GAGAAAATTCATTTACTAACGTCTGGAAAGACGACAAAACTTTAGATCGT
TACGCTAACTATGAGGGTTGTCTGTGGAATGCTACAGGCGTTGTAGTTTG
TACTGGTGACGAAACTCAGTGTTACGGTACATGGGTTCCTATTGGGCTTG
CTATCCGTGAAAATGAGGGTGGTGGCTCTGAGGGTGGCGGTTCTGAGGGT
GGCGGTTCTGAGGGTGGCGGTACTAAACCTCCTGAGTACGGTGATACACC
TATTCCGGGCTATACTTATATCAACCCTCTGGACGGCACTTATCCGCCTG
GTACTGAGCAAAACCGCGCTAATCCTAATCCTTCTCTTGAGGAGTCTCAG
CCTCTTAATACTTTCATGTTTCAGAATAATAGGTTCCGAAATAGGCAGGG
GGGATTAACTGTTTATACGGGCACTGTTACTCAAGGCACTGACCCCGTTA
AAACTTATTAGCAGTACACTCGTGTATCATCAAAAGCCATGTATGAGGCT
TACTGGAACGGTAAATTCAGAGACTGCGCTTTCCATTCTGGCTTTAATGA
GGATCCATTCGTTTGTGAATATCAAGGCCAATCGTGTGACCTGCCTCAAC
CTCCTGTCAATGCTGGCGGCGGCTCTGGTGGTGGTTCTGGTGGCGGCTCT
GAGGGTGGTGGCTCTGAGGGTGGCGGTTCTGAGGGTGGCGGCTCTGAGGG
AGGCGGTTCGGGTGGTGGCTCTGGTTCCGGTGATTTTGATTATGAAAAGA
TGGGAAACGCTAATAAGGGGGGTATGACCGAAAATGCCGATGAAAACGCG
CTACAGTCTGACGCTAAAGGCAAACTTGATTCTGTCGCTACTGATTACGG
TGCTGCTATCGATGGTTTCATTGGTGACGTTTCCGGCCTTGCTAATGGTA
ATGGTGCTACTGGTGATTTTGCTGGCTCTAATTCGCAAATGGCTCAAGTC
GGTGACGGTGATAATTCACCTTTAATGAATAATTTCCGTCAATATTTACC
TTCCCTCCCTCAATCGGTTGAATGTCGCCCTTTTGTCTTTAGCGCTGGTA
AACCATATGAATTTTCTATTGATTGTGACAAAATAAACTTATTCCGTGGT
GTCTTTGCGTTTCTTTTATATGTTGCCACCTTTATGTATGTATTTTCTAG
GTTTGCTAACATACTGCGTAATAAGGAGTCTTAAGGCGCGCCTGTAATGA
ACGGTCTCCAGCTTGGCTGTTTTGGCGGATGAGAGAAGATTTTCAGCCTG
ATACAGATTAAATCAGAACGCAGAAGCGGTCTGATAAAACAGAATTTGCC
TGGCGGCAGTAGCGCGGTGGTCCCACCTGACCCCATGCCGAACTCAGAAG
TGAAACGCCGTAGCGCCGATGGTAGTGTGGGGTCTCCCCATGCGAGAGTA
GGGAACTGCCAGGCATCAAATAAAACGAAAGGGTCAGTCGAAAGACTGGG
CCTTTCGTTTTATCTGTTGTTTGTCGGTGAACGCTCTCCTGAGTAGGACA
AATCCGCCGGGAGCGGATTTGAACGTTGCGAAGCAACGGCCCGGAGGGTG
GCGGGCAGGACGCCCGCCATAAACTGCCAGGCATCAAATTAAGCAGAAGG
CCATCCTGACGGATGGCCTTTTTGCGTTTCTACAAACTCTTTTTGTTTAT
TTTTCTAAATACATTCAAATATGTATCCGCTCATGAGACAATAACCCTGA
TAAATGCTTCAATAATATTGAAAAAGGAAGAGTATGAGTATTCAACATTT
CCGTGTCGCCCTTATTCCCTTTTTTGCGGCATTTTGCCTTCCTGTTTTTG
CTCACCCAGAAACGCTGGTGAAAGTAAAAGATGCTGAAGATCAGTTGGGT
GCACGAGTGGGTTACATCGAACTGGATCTCAACAGCGGTAAGATCCTTGA
GAGTTTTCGCCCCGAAGAACGTTTTCCAATGATGAGCACTTTTAAAGTTC
TGCTATGTGGCGCGGTATTATCCCGTGTTGACGCCGGGCAAGAGCAACTC
GGTCGCCGCATACACTATTCTCAGAATGACTTGGTTGAGTACTCACCAGT
CACAGAAAAGCATCTTACGGATGGCATGACAGTAAGAGAATTATGCAGTG
CTGCCATAACCATGAGTGATAACACTGCGGCCAACTTACTTCTGACAACG
ATCGGAGGACGGAAGGAGCTAACCGCTTTTTTGCACAACATGGGGGATCA
TGTAACTCGCCTTGATCGTTGGGAACCGGAGCTGAATGAAGCCATACCAA
ACGACGAGCGTGACACCACGATGCCTGTAGCAATGGCAACAACGTTGCGC
AAACTATTAACTGGCGAACTACTTACTCTAGCTTCCCGGCAACAATTAAT
AGACTGGATGGAGGCGGATAAAGTTGCAGGAGCACTTCTGCGCTCGGCCC
TTCCGGCTGGCTGGTTTATTGCTGATAAATCTGGAGCCGGTGAGCGTGGG
TCTCGCGGTATCATTGCAGCACTGGGGCCAGATGGTAAGCCCTCCCGTAT
CGTAGTTATCTACACGACGGGGAGTCAGGCAACTATGGATGAACGAAATA
GACAGATCGCTGAGATAGGTGCCTCACTGATTAAGCATTGGTAACTGTCA
GACCAAGTTTACTCATATATACTTTAGATTGATTTAAAACTTCATTTTTA
ATTTAAAAGGATCTAGGTGAAGATCCTTTTTGATAATCTCATGACCAAAA
TCCCTTAACGTGAGTTTTCGTTCCACTGAGCGTCAGACCCCGTAGAAAAG
ATCAAAGGATCTTCTTGAGATCCTTTTTTTCTGCGCGTAATCTGCTGGTT
GCAAACAAAAAAACCACCGCTACCAGCGGTGGTTTGTTTGCCGGATCAAG
AGCTACCAACTCTTTTTCCGAAGGTAACTGGCTTCAGCAGAGCGCAGATA
CCAAATACTGTCCTTCTAGTGTAGCCGTAGTTAGGCCACCACTTCAAGAA
CTCTGTAGCACCGCCTACATACCTCGCTCTGCTAATCCTGTTACCAGTGG
CTGCTGCCAGTGGCGATAAGTCGTGTCTTACCGGGTTGGACTCAAGACGA
TAGTTACCGGATAAGGCGCAGCGGTCGGGCTGAACGGGGGGTTCGTGCAC
ACAGCCCAGCTTGGAGCGAACGACCTACACCGAACTGAGATACCTACAGC
GTGAGCTATGAGAAAGCGCCACGCTTCCCGAAGGGAGAAAGGCGGACAGG
TATCCGGTAAGCGGCAGGGTCGGAACAGGAGAGCGCACGAGGGAGCTTCC
AGGGGGAAACGCCTGGTATCTTTATAGTCCTGTCGGGTTTCGCCACCTCT
GACTTGAGGGTCGATTTTTGTGATGCTCGTCAGGGGGGCGGAGCCTATGG
AAAAACGCCAGCAACGCGGCCTTTTTACGGTTCCTGGCCTTTTGCTGGCC
TTTTGCTCACATGTTCTTTCCTGCGTTATCCCCTGATTCTGTGGATAACC
GTATTACCGCCTTTGAGTGAGCTGATACCGCTCGCCGCAGCCGAACGACC
GAGCGCAGCGAGTCAGTGAGCGAGGAAGCGGAAGAGCGCCTGATGCGGTA
TTTTCTCCTTACGCATCTGTGCGGTATTTCACACCGCATATGGTGCACTC
TCAGTACAATGTGCTCTGATGCCGCATAGTTAAGCCAGTATACACTCCGG
TATCGCTACGTGACTGGGTCATGGCTGCGCCCCGACACCCGCCAACACCC
GCTGACGCGCCCTGACGGGCTTGTCTGCTCCCGGGATCCGCTTACAGACA
AGCTGTGACCGTCTCCGGGAGGTGCATGTGTCAGAGGTTTTCACCGTCAT
CACCGAAACGCGCGAGGCAGCAGATCAATTCGCGCGGGAAGGCGAAGCGG
CATGCATAATGTGCCTGTCAAATGGACGAAGCAGGGATTCTGCAAACCCT
ATGCTACTCCGTCAAGCCGTCAATTGTCTGATTCGTTACCAATTATGACA
ACTTGACGGCTACATCATTCACTTTTTCTTCACAACCGGCACGGAACTCG
CTCGGGCTGGCCCCGGTGCATTTTTTAAATACCCGCGAGAAATAGAGTTG
ATCGTCAAAACCAACATTGCGACCGACGGTGGCGATAGGCATCCGGGTGG
TGCTCAAAAGCAGCTTCGCCTGGCTGATACGTTGGTGCTCGCGCCAGCTT
AAGACGCTAATCCCTAACTGCTGGCGGAAAAGATGTGACAGACGGGACGG
CGACAAGCAAACATGCTGTGCGACGCTGGCGATATCAAAATTGCTGTCTG
CCAGGTGATCGCTGATGTACTGACAAGCCTCGCGTACCCGATTATCCATC
GGTGGATGGAGCGACTCGTTAATCGCTTGCATGCGCCGCAGTAACAATTG
CTCAAGCAGATTTATCGCCAGCAGCTCCGAATAGCGCCCTTCCCCTTGCC
CGGCGTTAATGATTTGCCCAAACAGGTCGCTGAAATGCGGCTGGTGCGCT
TCATCCGGGCGAAAGAACCCCGTATTGGCAAATATTGACGGCCAGTTAAG
CCATTCATGCCAGTAGGCGCGCGGACGAAAGTAAACCCACTGGTGATACC
ATTCGCGAGCCTCCGGATGACGACCGTAGTGATGAATCTCTCCTGGCGGG
AACAGGAAAATATCACCCGGTCGGCAAACAAATTCTCGTCCCTGATTTTT
CACCACCCCCTGACCGCGAATGGTGAGATTGAGAATATAACCTTTCATTC
CCAGCGGTCGGTCGATAAAAAAATCGAGATAACCGTTGGCCTCAATCGGC
GTTAAACCCGCCACCAGATGGGCATTAAACGAGTATCCCGGCAGCAGGGG
ATCATTTTGCGCTTCAGCCATACTTTTCATACTCCCGCCATTCAGAG
CM126-iMab100
TTCTCATGTTTGACAGCTTATCATCGATAAGCTTTAATGCGGTAGTTTAT
CACAGTTAAATTGCTAACGCAGTCAGGCACCGTGTATGAAATCTAACAAT
GCGCTCATCGTCATCCTCGGCACCGTCACCCTGGATGCTGTAGGCATAGG
CTTGGTTATGCCGGTACTGCCGGGCCTCTTGCGGGATATCGTCCATTCCG
AGAGCATCGCCAGTCACTATGGCGTGCTGCTAGCGCTATATGCGTTGATG
CAATTTCTATGCGCACCCGTTCTCGGAGCACTGTCCGACCGCTTTGGCCG
CCGCCCAGTCCTGCTCGCTTCGCTACTTGGAGCCACTATCGACTACGCGA
TCATGGCGACCACACCCGTCCTGTGGATATCCGGATATAGTTCCTCCTTT
CAGCAAAAAACCCCTCAAGACCCGTTTAGAGGCCCCAAGGGGTTATGCTA
GTTATTGGTCAGCGGTGGCAGCAGCCAACTCAGCTTCCTTTCGGGCTTTG
TTAGCAGCCGGATCCTTAGTGGTGATGGTGATGGTGGCTTTTGCCCAGGC
GGTTCATTTCTATATCGGTATAGCTGCCACCGCCACCGGCCGAGCTGGCC
GACGAGACGGTAACGTCGGTACCCTGACCACGGTAGTGGGTATCGTAGCC
GTAACCGCCGGTACTCAGGCCATGACGACATTCATAATAGCTCGCGTAAA
TGGTAGAATCACCTGCACAATTGTATTCTGCAGAGTCTTCCGGTTGCAGA
TCGTCCATCGATAAGGTAACGGTGTTGGACGCGTTGTCGCGACGGATATC
GAAGCGCTCTTTGACGGAGTCACCGTAGTACGTAATACCGCCACCCATGT
TGATCGTGGCCACGTTAGTACTGTCGTCGTTCGGCGCCTGACGGAACCAA
CCCATGCAGTACGGGCCAATGGTGTAACCTTCAGCACGGCACGTGAGCTT
AAGATCGTCATCGTTTTCGACGAAATTGCCACCTTTTTCAACCAGTTTCA
CATTCATATGTATATCTCCTTCTTAAAGTTAAACAAAATTATTTCTAGAG
GGAAACCGTTGTGGTCTCCCTATAGTGAGTCGTATTAATTTCGCGGGATC
GAGATCTCGATCCTCTACGCCGGAGGCATCGTGGCCGGCATCACCGGCGC
CACAGGTGCGGTTGCTGGCGCCTATATCGCCGACATCACCGATGGGGAAG
ATCGGGCTCGCCACTTCGGGCTCATGAGCGCTTGTTTCGGCGTGGGTATG
GTGGCAGGCCCCGTGGCCGGGGGACTGTTGGGCGCCATCTCCTTGCATGC
ACCATTCCTTGCGGCGGCGGTGCTCAACGGCCTCAACCTACTACTGGGCT
GCTTCCTAATGCAGGAGTCGCATAAGGGAGAGCGTCGATCGACCGATGCC
CTTGAGAGCCTTCAACCCAGTCAGCTCCTTCCGGTGGGCGCGGGGCATGA
CTATCGTCGCCGCACTTATGACTGTCTTCTTTATCATGCAACTCGTAGGA
CAGGTGCCGGGAGCGCTCTGGGTCATTTTCGGCGAGGACCGCTTTCGCTG
GAGCGCGACGATGATCGGCCTGTCGCTTGCGGTATTCGGAATCTTGCACG
CCCTCGCTCAAGCCTTCGTCACTGGTCCCGCCACCAAACGTTTCGGCGAG
AAGCAGGCCATTATCGCCGGCATGGCGGCCGACGCGCTGGGCTACGTCTT
GCTGGCGTTCGCGACGCGAGGCTGGATGGCCTTCCCCATTATGATTCTTC
TCGCTTCCGGCGGCATCGGGATGCCCGCGTTGCAGGCCATGCTGTCCAGG
CAGGTAGATGACGACCATCAGGGACAGCTTCAAGGATCGCTCGCGGCTCT
TACCAGCCTAACTTCGATCACTGGACCGCTGATCGTCACGGCGATTTATG
CCGCCTCGGCGAGCACATGGAACGGGTTGGCATGGATTGTAGGCGCCGCC
CTATACCTTGTCTGCCTCCCGGCGTTGCGTCGCGGTGCATGGAGCCGGGC
CACCTCGACCTGAATGGAAGCCGGCGGCACCTCGCTAACGGATTCACCAC
TCCAAGAATTGGAGGCAATCAATTCTTGCGGAGAACTGTGAATGCGCAAA
CCAACCCTTGGCAGAACATATGCATCGCGTCGGCGATCTCCACCAGCCGC
ACGCGGCGCATCTCGGGCAGCGTTGGGTCCTGGCCACGGGTGCGCATGAT
CGTGCTCCTGTCGTTGAGGACCCGGCTAGGGTGGCGGGGTTGCCTTACTG
GTTAGCAGAATGAATCACCGATACGCGAGCGAAGGTGAAGCGACTGCTGC
TGCAAAACGTCTGGGACCTGAGCAACAACATGAATGGTCTTCGGTTTCCG
TGTTTCGTAAAGTCTGGAAACGCGGAAGTCAGCGCCCTGCACCATTATGT
TCCGGATCTGCATCGCAGGATGCTGCTGGCTACCCTGTGGAACACCTACA
TCTGTATTAACGAAGCGCTGGCATTGACCCTGAGTGATTTTTCTCTGGTC
CCGCCGCATCCATACCGCCAGTTGTTTACCGTCACAACGTTCCAGTAACC
GGGCATGTTCATCATCAGTAACCCGTATCGTGAGCATCCTCTCTCGTTTC
ATCGGTATCATTACCCCCATGAACAGAAATCCCCCTTACACGGAGGCATC
AGTGACCAAACAGGAAAAAACCGCCCTTAACATGGCCCGCTTTATCAGAA
GCCAGACATTAACGGTTCTGGAGAAACTCAACGAGCTGGACGCGGATGAA
CAGGCAGACATCTGTGAATCGCTTCACGACCACGCTGATGAGCTTTACCG
CAGCTGCCTCGCGCGTTTCGGTGATGACGGTGAAAACGTCTGACACATGC
AGCTCCCGGAGACGGTCACAGCTTGTCTGTAAGCGGATGCCGGGAGCAGA
CAAGCCCGTCAGGGCGCGTCAGCGGGTGTTGGCGGGTGTCGGGGCGCAGC
CATGACCCAGTCACGTAGCGATAGCGGAGTGTATACTGGCTTAACTATGC
GGCATCAGAGCAGATTGTACTGAGAGTGCACCATATATGCGGTGTGAAAT
ACCGCACAGATGCGTAAGGAGAAAATACCGCATCAGGCGCTCTTCCGCTT
CCTCGCTCACTGACTCGCTGCGCTCGGTCGTTCGGCTGCGGCGAGCGGTA
TCAGCTCACTCAAAGGCGGTAATACGGTTATCCACAGAATCAGGGGATAA
CGGAGGAAAGAACATGTGAGCAAAAGGCCAGCAAAAGGCCAGGAACCGTA
AAAAGGCCGCGTTGCTGGCGTTTTTGCATAGGCTCCGCCCCCCTGACGAG
CATCACAAAAATCGACGCTCAAGTCAGAGGTGGCGAAACCCGACAGGACT
ATAAAGATACCAGGCGTTTCCCCCTGGAAGCTCCCTCGTGCGCTCTGCTG
TTGCGACCCTGCCGCTTACCGGATACCTGTCCGGCTTTGTCCCTTCGGGA
AGCGTGGCGCTTTCTCATAGCTCACGCTGTAGGTATCTCAGTTCGGTGTA
GGTCGTTCGCTCCAAGCTGGGCTGTGTGCACGAACCCCCCGTTCAGCCCG
ACCGCTGCGCCTTATCCGGTAACTATCGTCTTGAGTCCAACCCGGTAAGA
CACGACTTATCGCCACTGGCAGCAGCCACTGGTAACAGGATTAGCAGAGC
GAGGTATGTAGGCGGTGCTACAGAGTTCTTGAAGTGGTGGCCTAACTACG
GCTACACTAGAAGGACAGTATTTGGTATCTGCGCTCTGCTGAAGCCAGTT
ACCTTCGGAAAAAGAGTTGGTAGCTCTTGATCCGGCAAACAAACCACCGC
TGGTAGCGGTGGTTTTTTTGTTTGCAAGCAGCAGATTACGCGCAGAAAAA
AAGGATCTCAAGAAGATCCTTTGATCTTTTCTACGGGGTCTGACGCTGAG
TGGAACGAAAACTCACGTTAAGGGATTTTGGTCATGAGATTATCAAAAAG
GATCTTCACCTAGATCCTTTTAAATTAAAAATGAAGTTTTAAATCAATCT
AAAGTATATATGAGTAAACTTGGTCTGACAGTTACCAATGCTTAATCAGT
GAGGCACCTATCTCAGCGATCTGTCTATTTCGTTCATCCATAGTTGCCTG
ACTCCCCGTCGTGTAGATAACTACGATACGGGAGGGCTTACCATCTGGCC
CCAGTGCTGCAATGATACCGCGAGACCCACGCTCACCGGCTCCAGATTTA
TCAGCAATAAACCAGCCAGCCGGAAGGGCCGAGCGCAGAACTGGTCCTGC
AACTTTATCCGCCTCCATCCAGTCTATTAATTGTTGCCGGGAAGCTAGAG
TAAGTACTTCGCCAGTTAATAGTTTGCGCAACGTTGTTGCCATTGGTGCA
GGCATCGTGGTGTCACGCTCGTCGTTTGGTATGGCTTCATTCAGCTCCGG
TTCCCAACGATCAAGGCGAGTTACATGATCCCCCATGTTGTGCAAAAAAG
CGGTTAGCTCCTTCGGTCCTCCGATCGTTGTCAGAAGTAAGTTGGCCGCA
GTGTTATCACTCATGGTTATGGCAGCACTGCATAATTCTCTTACTGTCAT
GCCATCCGTAAGATGCTTTTCTGTGACTGGTGAGTACTCAACCAAGTCAT
TCTGAGAATAGTGTATGCGGCGACCGAGTTGCTCTTGCCCGGCGTCAACA
CGGGATAATACCGCGCCACATAGCAGAACTTTAAAAGTGCTCATCATTGG
AAAACGTTCTTCGGGGCGAAAACTCTCAAGGATCTTACCGCTGTTGAGAT
CCAGTTCGATGTAAGCCACTCGTGCACCCAACTGATGTTCAGCATCTTTT
ACTTTCACCAGCGTTTCTGGGTGAGCAAAAACAGGAAGGCAAAATGCCGC
AAAAAAGGGAATAAGGGCGACACGGAAATGTTGAATACTCATACTCTTCC
TTTTTCAATATTATTGAAGCATTTATCAGGGTTATTGTCTCATGAGCGGA
TACATATTTGAATGTATTTAGAAAAATAAACAAATAGGGGTTCCGCGCAC
ATTTCCCCGAAAAGTGCCACCTGACGTCTAAGAAACCATTATTATCATGA
CATTAACGTATAAAAATAGGCGTATCACGAGGCCCTTTCGTCTTCAAGAA

TABLE 19
C-8 Aldehyde in Headspace - FID %
Sample

| | | | | | 5 | 6 |
|---|---|---|---|---|---|---|
| | 1 | 2 | 3 | 4 | (Aldehyde control) | (Protein control) |
| HCl Added (M) | 0.01 | 0.1 | 0.5 | 1 | 0.1 | 0.1 |
| Pre-Release | 1.55 | 0.80 | 0.8 | 0.98 | 0.50 | 0.00 |
| Release | 3.28 | 4.24 | 2.81 | 3.95 | 0.26 | 0.00 |
| Difference (% in | 1.73 | 3.44 | 2.00 | 2.97 | −0.24 | 0.00 |
| Headspace) | | | | | | |

TABLE 20
VAPS binding to hair and/or skin

iMab143-xx-0029
MIKVYTDRENYGAVGSQVTLHCSASGYTIGPISFTWRYQPEGDRDAISIF
HYNMGDGSIVIHNLDYSDNGSFTCAARFVDALYEPKSCTSRNYAYGNSSQ
VTLYVFE
iMab143-xx-0030
MIKVYTDRENYGAVGSQVTLHCSASGYTIGPISFTWRYQPEGDRDAISIF
HYNMGDGSIVIHNLDYSDNGSFTCAAVFAVVTVATKPDPRFYDYGNSSQV
TLYVFEA
iMab143-xx-0031
MIKVYTDRENYGAVGSQVTLHCSASGYTIGPISFTWRYQPEGDRDAISIF
HYNMGDGSIVIHNLDYSDNGSFTCAATTPFIDYDPNDICPSWYEYDYGNS
SQVTLYVFE
iMab142-xx-0032
MNVKLVEKGGNFVENDDDLKLTCRAEGYTIGPYSMGWFRQAPNDDSTNVS
CINMGGGITYYGDSVKERFDIRRDNASNTVTLSMDDLQPEDSAEYNCAAT
LAPFSIATMYGGLLDTAFDSRGQGTDVTVSS
iMab143-xx-0033
MIKVYTDRENYGAVGSQVTLHCSASGYTIGPISFTWRYQPEGDRDAISIF
HYNMGDGSIVIHNLDYSDNGSFTCAADLHGLGLRRISTYEYGNSSQVTLY
VFE
iMab143-xx-0034
MIKVYTDRENYGAVGSQVTLHCSASGYTIGPISFTWRYQPEGDRDAISIF
HYNMGDGSIVIHNLDYSDNGSFTCAAYRIRSGGYYCFLTYLMDYGNSSQV
TLYVFE
iMab143-xx-0035
MIKVYTDRENYGAVGSQVTLHCSASGYTIGPISFTWRYQPEGDRDAISIF
HYNMGDGSIVIHNLDYSDNGSFTCAAGADCSDYGIMYGMDYGNSSQVTLY
VFE
iMab142-xx-0036
MNVKLVEKGGNFVENDDDLKLTCRAEGYTIGPYSMGWFRQAPNDDSTNVS
CINMGGGITYYGDSVKERFDIRRDNASNTVTLSMDDLQPEDSAEYNCAAN
DLLDYELDCIGMGPNEYEDRGQGTDVTVSS
iMab143-xx-0036
IKVYTDRENYGAVGSQVTLHCSASGYTIGPISFTWRYQPEGDRDAISIFH
YNMGDGSIVIHNLDYSDNGSFTCAANDLLDYELDCIGMGPNEYDDGNSSQ
VTLYVFE
iMab143-xx-0037
MIKVYTDRENYGAVGSQVTLHCSASGYTIGPISFTWRYQPEGDRDAISIF
HYNMGDGSIVIHNLDYSDNGSFTCAAVPGILDYELGTERQPPSCTTRRWD
YDYGNSSQVTLYVFE
iMab144-xx-0037
MLQVVIKPSQGEISVGESKFFLCQASGYTIGPSISWFSPNGEKLNMGSST
LTIYNANIDSAGIYNCAAVPGILDYELGTERQPPSCTTRRWDYDYSEASV
NVKIFQA
iMab142-xx-0038
NVKLVEKGGNFVENDDDLKLTCRAEGYTIGPYSMGWFRQAPNDDSTNVSC
INMGGGITYYGDSVKERFDIRRDNASNTVTLSMDDLQPEDSAEYNCATTL
APFGIATMYGPLNPAAFESRGQGTDVTVSS
iMab142-xx-0039
NVKLVEKGGNFVENDDDLKLTCRAEGYTIGPYSMGWFRQAPNDDSTNVSC
INMGGGITYYGDSVKERFDIRRDNASNTVTLSMDDLQPEDSAEYNCAADY
GRCSWLIRAYNYRGQGTDVTVSS

TABLE 21

iMab143-xx-0029
ATGATCAAAGTTTACACCGACCGTGAAAACTACGGTGCTGTTGGTTCCCA
GGTTACCCTGCACTGCTCCGCTTCCGGTTACACCATCGGTCCGATCAGCT
TCACCTGGCGTTACCAGCCGGAAGGTGACCGTGACGCTATCTCCATCTTC
CACTACAACATGGGTGACGGTTCCATCGTTATCCACAACCTGGACTACTC
CGACAACGGAAGCTTTACCTCCGCAGCACGTTTTGTCGACGCACTTTACG
AACCCAAATCCTGTACCTCCCGGAACTATGCCTACGGGAATTCCTCCCAG
GTTACCCTGTACGTTTTCGAGGCCAGCTCGGCC
iMab143-xx-0030
ATGATCAAAGTTTACACCGACCGTCAAAACTACGGTGCTGTTGGTTCCCA
GGTTACCCTGCACTGCTCCGCTTCCGGTTACACCATCGGTCCGATCAGCT
TCACCTGGCGTTACCAGCCGGAAGGTGACCGTGACGCTATCTCCATCTTC
CACTACAACATGGGTGACGGTTCCATCGTTATCCACAACCTGGACTACTC
CGACAACGGAAGCTTTACCTGTGCAGCAGTCTTTGCGGTCGTTACTGTAG
CGACTAAGCCTCATCCGCGATTTTATGACTACGGGAATTCCTCCCAGGTT
ACCCTGTACGTTTTCGAGGCCAGCTCGGCC
iMab143-xx-0031
ATGATCAAAGTTTACACCGACCGTGAAAACTACGGTGCTGTTGGTTCCCA
GGTTACCCTGCACTCCTCCGCTTCCGGTTACACCATCGGTCCGATCAGCT
TCACCTGGCGTTACCAGCCGGAAGGTGACCGTGACGCTATCTCCATCTTC
CACTACAACATGGGTGACGGTTCCATCGTTATCCACAACCTGGACTACTC
CGACAACGGAAGCTTTACTTGCGCAGCAACGACCCCCTTTATCGACTATG
ACCCAAATGATATCTGCCCCTCGTGGTATGAGTATGACTACGGGAATTCC
TCCCAGCTTACCCTGTACGTTTTCGAGGCCAGCTCGGCC
iMab143-xx-0032
ATGAATGTGAAACTGGTTGAAAAAGGTGGCAATTTCGTCGAAAACGATGA
CGATCTTAAGCTCACGTGCCGTGCTGAAGGTTACACCATTGGCCCGTACT
CCATGGGTTGGTTCCGTCAGGCGCCCAACGACCACAGTACTAACGTGTCC
TGCATCAACATGGGTGGCGGTATTACGTACTACGGTGACTCCGTCAAACA
GCGCTTCGATATCCGTCGCGACAACGCGTCCAACACCGTTACCTTATCGA
TGGACGATCTGCAACCGGAAGACTCTGCAGAATATAACTGTGCAGCAACT
CTAGCCCCTTTCAGTATAGCGACCATGTACGGAGGTTTATTGGACACAGC
TTTCGATTCCCGGGGCCAAGGTACCGACGTTACCGTCTCGTCGGCCAGCT
CGGCC
iMab143-xx-0033
ATGATCAAAGTTTACACCGACCGTGAAAACTACGGTGCTGTTGGTTCCCA
GCTTACCCTGCACTGCTCCGCTTCCGGTTACACCATCGGTCCGATCAGCT
TCACCTGGCGTTACCAGCCGGAAGGTGACCGTGACGCTATCTCCATCTTC
CACTACAACATGGGTGACGGTTCCATCGTTATCCACAACCTGGACTACTC
CGACAACGGAAGCTTTACTTGTGCAGCAGATCTTCACGGGTTGGGGTTGC
GAACGATATCTACGTATGAGTACGGGAATTCCTCCCAGGTTACCCTGTAC
GTTTTCGAGGCCAGCTCGGCC
iMab143-xx-0034
ATGATCAAAGTTTACACCGACCGTGAAAACTACGGTGCTGTTGGTTCCCA
GGTTACCCTGCACTGCTCCGCTTCCGGTTACACCATCGGTCCGATCAGCT
TCACCTGGCGTTACCAGCCGGAAGGTGACCGTGACGCTATCTCCATCTTC
CACTACAACATGGGTGACGGTTCCATCGTTATCCACAACCTGGACTACTC
CGACAACGGAAGCTTTACCTGTGCAGCCTATCGCATACGGAGCGGCGGTT
ACTACTGCTTTCTTACCTACCTCATGGACTACCGGAATTCCTCCCAGGTT
ACCCTGTACGTTTTCGAGGCCAGCTCGGCC
iMab143-xx-0035
ATGATCAAAGTTTACACCGACCGTGAAAACTACGGTGCTGTTGGTTCCCA
GGTTACCCTGCACTGCTCCGCTTCCGGTTACACCATCGGTCCGATCAGCT
TCACCTGGCGTTACCAGCCGGAAGGTGACCGTGACGCTATCTCCATCTTC
CACTACAACATGGGTGACGGTTCCATCGTTATCCACAACCTGGACTACTC
CGACAACGGAAGCTTTACTTGTGCAGCAGGGGCGGACTGTAGCGACTATG
GGATTATGTACGGCATGGACTACGGGAATTCCTCCCAGGTTACCCTGTAC
GTTTTCGAGGCCAGCTCGGCC
iMab142-xx-0036
ATGAATGTGAAACTGGTTGAAAAAGGTGGCAATTTCGTCGAAAACGATGA
CGATCTTAAGCTCACGTGCCGTGCTGAAGGTTACACCATTGGCCCGTACT
CCATGGGTTGGTTCCGTCAGGCGCCGAACGACGACAGTACTAACGTGTCC
TGCATCAACATGGGTGGCGGTATTACGTACTACGGTGACTCCCTCAAAGA
GCGCTTCGATATCCGTCGCGACAACGCGTCCAACACCGTTACCTTATCGA
TGGACGATCTGCAACCGGAAGACTCTGCAGAATATAACTGTGCAGCGAAT
GACCTCCTAGACTACGAATTCGACTGTATCGGAATGGGCCCTAACGAATA
CGAGGACCGGGGCCAGGGTACCCACGTTACCGTCTCGTCGGCCAGCTCGG
CC
iMab143-xx-0036
ATGATCAAACTTTACACCGACCGTGAAAACTACGCTGCTGTTGGTTCCCA
GGTTACCCTGCACTGCTCCGCTTCCGGTTACACCATCGGTCCGATCAGCT
TCACCTGCCGTTACCAGCCGGAAGGTCACCGTGACGCTATCTCCATCTTC
CACTACAACATGGGTGACGGTTCCATCGTTATCCACAACCTGGACTACTC
CCACAACGGAAGCTTTACCTGTGCAGCGAATGACCTCCTAGACTACGAAT
TGGACTGTATCGGAATGGGCCCTAACGAATACGACGACGGGAATTCCTCC
CAGGTTACCCTGTACGTTTTCGAGGCCAGCTCGGCC
iMab143-xx-0037
ATGATCAAAGTTTACACCGACCGTGAAAACTACGGTGCTGTTGGTTCCCA
GGTTACCCTGCACTGCTCCGCTTCCGGTTACACCATCGGTCCGATCAGCT
TCACCTGGCGTTACCAGCCGGAAGGTGACCGTGACGCTATCTCCATCTTC
CACTACAACATGGGTGACGGTTCCATCGTTATCCACAACCTGGACTACTC
CGACAACGGAAGCTTTACTTGTGCAGCTGTACCCGGAATCCTAGACTACG
AATTGGGGACCGAACGACAGCCCCCATCATGTACGACGAGAAGATGGGAC
TATGACTACGGGAATTCCTCCCAGGTTACCCTGTACGTTTTCGAGGCCAG
CTCGGCC
iMab144-xx-0037
ATGCTGCAAGTTCTCATCAAACCGTCCCAGGGTGAAATCTCCGTTGGTGA
ATCCAAATTCTTCCTGTGCCAGGCTTCCGGTTACACCATCGGTCCGAGCA
TCTCCTGGTTCTCCCCGAACGGTGAAAAACTGAACATGGGTTCCTCCACC
CTGACCATCTACAACGCTAACATCGACTCTGCAGGCATTTATAACTGTGC
AGCTGTACCCGGAATCCTAGACTACGAATTGGGCACCGAACGACAGCCCC
CATCATGTACGACGAGAAGATGGGACTATGACTACTCGGAAGCTTCCGTT
AACGTTAAAATCTTCCAGGCCAGCTCGGCC
iMab142-xx-38

| | |
|---|---|
| 1 | A ATGTGAAACT GGTTGAAAAA |
| | GGTGGCAATT TCGTCGAAAA CGATGACGAT CTTAAGCTCA |
| 81 | CGTGCCGTGC TGAAGGTTAC ACCATTGGCC CGTACTCCAT |
| | GGGTTGGTTC CGTCAGGCGC CGAACGACGA CAGTACTAAC |
| 161 | GTGTCCTGCA TCAACATGGG TGGCGGTATT ACGTACTACG |
| | GTGACTCCGT CAAAGAGCGC TTCGATATCC GTCGCGACAA |
| 241 | CGCGTCCAAC ACCGTTACCT TATCGATGGA CGATCTGCAA |
| | CCGGAAGACT CTGCAGAATA TAACTGTGCA ACAACTTTAG |
| 321 | CCCCTTTTGG AATAGCGACT ATGTACGGAC CTTTAAATCC |
| | AGCTGCTTTC GAATCCCGGG GCCAAGGTAC CGACGTTACC |
| 401 | GTCTCGTCGG CCAGCTCGGC |

iMab142-xx-31

| | |
|---|---|
| 1 | AAT GTGAAACTGG TTGAAAAAGG |
| | TGGCAATTTC GTCGAAAACG ATGACGATCT TAAGCTCACG |
| 81 | TGCCGTGCTG AAGGTTACAC CATTGGCCCG TACTCCATGG |
| | GTTGGTTCCG TCAGGCGCCG AACGACGACA GTACTAACGT |
| 161 | GTCCTGCATC AACATGGGTG GCGGTATTAC GTACTACGGT |
| | GACTCCGTCA AAGAGCGCTT CGATATCCGT CGCGACAACG |
| 241 | CGTCCAACAC CGTTACCTTA TCGATGGACG ATCTGCAACC |
| | GGAAGACTCT GCAGAATATA ACTGTGCAGC AGACTACGGG |
| 321 | AGATGTAGCT GGTTAATTCG CGCGTATAAC TACCGGGGCC |
| | AGGGTACCGA CGTTACCGTC TCGTCGGCCA GCTCGGCC |

TABLE 22

| | | | OD450 in Elisa | | | |
|---|---|---|---|---|---|---|
| | | | back- | 0.5% | back- | |
| iMab | OD280 | Dilution | ground | BSA | ground | Seablock |
| Skin | | | | | | |
| 143-xx-0029 | 0.585 | 200 x | 0.484 | 1.881 | 0.131 | 0.212 |
| 143-xx-0030 | 0.323 | 100 x | 0.484 | 2.018 | 0.131 | 0.560 |
| 142-xx-0032 | 0.399 | 100 x | 0.187 | 0.306 | 0.084 | 0.088 |
| 143-xx-0031 | 0.327 | 100 x | 0.484 | 2.619 | 0.131 | 2.883 |
| 143-xx-0033 | 0.828 | 250 x | 0.484 | 1.663 | 0.131 | 0.290 |
| 143-xx-0035 | 0.711 | 200 x | 0.484 | 0.824 | 0.131 | 1.787 |
| 143-xx-0034 | 0.357 | 100 x | 0.484 | 2.231 | 0.131 | 0.699 |
| Hair | | | | | | |
| 142-xx-0039 | 2.36 | 100 x | 0.095 | 1.324 | nd | nd |
| 143-xx-0029 | 3.60 | 100 x | 0.095 | 0.203 | nd | nd |
| 143-xx-0030 | 4.22 | 100 x | 0.095 | 0.262 | nd | nd |
| 143-xx-0031 | 2.82 | 100 x | 0.095 | 1.053 | nd | nd |
| 143-xx-0033 | 3.19 | 100 x | 0.095 | 0.442 | nd | nd |
| 142-xx-0038 | 1.09 | 100 x | 0.095 | 2.349 | nd | nd |
| 143-xx-0034 | 1.71 | 100 x | 0.095 | 0.736 | nd | nd |

Background = no iMab\a-VSV-HRP

TABLE 23

| iMab number | Binding to hair |
|---|---|
| iMab142-xx-0038 | − |
| iMab143-xx-0033 | + |
| iMab143-xx-0034 | ++ |
| iMab143-xx-0031 | + |
| iMab143-xx-0030 | ++ |
| iMab143-xx-0029 | − |
| iMab142-xx-0039 | − |

TABLE 24

| iMab number | AR4 |
|---|---|
| 143-xx-0029 | AARFVDALYEPKSCTSRNYAY |
| 143-xx-0030 | AAVFAVVTVATKPDPRFYDY |
| 143-xx-0031 | AATTPF IDYDPNDICP SWYEYDY |
| 142-xx-0032 | AATLAPFSIATMYGGLLDTAFDS |
| 143-xx-0033 | AADLHGLGLRRISTYEY |
| 143-xx-0034 | AAYRIRSGGYYCFLTYLMDY |
| 143-xx-0035 | AAGADC SDYGIMYGMD Y |
| 143-xx-0036 | AANDLLDYELDCIGMGPNEYED |
| 143-xx-0037 | AAVPGILDYELGTERQPPSCTTRRWDYDY |
| 142-xx-0038 | TTLAPFGIATMYGPLNPAAFES |
| 142-xx-0039 | AADYGRCSWLIRAYNY |

1. A method for applying a cosmetic substance to a desired target molecule, said method comprising:
providing a conjugate of a proteinaceous substance having specific affinity for said target molecule linked to a cosmetic substance, wherby wherein the resulting connection between said cosmetic substance and said target molecule can be disrupted upon the presence or application of a chemical and/or physical signal.

2. The method claim 1, wherein said proteinaceous substance comprises a synthetic or recombinant proteinaceous molecule comprising a binding peptide and a core, wherein:
said core comprises a beta-barrel comprising at least four strands,
said beta-barrel comprises at least two beta-sheets,
each of said beta-sheets comprises two of said strands,
said binding peptide is a peptide connecting two strands in said beta-barrel, and
said binding peptide is outside its natural context.

3. The method of claim 2, wherein said proteinaceous molecule comprises a beta-barrel comprising at least five strands, and wherein at least one of said beta-sheets comprises three of said strands.

4. The method of claim 3, wherein said beta-barrel comprises at least six strands, and wherein at least two of said beta-sheets comprises three of said strands.

5. The method of claim 1, wherein said beta-barrel comprises at least seven strands, and wherein at least one of said beta-sheets comprises four of said strands.

6. The method of claim 1, wherein said beta-barrel comprises at least eight strands, and wherein at least one of said beta-sheets comprises four of said strands.

7. The method of claim 1, wherein said beta-barrel comprises at least nine strands, and wherein at least one of said beta-sheets comprises four of said strands.

8. The method of claim 1 wherein said binding peptide connects two strands of said beta-barrel on the open side of said beta-barrel.

9. The method of claim 1 wherein said binding peptide connects said at least two beta-sheets of said beta-barrel.

10. The method of claim 1, wherein said proteinaceous molecule comprises at least one further binding peptide.

11. The method of claim 1, wherein said proteinaceous molecule comprises three binding peptides and three connecting peptide sequences.

12. The method of claim 1, wherein said proteinaceous molecule comprises at least four binding peptides.

13. The method of claim 12, wherein at least one binding peptide recognizes a target molecule other than at least one of the other binding peptides.

14. The method of claim 2, wherein said proteinaceous molecule has an altered binding property, said altered binding property selected for the physical and/or chemical circumstances wherein the conjugate is applied, said altered binding property comprising introducing an alteration in the core of said proteinaceous molecule and selecting from said proteinaceous molecule a proteinaceous molecule with said altered binding property.

15. The method of claim 2, wherein said proteinaceous molecule has an altered structural property, said altered structural property selected for the physical and/or chemical circumstances wherein the conjugate is applied, said altered structural property comprising introducing an alteration in the core of said proteinaceous molecule, and selecting from said proteinaceous molecule, a proteinaceous molecule with said altered structural property.

16. The method of claim 14, wherein said alteration comprises a post-translational modification.

17. The method of claim 14, wherein said alteration is introduced into a nucleic acid coding for said proteinaceous molecule, the method further comprising expressing said nucleic acid in an expression system that is capable of producing said proteinaceous molecule.

18. The method of claim 1, wherein said proteinaceous substance comprises an amino acid sequence as depicted in Table 2 Table 3, Table 10, Table 13, Table 16a or 16b, Table 20 or FIGS. 22A-22I, or a functional part, derivative and/or analogue thereof.

19. A conjugate comprising a proteinaceous substance having specific affinity for a target molecule linked to a cosmetic substance, wherein the resulting connection between cosmetic substance and target molecule can be disrupted upon the presence of a chemical and/or physical signal, and further wherein said proteinaceous substance comprises a synthetic or recombinant proteinaceous molecule comprising a binding peptide and a core, said core comprising a beta-barrel comprising at least four strands, wherein said beta-barrel comprises at least two beta-sheets, wherein each of said beta-sheets comprises two of said strands and wherein said binding peptide is a peptide connecting two strands in said beta-barrel and wherein said binding peptide is outside its natural context.

20. The conjugate of claim 19, wherein the proteinaceous molecule is derived from the immunoglobulin superfamily.

21. The conjugate of claim 19, wherein the exterior of the proteinaceous molecule is immunologically similar to the immunoglobulin superfamily molecule from which it was derived.

22. The conjugate of claim 19, comprising a fusion protein.

23. The conjugate of claim 19, wherein said proteinaceous substance is covalently linked to said cosmetic substance by an organic linker.

24. The conjugate of claim 23, wherein said organic linker is labile in certain physicochemical conditions and stable in different physicochemical conditions.

25. The conjugate of claim 19, wherein the link between said proteinaceous molecule and said cosmetic substance can be proteolyzed.

26. The conjugate of claim 19, wherein the link between said proteinaceous molecule and said cosmetic substance is labile under skin and/or hair conditions.

27. The conjugate of claim 19, which has specific affinity for a target molecule associated with the skin, hair or other body substances exposed to the exterior of said body.

28. The conjugate of claim 27, wherein said target molecule comprises an affinity region 4 of iMab143-xx-0029, 143-xx-0030, 143-xx-0031, 142-xx-0032, 143-xx-0033, 143-xx-0034, 143-xx-0035, 143-xx-0036, 143-xx-0037, 142-xx-0038, 142-xx-0039 Table 20 or FIGS. 22A-22I, or a functional part, derivative and/or analogue of such an affinity region 4.

29. The conjugate of claims 27 comprising an affinity region as depicted in Table 24, or a functional part, derivative and/or analogue thereof.

30. The conjugate of claim 27, which has a specific affinity for keratin.

31. The conjugate of claim 19, which has a specific affinity for a target molecule associated with textile fabric.

32. The conjugate of claim 19, wherein the cosmetic substance comprises a fragrant substance.

33. The conjugate of claim 19, wherein the cosmetic substance comprises a colored substance.

34. The conjugate of claim 19, wherein said conjugate is water soluble.

35. The conjugate of claim 19, comprising an amino acid sequence as depicted in Table 2, Table 3, Table 10, Table 13, Table 16a or 16, b, Table 20, or FIGS. 22A-22I, or a functional part, derivative and/or analogue thereof.

36. A cosmetic composition comprising the conjugate of claim 19.

37. The cosmetic composition of claim 36, wherein said cosmetic composition is a perfume, a deodorant, a mouth wash or a cleaning composition.

38. The cosmetic composition of claim 36, wherein said cosmetic composition is a hair dye composition, a lipstick, rouge or other skin-coloring composition.

39. A detergent and/or softener composition comprising the conjugate of claim 19.

40. A divalent or multivalent proteinaceous substance having specific affinity for at least two target molecules present in hair and wherein epitopes recognized on said at least two target molecules may be the same or different.

41. The divalent or multivalent proteinaceous substance of claim 40, wherein the divalent or multivalent proteinaceous substance is divalent.

42. The divalent or multivalent proteinaceous substance of claim 40, wherein said divalent or multivalent proteinaceous substance comprises an affinity region 4 of iMab143-xx-0029, 143-xx-0030, 143-xx-0031, 143-xx-0033, 143-xx-0034, 142-xx-0038, 142-xx-0039 of Table 20 or FIGS. 22A-22I, or a functional part, derivative and/or analogue thereof.

43. The divalent or multivalent proteinaceous substance of claim 42, comprising at least two affinity regions 4 of iMab143-xx-0029 143-xx-0030, 143-xx-0031, 143-xx-0033, 143-xx-0034, 142-xx-0038, 142-xx-0039 of Table 20 or FIGS. 22A-22I, or a functional part, derivative and/or analogue thereof.

44. The divalent or multivalent proteinaceous substance of claim 42, having the sequence of iMab143-xx-0029 143-xx-0030, 143-xx-0031, 143-xx-0033, 143-xx-0034, 142-xx-0038, 142-xx-0039 of of Table 20 or FIGS. 22A-22I, or a functional part, derivative and/or analogue thereof.

45. The method according to claim 15, wherein said alteration comprises a post-translational modification.

46. The method according to claim 15, wherein said alteration is introduced into a nucleic acid sequence encoding said proteinaceous molecule, and wherein the method further comprises expressing said nucleic acid sequence in an expression system able to produce said proteinaceous molecule.
